(19) United States
(12) Patent Application Publication
(10) Pub. No.: US 2025/0171463 A1
(43) Pub. Date: May 29, 2025

(54) EMOPAMIL-BINDING PROTEIN INHIBITORS AND USES THEREOF

(71) Applicant: BIOGEN MA INC., Cambridge, MA (US)

(72) Inventors: Martin Himmelbauer, Cambridge, MA (US); Felix Gonzalez Lopez de Turiso, Cambridge, MA (US); Edward Yin Shiang Lin, Ashland, MA (US); Vatee Pattaropong, Bedford, MA (US); Zhili Xin, Cambridge, MA (US); Teyu Chen, Charlestown, MA (US); John Howard Jones, Framingham, MA (US); Nupur Bansal, Medfield, MA (US); Robert Gilfillan, Ashland, MA (US)

(21) Appl. No.: 18/841,045

(22) PCT Filed: Feb. 23, 2023
(86) PCT No.: PCT/US2023/013717

§ 371 Date: Aug. 23, 2024

Related U.S. Application Data
(60) Provisional application No. 63/314,095, filed on Feb. 25, 2022.

(51) Int. Cl. C07D 498/10; A61K 31/397; A61K 31/407; A61K 31/4155; A61K 31/416; A61K 31/438; A61K 31/439; A61K 31/4433; A61K 31/5386; C07D 471/10; C07D 471/20; C07D 487/10

(52) U.S. Cl. CPC C07D 498/10; A61K 31/397; A61K 31/407; A61K 31/4155; A61K 31/416; A61K 31/438; A61K 31/439; A61K 31/4433; A61K 31/5386; C07D 471/10; C07D 471/20; C07D 487/10

(57) ABSTRACT

Provided are compounds of the Formula (I): or pharmaceutically acceptable salts thereof, which are useful for the inhibition of EBP and in the treatment of a variety of EBP mediated conditions or diseases, such as multiple sclerosis.

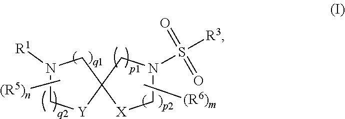

### RELATED APPLICATIONS

This application claims priority to U.S. Provisional Application No. 63/314,095, filed on Feb. 25, 2022. The entire contents of the foregoing application are expressly incorporated herein by reference.

### FIELD OF THE INVENTION

The present disclosure relates to inhibitors of Emopamil-Binding Protein (EBP), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the EBP inhibitor to a warm-blooded animal, especially a human.

### BACKGROUND OF THE INVENTION

Emopamil-Binding Protein (EBP) is a Δ8-Δ7 sterol isomerase enzyme which isomerizes the double bond in sterol molecules, moving the double bond from the 8-9 position to the 7-8 position. Specifically, EBP converts either zymostenol to lathosterol, or zymosterol to dehydrolathosterol, during the biosynthesis of cholesterol (Silve et al., 1996, J Biol Chem. 271 (37), 22434-22440). It has been shown that an accumulation of 8-9 unsaturated sterols activates oligodendrocyte formation and remyelination (Hubler et al., 2019, Nature 560 (7718), 372-376).

Myelin is lipid-based molecule which forms protective layers (myelin sheathes) around nerve cell axons and insulates the axons. Demyelinating diseases, or myelin-related diseases, are a result of these myelin sheathes being damaged, degraded, or reduced in thickness. The loss of the myelin sheathes disrupts the electronic signals from the brain and can lead to nerve damage, vision loss, numbness, muscle weakness, cognitive decline, loss of motor functions, and other similar symptoms. In some myelin-related diseases, such as multiple sclerosis, a subject's immune system targets and breaks down their own myelin sheathes. The ability to repair and regenerate the myelin sheathes is key to treating these myelin-related diseases. Due to its function converting 8-9 sterols, inhibition of EBP is a potential target for activating remyelination, as its inhibition leads to an increase of these 8-9 sterol starting materials (Theodoropoulous et al, 2020, J. Am. Chem. Soc., 142, (13), 6128-6138).

In addition to its role in remyelination, EBP has also been shown to be a key enzyme in certain colorectal cancers due to the reduction in essential lipids such as cholesterol (Theodoropoulous et al, 2020, J. Am. Chem. Soc., 142, (13), 6128-6138).

Thus, there is a need for EBP inhibitors as potential therapeutic agents for treating diseases or disorders that are responsive to EBP inhibition.

### SUMMARY OF THE INVENTION

The present disclosure provides compounds that are EBP inhibitors. In a first aspect, the present disclosure relates to compounds having the Formula I:

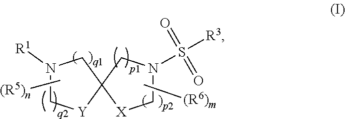

or a pharmaceutically acceptable salt thereof, wherein:
X is CH₂ or O;
Y is CH₂ or O;
provided only one of X and Y is O;
q1 is 1 or 2;
q2 is 0 or 1 when Y is CH₂, or q2 is 2 when Y is O;
p1 is 1 or 2;
p2 is 0 or 1 when X is CH₂, or p2 is 2 when X is O;
R¹ is C₂₋₆alkyl, Het, or —Z-Het, wherein the C₂₋₆alkyl is optionally substituted with one or more R^{A} and Het is optionally substituted with one or more R²;
Z is C₁₋₄alkyl optionally substituted with one or more halo or C₁₋₃alkoxy;
Het is C₃₋₆cycloalkyl, a 4 to 6 membered monocyclic heterocyclyl, or a 6 to 8-membered bicyclic heterocyclyl, each of which is optionally substituted with R²;
R^{A}, for each occurrence, is independently OR^{2a}, SR^{2a}, or C(O)OR^{2a};
R², for each occurrence, is independently C₁₋₆alkyl, halo, —CN or OR^{2a}, wherein the C₁₋₆alkyl is optionally substituted with one or more halo or C₁₋₃alkoxy;
R^{2a} is H C₁₋₆alkyl, or C₃₋₆cycloalkyl wherein the C₁₋₆alkyl is optionally substituted with one or more halo or C₁₋₃alkoxy;
R³ is C₁₋₄alkyl-phenyl, phenyl, 5 or 6-membered monocyclic heteroaryl, or 9 to 10-membered bicyclic heteroaryl, wherein the phenyl, 5 or 6-membered monocyclic heteroaryl and 9 to 10-membered bicyclic heteroaryl are each optionally substituted with one or more substituent R⁴;
R⁴, for each occurrence, is independently halo, —OR^{4a}, —CN, C₁₋₆alkyl, C₃₋₆cycloalkyl, C₂₋₆alkenyl, phenyl, or 5 or 6-membered monocyclic heteroaryl, wherein the C₁₋₆alkyl or C₃₋₆ cycloalkyl are each optionally substituted with one or more C₁₋₆alkyl, C₁₋₆haloalkyl, C₃₋₆ cycloalkyl, or halo, and wherein the or 5 or 6-membered monocyclic heteroaryl is optionally substituted with one or more C₁₋₆alkyl; or
two R⁴ together with their intervening atoms form a 5 to 7-membered heterocyclyl;
R^{4a} is H or C₁₋₆alkyl, wherein the C₁₋₆alkyl is optionally substituted with one or more halo;
n is 1 or 2;
m is 1 or 2;
R⁵, for each occurrence, is independently H, halo, C₁₋₃alkyl, or C₁₋₃haloalkyl; or two R⁵ together form a C₁₋₃alkylene;
R⁶, for each occurrence, is independently H, halo, C₁₋₃alkyl, or C₁₋₃haloalkyl; or two R⁶ together form a C₁₋₃alkylene;
provided that the compound is not:

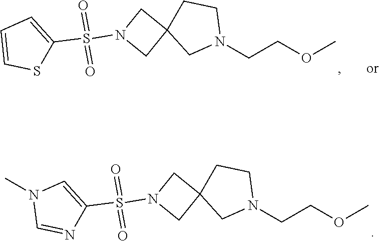

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a disease or disorder that is responsive to inhibition of EBP in a subject comprising administering to said subject an effective amount of at least one compound described herein or a pharmaceutically acceptable salt thereof. In some embodiments, the present disclosure provides a method for treating multiple sclerosis. In some embodiments, the present disclosure provides a method for promoting myelination in a subject with a myelin-related disorder.

Another aspect of the present disclosure relates to the use of at least one compound described herein or a pharmaceutically acceptable salt thereof, for the manufacture of a medicament for the treatment of a disease or disorder responsive to inhibition of EBP. Also provided is a compound described herein or a pharmaceutically acceptable salt thereof for use in treating a disease or disorder responsive to inhibition of EBP.

### DETAILED DESCRIPTION OF THE INVENTION

The present disclosure provides compounds and pharmaceutical compositions thereof that may be useful in the treatment of diseases or disorders through mediation of EBP function/activity, such as multiple sclerosis or other myelin-related disorders. In some embodiments, the compounds of present disclosure are EBP inhibitors.

### Compounds and Compositions

In a first embodiment, the present disclosure provides a compound of Formula (I):

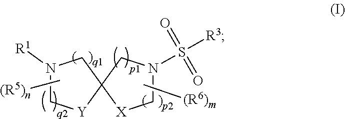

or a pharmaceutically acceptable salt thereof, wherein the variables in Formula (I) are as defined in the first embodiment above. In another embodiment, for the compounds of Formula (I):
X is CH₂ or O;
Y is CH₂ or O;
provided only one of X and Y is O;
q1 is 1 or 2;
q2 is 0 or 1 when Y is CH₂, or q2 is 2 when Y is O;
p1 is 1 or 2;
p2 is 0 or 1 when X is CH₂, or p2 is 2 when X is O;
R¹ is C₂₋₆alkyl, Het, or —Z-Het, wherein the C₂₋₆alkyl is substituted with one or more OR^{2a} and Het is optionally substituted with one or more R²;
Z is C₁₋₄alkyl optionally substituted with one or more halo;
Het is a 4 to 6 membered monocyclic heterocyclyl, or a 6 to 8-membered bicyclic heterocyclyl, each of which is optionally substituted with R²;
R², for each occurrence, is independently C₁₋₆alkyl or OR^{2a};
R^{2a} is H or C₁₋₆alkyl, wherein the C₁₋₆alkyl is optionally substituted with one or more halo;
R³ is phenyl, 5 or 6-membered monocyclic heteroaryl, or 9 to 10-membered bicyclic heteroaryl, wherein the phenyl, 5 or 6-membered monocyclic heteroaryl and 9 to 10-membered bicyclic heteroaryl are each optionally substituted with one or more substituent R⁴;
R⁴, for each occurrence, is independently halo, —OR^{4a}, —CN, C₁₋₆alkyl, C₃₋₆cycloalkyl, or 5 or 6-membered monocyclic heteroaryl, wherein the C₁₋₆alkyl is optionally substituted with one or more halo, and wherein the or 5 or 6-membered monocyclic heteroaryl is optionally substituted with one or more C₁₋₆alkyl; or
two R⁴ together with their intervening atoms form a 5 to 7-membered heterocyclyl;
R^{4a} is H or C₁₋₆alkyl, wherein the C₁₋₆alkyl is optionally substituted with one or more halo;
n is 1 or 2;
m is 1 or 2;
R⁵, for each occurrence, is independently H, halo, C₁₋₃alkyl, or C₁₋₃haloalkyl; or two R⁵ together form a C₁₋₃alkylene;
R⁶, for each occurrence, is independently H, halo, C₁₋₃alkyl, or C₁₋₃haloalkyl; or two R⁶ together form a C₁₋₃alkylene;
provided that the compound is not:

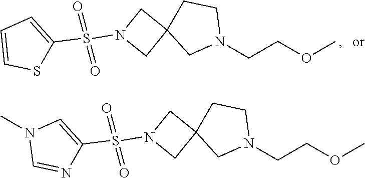

In a second embodiment, for the compounds of Formula (I), or a pharmaceutically acceptable salt thereof, Het is a 4 to 6 membered oxygen-containing monocyclic saturated heterocyclyl, or a 6 to 8-membered oxygen-containing bicyclic saturated heterocyclyl; and the remaining variables are as described in the first aspect or the first embodiment. In an alternative embodiment, for the compounds of Formula (I), or a pharmaceutically acceptable salt thereof, Het is C₃₋₆cycloalkyl, a 4 to 6 membered oxygen-containing monocyclic saturated heterocyclyl, or a 6 to 8-membered oxygen-containing bicyclic saturated heterocyclyl, each of which is optionally substituted with one to three R²; and the remaining variables are as described in the first aspect or the first embodiment. In a further alternative embodiment, for the compounds of Formula (I), or a pharmaceutically acceptable salt thereof, Het is cyclohexyl, oxetanyl, tetrahydrofuranyl, tetrahydropyranyl, dioxolanyl, dioxanyl, 2-oxaspiro[3.3]heptanyl, 2-oxaspiro[3.3]heptanyl, 2-oxabicyclo[2.1.1]hexanyl, 6-oxabicyclo[3.2.1]octanyl, or 2-oxabicyclo[3.1.1]heptanyl, each of which is optionally substituted with one to three R²; and the remaining variables are as described in the first aspect or the first embodiment.

In a third embodiment, the compound of the present disclosure is represented by Formula (II):

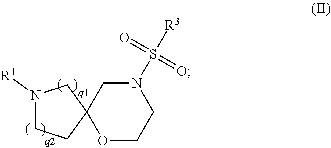

or a pharmaceutically acceptable salt thereof, wherein the variables in Formula (II) are as defined in the first aspect or the first or second embodiment above or any alternative embodiments described therein.

In a fourth embodiment, for the compounds of Formula (I) or (II), or a pharmaceutically acceptable salt thereof, R³ is phenyl, pyridyl, thiazoyl, or pyrazolyl, each of which is optionally substituted with one to three R⁴; and the remaining variables are as described in the first aspect or the first or second embodiment or any alternative embodiments described therein.

In a fifth embodiment, for the compounds of Formula (I) or (II), or a pharmaceutically acceptable salt thereof, R³ is

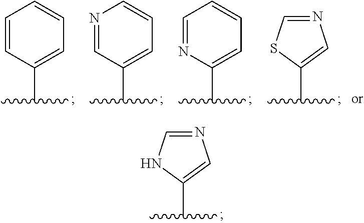

wherein each of the formula depicted above is optionally substituted with one to three R⁴; and the remaining variables are as described in the first aspect or the first or second embodiment or any alternative embodiments described therein.

In a sixth embodiment, for the compounds of Formula (I) or (II), or a pharmaceutically acceptable salt thereof, R³ is

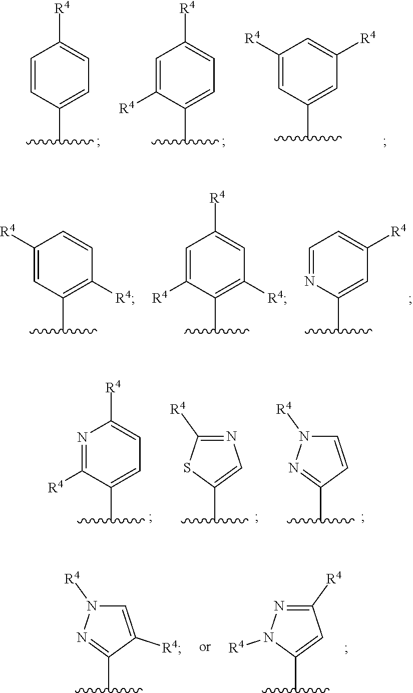

and the remaining variables are as described in the first aspect or the first or second embodiment or any alternative embodiments described therein.

In a seventh embodiment, for the compounds of Formula (I) or (II), or a pharmaceutically acceptable salt thereof, R⁴, for each occurrence, is independently selected from halo, —CN, —OR^{4a}, C₁₋₄alkyl, C₁₋₄haloalkyl, C₃₋₆cycloalkyl and 5 or 6-membered heteroaryl optionally substituted with C₁₋₃alkyl; and R^{4a} is C₁₋₃alkyl or C₁₋₃haloalkyl; and the remaining variables are as described in the first aspect or the first, second, third, fourth, fifth, or sixth embodiment or any alternative embodiments described therein.

In an eighth embodiment, for the compounds of Formula (I) or (II), or a pharmaceutically acceptable salt thereof, R⁴, for each occurrence, is independently selected from —CH₃, —CF₃, —OCHF₂, —OCH₃, —CN, —F, —Cl, isopropyl, cyclopropyl, and 4-methylpyridin-2-yl; and the remaining variables are as described in the first aspect or the first, second, third, fourth, fifth, or sixth embodiment or any alternative embodiments described therein.

In a ninth embodiment, for the compounds of Formula (I) or (II), or a pharmaceutically acceptable salt thereof, R¹ is C₂₋₄alkyl substituted with OR^{2a};

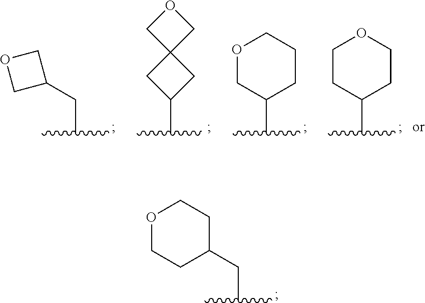

wherein each of the formula depicted above is optionally substituted with one to three R²; R², for each occurrence, is independently C₁₋₃alkyl or —OR^{2a}; R^{2a}, for each occurrence, is independently H or C₁₋₃alkyl; and the remaining variables are as described in the first aspect or the first, second, third, fourth, fifth, sixth, seventh, or eighth embodiment or any alternative embodiments described therein.

In a tenth embodiment, for the compounds of Formula (I) or (II), or a pharmaceutically acceptable salt thereof, R¹ is

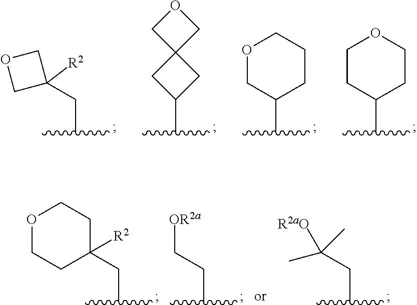

R², for each occurrence, is independently C₁₋₃alkyl or —OR^{2a}; R^{2a}, for each occurrence, is independently H or C₁₋₃alkyl; and the remaining variables are as described in the first aspect or the first, second, third, fourth, fifth, sixth, seventh, or eighth embodiment or any alternative embodiments described therein.

In an eleventh embodiment, for the compounds of Formula (I) or (II), or a pharmaceutically acceptable salt thereof, R², for each occurrence, is independently selected from —CH₃ and —OH; R^{2a}, for each occurrence, is independently H or —CH₃; and the remaining variables are as described in the first aspect or the first, second, third, fourth, fifth, sixth, seventh, eighth, ninth, or tenth embodiment or any alternative embodiments described therein.

In a twelfth embodiment, the compound of the present disclosure is represented by Formula (III):

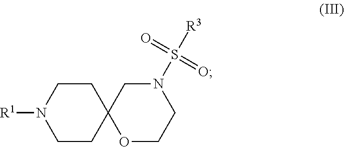

or a pharmaceutically acceptable salt thereof, wherein the variables in Formula (III) are as defined in the first aspect or the first, second, third, fourth, fifth, sixth, seventh, eighth, ninth, tenth, or eleventh embodiment above or any alternative embodiments described therein.

In a thirteenth embodiment, for the compounds of Formula (III), or a pharmaceutically acceptable salt thereof, R¹ is

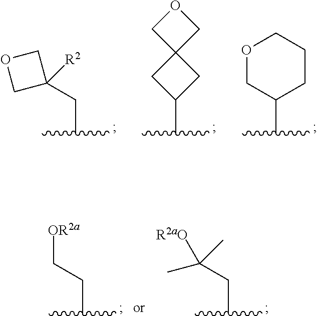

R², for each occurrence, is independently selected from —CH₃ and OH; R^{2a}, for each occurrence, is independently selected from H or —CH₃; R³ is

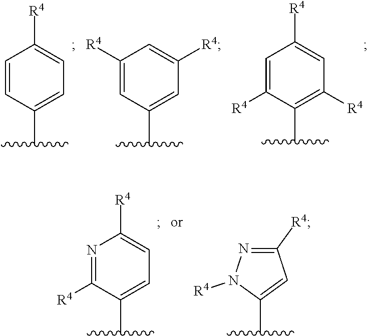

and R⁴, for each occurrence, is independently selected from —CH₃, —CF₃, —OCHF₂, —OCH₃, and —F.

In a fourteenth embodiment, the compound of the present disclosure is represented by Formula (IV):

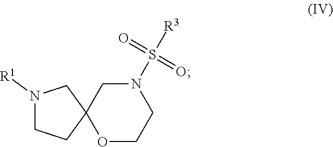

or a pharmaceutically acceptable salt thereof, wherein the variables in Formula (IV) are as defined in the first aspect or the first, second, third, fourth, fifth, sixth, seventh, eighth, ninth, tenth, or eleventh embodiment above or any alternative embodiments described therein.

In a fifteenth embodiment, the compound of the present disclosure is represented by Formula (IVA) or (IVB):

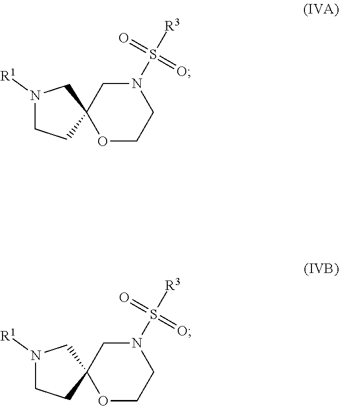

or a pharmaceutically acceptable salt thereof, wherein the variables in Formula (IVA) or (IVB) are as defined in the first aspect or the first, second, third, fourth, fifth, sixth, seventh, eighth, ninth, tenth, or eleventh embodiment above or any alternative embodiments described therein.

In a sixteenth embodiment, for the compounds of Formula (IV), (IVA), or (IVB), or a pharmaceutically acceptable salt thereof, R¹ is

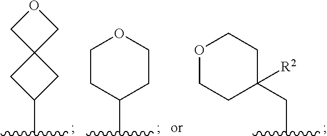

R² is OH; R³ is

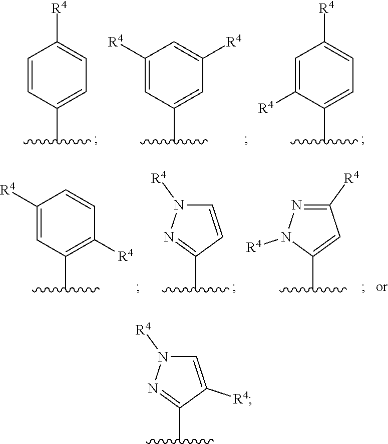

R⁴, for each occurrence, is independently selected from —CH₃, —CF₃, —OCH₃, —OCHF₂, —CN, isopropyl, —F, —Cl, and 4-methylpyridin-2-yl; and the remaining variables are as described in the first aspect or the first, second, third, fourth, fifth, sixth, seventh, eighth, ninth, tenth, or eleventh embodiment or any alternative embodiments described therein.

In a seventeenth embodiment, the compound of the present disclosure is represented by Formula (V):

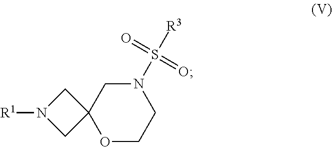

or a pharmaceutically acceptable salt thereof, wherein the variables in Formula (V) are as defined in the first aspect or the first, second, third, fourth, fifth, sixth, seventh, eighth, ninth, tenth, or eleventh embodiment above or any alternative embodiments described therein.

In an eighteenth embodiment, for the compounds of Formula (V), or a pharmaceutically acceptable salt thereof, R¹ is

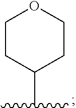 [SMILES: *C1CCOCC1]

### R³ is

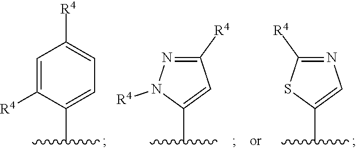

and R⁴, for each occurrence, is independently selected from —CH₃, —F, and cyclopropyl.

In a nineteenth embodiment, the compound of the present disclosure is represented by Formula (VI):

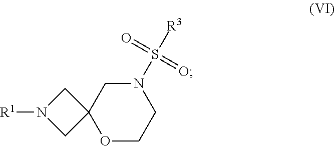

or a pharmaceutically acceptable salt thereof, wherein the variables in Formula (VI) are as defined in the first aspect or the first embodiment above.

In a twentieth embodiment, the compound of the present disclosure is represented by Formula (VII) or (VIII):

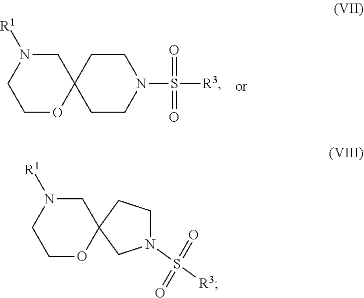

or a pharmaceutically acceptable salt thereof, wherein the variables in Formula (VII) or (VIII) are as defined in the first aspect or the first embodiment above.

In a twenty-first embodiment, for the compounds of Formula (VI), (VII), or (VIII), or a pharmaceutically acceptable salt thereof, R³ is phenyl optionally substituted with one to three R⁴; and the remaining variables are as described in the first aspect or the first embodiment.

In a twenty-second embodiment, for the compounds of Formula (VI), (VII), or (VIII), or a pharmaceutically acceptable salt thereof, R³ is

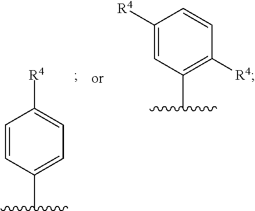

and the remaining variables are as described in the twenty-first embodiment.

In a twenty-third embodiment, for the compounds of Formula (VI), (VII), or (VIII), or a pharmaceutically acceptable salt thereof, R⁴, for each occurrence, is independently selected from halo, CN, and OR^{4a}; and R^{4a} is C₁₋₃alkyl or C₁₋₃haloalkyl; and the remaining variables are as described in the first aspect or the first, twenty-first, or twenty-second embodiment.

In a twenty-fourth embodiment, for the compounds of Formula (VI), (VII), or (VIII), or a pharmaceutically acceptable salt thereof, R⁴, for each occurrence, is independently selected from —OCHF₂, —F, and —CN; and the remaining variables are as described in the first aspect or the first, twenty-first, or twenty-second embodiment.

In a twenty-fifth embodiment, for the compounds of Formula (VI), (VII), or (VIII), or a pharmaceutically acceptable salt thereof, R¹ is C₂₋₄alkyl substituted with —OR^{2a} or

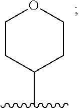 [SMILES: *C1CCOCC1]

optionally substituted with one to three R²; and the remaining variables are as described in the first aspect or the first, twenty-first, twenty-second, twenty-third, or twenty-fourth embodiment.

In a twenty-sixth embodiment, for the compounds of Formula (VI), (VII), or (VIII), or a pharmaceutically acceptable salt thereof, R¹ is

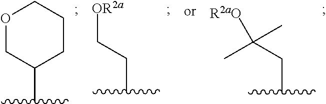

and the remaining variables are as described in the first aspect or the first, twenty-first, twenty-second, twenty-third, or twenty-fourth embodiment.

In a twenty-seventh embodiment, for the compounds of Formula (VI), (VII), or (VIII), or a pharmaceutically acceptable salt thereof, R², for each occurrence, is independently selected from —CH₃ and —OH; and R^{2a}, for each occurrence, is independently selected from H and —CH₃; and the remaining variables are as described in the first aspect or the first, twenty-first, twenty-second, twenty-third, twenty-fourth, twenty-fifth, or twenty-sixth embodiment.

In a twenty-eighth embodiment, the compound of the present disclosure is represented by Formula (IX):

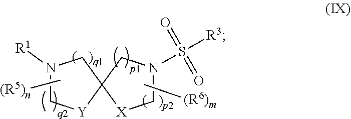

or a pharmaceutically acceptable salt thereof, wherein R⁵ is H, or two R⁵ together form a C₁₋₃alkylene; R⁶ is H, or two R⁶ together form a C₁₋₃alkylene; and wherein the remaining variables in Formula (IX) are as defined in the first aspect or the first embodiment above.

In a twenty-ninth embodiment, for the compounds of Formula (IX), or a pharmaceutically acceptable salt thereof, p1 is 1 and p2 is 0; or p1 is 1 and p2 is 1; or p1 is 2 and p2 is 1; and wherein the remaining variables in Formula (IX) are as defined in the twenty-eighth embodiment above.

In a thirtieth embodiment, for the compounds of Formula (IX), or a pharmaceutically acceptable salt thereof, R³ is C₁₋₄alkyl-phenyl, phenyl, pyridyl, thiazoyl, and pyrazolyl, each of which is optionally substituted with one to three R⁴; and the remaining variables are as described in the first aspect or the first, twenty-eighth, or twenty-ninth embodiment. In an alternative embodiment, for the compounds of Formula (IX), or a pharmaceutically acceptable salt thereof, R³ is phenyl, pyridyl, or pyrazolyl, each of which is optionally substituted with one to three R⁴; and the remaining variables are as described in the first aspect or the first, twenty-eighth, or twenty-ninth embodiment.

In a thirty-first embodiment, for the compounds of Formula (IX), or a pharmaceutically acceptable salt thereof, R³ is

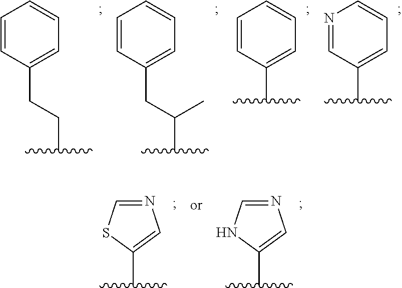

each of which is optionally substituted with one to three R⁴; or R³ is

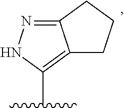 [SMILES: *c1[nH]nc2c1CCC2]

optionally substituted with one or two R⁴; and the remaining variables are as described in the first aspect or the first, twenty-eighth, or twenty-ninth embodiment. In an alternative embodiment, for the compounds of Formula (IX), or a pharmaceutically acceptable salt thereof, R³ is

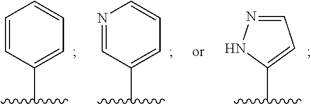

each of which is optionally substituted with one to three R⁴; or R³ is

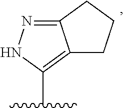 [SMILES: *c1[nH]nc2c1CCC2]

optionally substituted with one or two R⁴; and the remaining variables are as described in the first aspect or the first, twenty-eighth, or twenty-ninth embodiment.

In a thirty-second embodiment, for the compounds of Formula (IX), or a pharmaceutically acceptable salt thereof, R³ is

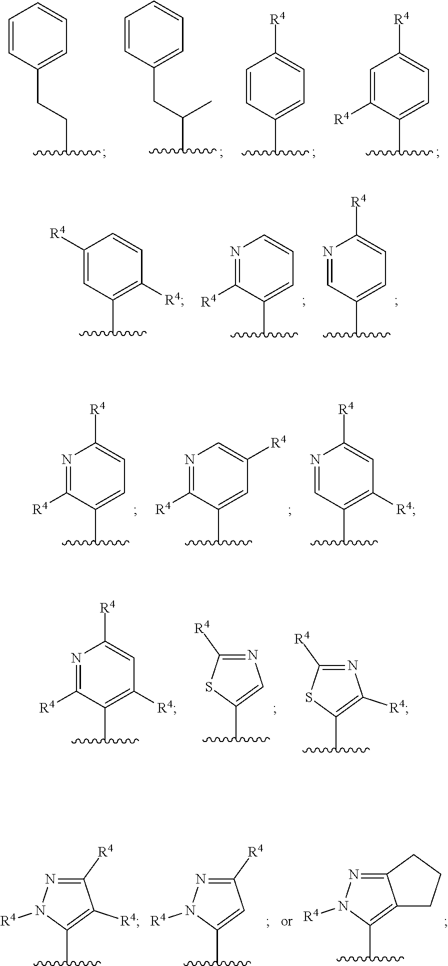

and the remaining variables are as described in the first aspect or the first, twenty-eighth, or twenty-ninth embodiment. In an alternative embodiment, for the compounds of Formula (IX), or a pharmaceutically acceptable salt thereof, R³ is

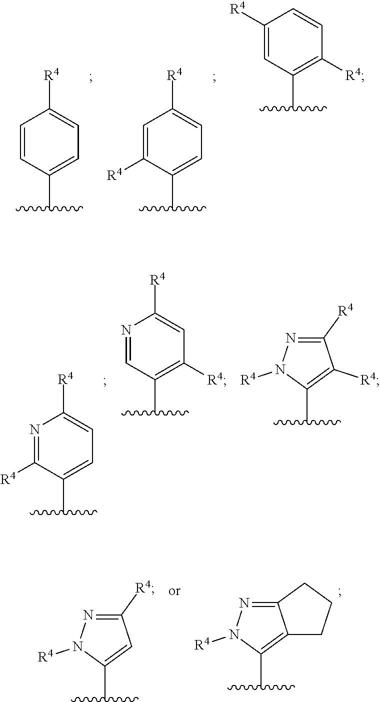

and the remaining variables are as described in the first aspect or the first, twenty-eighth, or twenty-ninth embodiment.

In a thirty-third embodiment, for the compounds of Formula (IX), or a pharmaceutically acceptable salt thereof, R⁴, for each occurrence, is independently selected from halo, CN, C₁₋₃alkyl, C₁₋₃haloalkyl, C₂₋₄alkenyl, C₃₋₆cycloalkyl, phenyl, and —OR^{4a}; wherein the C₃₋₆ cycloalkyl is optionally substituted with one to three halo or C₁₋₄haloalkyl and the C₁₋₃alkyl is optionally substituted with one or two C₃₋₄cycloalkyl; and R^{4a} is C₁₋₃alkyl or C₁₋₃haloalkyl; and the remaining variables are as described in the first aspect or the first, twenty-eighth, twenty-ninth, thirtieth, thirty-first, or thirty-second embodiment. In an alternative embodiment, for the compounds of Formula (IX), or a pharmaceutically acceptable salt thereof, R⁴, for each occurrence, is independently selected from halo, CN, C₁₋₃alkyl, C₁₋₃haloalkyl, C₃₋₆cycloalkyl, and —OR^{4a}; and R^{4a} is C₁₋₃alkyl or C₁₋₃haloalkyl; and the remaining variables are as described in the first aspect or the first, twenty-eighth, twenty-ninth, thirtieth, thirty-first, or thirty-second embodiment.

In a thirty-fourth embodiment, for the compounds of Formula (IX), or a pharmaceutically acceptable salt thereof, R⁴, for each occurrence, is independently selected from —CH₃, —CH₂CH₃, —CF₂CH₃, —CF(CH₃)₂, —CH═CH₂, —CHF₂, —CH(CH₃)₂, —CF₃, —OCH₃, —OCHF₂, —OCF₃, cyclopropyl,

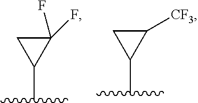

—CH₂-cyclopropyl, cyclobutyl, phenyl, —CN, —Cl, —Br, and —F; and the remaining variables are as described in the first aspect or the first, twenty-eighth, twenty-ninth, thirtieth, thirty-first, or thirty-second embodiment. In an alternative embodiment, for the compounds of Formula (IX), or a pharmaceutically acceptable salt thereof, R⁴, for each occurrence, is independently selected from —CH₃, —CHF₂, —CF₃, —OCH₃, —OCHF₂, —OCF₃, cyclopropyl, —CN, and —F; and the remaining variables are as described in the first aspect or the first, twenty-eighth, twenty-ninth, thirtieth, thirty-first, or thirty-second embodiment.

In a thirty-fifth embodiment, for the compounds of Formula (IX), or a pharmaceutically acceptable salt thereof, R¹ is

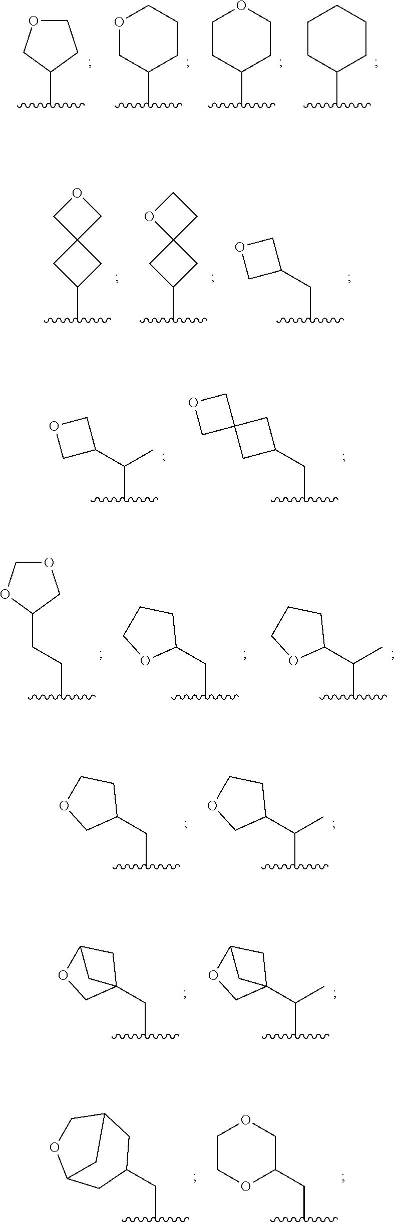

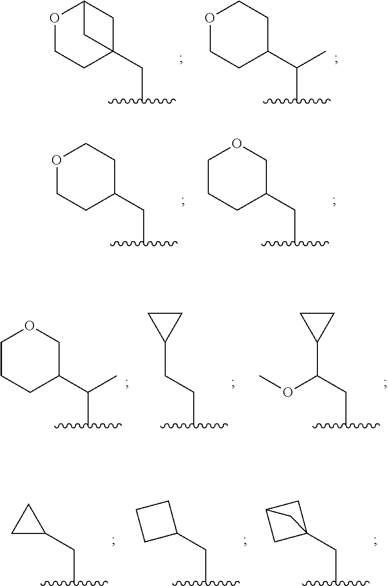

wherein each of the formula depicted above is optionally substituted with one to three R²; and the remaining variables are as described in the first aspect or the first, twenty-eighth, twenty-ninth, thirtieth, thirty-first, thirty-second, thirty-third, or thirty-fourth embodiment. In an alternative embodiment, for the compounds of Formula (IX), or a pharmaceutically acceptable salt thereof, R¹ is

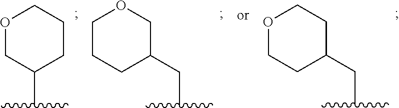

wherein each of the formula depicted above is optionally substituted with one to three R²; and the remaining variables are as described in the first aspect or the first, twenty-eighth, twenty-ninth, thirtieth, thirty-first, thirty-second, thirty-third, or thirty-fourth embodiment.

In a thirty-sixth embodiment, for the compounds of Formula (IX), or a pharmaceutically acceptable salt thereof, R¹ is

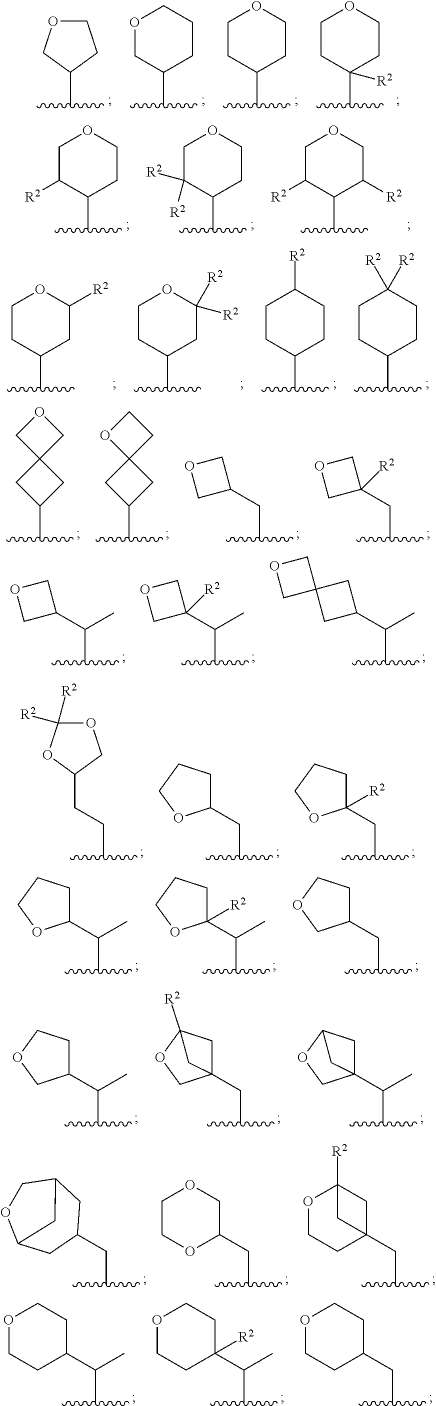

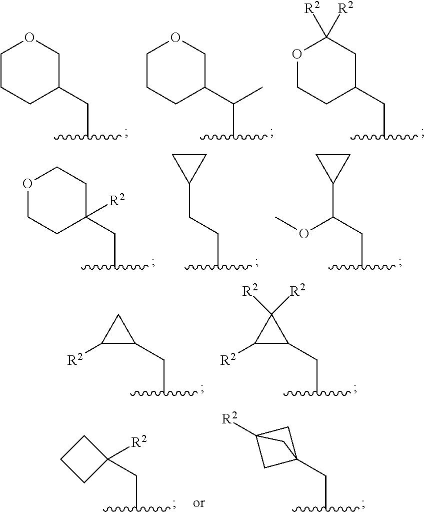

and the remaining variables are as described in the first aspect or the first, twenty-eighth, twenty-ninth, thirtieth, thirty-first, thirty-second, thirty-third, or thirty-fourth embodiment or any alternative embodiments described therein. In an alternative embodiment, for the compounds of Formula (IX), or a pharmaceutically acceptable salt thereof, R¹ is

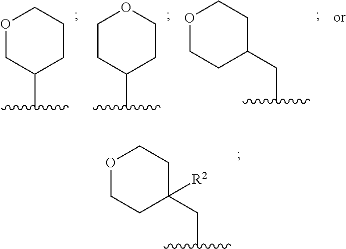

and the remaining variables are as described in the first aspect or the first, twenty-eighth, twenty-ninth, thirtieth, thirty-first, thirty-second, thirty-third, or thirty-fourth embodiment or any alternative embodiments described therein.

In a thirty-seventh embodiment, for the compounds of Formula (IX), or a pharmaceutically acceptable salt thereof, R² is —CH₃, —F, —OCH₃—CN, —CH₂CH₂OCH₃ or —OH; and the remaining variables are as described in the first aspect or the first, twenty-eighth, twenty-ninth, thirtieth, thirty-first, thirty-second, thirty-third, thirty-fourth, thirty-fifth, or thirty-sixth embodiment. In an alternative embodiment, for the compounds of Formula (IX), or a pharmaceutically acceptable salt thereof, R² is OH; and the remaining variables are as described in the first aspect or the first, twenty-eighth, twenty-ninth, thirtieth, thirty-first, thirty-second, thirty-third, thirty-fourth, thirty-fifth, or thirty-sixth embodiment or any alternative embodiments described therein.

In a thirty-eighth embodiment, for the compounds of Formula (IX), or a pharmaceutically acceptable salt thereof, R¹ is C₂₋₆alkyl optionally substituted with one or two R^{A}; R^{A}, for each occurrence, is independently OR^{2a}, SR^{2a}, or C(O)OR^{2a}; and R^{2a} is H, C₁₋₄alkyl, or C₃₋₆cycloalkyl wherein the C₁₋₄alkyl is optionally substituted with one or two C₁₋₃alkoxy; and the remaining variables are as described in the first aspect or the first, twenty-eighth, twenty-ninth, thirtieth, thirty-first, thirty-second, thirty-third, or thirty-fourth embodiment. In one embodiment, for the compounds of Formula (IX), or a pharmaceutically acceptable salt thereof, R¹ is —CH₂CH₃, —CH₂CH₂CH₂SCH₂CH₃, —CH₂CH₂CH₂O-cyclopentyl, —CH₂CH₂CH₂CH₂CH₂OCH₃, —CH₂CH₂OC(CH₃)₃, —CH₂CH₂CH₂CH(CH₃)OCH₃, —CH₂CH(CH₃)CH₂CH₂C(O)OCH₃, —CH₂CH₂C(CH₃)₂C(O)OCH₃, or —CH₂CH₂CH₂OCH₂CH₂OCH₃.

In a thirty-ninth embodiment, the compound of the present disclosure is represented by Formula (X):

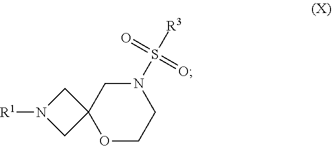

or a pharmaceutically acceptable salt thereof, wherein the variables in Formula (X) are as defined in the first aspect or the first, twenty-eighth, twenty-ninth, thirtieth, thirty-first, thirty-second, thirty-third, thirty-fourth, thirty-fifth, thirty-sixth, thirty-seventh or thirty eighth embodiment above or any alternative embodiments described therein.

In a fortieth embodiment, for the compounds of Formula (X), or a pharmaceutically acceptable salt thereof, R¹ is

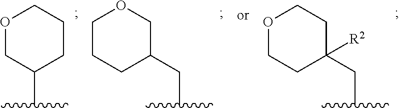

R² is OH; R³ is represented by the following formula:

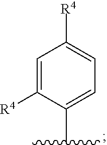

and R⁴ is —CH₃; the remaining variables are as defined in the thirty-eighth embodiment.

In a forty-first embodiment, the compound of the present disclosure is represented by Formula (XI):

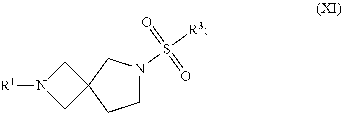

or a pharmaceutically acceptable salt thereof, wherein the variables in Formula (XI) are as defined in the first aspect or the first, twenty-eighth, twenty-ninth, thirtieth, thirty-first, thirty-second, thirty-third, thirty-fourth, thirty-fifth, thirty-sixth, thirty-seventh or thirty-eighth embodiment above or any alternative embodiments described therein.

In a forty-second embodiment, the compound of the present disclosure is represented by Formula (XIA) or (XIB):

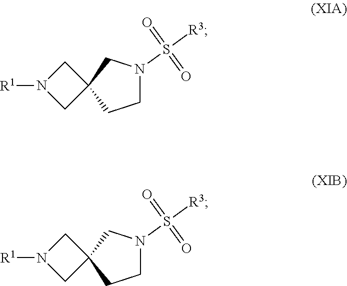

or a pharmaceutically acceptable salt thereof, wherein the variables in Formula (XIA) or (XIB) are as defined in the first aspect or the first, twenty-eighth, twenty-ninth, thirtieth, thirty-first, thirty-second, thirty-third, thirty-fourth, thirty-fifth, thirty-sixth, thirty-seventh or thirty-eighth embodiment above or any alternative embodiments described therein.

In a forty-third embodiment, for the compounds of Formula (X), (XIA), or (XIB), or a pharmaceutically acceptable salt thereof, R¹ is

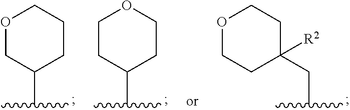

R² is OH; R³ is represented by the following formula:

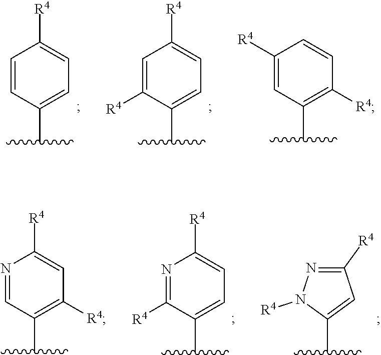

and R⁴, for each occurrence, is independently selected from —CH₃, —CF₃, —OCH₃, —OCHF₂, —OCF₃, —CN, —F, and cyclopropyl; the remaining variables are as defined in the forty-first or forty-second embodiment.

In a forty-fourth embodiment, the compound of the present disclosure is represented by Formula (XII):

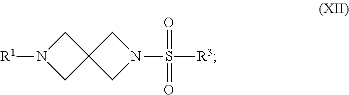

or a pharmaceutically acceptable salt thereof, wherein the variables in Formula (XII) are as defined in the first aspect or the first, twenty-eighth, twenty-ninth, thirtieth, thirty-first, thirty-second, thirty-third, thirty-fourth, thirty-fifth, thirty-sixth, thirty-seventh or thirty-eighth embodiment above or any alternative embodiments described therein.

In a forty-fifth embodiment, for the compounds of Formula (XII), or a pharmaceutically acceptable salt thereof, R¹ is

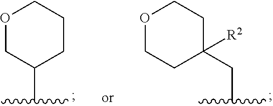

R² is OH; R³ is represented by the following formula:

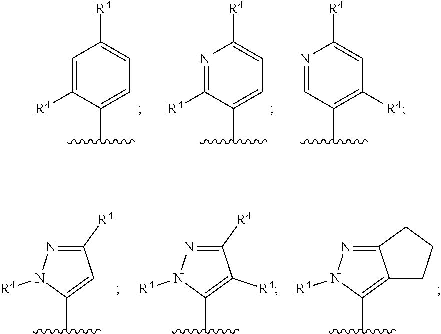

and R⁴, for each occurrence, is independently selected from —CH₃, —CHF₂, —CF₃, —OCH₃, —OCHF₂, and —OCF₃; the remaining variables are as defined in the forty-fourth embodiment.

In a forty-sixth embodiment, the compound of the present disclosure is represented by Formula (XII):

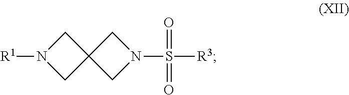

or a pharmaceutically acceptable salt thereof, wherein:
R¹ is Het or Z-Het;
Z is CH₂;
Het is represented by the following formula:

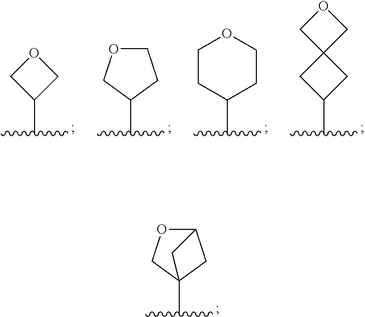

wherein each of the formula depicted above is optionally substituted with one to two R²;
R², for each occurrence, is independently C₁₋₄alkyl, —OH, or halo;
R³ is pyridinyl or pyrazolyl, each of which is optionally substituted with one to two R⁴;
R⁴, for each occurrence, is independently C₁₋₄alkyl or C₁₋₄haloalkyl.

In a forty-seventh embodiment, for the compounds of Formula (XII), or a pharmaceutically acceptable salt thereof, R³ is represented by the following formula:

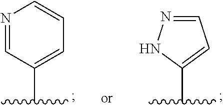 [SMILES: *c1cccnc1 ; *c1ccn[nH]1]

each of which is optionally substituted with one to two R⁴; and the remaining variables are as defined in the forty-sixth embodiment.

In a forty-eighth embodiment, for the compounds of Formula (XII), or a pharmaceutically acceptable salt thereof, R³ is represented by the following formula:

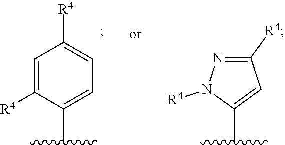

and the remaining variables are as defined in the forty-sixth or forty-seventh embodiment.

In a forty-ninth embodiment, for the compounds of Formula (XII), or a pharmaceutically acceptable salt thereof, R⁴, for each occurrence, is independently —CH₃, —CF₃, or —CF₂CH₃; and the remaining variables are as defined in the forty-sixth, forty-seventh, or forty-eighth embodiment.

In a fiftieth embodiment, the compound of the present disclosure is represented by Formula (XIIA):

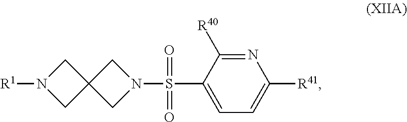

or a pharmaceutically acceptable salt thereof, wherein R⁴⁰ is C₁₋₃alkyl and R⁴¹ is C₁₋₃haloalkyl; and the remaining variables are as defined in the forty-eighth embodiment.

In a fifty-first embodiment, for the compounds of Formula (XIIA), or a pharmaceutically acceptable salt thereof, R⁴⁰ is —CH₃ and R⁴¹ is —CF₃ or —CF₂CH₃; and the remaining variables are as defined in the fiftieth embodiment.

In a fifty-second embodiment, for the compounds of Formula (XII) or (XIIA), or a pharmaceutically acceptable salt thereof, R¹ is represented by the following formula:

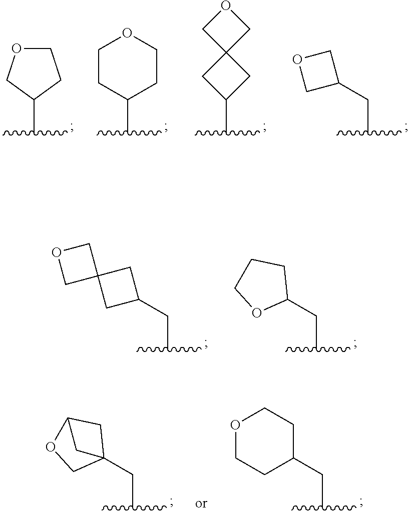

wherein each of the formula depicted above is optionally substituted with one to two R²; and the remaining variables are as defined in the forty-sixth, forty-seventh, forty-eighth, forty-ninth, fiftieth or fifty-first embodiment.

In a fifty-third embodiment, for the compounds of Formula (XII) or (XIIA), or a pharmaceutically acceptable salt thereof, R¹ is represented by the following formula:

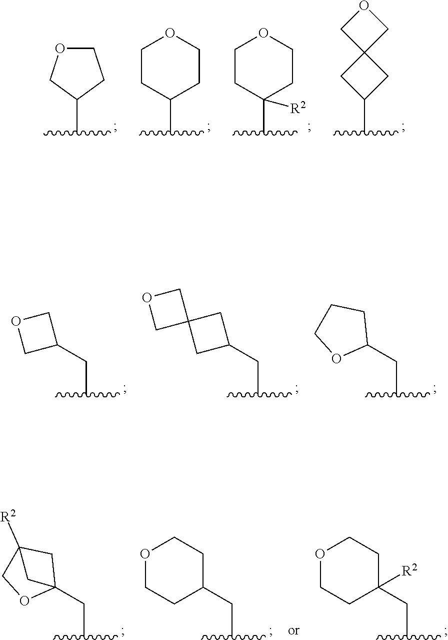

and the remaining variables are as defined in fifty-second embodiment.

In a fifty-fourth embodiment, for the compounds of Formula (XII), or a pharmaceutically acceptable salt thereof, R², for each occurrence, is independently —CH₃, OH, or —F; and the remaining variables are as defined in the forty-sixth, forty-seventh, forty-eighth, forty-ninth, fiftieth, fifty-first, fifty-second or fifty-third embodiment.

In a fifty-fifth embodiment, the present disclosure provides a compound described herein (e.g., a compound of any one of Examples 1 to 229), or a pharmaceutically acceptable salt thereof.

In a fifty-sixth embodiment, the present disclosure provides a compound selected from the group consisting of:
8-((4-(Difluoromethoxy)phenyl)sulfonyl)-2-(tetrahydro-2H-pyran-4-yl)-5-oxa-2,8-diazaspiro[3.5]nonane;
9-((4-(Difluoromethoxy)phenyl)sulfonyl)-2-(tetrahydro-2H-pyran-4-yl)-6-oxa-2,9-diazaspiro[4.5]decane;
(S)-9-((4-(Difluoromethoxy)phenyl)sulfonyl)-2-(tetrahydro-2H-pyran-4-yl)-6-oxa-2,9-diazaspiro[4.5]decane;
(R)-9-((4-(Difluoromethoxy)phenyl)sulfonyl)-2-(tetrahydro-2H-pyran-4-yl)-6-oxa-2,9-diazaspiro[4.5]decane;
4-((4-(Difluoromethoxy)phenyl)sulfonyl)-9-(tetrahydro-2H-pyran-4-yl)-1-oxa-4,9-diazaspiro[5.5]undecane;
4-((4-(Difluoromethoxy)phenyl)sulfonyl)-9-(tetrahydrofuran-3-yl)-1-oxa-4,9-diazaspiro[5.5]undecane;
4-((4-(Difluoromethoxy)phenyl)sulfonyl)-9-(2-methoxyethyl)-1-oxa-4,9-diazaspiro[5.5]undecane;
9-((4-(Difluoromethoxy)phenyl)sulfonyl)-2-((S)-tetrahydro-2H-pyran-3-yl)-6-oxa-2,9-diazaspiro[4.5]decane;
(±)—(R)-9-((4-(Difluoromethoxy)phenyl)sulfonyl)-2-((S)-tetrahydro-2H-pyran-3-yl)-6-oxa-2,9-diazaspiro[4.5]decane;
(±)—(R)-9-((4-(Difluoromethoxy)phenyl)sulfonyl)-2-((R)-tetrahydro-2H-pyran-3-yl)-6-oxa-2,9-diazaspiro[4.5]decane;
1-(4-((4-(Difluoromethoxy)phenyl)sulfonyl)-1-oxa-4,9-diazaspiro[5.5]undecan-9-yl)-2-methylpropan-2-ol;
4-((4-(Difluoromethoxy)phenyl)sulfonyl)-9-(2-methoxy-2-methylpropyl)-1-oxa-4,9-diazaspiro[5.5]undecane;
9-((2,4-Difluorophenyl)sulfonyl)-2-(tetrahydro-2H-pyran-4-yl)-6-oxa-2,9-diazaspiro[4.5]decane;
9-((3,5-Difluorophenyl)sulfonyl)-2-(tetrahydro-2H-pyran-4-yl)-6-oxa-2,9-diazaspiro[4.5]decane;
(S)-9-((3,5-Difluorophenyl)sulfonyl)-2-(tetrahydro-2H-pyran-4-yl)-6-oxa-2,9-diazaspiro[4.5]decane;
(R)-9-((3,5-Difluorophenyl)sulfonyl)-2-(tetrahydro-2H-pyran-4-yl)-6-oxa-2,9-diazaspiro[4.5]decane;
9-((2-Methoxy-5-methylpyridin-3-yl)sulfonyl)-2-(tetrahydro-2H-pyran-4-yl)-6-oxa-2,9-diazaspiro[4.5]decane;
9-((4-(Difluoromethoxy)phenyl)sulfonyl)-2-(oxetan-3-ylmethyl)-6-oxa-2,9-diazaspiro[4.5]decane;
9-((4-(Difluoromethoxy)phenyl)sulfonyl)-2-((tetrahydro-2H-pyran-4-yl)methyl)-6-oxa-2,9-diazaspiro[4.5]decane;
4-((4-(Difluoromethoxy)phenyl)sulfonyl)-9-((tetrahydro-2H-pyran-4-yl)methyl)-1-oxa-4,9-diazaspiro[5.5]undecane;
4-((4-(Difluoromethoxy)phenyl)sulfonyl)-9-((3-methyloxetan-3-yl)methyl)-1-oxa-4,9-diazaspiro[5.5]undecane;
4-((4-(Difluoromethoxy)phenyl)sulfonyl)-9-(2,2-dimethyltetrahydro-2H-pyran-4-yl)-1-oxa-4,9-diazaspiro[5.5]undecane;
8-((2-Cyclopropylthiazol-5-yl)sulfonyl)-2-(tetrahydro-2H-pyran-4-yl)-5-oxa-2,8-diazaspiro[3.5]nonane;
9-((2-Cyclopropylthiazol-5-yl)sulfonyl)-2-(tetrahydro-2H-pyran-4-yl)-6-oxa-2,9-diazaspiro[4.5]decane;
9-((2-Cyclopropylthiazol-5-yl)sulfonyl)-2-(2-oxaspiro[3.3]heptan-6-yl)-6-oxa-2,9-diazaspiro[4.5]decane;
9-((4-(Difluoromethoxy)phenyl)sulfonyl)-2-(2-oxaspiro[3.3]heptan-6-yl)-6-oxa-2,9-diazaspiro[4.5]decane;
(R)-9-((4-(Difluoromethoxy)phenyl)sulfonyl)-2-(2-oxaspiro[3.3]heptan-6-yl)-6-oxa-2,9-diazaspiro[4.5]decane;
(S)-9-((4-(Difluoromethoxy)phenyl)sulfonyl)-2-(2-oxaspiro[3.3]heptan-6-yl)-6-oxa-2,9-diazaspiro[4.5]decane;
9-((3,5-Difluorophenyl)sulfonyl)-2-(2-oxaspiro[3.3]heptan-6-yl)-6-oxa-2,9-diazaspiro[4.5]decane;
4-((3,5-Difluorophenyl)sulfonyl)-9-((3-methyloxetan-3-yl)methyl)-1-oxa-4,9-diazaspiro[5.5]undecane;
9-((4-(Difluoromethoxy)phenyl)sulfonyl)-4-(tetrahydro-2H-pyran-4-yl)-1-oxa-4,9-diazaspiro[5.5]undecane;
7-((2,4-Dimethylphenyl)sulfonyl)-2-(tetrahydro-2H-pyran-4-yl)-2,7-diazaspiro[3.5]nonane;
7-((4-(Difluoromethoxy)phenyl)sulfonyl)-2-(tetrahydro-2H-pyran-4-yl)-2,7-diazaspiro[3.5]nonane;
7-((2-methyl-6-(trifluoromethyl)pyridin-3-yl)sulfonyl)-2-(tetrahydro-2H-pyran-4-yl)-2,7-diazaspiro[3.5]nonane;
1-((7-((2,4-Dimethylphenyl)sulfonyl)-2,7-diazaspiro[3.5]nonan-2-yl)methyl)tetrahydro-2H-pyran-4-ol;
7-((2,4-Dimethylphenyl)sulfonyl)-2-((tetrahydro-2H-pyran-4-yl)methyl)-2,7-diazaspiro[3.5]nonane;
8-((2-Chloro-4-methylphenyl)sulfonyl)-2-(tetrahydro-2H-pyran-4-yl)-2,8-diazaspiro[4.5]decane;
8-((1,3-Dimethyl-1H-pyrazol-5-yl)sulfonyl)-2-(tetrahydro-2H-pyran-4-yl)-2,8-diazaspiro[4.5]decane;
8-((4-Methyl-6-(trifluoromethyl)pyridin-3-yl)sulfonyl)-2-(tetrahydro-2H-pyran-4-yl)-2,8-diazaspiro[4.5]decane;
8-((4-(Difluoromethoxy)phenyl)sulfonyl)-2-(tetrahydro-2H-pyran-4-yl)-2,8-diazaspiro[4.5]decane;
8-((1-Methyl-3-(trifluoromethyl)-1H-pyrazol-5-yl)sulfonyl)-2-(tetrahydro-2H-pyran-4-yl)-2,8-diazaspiro[4.5]decane;
8-((2-Methyl-4-(trifluoromethoxy)phenyl)sulfonyl)-2-(tetrahydro-2H-pyran-4-yl)-2,8-diazaspiro[4.5]decane;
8-((2-Methyl-6-(trifluoromethyl)pyridin-3-yl)sulfonyl)-2-(tetrahydro-2H-pyran-4-yl)-2,8-diazaspiro[4.5]decane;
8-((6-Methoxy-2-methylpyridin-3-yl)sulfonyl)-2-(tetrahydro-2H-pyran-4-yl)-2,8-diazaspiro[4.5]decane;
2-((2,4-Dimethylphenyl)sulfonyl)-6-(tetrahydro-2H-pyran-4-yl)-2,6-diazaspiro[3.3]heptane;
4-((6-((2,4-Dimethylphenyl)sulfonyl)-2,6-diazaspiro[3.3]heptan-2-yl)methyl)tetrahydro-2H-pyran-4-ol;
2-((1-Methyl-3-(trifluoromethyl)-1H-pyrazol-5-yl)sulfonyl)-6-(tetrahydro-2H-pyran-4-yl)-2,6-diazaspiro[3.3]heptane;
2-((6-Methoxy-2-methylpyridin-3-yl)sulfonyl)-6-(tetrahydro-2H-pyran-4-yl)-2,6-diazaspiro[3.3]heptane;
2-((4-Methyl-6-(trifluoromethyl)pyridin-3-yl)sulfonyl)-6-(tetrahydro-2H-pyran-4-yl)-2,6-diazaspiro[3.3]heptane;
2-((4-(Difluoromethyl)-1,3-dimethyl-1H-pyrazol-5-yl)sulfonyl)-6-(tetrahydro-2H-pyran-4-yl)-2,6-diazaspiro[3.3]heptane;
2-((2-Methyl-4-(trifluoromethoxy)phenyl)sulfonyl)-6-(tetrahydro-2H-pyran-4-yl)-2,6-diazaspiro[3.3]heptane;
2-((2-Methyl-6-(trifluoromethyl)pyridin-3-yl)sulfonyl)-6-(tetrahydro-2H-pyran-4-yl)-2,6-diazaspiro[3.3]heptane;
2-((2,4-Dimethylphenyl)sulfonyl)-6-(tetrahydro-2H-pyran-4-yl)-2,6-diazaspiro[3.4]octane;
2-((4,6-Dimethylpyridin-3-yl)sulfonyl)-6-(tetrahydro-2H-pyran-4-yl)-2,6-diazaspiro[3.4]octane;
2-((4-(Difluoromethoxy)phenyl)sulfonyl)-6-(tetrahydro-2H-pyran-4-yl)-2,6-diazaspiro[3.4]octane;
2-((1-Methyl-3-(trifluoromethyl)-1H-pyrazol-5-yl)sulfonyl)-6-(tetrahydro-2H-pyran-4-yl)-2,6-diazaspiro[3.4]octane;
2-((2-Methyl-6-(trifluoromethyl)pyridin-3-yl)sulfonyl)-6-(tetrahydro-2H-pyran-4-yl)-2,6-diazaspiro[3.4]octane;
2-((4-Methyl-6-(trifluoromethyl)pyridin-3-yl)sulfonyl)-6-(tetrahydro-2H-pyran-4-yl)-2,6-diazaspiro[3.4]octane;
2-((2-Methyl-4-(trifluoromethoxy)phenyl)sulfonyl)-6-(tetrahydro-2H-pyran-4-yl)-2,6-diazaspiro[3.4]octane;
2-((3-Cyclopropyl-1-methyl-1H-pyrazol-5-yl)sulfonyl)-6-(tetrahydro-2H-pyran-4-yl)-2,6-diazaspiro[3.4]octane;
6-((1-methyl-3-(Trifluoromethyl)-1H-pyrazol-5-yl)sulfonyl)-2-(tetrahydro-2H-pyran-4-yl)-2,6-diazaspiro[3.4]octane;
(1′R,5′S)-8′-((4-(Difluoromethoxy)phenyl)sulfonyl)-1-(tetrahydro-2H-pyran-4-yl)-8′-azaspiro[azetidine-3,3′-bicyclo[3.2.1]octane];
1-((1′R,5′S)-1-((2,4-Dimethylphenyl)sulfonyl)-8′-azaspiro[azetidine-3,3′-bicyclo[3.2.1]octan]-8′-yl)-2-methylpropan-2-ol;
(1′R,5′S)-8′-((4-(Difluoromethoxy)phenyl)sulfonyl)-1-(2-methoxy-2-methylpropyl)-8′-azaspiro[azetidine-3,3′-bicyclo[3.2.1]octane];
(1′R,5′S)-1-((4-(Difluoromethoxy)phenyl)sulfonyl)-8′-(2-methoxy-2-methylpropyl)-8′-azaspiro[azetidine-3,3′-bicyclo[3.2.1]octane];
2-((1-(cyclopropylmethyl)-3-(trifluoromethyl)-1H-pyrazol-5-yl)sulfonyl)-6-(tetrahydro-2H-pyran-4-yl)-2,6-diazaspiro[3.3]heptane;
2-((1-(cyclopropylmethyl)-3-(trifluoromethyl)-1H-pyrazol-5-yl)sulfonyl)-6-((tetrahydro-2H-pyran-4-yl)methyl)-2,6-diazaspiro[3.3]heptane;
2-((6-bromo-2-methylpyridin-3-yl)sulfonyl)-6-(tetrahydro-2H-pyran-4-yl)-2,6-diazaspiro[3.3]heptane;
2-((2-methyl-6-(trifluoromethyl)pyridin-3-yl)sulfonyl)-6-(2-oxaspiro[3.3]heptan-6-yl)-2,6-diazaspiro[3.3]heptane;
2-((2-methyl-6-(trifluoromethyl)pyridin-3-yl)sulfonyl)-6-(1-oxaspiro[3.3]heptan-6-yl)-2,6-diazaspiro[3.3]heptane;
2-((2-methyl-6-(trifluoromethyl)-1,4-dihydropyridin-3-yl)sulfonyl)-6-(1-oxaspiro[3.3]heptan-6-yl)-2,6-diazaspiro[3.3]heptane;
2-((2-methyl-6-(trifluoromethyl)pyridin-3-yl)sulfonyl)-6-(1-oxaspiro[3.3]heptan-6-yl)-2,6-diazaspiro[3.3]heptane;
2-((1-methyl-3-(trifluoromethyl)-1H-pyrazol-5-yl)sulfonyl)-6-((4-methyltetrahydro-2H-pyran-4-yl)methyl)-2,6-diazaspiro[3.3]heptane;
2-((1-methyl-2-oxabicyclo[2.1.1]hexan-4-yl)methyl)-6-((2-methyl-6-(trifluoromethyl)pyridin-3-yl)sulfonyl)-2,6-diazaspiro[3.3]heptane;
2-((1-methyl-2-oxabicyclo[3.1.1]heptan-5-yl)methyl)-6-((2-methyl-6-(trifluoromethyl)-1,6-dihydropyridin-3-yl)sulfonyl)-2,6-diazaspiro[3.3]heptane;
4-((6-((6-(1,1-difluoroethyl)-2-methylpyridin-3-yl)sulfonyl)-2,6-diazaspiro[3.3]heptan-2-yl)methyl)tetrahydro-2H-pyran-4-ol;
2-((1-methyl-3-(trifluoromethyl)-1H-pyrazol-5-yl)sulfonyl)-6-(4-methyltetrahydro-2H-pyran-4-yl)-2,6-diazaspiro[3.3]heptane;
2-((2-methyl-6-(trifluoromethyl)-1,4-dihydropyridin-3-yl)sulfonyl)-6-(4-methyltetrahydro-2H-pyran-4-yl)-2,6-diazaspiro[3.3]heptane;
2-((6-(1,1-difluoroethyl)-2-methylpyridin-3-yl)sulfonyl)-6-(2-oxaspiro[3.3]heptan-6-yl)-2,6-diazaspiro[3.3]heptane;
2-((6-(1,1-difluoroethyl)-2-methylpyridin-3-yl)sulfonyl)-6-(tetrahydro-2H-pyran-4-yl)-2,6-diazaspiro[3.3]heptane;
(R)-2-((6-(1,1-difluoroethyl)-2-methylpyridin-3-yl)sulfonyl)-6-(tetrahydro-2H-pyran-3-yl)-2,6-diazaspiro[3.3]heptane;
2-((6-(1,1-difluoroethyl)-2-methyl-3,4-dihydropyridin-3-yl)sulfonyl)-6-((tetrahydro-2H-pyran-4-yl)methyl)-2,6-diazaspiro[3.3]heptane;
(S)-2-((6-(1,1-difluoroethyl)-2-methylpyridin-3-yl)sulfonyl)-6-(1-(tetrahydro-2H-pyran-4-yl)ethyl)-2,6-diazaspiro[3.3]heptane;
2-((6-(1,1-difluoroethyl)-2-methylpyridin-3-yl)sulfonyl)-6-((4-methyltetrahydro-2H-pyran-4-yl)methyl)-2,6-diazaspiro[3.3]heptane;
(R)-2-((6-(1,1-difluoroethyl)-2-methylpyridin-3-yl)sulfonyl)-6-(tetrahydrofuran-3-yl)-2,6-diazaspiro[3.3]heptane;
(S)-2-((6-(1,1-difluoroethyl)-2-methylpyridin-3-yl)sulfonyl)-6-((tetrahydrofuran-3-yl)methyl)-2,6-diazaspiro[3.3]heptane;
2-((6-(1,1-difluoroethyl)-2-methylpyridin-3-yl)sulfonyl)-6-((S)-1-((S)-tetrahydrofuran-3-yl)ethyl)-2,6-diazaspiro[3.3]heptane;
2-((6-(1,1-difluoroethyl)-2-methylpyridin-3-yl)sulfonyl)-6-(oxetan-3-ylmethyl)-2,6-diazaspiro[3.3]heptane;
(S)-2-((6-(1,1-difluoroethyl)-2-methylpyridin-3-yl)sulfonyl)-6-(1-(oxetan-3-yl)ethyl)-2,6-diazaspiro[3.3]heptane;
2-((6-(1,1-difluoroethyl)-2-methylpyridin-3-yl)sulfonyl)-6-((3-methyloxetan-3-yl)methyl)-2,6-diazaspiro[3.3]heptane;
2-((6-(difluoromethyl)-2-methylpyridin-3-yl)sulfonyl)-6-(tetrahydro-2H-pyran-4-yl)-2,6-diazaspiro[3.3]heptane;
2-((6-(difluoromethyl)-2-methylpyridin-3-yl)sulfonyl)-6-(2-oxaspiro[3.3]heptan-6-yl)-2,6-diazaspiro[3.3]heptane;
2-((6-(2-fluoropropan-2-yl)-2-methyl-1,4-dihydropyridin-3-yl)sulfonyl)-6-(2-oxaspiro[3.3]heptan-6-yl)-2,6-diazaspiro[3.3]heptane;
2-((6-(2-fluoropropan-2-yl)-2-methylpyridin-3-yl)sulfonyl)-6-(tetrahydro-2H-pyran-4-yl)-2,6-diazaspiro[3.3]heptane;
2-((6-(difluoromethoxy)-2-methyl-3,4-dihydropyridin-3-yl)sulfonyl)-6-(tetrahydro-2H-pyran-4-yl)-2,6-diazaspiro[3.3]heptane;
2-((6-(difluoromethoxy)-2-methylpyridin-3-yl)sulfonyl)-6-(2-oxaspiro[3.3]heptan-6-yl)-2,6-diazaspiro[3.3]heptane;
2-((5-(difluoromethyl)-2-methyl-1H-2l4-pyrazol-3-yl)sulfonyl)-6-(tetrahydro-2H-pyran-4-yl)-2,6-diazaspiro[3.3]heptane;
2-((3-methoxy-1-methyl-1H-pyrazol-5-yl)sulfonyl)-6-(tetrahydro-2H-pyran-4-yl)-2,6-diazaspiro[3.3]heptane;
2-((2-chloro-6-methoxypyridin-3-yl)sulfonyl)-6-(tetrahydro-2H-pyran-4-yl)-2,6-diazaspiro[3.3]heptane;
2-((5-chloro-2-methoxypyridin-3-yl)sulfonyl)-6-(tetrahydro-2H-pyran-4-yl)-2,6-diazaspiro[3.3]heptane;
2-((2,4-dimethyl-6-(trifluoromethyl)pyridin-3-yl)sulfonyl)-6-(tetrahydro-2H-pyran-4-yl)-2,6-diazaspiro[3.3]heptane;
2-((2,4-dimethyl-6-(trifluoromethyl)pyridin-3-yl)sulfonyl)-6-(2-oxaspiro[3.3]heptan-6-yl)-2,6-diazaspiro[3.3]heptane;
2-((2,4-dimethyl-6-(trifluoromethyl)pyridin-3-yl)sulfonyl)-6-((tetrahydro-2H-pyran-4-yl)methyl)-2,6-diazaspiro[3.3]heptane;
(S)-2-((2,4-dimethyl-6-(trifluoromethyl)pyridin-3-yl)sulfonyl)-6-((tetrahydrofuran-3-yl)methyl)-2,6-diazaspiro[3.3]heptane;
2-((2,4-dimethyl-6-(trifluoromethyl)pyridin-3-yl)sulfonyl)-6-(oxetan-3-ylmethyl)-2,6-diazaspiro[3.3]heptane;
2-((6-methyl-2-(trifluoromethyl)-1,4-dihydropyridin-3-yl)sulfonyl)-6-(tetrahydro-2H-pyran-4-yl)-2,6-diazaspiro[3.3]heptane;
2-((6-methyl-2-(trifluoromethyl)pyridin-3-yl)sulfonyl)-6-((tetrahydro-2H-pyran-4-yl)methyl)-2,6-diazaspiro[3.3]heptane;
2-((6-cyclopropyl-2-(trifluoromethyl)pyridin-3-yl)sulfonyl)-6-(tetrahydro-2H-pyran-4-yl)-2,6-diazaspiro[3.3]heptane;
2-((6-cyclopropyl-2-(trifluoromethyl)pyridin-3-yl)sulfonyl)-6-((tetrahydro-2H-pyran-4-yl)methyl)-2,6-diazaspiro[3.3]heptane;
2-((2,6-bis(trifluoromethyl)pyridin-3-yl)sulfonyl)-6-(tetrahydro-2H-pyran-4-yl)-2,6-diazaspiro[3.3]heptane;
2-((2,6-bis(trifluoromethyl)-1,4-dihydropyridin-3-yl)sulfonyl)-6-(2-oxaspiro[3.3]heptan-6-yl)-2,6-diazaspiro[3.3]heptane;
2-((2,6-bis(trifluoromethyl)pyridin-3-yl)sulfonyl)-6-((tetrahydro-2H-pyran-4-yl)methyl)-2,6-diazaspiro[3.3]heptane;
2-((2,6-bis(trifluoromethyl)pyridin-3-yl)sulfonyl)-6-(oxetan-3-ylmethyl)-2,6-diazaspiro[3.3]heptane;
2-((2-oxaspiro[3.3]heptan-6-yl)methyl)-6-((2-methyl-6-(trifluoromethyl)pyridin-3-yl)sulfonyl)-2,6-diazaspiro[3.3]heptane;
2-((2-oxaspiro[3.3]heptan-6-yl)methyl)-6-((6-(1,1-difluoroethyl)-2-methylpyridin-3-yl)sulfonyl)-2,6-diazaspiro[3.3]heptane;
2-((2-oxaspiro[3.3]heptan-6-yl)methyl)-6-((2-methyl-5-(trifluoromethyl)-2,5-dihydro-1H-pyrazol-3-yl)sulfonyl)-2,6-diazaspiro[3.3]heptane;
2-((2-cyclopropyl-6-(difluoromethyl)pyridin-3-yl)sulfonyl)-6-(tetrahydro-2H-pyran-4-yl)-2,6-diazaspiro[3.3]heptane;
2-((2-cyclopropyl-6-(difluoromethyl)-1,4-dihydropyridin-3-yl)sulfonyl)-6-((1s,4s)-4-methoxycyclohexyl)-2,6-diazaspiro[3.3]heptane;
2-((2-cyclopropyl-6-(difluoromethyl)pyridin-3-yl)sulfonyl)-6-(4-methoxycyclohexyl)-2,6-diazaspiro[3.3]heptane;
2-((2-cyclopropyl-6-(difluoromethyl)pyridin-3-yl)sulfonyl)-6-(2-oxaspiro[3.3]heptan-6-yl)-2,6-diazaspiro[3.3]heptane;
2-((1-cyclopropyl-3-(difluoromethyl)-1H-pyrazol-5-yl)sulfonyl)-6-(tetrahydro-2H-pyran-4-yl)-2,6-diazaspiro[3.3]heptane;
2-((4-cyclopropyl-6-(trifluoromethyl)pyridin-3-yl)sulfonyl)-6-(2-oxaspiro[3.3]heptan-6-yl)-2,6-diazaspiro[3.3]heptane;
2-((4-cyclopropyl-6-(trifluoromethyl)pyridin-3-yl)sulfonyl)-6-((tetrahydro-2H-pyran-4-yl)methyl)-2,6-diazaspiro[3.3]heptane;
(1r,4r)-4-(6-((4-cyclopropyl-6-(trifluoromethyl)-2,3-dihydropyridin-3-yl)sulfonyl)-2,6-diazaspiro[3.3]heptan-2-yl)-1-methylcyclohexan-1-ol;
4-(6-((4-cyclopropyl-6-(trifluoromethyl)pyridin-3-yl)sulfonyl)-2,6-diazaspiro[3.3]heptan-2-yl)-1-methylcyclohexan-1-ol;
2-((4-ethyl-6-(trifluoromethyl)pyridin-3-yl)sulfonyl)-6-(2-oxaspiro[3.3]heptan-6-yl)-2,6-diazaspiro[3.3]heptane;
2-((4-ethyl-6-(trifluoromethyl)pyridin-3-yl)sulfonyl)-6-((tetrahydro-2H-pyran-4-yl)methyl)-2,6-diazaspiro[3.3]heptane;
(1r,4r)-4-(6-((4-ethyl-6-(trifluoromethyl)pyridin-3-yl)sulfonyl)-2,6-diazaspiro[3.3]heptan-2-yl)-1-methylcyclohexan-1-ol;
(1s,4s)-4-(6-((4-ethyl-6-(trifluoromethyl)pyridin-3-yl)sulfonyl)-2,6-diazaspiro[3.3]heptan-2-yl)-1-methylcyclohexan-1-ol;
2-((2-methyl-6-(trifluoromethyl)pyridin-3-yl)sulfonyl)-6-((4-methyltetrahydro-2H-pyran-4-yl)methyl)-2,6-diazaspiro[3.3]heptane;
2-((2-methyl-6-(trifluoromethyl)-1,4-dihydropyridin-3-yl)sulfonyl)-6-((1R)-1-(tetrahydrofuran-3-yl)ethyl)-2,6-diazaspiro[3.3]heptane;
(S)-2-((2-methyl-6-(trifluoromethyl)-1,4-dihydropyridin-3-yl)sulfonyl)-6-(1-(4-methyltetrahydro-2H-pyran-4-yl)ethyl)-2,6-diazaspiro[3.3]heptane;
(S)-2-((2-methyl-6-(trifluoromethyl)pyridin-3-yl)sulfonyl)-6-(1-(3-methyloxetan-3-yl)ethyl)-2,6-diazaspiro[3.3]heptane;
(S)-2-((2-methyl-6-(trifluoromethyl)-1,4-dihydropyridin-3-yl)sulfonyl)-6-((tetrahydrofuran-3-yl)methyl)-2,6-diazaspiro[3.3]heptane;
(R)-2-((2-methyl-6-(trifluoromethyl)pyridin-3-yl)sulfonyl)-6-((2-methyltetrahydrofuran-2-yl)methyl)-2,6-diazaspiro[3.3]heptane;
2-((2-methyl-6-(trifluoromethyl)pyridin-3-yl)sulfonyl)-6-((S)-1-((S)-2-methyltetrahydrofuran-2-yl)ethyl)-2,6-diazaspiro[3.3]heptane;
(S)-2-((2-methyl-6-(trifluoromethyl)pyridin-3-yl)sulfonyl)-6-((tetrahydro-2H-pyran-3-yl)methyl)-2,6-diazaspiro[3.3]heptane;
2-((2-methyl-6-(trifluoromethyl)pyridin-3-yl)sulfonyl)-6-((S)-1-((S)-tetrahydro-2H-pyran-3-yl)ethyl)-2,6-diazaspiro[3.3]heptane;
2-((2-methyl-6-(trifluoromethyl)pyridin-3-yl)sulfonyl)-6-((R)-1-((S)-tetrahydrofuran-2-yl)ethyl)-2,6-diazaspiro[3.3]heptane;
(S)-2-((2-methyl-6-(trifluoromethyl)pyridin-3-yl)sulfonyl)-6-((tetrahydrofuran-2-yl)methyl)-2,6-diazaspiro[3.3]heptane;
(R)-2-((2-methyl-6-(trifluoromethyl)pyridin-3-yl)sulfonyl)-6-(tetrahydrofuran-3-yl)-2,6-diazaspiro[3.3]heptane;
2-((2-methyl-6-(trifluoromethyl)pyridin-3-yl)sulfonyl)-6-((3-methyloxetan-3-yl)methyl)-2,6-diazaspiro[3.3]heptane;
(R)-2-(3,3-dimethyltetrahydro-2H-pyran-4-yl)-6-((2-methyl-6-(trifluoromethyl)pyridin-3-yl)sulfonyl)-2,6-diazaspiro[3.3]heptane;
2-((2-methyl-6-(trifluoromethyl)pyridin-3-yl)sulfonyl)-6-((2S,4S)-2-methyltetrahydro-2H-pyran-4-yl)-2,6-diazaspiro[3.3]heptane;
2-((2-methyl-6-(trifluoromethyl)pyridin-3-yl)sulfonyl)-6-((2S,4R)-2-methyltetrahydro-2H-pyran-4-yl)-2,6-diazaspiro[3.3]heptane;
2-((2-methyl-6-(trifluoromethyl)pyridin-3-yl)sulfonyl)-6-((3R,4R)-3-methyltetrahydro-2H-pyran-4-yl)-2,6-diazaspiro[3.3]heptane;
2-((3R,4R)-3-fluorotetrahydro-2H-pyran-4-yl)-6-((2-methyl-6-(trifluoromethyl)pyridin-3-yl)sulfonyl)-2,6-diazaspiro[3.3]heptane;
2-((3R,4S)-3-fluorotetrahydro-2H-pyran-4-yl)-6-((2-methyl-6-(trifluoromethyl)pyridin-3-yl)sulfonyl)-2,6-diazaspiro[3.3]heptane;
2-((3R,5R)-3,5-dimethyltetrahydro-2H-pyran-4-yl)-6-((2-methyl-6-(trifluoromethyl)pyridin-3-yl)sulfonyl)-2,6-diazaspiro[3.3]heptane;
2-((3R,5S)-3,5-dimethyltetrahydro-2H-pyran-4-yl)-6-((2-methyl-6-(trifluoromethyl)-1,4-dihydropyridin-3-yl)sulfonyl)-2,6-diazaspiro[3.3]heptane;
2-(4,4-difluorocyclohexyl)-6-((2-methyl-6-(trifluoromethyl)pyridin-3-yl)sulfonyl)-2,6-diazaspiro[3.3]heptane;
(R)-2-((2-methyl-6-(trifluoromethyl)-1,4-dihydropyridin-3-yl)sulfonyl)-6-(tetrahydro-2H-pyran-3-yl)-2,6-diazaspiro[3.3]heptane;
2-((2-methyl-6-(trifluoromethyl)-1,4-dihydropyridin-3-yl)sulfonyl)-6-((tetrahydro-2H-pyran-4-yl)methyl)-2,6-diazaspiro[3.3]heptane;
(S)-2-((2-methyl-6-(trifluoromethyl)-1,4-dihydropyridin-3-yl)sulfonyl)-6-(1-(tetrahydro-2H-pyran-4-yl)ethyl)-2,6-diazaspiro[3.3]heptane;
2-((2-methyl-6-(trifluoromethyl)pyridin-3-yl)sulfonyl)-6-(oxetan-3-ylmethyl)-2,6-diazaspiro[3.3]heptane;
(S)-2-((2-methyl-6-(trifluoromethyl)pyridin-3-yl)sulfonyl)-6-(1-(oxetan-3-yl)ethyl)-2,6-diazaspiro[3.3]heptane;
(R)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane;
(R)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane;
(R)-2-((2-methyl-6-(trifluoromethyl)pyridin-3-yl)sulfonyl)-6-(1-(oxetan-3-yl)ethyl)-2,6-diazaspiro[3.3]heptane;
(R)-2-((2-methyl-6-(trifluoromethyl)pyridin-3-yl)sulfonyl)-6-(1-(oxetan-3-yl)ethyl)-2,6-diazaspiro[3.3]heptane;
(R)-2-((2-methyl-6-(trifluoromethyl)pyridin-3-yl)sulfonyl)-6-((tetrahydrofuran-3-yl)methyl)-2,6-diazaspiro[3.3]heptane;
(R)-2-((2-methyl-6-(trifluoromethyl)pyridin-3-yl)sulfonyl)-6-((tetrahydrofuran-3-yl)methyl)-2,6-diazaspiro[3.3]heptane;
2-(tetrahydro-2H-pyran-4-yl)-6-((2-(trifluoromethyl)pyridin-3-yl)sulfonyl)-2,6-diazaspiro[3.3]heptane;
2-((6-(2,2-difluorocyclopropyl)-2-methylpyridin-3-yl)sulfonyl)-6-(tetrahydro-2H-pyran-4-yl)-2,6-diazaspiro[3.3]heptane;
2-((2-methylpyridin-3-yl)sulfonyl)-6-(tetrahydro-2H-pyran-4-yl)-2,6-diazaspiro[3.3]heptane;
2-((6-chloro-5-(trifluoromethyl)pyridin-3-yl)sulfonyl)-6-(tetrahydro-2H-pyran-4-yl)-2,6-diazaspiro[3.3]heptane;
2-((3-(1,1-difluoroethyl)-1-methyl-1H-pyrazol-5-yl)sulfonyl)-6-(tetrahydro-2H-pyran-4-yl)-2,6-diazaspiro[3.3]heptane;
2-(1-(1-methyl-2-oxabicyclo[2.1.1]hexan-4-yl)ethyl)-6-((2-methyl-6-(trifluoromethyl)pyridin-3-yl)sulfonyl)-2,6-diazaspiro[3.3]heptane;
2-((6-methoxy-4-methylpyridin-3-yl)sulfonyl)-6-(tetrahydro-2H-pyran-4-yl)-2,6-diazaspiro[3.3]heptane;
2-methyl-3-((6-(tetrahydro-2H-pyran-4-yl)-2,6-diazaspiro[3.3]heptan-2-yl)sulfonyl)-2,4,5,6-tetrahydrocyclopenta[c]pyrazole;
2-ethyl-6-((2-methyl-6-(trifluoromethyl)pyridin-3-yl)sulfonyl)-2,6-diazaspiro[3.3]heptane;
2-((6-((2-methyl-6-(trifluoromethyl)pyridin-3-yl)sulfonyl)-2,6-diazaspiro[3.3]heptan-2-yl)methyl)cyclopropane-1-carbonitrile;
2-(3-(2-methoxyethoxy)propyl)-6-((2-methyl-6-(trifluoromethyl)-1,2-dihydropyridin-3-yl)sulfonyl)-2,6-diazaspiro[3.3]heptane;
(R)-2-(2-(2,2-dimethyl-1,3-dioxolan-4-yl)ethyl)-6-((2-methyl-6-(trifluoromethyl)pyridin-3-yl)sulfonyl)-2,6-diazaspiro[3.3]heptane;
(1R,3R)-2,2-dimethyl-3-((6-((2-methyl-6-(trifluoromethyl)pyridin-3-yl)sulfonyl)-2,6-diazaspiro[3.3]heptan-2-yl)methyl)cyclopropane-1-carbonitrile;
2-(((1S,2R,5S)-6-oxabicyclo[3.2.1]octan-2-yl)methyl)-6-((2-methyl-6-(trifluoromethyl)pyridin-3-yl)sulfonyl)-2,6-diazaspiro[3.3]heptane;
2-(3-(cyclopentyloxy)propyl)-6-((2-methyl-6-(trifluoromethyl)pyridin-3-yl)sulfonyl)-2,6-diazaspiro[3.3]heptane;
methyl 2,2-dimethyl-4-(6-((2-methyl-6-(trifluoromethyl)-1,4-dihydropyridin-3-yl)sulfonyl)-2,6-diazaspiro[3.3]heptan-2-yl)butanoate;
2-(2-cyclopropyl-2-methoxyethyl)-6-((2-methyl-6-(trifluoromethyl)pyridin-3-yl)sulfonyl)-2,6-diazaspiro[3.3]heptane;
2-(5-methoxypentyl)-6-((2-methyl-6-(trifluoromethyl)pyridin-3-yl)sulfonyl)-2,6-diazaspiro[3.3]heptane;
2-(4-methoxypentyl)-6-((2-methyl-6-(trifluoromethyl)pyridin-3-yl)sulfonyl)-2,6-diazaspiro[3.3]heptane;
2-((1-(2-methoxyethyl)cyclobutyl)methyl)-6-((2-methyl-6-(trifluoromethyl)pyridin-3-yl)sulfonyl)-2,6-diazaspiro[3.3]heptane;
methyl 4-methyl-5-(6-((2-methyl-6-(trifluoromethyl)pyridin-3-yl)sulfonyl)-2,6-diazaspiro[3.3]heptan-2-yl)pentanoate;
2-(3-(ethylthio)propyl)-6-((2-methyl-6-(trifluoromethyl)pyridin-3-yl)sulfonyl)-2,6-diazaspiro[3.3]heptane;
2-(2-cyclopropylethyl)-6-((2-methyl-6-(trifluoromethyl)pyridin-3-yl)sulfonyl)-2,6-diazaspiro[3.3]heptane;
2-(2-(tert-butoxy)ethyl)-6-((2-methyl-6-(trifluoromethyl)-1,2-dihydropyridin-3-yl)sulfonyl)-2,6-diazaspiro[3.3]heptane;
2-((2,2-dimethyltetrahydro-2H-pyran-4-yl)methyl)-6-((2-methyl-6-(trifluoromethyl)pyridin-3-yl)sulfonyl)-2,6-diazaspiro[3.3]heptane;
4-((6-((2-methyl-6-(trifluoromethyl)pyridin-3-yl)sulfonyl)-2,6-diazaspiro[3.3]heptan-2-yl)methyl)tetrahydro-2H-pyran-4-ol;
2-((4-fluorotetrahydro-2H-pyran-4-yl)methyl)-6-((2-methyl-6-(trifluoromethyl)pyridin-3-yl)sulfonyl)-2,6-diazaspiro[3.3]heptane;
2-((3-isopropyl-1-methyl-1H-pyrazol-5-yl)sulfonyl)-6-(tetrahydro-2H-pyran-4-yl)-2,6-diazaspiro[3.3]heptane;
2-((2-cyclopropyl-6-(trifluoromethyl)pyridin-3-yl)sulfonyl)-6-((tetrahydro-2H-pyran-4-yl)methyl)-2,6-diazaspiro[3.3]heptane;
2-((2-methyl-6-(1-(trifluoromethyl)cyclopropyl)-1,4-dihydropyridin-3-yl)sulfonyl)-6-((tetrahydro-2H-pyran-4-yl)methyl)-2,6-diazaspiro[3.3]heptane;
2-((2-chloro-6-(trifluoromethyl)pyridin-3-yl)sulfonyl)-6-(tetrahydro-2H-pyran-4-yl)-2,6-diazaspiro[3.3]heptane;
2-((2-cyclopropyl-6-(trifluoromethyl)pyridin-3-yl)sulfonyl)-6-(tetrahydro-2H-pyran-4-yl)-2,6-diazaspiro[3.3]heptane;
2-((2-methyl-4-(trifluoromethyl)phenyl)sulfonyl)-6-((tetrahydro-2H-pyran-4-yl)methyl)-2,6-diazaspiro[3.3]heptane;
(1s,4s)-4-(6-((2-cyclopropyl-6-(trifluoromethyl)pyridin-3-yl)sulfonyl)-2,6-diazaspiro[3.3]heptan-2-yl)-1-methylcyclohexan-1-ol;
(1s,4s)-4-(6-((2-cyclopropyl-6-(trifluoromethyl)pyridin-3-yl)sulfonyl)-2,6-diazaspiro[3.3]heptan-2-yl)-1-methylcyclohexan-1-ol;
2-((2-methyl-5-(trifluoromethyl)-2,5-dihydro-1H-pyrazol-3-yl)sulfonyl)-6-(((R)-tetrahydrofuran-3-yl)methyl)-2,6-diazaspiro[3.3]heptane;
(R)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane;
2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane;
(S)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane;
2-((tetrahydro-2H-pyran-4-yl)methyl)-6-((2-(trifluoromethyl)-3,4-dihydropyridin-3-yl)sulfonyl)-2,6-diazaspiro[3.3]heptane;
2-((1-methyl-3-(trifluoromethyl)-1H-pyrazol-5-yl)sulfonyl)-6-((tetrahydro-2H-pyran-4-yl)methyl)-2,6-diazaspiro[3.3]heptane;
2-(tetrahydro-2H-pyran-4-yl)-6-((6-(trifluoromethyl)-2-vinylpyridin-3-yl)sulfonyl)-2,6-diazaspiro[3.3]heptane;
2-((2-ethyl-6-(trifluoromethyl)pyridin-3-yl)sulfonyl)-6-(tetrahydro-2H-pyran-4-yl)-2,6-diazaspiro[3.3]heptane;
2-((6-(difluoromethyl)-2-ethylpyridin-3-yl)sulfonyl)-6-(tetrahydro-2H-pyran-4-yl)-2,6-diazaspiro[3.3]heptane;
2-((6-(difluoromethyl)-2-ethylpyridin-3-yl)sulfonyl)-6-(2-oxaspiro[3.3]heptan-6-yl)-2,6-diazaspiro[3.3]heptane;
(1r,4r)-4-(6-((6-(difluoromethyl)-2-ethylpyridin-3-yl)sulfonyl)-2,6-diazaspiro[3.3]heptan-2-yl)-1-methylcyclohexan-1-ol;
(1s,4s)-4-(6-((6-(difluoromethyl)-2-ethylpyridin-3-yl)sulfonyl)-2,6-diazaspiro[3.3]heptan-2-yl)-1-methylcyclohexan-1-ol;
2-((2-cyclopropyl-6-(trifluoromethyl)pyridin-3-yl)sulfonyl)-6-(oxetan-3-ylmethyl)-2,6-diazaspiro[3.3]heptane;
2-((3-(1,1-difluoroethyl)-1-methyl-1H-pyrazol-5-yl)sulfonyl)-6-((tetrahydro-2H-pyran-4-yl)methyl)-2,6-diazaspiro[3.3]heptane;
2-((2,4-bis(trifluoromethyl)phenyl)sulfonyl)-6-((tetrahydro-2H-pyran-4-yl)methyl)-2,6-diazaspiro[3.3]heptane;
(R)-2-((1-methyl-3-(trifluoromethyl)-1H-pyrazol-5-yl)sulfonyl)-6-(1-(tetrahydro-2H-pyran-4-yl)ethyl)-2,6-diazaspiro[3.3]heptane;
2-((6-chloro-2-(trifluoromethyl)-1,4-dihydropyridin-3-yl)sulfonyl)-6-(tetrahydro-2H-pyran-4-yl)-2,6-diazaspiro[3.3]heptane;
2-((2-fluoro-4-(trifluoromethoxy)phenyl)sulfonyl)-6-(tetrahydro-2H-pyran-4-yl)-2,6-diazaspiro[3.3]heptane;
(R)-2-((1,4-dioxan-2-yl)methyl)-6-((1-methyl-3-(trifluoromethyl)-1H-pyrazol-5-yl)sulfonyl)-2,6-diazaspiro[3.3]heptane;
(R)-2-((1,4-dioxan-2-yl)methyl)-6-((2-methyl-6-(trifluoromethyl)pyridin-3-yl)sulfonyl)-2,6-diazaspiro[3.3]heptane;
2-((4-methyl-6-(trifluoromethyl)pyridin-3-yl)sulfonyl)-6-((tetrahydro-2H-pyran-4-yl)methyl)-2,6-diazaspiro[3.3]heptane;
2-((1-methyl-3-(trifluoromethyl)-1H-pyrazol-5-yl)sulfonyl)-6-(oxetan-3-ylmethyl)-2,6-diazaspiro[3.3]heptane;
2-((3-cyclopropyl-1-methyl-1H-pyrazol-5-yl)sulfonyl)-6-((tetrahydro-2H-pyran-4-yl)methyl)-2,6-diazaspiro[3.3]heptane;
2-((2-fluoro-4-(trifluoromethoxy)phenyl)sulfonyl)-6-((tetrahydro-2H-pyran-4-yl)methyl)-2,6-diazaspiro[3.3]heptane;
2-((3-methyl-5-(trifluoromethyl)pyridin-2-yl)sulfonyl)-6-(tetrahydro-2H-pyran-4-yl)-2,6-diazaspiro[3.3]heptane;
(1s,4s)-1-methyl-4-(6-((2-methyl-6-(trifluoromethyl)pyridin-3-yl)sulfonyl)-2,6-diazaspiro[3.3]heptan-2-yl)cyclohexan-1-ol;
(1s,4s)-1-methyl-4-(6-((2-methyl-6-(trifluoromethyl)pyridin-3-yl)sulfonyl)-2,6-diazaspiro[3.3]heptan-2-yl)cyclohexan-1-ol;
2-((3-methyl-5-(trifluoromethyl)pyridin-2-yl)sulfonyl)-6-((tetrahydro-2H-pyran-4-yl)methyl)-2,6-diazaspiro[3.3]heptane;
2-((4-(difluoromethoxy)-2,6-difluorophenyl)sulfonyl)-6-((tetrahydro-2H-pyran-4-yl)methyl)-2,6-diazaspiro[3.3]heptane;
2-((4-(difluoromethoxy)-2-fluorophenyl)sulfonyl)-6-((tetrahydro-2H-pyran-4-yl)methyl)-2,6-diazaspiro[3.3]heptane;
2-((4-(difluoromethoxy)-2-fluorophenyl)sulfonyl)-6-(tetrahydro-2H-pyran-4-yl)-2,6-diazaspiro[3.3]heptane;
2-((6-((tetrahydro-2H-pyran-4-yl)methyl)-2,6-diazaspiro[3.3]heptan-2-yl)sulfonyl)-5-(trifluoromethyl)benzonitrile; and
2-((tetrahydro-2H-pyran-4-yl)methyl)-6-((6-(trifluoromethyl)-2,3-dihydropyridin-3-yl)sulfonyl)-2,6-diazaspiro[3.3]heptane;

or a or a pharmaceutically acceptable salt thereof.

In a fifty-seventh embodiment, the present disclosure provides a pharmaceutical composition comprising a compound according to any one of the preceding embodiments, or a pharmaceutically acceptable salt thereof.

In a fifty-eighth embodiment, the present disclosure provides a method of treating a disease or disorder mediated by EBP comprising administering to a subject an effective amount of a compound according to any one of embodiments one to fifty-six, or a pharmaceutically acceptable salt thereof, or the pharmaceutical composition of the fifty-seventh embodiment.

In a fifty-ninth embodiment, the present disclosure provides a compound according to any one of embodiments one to fifty-six, for use in the treatment of a disease or disorder mediated by EBP.

In a sixtieth embodiment, the present disclosure provides the use of a compound according to any one of embodiments one to fifty-six in the manufacture of a medicament for the treatment of a disease or disorder mediated by EBP.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or IPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have EBP inhibitory activity. As used herein, “EBP inhibitory activity” refers to the ability of a compound or composition to induce a detectable decrease in EBP activity in vivo or in vitro (e.g., at least 10% decrease in EBP activity as measured by a given assay such as the bioassay described in the examples and known in the art).

In certain embodiments, the present disclosure provides a method of treating a disease or disorder responsive to inhibition of EBP activity (referred herein as “EBP mediated disease or disorder” or “disease or disorder mediated by EBP”) in a subject in need of the treatment. The method comprises administering to the subject a compound described herein (e.g., a compound described in any one of the first to fifty-sixth embodiments) or a pharmaceutically acceptable salt thereof or a pharmaceutical composition thereof.

In certain embodiments, the present disclosure provides the use of a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for the manufacture of a medicament for the treatment of a EBP mediated disorder or disease in a subject in need of the treatment.

In certain embodiments, the present disclosure provides a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for use in the treatment of a EBP mediated disorder or disease in a subject in need of the treatment.

In certain embodiments, the EBP mediated disorder is colorectal cancer.

In certain embodiments, the present disclosure provides a method of treating an autoimmune disease in a subject in need of the treatment. The method comprises administering to the subject a compound described herein (e.g., a compound described in any one of the first to fifty-sixth embodiments) or a pharmaceutically acceptable salt thereof or a pharmaceutical composition thereof.

In certain embodiments, the present disclosure provides the use of a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for the manufacture of a medicament for the treatment of an autoimmune disease in a subject in need of the treatment.

In certain embodiments, the present disclosure provides a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for use in the treatment of an autoimmune disease in a subject in need of the treatment.

In certain embodiments, the autoimmune disease is multiple sclerosis (MS). The compounds of the present disclosure can be used for treating all stages of MS, including relapsing multiple sclerosis (or relapsing form(s) of multiple sclerosis), relapsing-remitting multiple sclerosis, primary progress multiple sclerosis, secondary progressive multiple sclerosis and clinically isolated syndrome (hereinafter “CIS”).

Relapsing multiple sclerosis (or relapsing form(s) of multiple sclerosis) includes clinically isolated syndrome, relapsing-remitting multiple sclerosis and active secondary progressive multiple sclerosis.

Relapsing-remitting multiple sclerosis is a stage of MS characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

Primary progressive multiple sclerosis is characterized by the same symptoms of secondary progressive multiple sclerosis, i.e., progressive neurologic decline between acute attacks without any definite periods of remission, without the prior relapsing-remitting stage.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

In certain embodiments, the present disclosure provides a method of promoting myelination in a subject with a myelin-related disease or disorder in a subject in need of the treatment. The method comprises administering to the subject a compound described herein (e.g., a compound described in any one of the first to fifty-sixth embodiments) or a pharmaceutically acceptable salt thereof or a pharmaceutical composition thereof.

In certain embodiments, the present disclosure provides the use of a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for the manufacture of a medicament for promoting myelination in a subject with a myelin-related disease or disorder in a subject in need of the treatment.

In certain embodiments, the present disclosure provides a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for use in promoting myelination in a subject with a myelin-related disease or disorder in a subject in need of the treatment.

In certain embodiments, the myelin-related disease or disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), acute disseminated encephalomyelitis (ADEM), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, autism, metachromatic leukodystrophy, trigeminal neuralgia, acute disseminated encephalitis, chronic inflammatory demyelinating polyneuropathy, Guillian-Barre syndrome, Charcot-Marie-Tooth disease, Bell's palsy and radiation-induced demyelination, for example, neuromyelitis optica (NMO), optic neuritis, pediatric leukodystrophies, neonatal white matter injury, age-related dementia, and schizophrenia.

In certain embodiments, the present disclosure provides a method of treating cancer in a subject in need of the treatment. The method comprises administering to the subject a compound described herein (e.g., a compound described in any one of the first to fifty-sixth embodiments) or a pharmaceutically acceptable salt thereof or a pharmaceutical composition thereof.

In certain embodiments, the present disclosure provides the use of a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for the manufacture of a medicament for the treatment of cancer in a subject in need of the treatment.

In certain embodiments, the present disclosure provides a compound described herein (e.g., a compound described in any one of the first to fifty-sixth embodiments) or a pharmaceutically acceptable salt thereof or a pharmaceutical composition comprising a compound described herein or a pharmaceutically acceptable salt thereof for use in treating cancer in a subject in need of the treatment.

In certain embodiments, the cancer is colorectal cancer.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art. The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O— C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄alkyl group as defined herein, wherein at least of the hydrogen atoms is replaced by an C₁₋₄alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indolyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### EXEMPLIFICATION

### Abbreviations

PE=petroleum ether
EtOAc=EA=ethyl acetate
ESI=electrospray ionisation
MeOH=methanol
EtOH=ethanol
DCE=1,2-dichloroethane
DCM=dichloromethane
CHCl₃=chloroform
HCl=hydrochloric acid
H₂O=water
IPA=isopropyl alcohol
LCMS=liquid chromatography mass spectrometry
HFIP=hexafluoro-2-propanol
HPLC=high pressure liquid chromatography
THF=tetrahydrofuran
MeCN=ACN=acetonitrile
MgSO₄=magnesium sultate
DMSO=dimetylsulfoxide
AcOH=acetic acid
TFA=trifluoroacetic acid
DIPEA=diisopropylethyl amine
N₂=Nitrogen
NH₄HCO₃=Ammonium Bicarbonate
t-BuOH=tert-butanol
NH₄Cl=ammonium chloride
NaH=sodium hydride
Na₂SO₄=sodium sulfate
K₂CO₃=potassium carbonate
NaHCO₃=sodium bicarbonate
NaBH(OAc)₃=STAB=sodium triacetoxyborohydride
SiO₂=silicon dioxide or silica
PDA=Photo Diode Array Detection
TosMIC=toluenesulfonylmethyl isocyanide
TLC=Thin Layer Chromatography
LiHMDS=Lithium bis(trimethylsilyl)amide
DMA=dimethtyl amine
DAST=diethylaminosulfur trifluoride
ee=enantiomeric excess
t_{R}=Retention time

### General Methods

### LCMS Instrumentation Specifications:

Agilent Technologies 1200 Series LC/MSD system: DAD\ELSD Alltech 3300 and Agilent LC\MSD G6130A, G6120B mass-spectrometer.
Agilent Technologies 1260 Infinity LC/MSD system: DAD\ELSD Alltech 3300 and Agilent LC\MSD G6120B mass-spectrometer.
Agilent Technologies 1260 Infinity II LC/MSD system: DAD\ELSD G7102A 1290 Infinity II and Agilent LC\MSD G6120B mass-spectrometer.
Agilent 1260 Series LC/MSD system: DAD\ELSD and Agilent LC\MSD (G6120B) mass-spectrometer.
UHPLC Agilent 1290 Series LC/MSD system: DAD\ELSD and Agilent LC\MSD (G6125B) mass-spectrometer.

### HPLC Analytical Method Specifications:

Column: Agilent Poroshell 120 SB—C18 4.6×30 mm 2.7 μm, with UHPLC Guard Infinity Lab Poroshell 120 SB—C18 4.6×5 mm 2.7 μm
Column Temperature, 60 C
Injection volume 0.5 μL
Modifier: Formic acid 0.1% (v/v) conc.
Method: 99% water/1% MeCN (initial conditions), HOLD initial conditions for 0.1 min, linear gradient to 0% water/100% MeCN at 1.5 min, HOLD 0% water/100% MeCN to 1.73 min, linear gradient to 99% water/1% MeCN at 1.74 min. Flow rate, 3.0 mL/min.
UV scan: 207-223 nm, 246-262 nm, 272-288 nm

### QC Analysis LC/MS Method Conditions:

### Ammonium Hydroxide (Basic pH) Conditions

MS mode: MS:ESI+ scan range 165-650 daltons
PDA: 200-400 nm scan range
Column: Waters ACQUITY UPLC BEH C18 2.1×50 mm, 1.7 μm; Part No. 186002350
Modifier: Ammonium hydroxide 0.2% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### Trifluoroacetic Acid (Acidic pH) Conditions

MS mode: MS:ESI+ scan range 165-650 daltons
PDA: 200-400 nm scan range
Column: Waters ACQUITY UPLC BEH C18 2.1×50 mm, 1.7 μm; Part No. 186002350
Modifier: Trifluoroacetic acid 0.1% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### General Prep HPLC Conditions:

### Ammonium Hydroxide (Basic pH) Conditions

Flow rate: 30 mL/min
MS mode: MS:ESI+ scan range 165-650 daltons
PDA: 200-400 nm scan range
Column: Waters XSELECT CSH C18 PREP 19×100 mm, 5 μm; Part No. 186005421
Modifier: 0.2% Ammonium hydroxide (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate: 50 mL/min
MS mode: MS:ESI+ scan range 165-650 daltons
PDA: 200-400 nm scan range
Column: Waters XSELECT CSH C18 PREP 30×100 mm, 5 μm; Part No. 186005425
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+ scan range 165-650 daltons
PDA: 200-400 nm scan range
Column: Waters XSELECT CSH C18 PREP 30×50 mm, 5 μm; Part No. 186005423
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Trifluoroacetic Acid (Acidic pH) Conditions

Flow rate, 30 mL/min
MS mode: MS:ESI+ scan range 165-650 daltons
PDA: 200-400 nm scan range
Column: Waters Sunfire OBD C18 PREP 19×100 mm, 5 μm; Part No. 186002567
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 50 mL/min
MS mode: MS:ESI+ scan range 165-650 daltons
PDA: 200-400 nm scan range
Column: Waters Sunfire OBD C18 PREP 30×100 mm, 5 μm; Part No. 186002572
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+ scan range 165-650 daltons
PDA: 200-400 nm scan range
Column: Waters Sunfire OBD C18 PREP 30×50 mm, 5 μm; Part No. 186002570
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Example 1: 8-((4-(Difluoromethoxy)phenyl)sulfonyl)-2-(tetrahydro-2H-pyran-4-yl)-5-oxa-2,8-diazaspiro[3.5]nonane

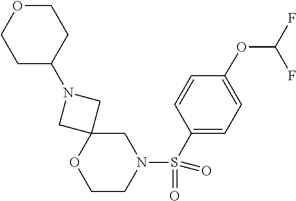 [SMILES: O=S(=O)(c1ccc(OC(F)F)cc1)N1CCOC2(CN(C3CCOCC3)C2)C1]

### 1. Synthesis of tert-butyl 8-((4-(difluoromethoxy)phenyl)sulfonyl)-5-oxa-2,8-diazaspiro[3.5]nonane-2-carboxylate

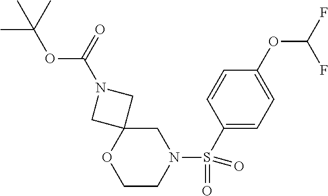 [SMILES: CC(C)(C)OC(=O)N1CC2(C1)CN(S(=O)(=O)c1ccc(OC(F)F)cc1)CCO2]

To a solution of tert-butyl 5-oxa-2,8-diazaspiro[3.5]nonane-2-carboxylate (commercial, 200 mg, 0.9 mmol), DIPEA (460 μL, 2.6 mmol) and DMAP (21 mg, 0.2 mmol) in DCM (5 mL) was added 4-(difluoromethoxy)benzenesulfonyl chloride (234 mg, 1.0 mmol) and the reaction stirred at room temperature for 1 h. The reaction mixture was washed with sat. aq. NH₄Cl, water and brine. The organic layer was dried over Na₂SO₄, filtered and evaporated under reduced pressure to give tert-butyl 8-((4-(difluoromethoxy)phenyl)sulfonyl)-5-oxa-2,8-diazaspiro[3.5]nonane-2-carboxylate that was used without purification. LCMS m/z=379.1 (M+H−^{t}Bu)⁺.

### 2. Synthesis of 8-((4-(difluoromethoxy)phenyl)sulfonyl)-5-oxa-2,8-diazaspiro[3.5]nonane trifluoroacetate

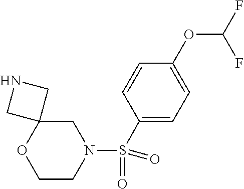 [SMILES: O=S(=O)(c1ccc(OC(F)F)cc1)N1CCOC2(CNC2)C1]

To a solution of tert-butyl 8-((4-(difluoromethoxy)phenyl)sulfonyl)-5-oxa-2,8-diazaspiro[3.5]nonane-2-carboxylate (380 mg, 0.9 mmol) in DCM (4 mL) was added TFA (540 μL, 7.0 mmol) and the reaction mixture was stirred at room temperature overnight. The mixture was concentrated in vacuo and the residue was purified by column chromatography on silica gel (0-100% EtOH:EtOAc (2% NH₄OH)1:3 in heptane) to give the trifluoroacetate salt of 8-((4-(difluoromethoxy)phenyl)sulfonyl)-5-oxa-2,8-diazaspiro[3.5]nonane (302 mg, 77%). LCMS m/z=335 (M+H)⁺.

### 3. Synthesis of 8-((4-(difluoromethoxy)phenyl)sulfonyl)-2-(tetrahydro-2H-pyran-4-yl)-5-oxa-2,8-diazaspiro[3.5]nonane

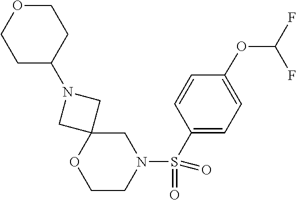 [SMILES: O=S(=O)(c1ccc(OC(F)F)cc1)N1CCOC2(CN(C3CCOCC3)C2)C1]

To the trifluoroacetate salt of 8-((4-(difluoromethoxy)phenyl)sulfonyl)-5-oxa-2,8-diazaspiro[3.5]nonane (50 mg, 0.1 mmol) and tetrahydropyran-4-one (12 mg, 0.1 mmol) in DCM (2 mL) was added AcOH (10 μL, 0.2 mmol) and the reaction was stirred for 30 min. NaBH(OAc)₃ (95 mg, 0.5 mmol) was added, the reaction mixture was stirred at room temperature and monitored to completion. The reaction mixture was concentrated in vacuo and the residue purified by column chromatography on silica gel (0-100% EtOH:EtOAc (2% NH₄OH)1:3 in heptane) to give 8-((4-(difluoromethoxy)phenyl)sulfonyl)-2-(tetrahydro-2H-pyran-4-yl)-5-oxa-2,8-diazaspiro[3.5]nonane (39 mg, 83%). LCMS m/z=419.1 (M+H)⁺. ¹H NMR (500 MHz, CD₃OD) δ (ppm): 7.89-7.80 (m, 2H), 7.38 (d, J=8.6 Hz, 2H), 7.21-6.86 (m, 1H), 3.99-3.88 (m, 2H), 3.72-3.65 (m, 2H), 3.45-3.33 (m, 4H), 3.11 (s, 2H), 3.03 (d, J=9.2 Hz, 2H), 2.97-2.92 (m, 2H), 2.39 (tt, J=4.0, 10.6 Hz, 1H), 1.72 (br dd, J=1.8, 12.2 Hz, 2H), 1.37-1.21 (m, 2H).

### Example 2: 9-((4-(Difluoromethoxy)phenyl)sulfonyl)-2-(tetrahydro-2H-pyran-4-yl)-6-oxa-2,9-diazaspiro[4.5]decane (racemate)

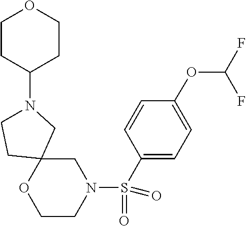 [SMILES: O=S(=O)(c1ccc(OC(F)F)cc1)N1CCOC2(CCN(C3CCOCC3)C2)C1]

### 1. Synthesis of tert-butyl 9-((4-(difluoromethoxy)phenyl)sulfonyl)-6-oxa-2,9-diazaspiro[4.5]decane-2-carboxylate

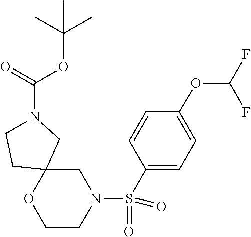 [SMILES: CC(C)(C)OC(=O)N1CCC2(C1)CN(S(=O)(=O)c1ccc(OC(F)F)cc1)CCO2]

To a solution of tert-butyl 6-oxa-2,9-diazaspiro[4.5]decane-2-carboxylate (commercial, 200 mg, 0.8 mmol), DIPEA (430 μL, 2.5 mmol) and DMAP (20 mg, 0.2 mmol) in DCM (5 mL) was added 4-(difluoromethoxy)benzenesulfonyl chloride (220 mg, 0.9 mmol) at room temperature. After 1 h at room temperature the reaction mixture was washed with sat. aq. NH₄Cl, water and brine. The organic phase was dried with Na₂SO₄, filtered and concentrated to yield tert-butyl 9-((4-(difluoromethoxy)phenyl)sulfonyl)-6-oxa-2,9-diazaspiro[4.5]decane-2-carboxylate (370 mg). The crude was used in the next step without further purification. LCMS m/z=434.1 (M+H—CH₃)⁺.

### 2. Synthesis of 9-((4-(difluoromethoxy)phenyl)sulfonyl)-6-oxa-2,9-diazaspiro[4.5]decane

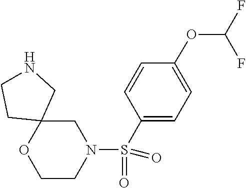 [SMILES: O=S(=O)(c1ccc(OC(F)F)cc1)N1CCOC2(CCNC2)C1]

To a solution of tert-butyl 9-((4-(difluoromethoxy)phenyl)sulfonyl)-6-oxa-2,9-diazaspiro[4.5]decane-2-carboxylate (370 mg, 0.8 mmol) in DCM (4 mL) was added TFA (500 μL, 6.6 mmol). The reaction mixture was stirred at room temperature overnight, concentrated and the resulting residue was purified by column chromatography over silica gel (24 g, 0-100% EtOH:EtOAc (2% NH₄OH)1:3 in heptane) to yield the trifluoroacetate salt of 9-((4-(difluoromethoxy)phenyl)sulfonyl)-6-oxa-2,9-diazaspiro[4.5]decane (568 mg). LCMS m/z=349.0 (M+H)⁺.

### 3. Synthesis of 9-((4-(difluoromethoxy)phenyl)sulfonyl)-2-(tetrahydro-2H-pyran-4-yl)-6-oxa-2,9-diazaspiro[4.5]decane

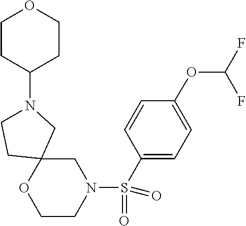 [SMILES: O=S(=O)(c1ccc(OC(F)F)cc1)N1CCOC2(CCN(C3CCOCC3)C2)C1]

The title compound was prepared using a similar method to that described in step 3 for Example 1 from the trifluoroacetate salt of 9-((4-(difluoromethoxy)phenyl)sulfonyl)-6-oxa-2,9-diazaspiro[4.5]decane (50 mg, 0.1 mmol) and tetrahydropyran-4-one (12 mg, 0.1 mmol). The crude was purified by column chromatography on silica gel (24 g, 0-100% EtOH:EtOAc (2% NH₄OH)1:3 in heptane) to afford 9-((4-(difluoromethoxy)phenyl)sulfonyl)-2-(tetrahydro-2H-pyran-4-yl)-6-oxa-2,9-diazaspiro[4.5]decane (17 mg, 35%). LCMS m/z=433.1 (M+H)⁺. ¹H NMR (500 MHz, CD₃OD) δ (ppm): 7.83 (d, J=8.6 Hz, 2H), 7.38 (d, J=9.2 Hz, 2H), 7.20-6.86 (m, 1H), 4.00-3.89 (m, 2H), 3.81-3.67 (m, 2H), 3.40 (br t, J=11.9 Hz, 2H), 3.03-2.89 (m, 3H), 2.87-2.80 (m, 1H), 2.79-2.75 (m, 1H), 2.75-2.66 (m, 3H), 2.29 (tt, J=4.0, 11.0 Hz, 1H), 1.93-1.88 (m, 2H), 1.87-1.78 (m, 2H), 1.56-1.44 (m, 2H).

### Example 3 and 4: (S)-9-((4-(Difluoromethoxy)phenyl)sulfonyl)-2-(tetrahydro-2H-pyran-4-yl)-6-oxa-2,9-diazaspiro[4.5]decane and (R)-9-((4-(difluoromethoxy)phenyl)sulfonyl)-2-(tetrahydro-2H-pyran-4-yl)-6-oxa-2,9-diazaspiro[4.5]decane

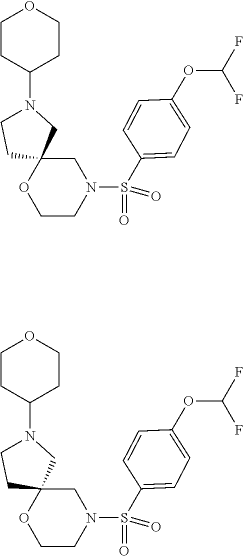 [SMILES: O=S(=O)(c1ccc(OC(F)F)cc1)N1CCO[C@]2(CCN(C3CCOCC3)C2)C1 ; O=S(=O)(c1ccc(OC(F)F)cc1)N1CCO[C@@]2(CCN(C3CCOCC3)C2)C1]

The title compound was prepared using a similar method to that described in step 3 for Example 1 from the trifluoroacetate salt of 9-((4-(difluoromethoxy)phenyl)sulfonyl)-6-oxa-2,9-diazaspiro[4.5]decane (repeat of Example 2 step 3, 432 mg, 1.2 mmol) and tetrahydropyran-4-one (186 mg, 1.9 mmol) to afford racemic 9-((4-(difluoromethoxy)phenyl)sulfonyl)-2-(tetrahydro-2H-pyran-4-yl)-6-oxa-2,9-diazaspiro[4.5]decane (Example 2, 310 mg).

The racemate was separated into its enantiomers with arbitrarily assigned stereochemistry by SFC separation using CHIRALPAK AD-H 30×250 mm, 5 μm; 30% MeOH w/ 0.1% DEA in CO₂ (flow rate: 100 mL/min, ABPR 120 bar, MBPR 40 psi, column temp 40° C.) to yield:

peak 1, enantiomer 1 (S)-9-((4-(Difluoromethoxy)phenyl)sulfonyl)-2-(tetrahydro-2H-pyran-4-yl)-6-oxa-2,9-diazaspiro[4.5]decane (85 mg, 16%, t_{R}=2.02 min, 100% ee, LCMS m/z=433.1 (M+H)⁺)

peak 2, enantiomer 2 (R)-9-((4-(Difluoromethoxy)phenyl)sulfonyl)-2-(tetrahydro-2H-pyran-4-yl)-6-oxa-2,9-diazaspiro[4.5]decane (84 mg, 16%, t_{R}=2.43 min, 95.6% ee, LCMS m/z=433.1 (M+H)⁺).

For both enantiomers: ¹H NMR (500 MHz, CD₃OD) δ (ppm): 7.83 (d, J=8.55 Hz, 2H), 7.38 (d, J=9.16 Hz, 2H), 7.20-6.86 (m, 1H), 4.00-3.89 (m, 2H), 3.81-3.67 (m, 2H), 3.40 (br t, J=11.9 Hz, 2H), 3.03-2.89 (m, 3H), 2.87-2.80 (m, 1H), 2.79-2.75 (m, 1H), 2.75-2.66 (m, 3H), 2.29 (tt, J=4.0, 11.0 Hz, 1H), 1.93-1.88 (m, 2H), 1.87-1.78 (m, 2H), 1.56-1.44 (m, 2H).

### Example 5: 4-((4-(Difluoromethoxy)phenyl)sulfonyl)-9-(tetrahydro-2H-pyran-4-yl)-1-oxa-4,9-diazaspiro[5.5]undecane

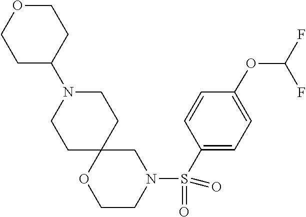 [SMILES: O=S(=O)(c1ccc(OC(F)F)cc1)N1CCOC2(CCN(C3CCOCC3)CC2)C1]

### 1. Synthesis of 4-((4-(difluoromethoxy)phenyl)sulfonyl)-1-oxa-4,9-diazaspiro[5.5]undecane

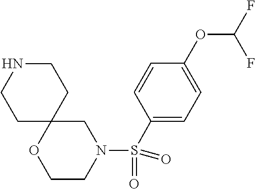 [SMILES: O=S(=O)(c1ccc(OC(F)F)cc1)N1CCOC2(CCNCC2)C1]

To a suspension of tert-butyl 1-oxa-4,9-diazaspiro[5.5]undecane-9-carboxylate (commercial, 1.0 g, 3.9 mmol) and DIPEA (1.0 mL, 5.9 mmol) in DCM (20 mL) was added 4-(difluoromethoxy)benzenesulfonyl chloride (1.0 g, 4.3 mmol) at room temperature. The reaction was monitored to completion. The mixture was diluted with DCM (5 mL) and washed with sat. aq. NH₄Cl, water and brine. The organic layer was dried over Na₂SO₄, filtered and concentrated. The residue was dissolved in EtOAc (40 mL) and HCl (4 M, 5.0 mL) was added at room temperature. The mixture was stirred overnight and a white precipitate formed. Heptane (20 mL) was added, the solids were collected by filtration and dried to afford 4-((4-(difluoromethoxy)phenyl)sulfonyl)-1-oxa-4,9-diazaspiro[5.5]undecane (1.17 g, 75%) which was used without further purification. LCMS m/z=363.1 (M+H)⁺.

### 2. Synthesis of 4-((4-(difluoromethoxy)phenyl)sulfonyl)-9-(tetrahydro-2H-pyran-4-yl)-1-oxa-4,9-diazaspiro[5.5]undecane

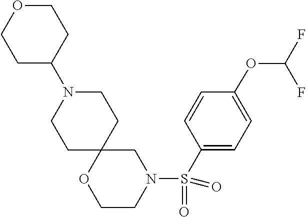 [SMILES: O=S(=O)(c1ccc(OC(F)F)cc1)N1CCOC2(CCN(C3CCOCC3)CC2)C1]

The title compound was prepared using a similar method described in step 3 for Example 1 from the hydrochloride salt of 4-((4-(difluoromethoxy)phenyl)sulfonyl)-1-oxa-4,9-diazaspiro[5.5]undecane (75 mg, 0.2 mmol) and tetrahydropyran-4-one (100 mg, 0.2 mmol). The mixture was purified by column chromatography on silica gel (12 g, 10-100% EtOH:EtOAc 1:3 in heptane) to afford 4-((4-(difluoromethoxy)phenyl)sulfonyl)-9-(tetrahydro-2H-pyran-4-yl)-1-oxa-4,9-diazaspiro[5.5]undecane (51 mg, 61%). LCMS m/z=447.2 (M+H)⁺. ¹H NMR (500 MHz, CD₃OD) δ (ppm): 7.96-7.71 (m, 2H), 7.36 (d, J=9.2 Hz, 2H), 7.22-6.80 (m, 1H), 3.98 (br dd, J=4.3, 11.6 Hz, 2H), 3.80 3.70-3.70 (m, 2H), 3.39 (br t, J=11.3 Hz, 2H), 2.98-2.92 (m, 2H), 2.78 (s, 2H), 2.71-2.63 (m, 2H), 2.57-2.45 (m, 3H), 1.98-1.76 (m, 4H), 1.67-1.47 (m, 4H).

### Example 6: 4-((4-(Difluoromethoxy)phenyl)sulfonyl)-9-(tetrahydrofuran-3-yl)-1-oxa-4,9-diazaspiro[5.5]undecane (racemate)

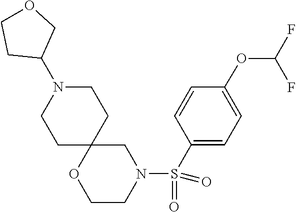 [SMILES: O=S(=O)(c1ccc(OC(F)F)cc1)N1CCOC2(CCN(C3CCOC3)CC2)C1]

To a solution of the hydrochloride salt of 4-((4-(difluoromethoxy)phenyl)sulfonyl)-1-oxa-4,9-diazaspiro[5.5]undecane (Example 5, step 1, 100 mg, 0.3 mmol) and tetrahydrofuran-3-one (27 mg, 0.3 mmol) in DCM (2.5 mL) and AcOH (45 mg, 0.8 mmol) was added NaBH(OAc)₃ (159 mg, 0.8 mmol). The reaction mixture was stirred at room temperature for 2 h. An additional 1.25 equiv. of ketone were added and stirring was continued for 1 h. The reaction mixture was diluted with DCM (5 mL) and washed with sat. aq. NaHCO₃, water and brine. The organic layer was dried with Na₂SO₄, filtered and concentrated in vacuo. Purification by column chromatography on silica gel (24 g, 0-100% EtOH:EtOAc (2% NH₄OH)1:3 in heptane) afforded 4-((4-(difluoromethoxy)phenyl)sulfonyl)-9-(tetrahydrofuran-3-yl)-1-oxa-4,9-diazaspiro[5.5]undecane (90 mg, 83%). LCMS m/z=433.1 (M+H)⁺. ¹H NMR (500 MHz, CD₃OD) δ (ppm): 7.80-7.66 (m, 2H), 7.27 (d, J=8.6 Hz, 2H), 7.12-6.74 (m, 1H), 3.86-3.73 (m, 2H), 3.70-3.61 (m, 3H), 3.53-3.49 (m, 1H), 2.90 (t, J=7.3 Hz, 1H), 2.87-2.80 (m, 2H), 2.69 (s, 2H), 2.60-2.49 (m, 1H), 2.42-2.22 (m, 3H), 2.05-1.95-(m, 1H), 1.82 (br dd, J=3.7, 14.7 Hz, 2H), 1.72 (qd, J=8.3, 12.3 Hz, 1H), 1.56-1.45 (m, 2H).

### Example 7: 4-((4-(Difluoromethoxy)phenyl)sulfonyl)-9-(2-methoxyethyl)-1-oxa-4,9-diazaspiro[5.5]undecane

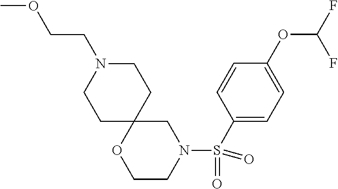 [SMILES: COCCN1CCC2(CC1)CN(S(=O)(=O)c1ccc(OC(F)F)cc1)CCO2]

A mixture of 1-bromo-2-methoxy-ethane (22.5 mg, 162 μmol), 4-((4-(difluoromethoxy)phenyl)sulfonyl)-1-oxa-4,9-diazaspiro[5.5]undecane (Example 5 step 1, 58.7 mg, 162 μmol) and DIPEA (90 μL, 0.5 mmol) in DMF (2 mL) was stirred at room temperature. After 1 d, additional base (3 equiv.) and 1-bromo-2-methoxyethane (1 equiv.) were added. After 1 h, the reaction was quenched with sat. aq. NaHCO₃ solution and extracted with EtOAc (2×). The combined organic layers were washed with sat. aq. NaCl solution, dried with MgSO₄, filtered, evaporated and purified by HPLC (basic conditions) to afford 4-((4-(difluoromethoxy)phenyl)sulfonyl)-9-(2-methoxyethyl)-1-oxa-4,9-diazaspiro[5.5]undecane (17 mg, 25%, 100% purity). LCMS m/z=421.1 (M+H)⁺. LCMS t_{R} (4 min)=1.42 min.

### Examples 8 and 9: (±)—(R)-9-((4-(Difluoromethoxy)phenyl)sulfonyl)-2-((S)-tetrahydro-2H-pyran-3-yl)-6-oxa-2,9-diazaspiro[4.5]decane and (±)—(R)-9-((4-(Difluoromethoxy)phenyl)sulfonyl)-2-((R)-tetrahydro-2H-pyran-3-yl)-6-oxa-2,9-diazaspiro[4.5]decane

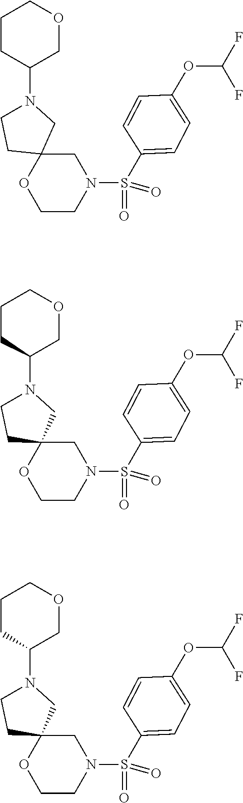 [SMILES: O=S(=O)(c1ccc(OC(F)F)cc1)N1CCOC2(CCN(C3CCCOC3)C2)C1 ; O=S(=O)(c1ccc(OC(F)F)cc1)N1CCO[C@@]2(CCN([C@H]3CCCOC3)C2)C1 ; O=S(=O)(c1ccc(OC(F)F)cc1)N1CCO[C@@]2(CCN([C@@H]3CCCOC3)C2)C1]

The title compound was prepared using a similar method described for Example 1 from 9-((4-(difluoromethoxy)phenyl)sulfonyl)-6-oxa-2,9-diazaspiro[4.5]decane (Example 2 step 2, 150 mg, 0.4 mmol) and tetrahydropyran-3-one (65 mg, 0.7 mmol) to afford the diastereomeric mixture of 9-((4-(difluoromethoxy)phenyl)sulfonyl)-2-(tetrahydro-2H-pyran-3-yl)-6-oxa-2,9-diazaspiro[4.5]decane (200 mg crude). LCMS m/z=433.2 (M+H)⁺.

SFC Chiral separation using CHIRALPAK IC 30×250 mm, 5 μm (40% IPA w/ 0.1% DEA in CO₂ (flow rate: 100 mL/min, ABPR 120 bar, MBPR 60 psi, column temp 40° C.)) resulted in 9-((4-(difluoromethoxy)phenyl)sulfonyl)-2-(tetrahydro-2H-pyran-3-yl)-6-oxa-2,9-diazaspiro[4.5]decane as two diastereomeric pairs of arbitrarily assigned stereochemistry: D1 (50 mg, 27%, t_{R}=1.92 min, 100% de) and D2 (50 mg, 27%, t_{R}=2.15 min, 88.1% de).

### Example 10 and Example 11: 1-(4-((4-(Difluoromethoxy)phenyl)sulfonyl)-1-oxa-4,9-diazaspiro[5.5]undecan-9-yl)-2-methylpropan-2-ol and 4-((4-(Difluoromethoxy)phenyl)sulfonyl)-9-(2-methoxy-2-methylpropyl)-1-oxa-4,9-diazaspiro[5.5]undecane

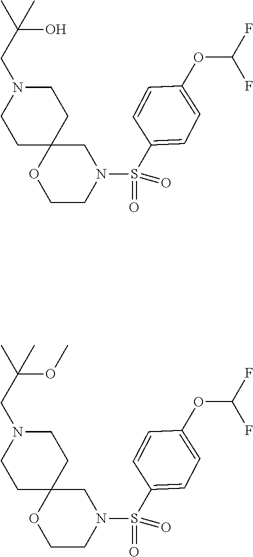 [SMILES: CC(C)(O)CN1CCC2(CC1)CN(S(=O)(=O)c1ccc(OC(F)F)cc1)CCO2 ; COC(C)(C)CN1CCC2(CC1)CN(S(=O)(=O)c1ccc(OC(F)F)cc1)CCO2]

### 1. Synthesis of ethyl 2-(4-((4-(difluoromethoxy)phenyl)sulfonyl)-1-oxa-4,9-diazaspiro[5.5]undecan-9-yl)acetate

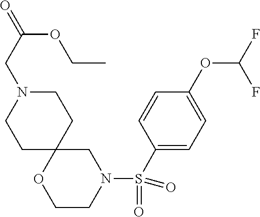 [SMILES: CCOC(=O)CN1CCC2(CC1)CN(S(=O)(=O)c1ccc(OC(F)F)cc1)CCO2]

To a solution of 4-((4-(difluoromethoxy)phenyl)sulfonyl)-1-oxa-4,9-diazaspiro[5.5]undecane hydrochloride (Example 5, step 1, 250 mg, 0.6 mmol) and NEt₃ (110 μL, 0.8 mmol) in THE (2 mL) was added ethyl 2-bromoacetate (115 mg, 0.7 mmol) until complete consumption of the starting material as determined by LCMS. The reaction mixture was diluted with EtOAc (10 mL) and washed with sat. aq. NH₄Cl, water and brine. The organic phase was dried over Na₂SO₄, filtered and evaporated to dryness to afford ethyl 2-(4-((4-(difluoromethoxy)phenyl)sulfonyl)-1-oxa-4,9-diazaspiro[5.5]undecan-9-yl)acetate (296 mg) which was used without further purification. LCMS m/z=448.8 (M+H)⁺.

### 2. Synthesis of 1-(4-((4-(difluoromethoxy)phenyl)sulfonyl)-1-oxa-4,9-diazaspiro[5.5]undecan-9-yl)-2-methylpropan-2-ol

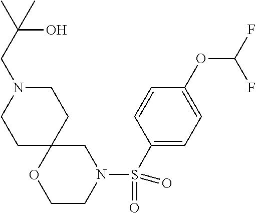 [SMILES: CC(C)(O)CN1CCC2(CC1)CN(S(=O)(=O)c1ccc(OC(F)F)cc1)CCO2]

To a solution of ethyl 2-(4-((4-(difluoromethoxy)phenyl)sulfonyl)-1-oxa-4,9-diazaspiro[5.5]undecan-9-yl)acetate (271 mg, 0.6 mmol) in THE (6 mL) was added MeMgBr (2 M, 900 μL) at room temperature. The reaction mixture was stirred for 30 min, then diluted with EtOAc (5 mL) and washed with sat. aq. NaHCO₃, water and brine. The organic phase was dried over Na₂SO₄, filtered, concentrated and purified by HPLC (acidic conditions) to afford the trifluoroacetate salt of 1-(4-((4-(difluoromethoxy)phenyl)sulfonyl)-1-oxa-4,9-diazaspiro[5.5]undecan-9-yl)-2-methylpropan-2-ol (49 mg, 15%). LCMS m/z=435.9 (M+H)⁺. ¹H NMR (500 MHz, DMSO-d₆) δ (ppm) 7.79 (br d, J=8.6 Hz, 2H), 7.60-7.24 (m, 3H), 4.21 (q, J=7.1 Hz, 4H), 3.70 (t, J=4.9 Hz, 2H), 3.41-2.97 (m, 1H), 2.93-2.69 (m, 4H), 2.52-2.47 (m, 2H), 2.23-1.58 (m, 5H), 1.22 (t, J=7.0 Hz, 3H).

### 3. Synthesis of 4-((4-(difluoromethoxy)phenyl)sulfonyl)-9-(2-methoxy-2-methylpropyl)-1-oxa-4,9-diazaspiro[5.5]undecane

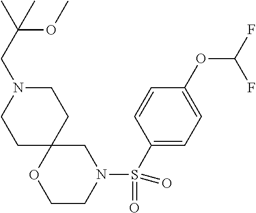 [SMILES: COC(C)(C)CN1CCC2(CC1)CN(S(=O)(=O)c1ccc(OC(F)F)cc1)CCO2]

To a solution of Example 12, the trifluoroacetate salt of 1-(4-((4-(difluoromethoxy)phenyl)sulfonyl)-1-oxa-4,9-diazaspiro[5.5]undecan-9-yl)-2-methylpropan-2-ol (50 mg, 0.1 mmol) and iodomethane (20 μL, 0.3 mmol) in THE (2 mL) was added sodium hydride (10 mg, 0.3 mmol, 60% purity) at room temperature. The reaction mixture was stirred for 3 d, then diluted with EtOAc (5 mL) and washed with sat. aq. NH₄Cl, water and brine. The organic phase was dried over Na₂SO₄, filtered and concentrated and purified by HPLC (basic conditions) to afford 4-((4-(difluoromethoxy)phenyl)sulfonyl)-9-(2-methoxy-2-methylpropyl)-1-oxa-4,9-diazaspiro[5.5]undecane (52 mg, 100%). LCMS m/z=449.1 (M+H)⁺. ¹H NMR (500 MHz, CD₃OD) δ (ppm): 7.90-7.73 (m, 2H), 7.37 (d, J=8.6 Hz, 2H), 7.26-6.80 (m, 1H), 3.82-3.69 (m, 2H), 3.20 (s, 3H), 2.98-2.91 (m, 2H), 2.78 (s, 2H), 2.71-2.49 (m, 4H), 2.39 (br s, 2H), 1.83 (br d, J=13.4 Hz, 2H), 1.72-1.53 (m, 2H), 1.17 (s, 6H).

### Examples 12 and 13

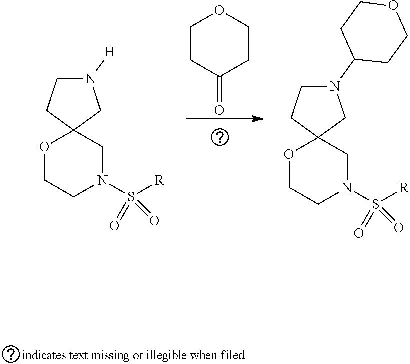

The Examples 12 and 13 in Table A were prepared using a similar method described in step 3 for Example 1 from tetrahydropyran-4-one (26 mg, 0.3 mmol) and the corresponding amine. The sulfonamide starting materials were prepared using the standard methods as described above, e.g. in Example 1, step 1 and 2 (1.1 equiv. sulfonyl chloride). Purification of the final products after reductive amination were conducted by HPLC, either under acidic or basic conditions.

TABLE A

| Example | R = | Product | Data |
|---|---|---|---|
| 12 | 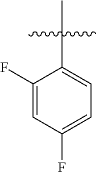 [SMILES: *c1ccc(F)cc1F] 9-((2,4- difluorophenyl)sulfonyl)- 6-oxa-2,9- diazaspiro[4.5]decane (66 mg, 0.2 mmol) LCMS m/z = 319.1 (M + H)⁺. | 9-((2,4- Difluorophenyl)sulfonyl)- 2-(tetrahydro-2H-pyran-4- yl)-6-oxa-2,9- diazaspiro[4.5]decane 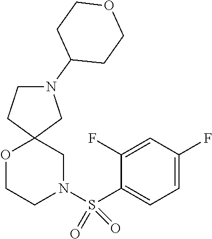 [SMILES: O=S(=O)(c1ccc(F)cc1F)N1CCOC2(CCN(C3CCOCC3)C2)C1] | Yield: 57 mg, 68%; trifluoroacetate salt LCMS m/z = 403.0 (M + H)⁺ ¹H NMR (600 MHz, DMSO-d₆) δ (ppm) 7.95-7.78 (m, 1H), 7.63 (br t, J = 8.7 Hz, 1H), 7.37 (br t, J = 8.4 Hz, 1H), 3.94 (br d, J = 8.00 Hz, 3H), 3.88- 3.74 (m, 3H), 3.41- 2.97 (m, 7H), 2.14 (br d, J = 5.8 Hz, 2H), 2.04- 1.79 (m, 3H), 1.70- 1.46 (m, 3H). |
| 13 | 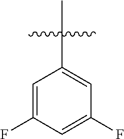 [SMILES: *c1cc(F)cc(F)c1] 9-((3,5- difluorophenyl)sulfonyl)- 6-oxa-2,9- diazaspiro[4.5]decane (66 mg, 0.2 mmol) LCMS m/z = 319.1 (M + H)⁺. | 9-((3,5- Difluorophenyl)sulfonyl)- 2-(tetrahydro-2H-pyran-4- yl)-6-oxa-2,9- diazaspiro[4.5]decane 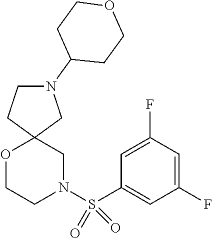 [SMILES: O=S(=O)(c1cc(F)cc(F)c1)N1CCOC2(CCN(C3CCOCC3)C2)C1] | Yield: 54 mg, 66% LCMS m/z = 403.0 (M + H)⁺. ¹H NMR (600 MHz, DMSO-d₆) δ (ppm) 7.72 (br t, J = 9.1 Hz, 1H), 7.52 (br d, J = 4.4 Hz, 2H), 3.84 (br d, J = 10.2 Hz, 4H), 3.73- 3.56 (m, 2H), 3.06- 2.74 (m, 3H), 1.84- 1.66 (m, 8H), 1.34 (br d, J = 8.0 Hz, 4H). |

### Examples 14 and 15: (S)-9-((3,5-Difluorophenyl)sulfonyl)-2-(tetrahydro-2H-pyran-4-yl)-6-oxa-2,9-diazaspiro[4.5]decane and (R)-9-((3,5-Difluorophenyl)sulfonyl)-2-(tetrahydro-2H-pyran-4-yl)-6-oxa-2,9-diazaspiro[4.5]decane

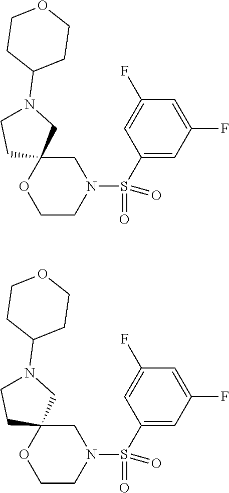 [SMILES: O=S(=O)(c1cc(F)cc(F)c1)N1CCO[C@]2(CCN(C3CCOCC3)C2)C1 ; O=S(=O)(c1cc(F)cc(F)c1)N1CCO[C@@]2(CCN(C3CCOCC3)C2)C1]

The racemic Example 15 was separated into its enantiomers of arbitrarily assigned stereochemistry by chiral separation using CHIRALPAK AD-H 30×250 mm, 5 μm; 20% MeOH w/ 0.1% DMEA in CO₂ (flow rate: 100 mL/min, ABPR 120 bar, MBPR 40 psi, column temp 40° C.) to yield peak 1, enantiomer 1 (10.2 mg, 20%, t_{R}=1.90 min, 100% ee) and peak 2, enantiomer 2 (11.6 mg, 23%, t_{R}=2.03 min, 85.8% ee).

E1 ¹H NMR (600 MHz, DMSO-d₆) δ (ppm) 7.72 (br t, J=9.1 Hz, 1H), 7.52 (br d, J=4.4 Hz, 2H), 3.84 (br d, J=10.2 Hz, 4H), 3.76-3.56 (m, 2H), 3.05-2.72 (m, 3H), 1.88-1.65 (m, 8H), 1.34 (br d, J=8.7 Hz, 4H).

E2 ¹H NMR (600 MHz, DMSO-d₆) δ (ppm) 7.72 (br t, J=9.1 Hz, 1H), 7.52 (br d, J=4.4 Hz, 2H), 3.84 (br d, J=10.2 Hz, 4H), 3.73-3.56 (m, 2H), 3.06-2.74 (m, 3H), 1.84-1.66 (m, 8H), 1.34 (br d, J=8.0 Hz, 4H).

### Example 16: 9-((2-Methoxy-5-methylpyridin-3-yl)sulfonyl)-2-(tetrahydro-2H-pyran-4-yl)-6-oxa-2,9-diazaspiro[4.5]decane

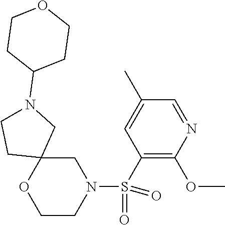 [SMILES: COc1ncc(C)cc1S(=O)(=O)N1CCOC2(CCN(C3CCOCC3)C2)C1]

### 1: Synthesis of tert-butyl 9-((2-methoxy-5-methylpyridin-3-yl)sulfonyl)-6-oxa-2,9-diazaspiro[4.5]decane-2-carboxylate

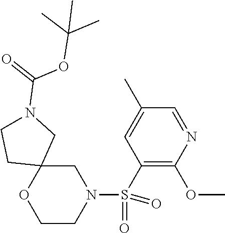 [SMILES: COc1ncc(C)cc1S(=O)(=O)N1CCOC2(CCN(C(=O)OC(C)(C)C)C2)C1]

The title compound was prepared using a similar method described for the preparation of the intermediate 4-((4-(difluoromethoxy)phenyl)sulfonyl)-1-oxa-4,9-diazaspiro[5.5]undecane (Example 5, step 1) from tert-butyl 6-oxa-2,9-diazaspiro[4.5]decane-2-carboxylate (500 mg, 2.1 mmol) and 2-methoxy-5-methylpyridine-3-sulfonyl chloride (457 mg, 2.1 mmol) to afford tert-butyl 9-((2-methoxy-5-methylpyridin-3-yl)sulfonyl)-6-oxa-2,9-diazaspiro[4.5]decane-2-carboxylate (1.6 g).

### 2: Synthesis of 9-((2-methoxy-5-methylpyridin-3-yl)sulfonyl)-6-oxa-2,9-diazaspiro[4.5]decane

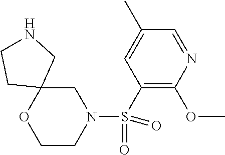 [SMILES: COc1ncc(C)cc1S(=O)(=O)N1CCOC2(CCNC2)C1]

To a solution of tert-butyl 9-((2-methoxy-5-methylpyridin-3-yl)sulfonyl)-6-oxa-2,9-diazaspiro[4.5]decane-2-carboxylate (300 mg, 0.7 mmol) in 1,1,1,3,3,3-hexafluoropropan-2-ol (15 mL) was added TFA (160 μL, 2.1 mmol) at 25° C. The mixture was stirred at 25° C. for 2 hours. The mixture was concentrated to give the title compound (220 mg, 96%) as light yellow oil. LCMS: 328.3 (M+H)⁺.

### Step 3: 9-((2-methoxy-5-methylpyridin-3-yl)sulfonyl)-2-(tetrahydro-2H-pyran-4-yl)-6-oxa-2,9-diazaspiro[4.5]decane

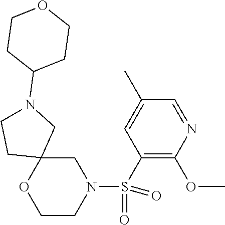 [SMILES: COc1ncc(C)cc1S(=O)(=O)N1CCOC2(CCN(C3CCOCC3)C2)C1]

A solution of 9-((2-methoxy-5-methylpyridin-3-yl)sulfonyl)-6-oxa-2,9-diazaspiro[4.5]decane (110 mg, 0.3 mmol) and tetrahydropyran-4-one (34 mg, 0.3 mmol) in MeOH (10 mL) was adjusted to pH=5-6 with AcOH before NaBH₃CN (63 mg, 1.0 mmol) was added. The reaction mixture was stirred at 95° C. for 12 h. The mixture was concentrated to give a residue which was purified by prep HPLC (Column: Phenomenex C18 150×25 mm×10 μm; Condition: water (NH₄HCO₃)-ACN Begin B 18%; End B: 48% Gradient Time (min): 8; 100% B Hold Time (min): 2; Flow Rate (mL/min): 30) to afford 9-((2-methoxy-5-methylpyridin-3-yl)sulfonyl)-2-(tetrahydro-2H-pyran-4-yl)-6-oxa-2,9-diazaspiro[4.5]decane as an off-white solid. (29 mg, 20%). LCMS: 412.1 (M+H)⁺. ¹H NMR: (400 MHz, CD₃OD) δ (ppm): 8.22 (d, J=1.6 Hz, 1H), 8.00 (d, J=1.6 Hz, 1H), 4.04 (s, 3H), 3.96-3.90 (m, 2H), 3.76-3.74 (m, 2H), 3.46-3.37 (m, 2H), 3.30-3.22 (m, 2H), 3.21-3.16 (m, 1H), 3.12-3.03 (m, 1H), 2.79-2.63 (m, 4H), 2.37-2.23 (m, 4H), 1.95-1.76 (m, 4H), 1.55-1.40 (m, 2H).

### Examples 17 and 18

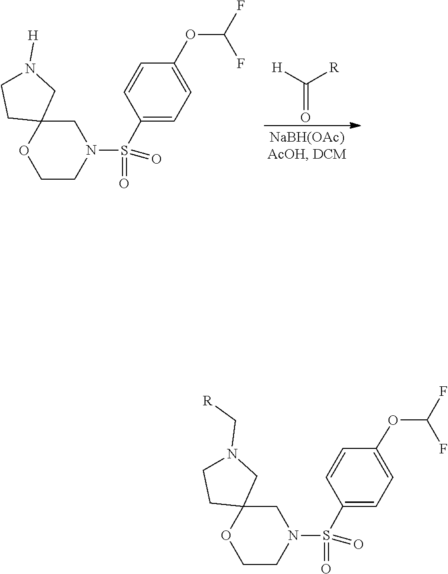

The Examples 17 and 18 in Table B were prepared using a similar method described in step 3 for Example 1 from the hydrochloride salt of 9-((4-(difluoromethoxy)phenyl)sulfonyl)-6-oxa-2,9-diazaspiro[4.5]decane (Example 2, step 2, 50 mg, 0.1 mmol) and the corresponding aldehyde. Purification by HPLC, basic conditions.

TABLE B

| Example | Aldehyde | Product | Data |
|---|---|---|---|
| 17 | 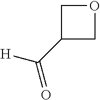 Oxetane-3- carbaldehyde (14 mg, 0.2 mmol) | 9-((4- (Difluoromethoxy)phenyl)sulfonyl)-2- (oxetan-3-ylmethyl)-6-oxa-2,9- diazaspiro[4.5]decane 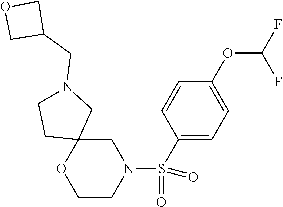 [SMILES: O=S(=O)(c1ccc(OC(F)F)cc1)N1CCOC2(CCN(CC3COC3)C2)C1] | Yield: 39 mg, 72% LCMS m/z = 419.2 (M + H)⁺. ¹H NMR (600 MHz, DMSO- d₆) δ (ppm) 7.88 (d, J = 8.7 Hz, 2H), 7.49 (d, J = 8.0 Hz, 3H), 4.71-4.65 (m, 2H), 4.30 (t, J = 6.2 Hz, 2H), 3.76-3.61 (m, 2H), 2.99-2.80 (m, 3H), 2.80- 2.65 (m, 4H), 2.65-2.38- (m, 4H), 1.86-1.74 (m, 2H) |
| 18 | 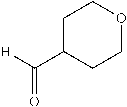 Tetrahydropyran- 4-carbaldehyde (19 mg, 0.2 mmol) | 9-((4- (Difluoromethoxy)phenyl)sulfonyl)-2- ((tetrahydro-2H-pyran-4-yl)methyl)-6- oxa-2,9-diazaspiro[4.5]decane 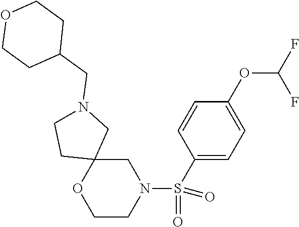 [SMILES: O=S(=O)(c1ccc(OC(F)F)cc1)N1CCOC2(CCN(CC3CCOCC3)C2)C1] | Yield: 43 mg, 74% LCMS m/z = 447.2 (M + H)⁺. ¹H NMR (600 MHz, DMSO- d₆) δ (ppm) 7.81 (d, J = 8.0 Hz, 2H), 7.61-7.18 (m, 3H), 3.82 (br d, J = 10.2 Hz, 3H), 3.76- 3.52 (m, 1H), 3.43-3.20 (m, 2H), 3.00-2.56 (m, 4H), 2.50 (d, J = 1.5 Hz, 1H), 1.77 (br s, 4H), 1.59 (br d, J = 12.4 Hz, 4H), 1.20-1.01 (m, 4H). |

### Examples 19 and 20

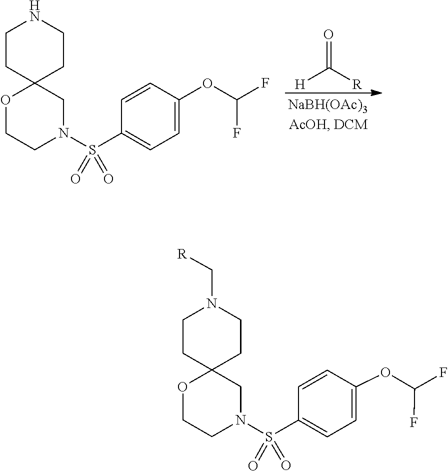

The Examples 19 and 20 in Table C were prepared using a similar method described in step 3 for Example 1 from 4-((4-(difluoromethoxy)phenyl)sulfonyl)-1-oxa-4,9-diazaspiro[5.5]undecane (Example 5, step 1, 50 mg, 0.1 mmol) and the corresponding aldehyde. Purified by HPLC, either acidic or basic conditions.

TABLE C

| Example | Aldehyde | Product | Data |
|---|---|---|---|
| 19 | 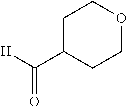 Tetrahydropyran- 4-carbaldehyde (20 mg, 0.2 mmol) | 4-((4-(Difluoromethoxy)phenyl) sulfonyl)-9- ((tetrahydro-2H-pyran-4- yl)methyl)- 1-oxa-4,9-diazaspiro[5.5]undecane 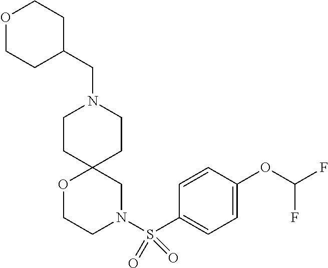 [SMILES: O=S(=O)(c1ccc(OC(F)F)cc1)N1CCOC2(CCN(CC3CCOCC3)CC2)C1] | Yield: 63 mg, 99% Trifluoroacetate salt. LCMS m/z = 461.3 (M + H)⁺. ¹H NMR (600 MHz, DMSO-d₆) δ (ppm) 7.81 (d, J = 8.7 Hz, 2H), 7.58-7.22 (m, 3H), 3.84 (br d, J = 9.5 Hz, 3H), 3.72 (br t, J = 4.4 Hz, 2H), 3.39-3.24 (m, 1H), 3.15-2.84 (m, 4H), 2.49 (s, 2H), 2.09 (br d, J = 14.5 Hz, 2H), 2.01 (br dd, J = 4.4, 7.3 Hz, 2H), 1.87- 1.58 (m, 6H), 1.30-1.13 (m, 3H). |
| 20 | 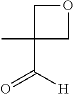 3-Methyloxetane- 3-carbaldehyde (17.3 mg, 172 μmol) | 4-((4- (Difluoromethoxy)phenyl)sulfonyl)- 9-((3-methyloxetan-3-yl)methyl)- 1-oxa-4,9-diazaspiro[5.5]undecane 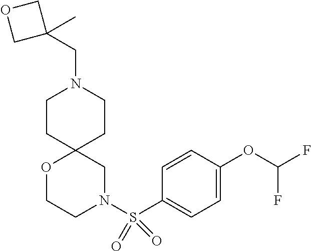 [SMILES: CC1(CN2CCC3(CC2)CN(S(=O)(=O)c2ccc(OC(F)F)cc2)CCO3)COC1] | Yield: 34 mg, 55% LCMS m/z = 447.3 (M + H)⁺. ¹H NMR (600 MHz, DMSO-d₆) δ (ppm) 7.88 (br d, J = 8.0 Hz, 2H), 7.70- 7.19 (m, 3H), 4.65-4.12 (m, 5H), 3.77 (br s, 2H), 3.23-2.69 (m, 4H), 2.56 (s, 2H), 2.36-1.98 (m, 2H), 1.77 (br s, 2H), 1.66-1.20 (m, 6H). |

### Example 21: 4-((4-(Difluoromethoxy)phenyl)sulfonyl)-9-(2,2-dimethyltetrahydro-2H-pyran-4-yl)-1-oxa-4,9-diazaspiro[5.5]undecane

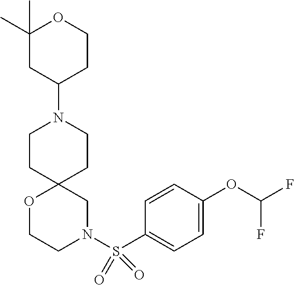 [SMILES: CC1(C)CC(N2CCC3(CC2)CN(S(=O)(=O)c2ccc(OC(F)F)cc2)CCO3)CCO1]

The title compound was prepared using a similar method described in step 3 for Example 1 from 2,2-dimethyltetrahydropyran-4-one (21 mg, 0.2 mmol) and the hydrochloride salt of 4-((4-(difluoromethoxy)phenyl)sulfonyl)-1-oxa-4,9-diazaspiro[5.5]undecane (Example 5, step 1, 52 mg, 0.1 mmol) to afford 4-((4-(difluoromethoxy)phenyl)sulfonyl)-9-(2,2-dimethyltetrahydro-2H-pyran-4-yl)-1-oxa-4,9-diazaspiro[5.5]undecane (7 mg, 9%). LCMS m/z=475.3 (M+H)⁺. ¹H NMR (500 MHz, CD₃OD) δ (ppm) 7.88-7.79 (m, 2H), 7.42-7.31 (m, 2H), 7.21-6.84 (m, 1H), 3.78-3.63 (m, 4H), 3.01-2.79 (m, 4H), 2.79-2.63 (m, 4H), 2.46 (tt, J=4.0, 11.6 Hz, 1H), 1.93-1.76 (m, 4H), 1.42-1.31 (m, 1H), 1.30-1.14 (m, 9H).

### Example 22: 8-((2-Cyclopropylthiazol-5-yl)sulfonyl)-2-(tetrahydro-2H-pyran-4-yl)-5-oxa-2,8-diazaspiro[3.5]nonane

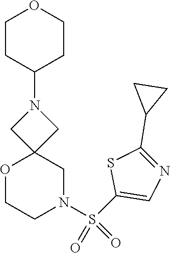 [SMILES: O=S(=O)(c1cnc(C2CC2)s1)N1CCOC2(CN(C3CCOCC3)C2)C1]

### 1. Synthesis of 8-((2-cyclopropylthiazol-5-yl)sulfonyl)-5-oxa-2,8-diazaspiro[3.5]nonane

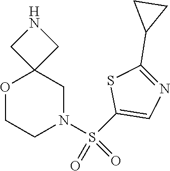 [SMILES: O=S(=O)(c1cnc(C2CC2)s1)N1CCOC2(CNC2)C1]

The title compound was prepared using a similar method described for Example 5, step 1, from tert-butyl 5-oxa-2,8-diazaspiro[3.5]nonane-2-carboxylate (commercial, 60 mg, 0.3 mmol) and 2-cyclopropylthiazole-5-sulfonyl chloride (65 mg, 0.3 mmol) to afford tert-butyl 8-((2-cyclopropylthiazol-5-yl)sulfonyl)-5-oxa-2,8-diazaspiro[3.5]nonane-2-carboxylate. The crude was stirred with 1N HCl to afford the hydrochloride salt of 8-((2-cyclopropylthiazol-5-yl)sulfonyl)-5-oxa-2,8-diazaspiro[3.5]nonane (83 mg) which was used without further purification. LCMS m/z=316.0 (M+H)⁺.

### 2. Synthesis of 8-((2-cyclopropylthiazol-5-yl)sulfonyl)-2-(tetrahydro-2H-pyran-4-yl)-5-oxa-2,8-diazaspiro[3.5]nonane

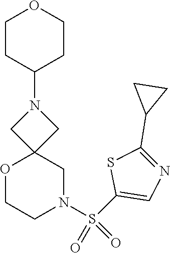 [SMILES: O=S(=O)(c1cnc(C2CC2)s1)N1CCOC2(CN(C3CCOCC3)C2)C1]

The title compound was prepared using a similar method described in step 3 for Example 1 from tetrahydropyran-4-one (26 mg, 0.3 mmol) and the hydrochloride salt of 8-((2-cyclopropylthiazol-5-yl)sulfonyl)-5-oxa-2,8-diazaspiro[3.5]nonane (83 mg). Purification by column chromatography on silica gel (12 g, 10-100% EtOH:EtOAc 1:3 in heptane) afforded 8-((2-cyclopropylthiazol-5-yl)sulfonyl)-2-(tetrahydro-2H-pyran-4-yl)-5-oxa-2,8-diazaspiro[3.5]nonane (39 mg, 37%). LCMS m/z=400.2 (M+H)⁺. ¹H NMR (500 MHz, CD₃OD) δ (ppm) 8.00 (s, 1H), 3.97-3.86 (m, 2H), 3.77-3.64 (m, 2H), 3.47-3.33 (m, 4H), 3.07-2.96 (m, 4H), 2.50-2.32 (m, 2H), 1.76-1.67 (m, 3H), 1.34-1.22 (m, 5H), 1.19-1.12 (m, 2H).

### Example 23: 9-((2-Cyclopropylthiazol-5-yl)sulfonyl)-2-(tetrahydro-2H-pyran-4-yl)-6-oxa-2,9-diazaspiro[4.5]decane

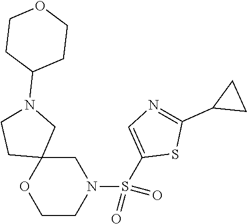 [SMILES: O=S(=O)(c1cnc(C2CC2)s1)N1CCOC2(CCN(C3CCOCC3)C2)C1]

### 1. Synthesis of 9-((2-cyclopropylthiazol-5-yl)sulfonyl)-6-oxa-2,9-diazaspiro[4.5]decane

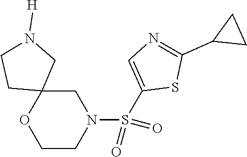 [SMILES: O=S(=O)(c1cnc(C2CC2)s1)N1CCOC2(CCNC2)C1]

To a solution of tert-butyl 6-oxa-2,9-diazaspiro[4.5]decane-2-carboxylate (50 mg, 0.2 mmol) and DIPEA (70 μL, 0.4 mmol) in DCM (2 mL) was added 2-cyclopropylthiazol-5-sulfonylchloride (46 mg, 0.2 mmol) at room temperature. The reaction mixture was diluted with EtOAc (5 mL) and washed with sat. aq. NH₄Cl, water and brine. The combined organic layer was dried over Na₂SO₄, filtered and concentrated. LCMS m/z=374.1 (M+H-^{t}Bu)⁺. The crude was dissolved in EtOAc (2 mL) and HCl (1 M in EtOAc, 1 mL) was added. The reaction mixture was stirred overnight, the organic layers were removed under reduced pressure and the resulting hydrochloride salt of 9-((2-cyclopropylthiazol-5-yl)sulfonyl)-6-oxa-2,9-diazaspiro[4.5]decane was used without further purification in the next step assuming 100% yield (88.6 mg). LCMS m/z=330.0 (M+H)⁺.

### 2. Synthesis of 9-((2-cyclopropylthiazol-5-yl)sulfonyl)-2-(tetrahydro-2H-pyran-4-yl)-6-oxa-2,9-diazaspiro[4.5]decane

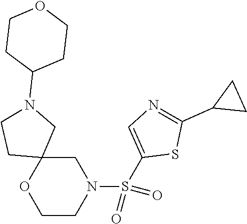 [SMILES: O=S(=O)(c1cnc(C2CC2)s1)N1CCOC2(CCN(C3CCOCC3)C2)C1]

The title compound was prepared using a similar method described in step 3 for Example 1 from tetrahydropyran-4-one (21 mg, 0.2 mmol) and the hydrochloride salt of 9-((2-cyclopropylthiazol-5-yl)sulfonyl)-6-oxa-2,9-diazaspiro[4.5]decane (45 mg, 0.1 mmol) to afford, after purification by HPLC (basic conditions), 9-((2-cyclopropylthiazol-5-yl)sulfonyl)-2-(tetrahydro-2H-pyran-4-yl)-6-oxa-2,9-diazaspiro[4.5]decane (29 mg, 51%). LCMS m/z=414.2 (M+H)⁺. ¹H NMR (500 MHz, DMSO-d₆) δ (ppm) 8.10 (s, 1H), 3.82 (br d, J=11.6 Hz, 2H), 3.60-3.77 (m, 2H), 3.28 (br s, 1H), 3.01-2.84 (m, 3H), 2.82-2.62 (m, 3H), 2.59-2.55 (m, 1H), 2.52-2.46 (m, 2H), 2.43 (br d, J=9.8 Hz, 1H), 2.26-2.13 (m, 1H), 1.82-1.66 (m, 4H), 1.40-1.26 (m, 2H), 1.26-1.19 (m, 2H), 1.15-1.06 (m, 2H).

### Example 24: 9-((2-Cyclopropylthiazol-5-yl)sulfonyl)-2-(2-oxaspiro[3.3]heptan-6-yl)-6-oxa-2,9-diazaspiro[4.5]decane

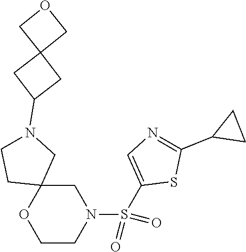 [SMILES: O=S(=O)(c1cnc(C2CC2)s1)N1CCOC2(CCN(C3CC4(COC4)C3)C2)C1]

The title compound was prepared using a similar method described in step 3 for Example 1 from 2-oxa-spiro[3.3]heptan-6-one (23 mg, 0.2 mmol) and the hydrochloride salt of 9-((2-cyclopropylthiazol-5-yl)sulfonyl)-6-oxa-2,9-diazaspiro[4.5]decane (Example 23, step 1, 45 mg, 0.1 mol) to afford 9-((2-cyclopropylthiazol-5-yl)sulfonyl)-2-(2-oxaspiro[3.3]heptan-6-yl)-6-oxa-2,9-diazaspiro[4.5]decane (6.4 mg, 15%). LCMS m/z=426.2 (M+H)⁺. ¹H NMR (500 MHz, DMSO-d₆) δ (ppm) 8.10 (s, 1H), 4.55 (s, 2H), 4.45 (s, 2H), 3.75-3.57 (m, 2H), 3.06-2.91 (m, 2H), 2.90-2.81 (m, 1H), 2.75-2.64 (m, 2H), 2.59-2.52 (m, 1H), 2.50 (td, J=1.8, 3.7 Hz, 2H), 2.38-2.18 (m, 4H), 1.97 (dt, J=7.9, 10.7 Hz, 2H), 1.75 (t, J=6.7 Hz, 2H), 1.30-1.18 (m, 2H), 1.15-1.02 (m, 2H).

### Example 25, 26, and 27: racemic 9-((4-(Difluoromethoxy)phenyl)sulfonyl)-2-(2-oxaspiro[3.3]heptan-6-yl)-6-oxa-2,9-diazaspiro[4.5]decane, (R)-9-((4-(Difluoromethoxy)phenyl)sulfonyl)-2-(2-oxaspiro[3.3]heptan-6-yl)-6-oxa-2,9-diazaspiro[4.5]decane, and (S)-9-((4-(Difluoromethoxy)phenyl)sulfonyl)-2-(2-oxaspiro[3.3]heptan-6-yl)-6-oxa-2,9-diazaspiro[4.5]decane

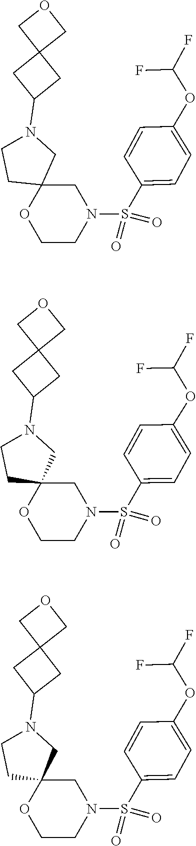 [SMILES: O=S(=O)(c1ccc(OC(F)F)cc1)N1CCOC2(CCN(C3CC4(COC4)C3)C2)C1 ; O=S(=O)(c1ccc(OC(F)F)cc1)N1CCO[C@@]2(CCN(C3CC4(COC4)C3)C2)C1 ; O=S(=O)(c1ccc(OC(F)F)cc1)N1CCO[C@]2(CCN(C3CC4(COC4)C3)C2)C1]

### 1. Synthesis of 9-((4-(difluoromethoxy)phenyl)sulfonyl)-6-oxa-2,9-diazaspiro[4.5]decane

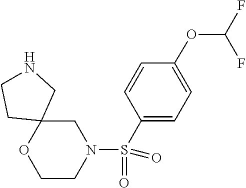 [SMILES: O=S(=O)(c1ccc(OC(F)F)cc1)N1CCOC2(CCNC2)C1]

To a solution of tert-butyl 6-oxa-2,9-diazaspiro[4.5]decane-2-carboxylate (750 mg, 3.1 mmol), DIPEA (800 mg, 6.2 mmol) and HCl (4 M, 3 mL) in DCM (25 mL) was added 4-(difluoromethoxy)benzenesulfonyl chloride (788 mg, 3.3 mmol) at room temperature. After 1 h at room temperature the reaction mixture was washed with sat. aq. 0.5 M HCl, water and brine. The organic phase was dried with Na₂SO₄, filtered and concentrated. The crude residue (200 mg) was dissolved in DCM (5 mL) and TFA (152 mg, 1.3 mmol) was added and stirred for 3 h at room temperature. The product was isolated by filtration to afford the trifluoroacetate salt of 9-((4-(difluoromethoxy)phenyl)sulfonyl)-6-oxa-2,9-diazaspiro[4.5]decane as a white solid which was used without further purification in the next step assuming 100% yield. LCMS: 349.1 (M+H)⁺.

### 2. Synthesis of racemic 9-((4-(difluoromethoxy)phenyl)sulfonyl)-2-(2-oxaspiro[3.3]heptan-6-yl)-6-oxa-2,9-diazaspiro[4.5]decane

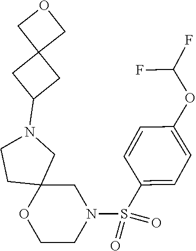 [SMILES: O=S(=O)(c1ccc(OC(F)F)cc1)N1CCOC2(CCN(C3CC4(COC4)C3)C2)C1]

A solution of 9-((4-(difluoromethoxy)phenyl)sulfonyl)-6-oxa-2,9-diazaspiro[4.5]decane trifluoroacetate (200 mg, 0.6 mmol) and 2-oxaspiro[3.3]heptan-6-one (64 mg, 0.6 mmol) in MeOH (10 mL) was stirred at 18° C. for 10 min and adjusted to pH=5-6 with AcOH before NaBH₃CN (288 mg, 4.6 mmol) was added. The reaction mixture mixture was stirred at 18° C. for 16 h. The mixture was diluted with water (20 mL), extracted with DCM (20 mL, 3×). The combined organic layers were washed with brine (20 mL), dried with anhydrous Na₂SO₄, filtered and concentrated in vacuo. Purification by prep-HPLC (Column Welch Xtimate C18 150×25 mm×5 μm; Condition Water (NH₄HCO₃)-ACN Begin B 35% End B 65% Gradient Time (min) 11 100% B Hold Time (min) 2; FlowRate (ml/min) 25) afforded 9-((4-(difluoromethoxy)phenyl)sulfonyl)-2-(2-oxaspiro[3.3]heptan-6-yl)-6-oxa-2,9-diazaspiro[4.5]decane as a yellow oil (111 mg, 43%, 100% purity). LCMS: 445.2 (M+H)⁺. ¹H NMR: (500 MHz, CD₃Cl) δ (ppm): 7.80-7.73 (m, 2H), 7.29 (d, J=8.7 Hz, 2H), 6.78-6.46 (m, 1H), 4.74-4.69 (m, 2H), 4.60 (s, 2H), 3.83-3.69 (m, 2H), 3.03-2.90 (m, 3H), 2.81 (br d, J=10.1 Hz, 1H), 2.67 (br t, J=7.5 Hz, 1H), 2.61-2.44 (m, 4H), 2.36 (br dd, J=6.9, 11.4 Hz, 2H), 2.16-2.03 (m, 2H), 1.90 (br t, J=6.8 Hz, 2H).

### 3. Chiral Separation of 9-((4-(difluoromethoxy)phenyl)sulfonyl)-2-(2-oxaspiro[3.3]heptan-6-yl)-6-oxa-2,9-diazaspiro[4.5]decane

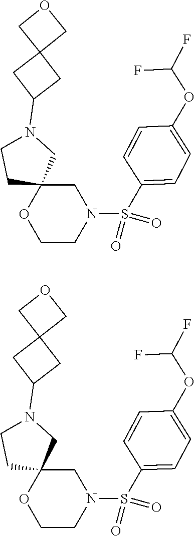 [SMILES: O=S(=O)(c1ccc(OC(F)F)cc1)N1CCO[C@@]2(CCN(C3CC4(COC4)C3)C2)C1 ; O=S(=O)(c1ccc(OC(F)F)cc1)N1CCO[C@]2(CCN(C3CC4(COC4)C3)C2)C1]

The racemic 9-((4-(difluoromethoxy)phenyl)sulfonyl)-2-(2-oxaspiro[3.3]heptan-6-yl)-6-oxa-2,9-diazaspiro[4.5]decane was separated into its enantiomers using CHIRALPAK AD-H 30×250 mm, 5 μm, 40% MeOH w/ 0.1% DEA in CO₂ (flow rate: 100 mL/min, ABPR 120 bar, MBPR 40 psi, column temp 40° C.) to yield two enantiomers of arbitrarily assigned stereochemistry:

Peak 1, enantiomer 1 (R)-9-((4-(difluoromethoxy)phenyl)sulfonyl)-2-(2-oxaspiro[3.3]heptan-6-yl)-6-oxa-2,9-diazaspiro[4.5]decane (33 mg, t_{R}=1.57 min, 99.9% ee).

Peak 2, enantiomer 2 (S)-9-((4-(difluoromethoxy)phenyl)sulfonyl)-2-(2-oxaspiro[3.3]heptan-6-yl)-6-oxa-2,9-diazaspiro[4.5]decane (35 mg, t_{R}=1.94 min, 98.8% ee).

### Example 28: 9-((3,5-Difluorophenyl)sulfonyl)-2-(2-oxaspiro[3.3]heptan-6-yl)-6-oxa-2,9-diazaspiro[4.5]decane

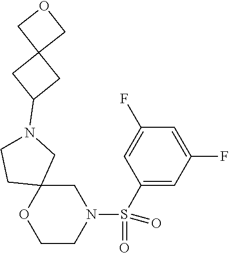 [SMILES: O=S(=O)(c1cc(F)cc(F)c1)N1CCOC2(CCN(C3CC4(COC4)C3)C2)C1]

The title compound was prepared using a similar method described for step 2 in Example 27 from the trifluoroacetate salt of 9-(3,5-difluorophenyl)sulfonyl-6-oxa-2,9-diazaspiro[4.5]decane (200 mg, 0.5 mmol) and 2-oxaspiro[3.3]heptan-6-one (52 mg, 0.5 mmol) in MeOH (10 mL) to give 9-((3,5-difluorophenyl)sulfonyl)-2-(2-oxaspiro[3.3]heptan-6-yl)-6-oxa-2,9-diazaspiro[4.5]decane as a yellow oil (67 mg, 35%, 100% purity). LCMS m/z=415.1 (M+H)⁺. ¹H NMR (500 MHz, CD₃OD) δ (ppm): 7.48-7.42 (m, 2H), 7.41-7.36 (m, 1H), 4.73 (s, 2H), 4.64-4.58 (m, 2H), 3.81-3.70 (m, 2H), 3.11-2.95 (m, 3H), 2.89 (br d, J=11.6 Hz, 1H), 2.80 (quin, J=7.6 Hz, 1H), 2.67 (d, J=10.5 Hz, 1H), 2.62-2.54 (m, 3H), 2.46-2.36 (m, 2H), 2.16-2.09 (m, 2H), 1.96-1.87 (m, 2H).

### Example 29: 4-((3,5-Difluorophenyl)sulfonyl)-9-((3-methyloxetan-3-yl)methyl)-1-oxa-4,9-diazaspiro[5.5]undecane

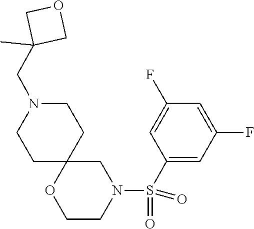 [SMILES: CC1(CN2CCC3(CC2)CN(S(=O)(=O)c2cc(F)cc(F)c2)CCO3)COC1]

### 1. Synthesis of 4-((3,5-difluorophenyl)sulfonyl)-1-oxa-4,9-diazaspiro[5.5]undecane

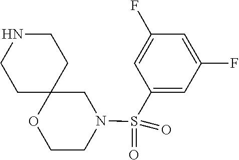 [SMILES: O=S(=O)(c1cc(F)cc(F)c1)N1CCOC2(CCNCC2)C1]

The title compound was prepared using a similar method described for step 1 for Example 25 from tert-butyl 6-oxa-2,9-diazaspiro[4.5]decane-2-carboxylate (commercial, 100 mg, 0.4 mmol) and 3,5-difluorobenzenesulfonylchloride (91 mg, 0.4 mmol) followed by HCl deprotection to yield the hydrochloride salt of 4-((3,5-difluorophenyl)sulfonyl)-1-oxa-4,9-diazaspiro[5.5]undecane (129 mg theoretical yield) which was used without further purification. LCMS m/z=333.1 (M+H)⁺.

### 2. Synthesis of 4-((3,5-difluorophenyl)sulfonyl)-9-((3-methyloxetan-3-yl)methyl)-1-oxa-4,9-diazaspiro[5.5]undecane

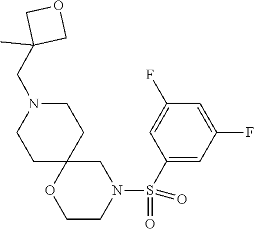 [SMILES: CC1(CN2CCC3(CC2)CN(S(=O)(=O)c2cc(F)cc(F)c2)CCO3)COC1]

The title compound was prepared using a similar method described for Example 1 from 4-((3,5-difluorophenyl)sulfonyl)-1-oxa-4,9-diazaspiro[5.5]undecane (65 mg, 0.2 mmol) and 3-methyloxetane-3-carbaldehyde (25 mg, 0.2 mmol) to yield 4-((3,5-difluorophenyl)sulfonyl)-9-((3-methyloxetan-3-yl)methyl)-1-oxa-4,9-diazaspiro[5.5]undecane (25 mg, 31%). LCMS m/z=417.2 (M+H)⁺. LCMS t_{R} (4 min)=1.25 min.

### Example 30: 9-((4-(Difluoromethoxy)phenyl)sulfonyl)-4-(tetrahydro-2H-pyran-4-yl)-1-oxa-4,9-diazaspiro[5.5]undecane

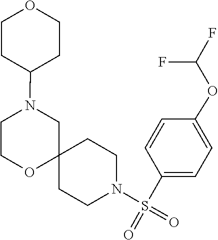 [SMILES: O=S(=O)(c1ccc(OC(F)F)cc1)N1CCC2(CC1)CN(C1CCOCC1)CCO2]

### 1. Synthesis of tert-butyl 4-(tetrahydro-2H-pyran-4-yl)-1-oxa-4,9-diazaspiro[5.5]undecane-9-carboxylate

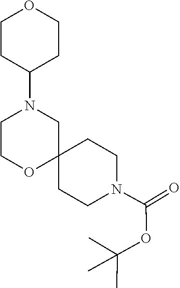 [SMILES: CC(C)(C)OC(=O)N1CCC2(CC1)CN(C1CCOCC1)CCO2]

To a vial containing tert-butyl 1-oxa-4,9-diazaspiro[5.5]undecane-9-carboxylate (commercial, 300 mg, 1.2 mmol) in anhydrous DCM (6 mL) was added tetrahydropyran-4-one (128 mg, 1.3 mmol) and NEt₃ (360 μL, 2.6 mmol) dropwise at 23° C., followed after 15 min by AcOH (170 μL, 2.9 mmol). The reaction mixture was stirred at 23° C. for 30 min, then NaBH(OAc)₃ (992 mg, 4.7 mmol) was added portion wise. The reaction was stirred at 23° C. After 6 d, the reaction was quenched with slow addition of sat. aq. NaHCO₃ solution, stirred at 23° C. for 10 min, then extracted with EtOAC (3×). The combined organic layers were washed with sat. aq. NaCl solution, dried with anhydrous Na₂SO₄, filtered and concentrated under reduced pressure to give the crude tert-butyl 4-(tetrahydro-2H-pyran-4-yl)-1-oxa-4,9-diazaspiro[5.5]undecane-9-carboxylate (446 mg). LCMS m/z=341.2 (M+H)⁺.

### 2. Synthesis of 4-(tetrahydro-2H-pyran-4-yl)-1-oxa-4,9-diazaspiro[5.5]undecane

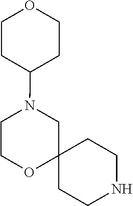 [SMILES: C1CC2(CCN1)CN(C1CCOCC1)CCO2]

The crude tert-butyl 4-(tetrahydro-2H-pyran-4-yl)-1-oxa-4,9-diazaspiro[5.5]undecane-9-carboxylate (50 mg) in EtOAc (1 mL) was added dropwise to a stirred solution of HCl (1 M, 2.9 mL).

After 1 d, the reaction mixture was evaporated to dryness to afford the hydrochloride salt of 4-(tetrahydro-2H-pyran-4-yl)-1-oxa-4,9-diazaspiro[5.5]undecane (35 mg) which was used without further purification. LCMS m/z=241.1 (M+H)⁺.

### 3. Synthesis of 9-((4-(difluoromethoxy)phenyl)sulfonyl)-4-(tetrahydro-2H-pyran-4-yl)-1-oxa-4,9-diazaspiro[5.5]undecane

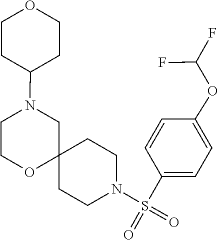 [SMILES: O=S(=O)(c1ccc(OC(F)F)cc1)N1CCC2(CC1)CN(C1CCOCC1)CCO2]

To a vial containing the hydrochloride salt of 4-(tetrahydro-2H-pyran-4-yl)-1-oxa-4,9-diazaspiro[5.5]undecane (35 mg, 0.2 mmol) in THF (1.5 mL) was added DIPEA (180 μL, 1.0 mmol) dropwise and DMAP (2 mg, 0.02 mmol) followed after stirring for 5 min by 4-(difluoromethoxy)benzenesulfonyl chloride (53 mg, 0.2 mmol). After 1 d, the reaction was quenched with sat. aq. NaHCO₃ solution and extracted with EtOAc (2×). The combined organic layers were washed with sat. aq. NaCl solution, dried over MgSO₄, filtered, evaporated and purified by HPLC (basic conditions) to yield 9-((4-(difluoromethoxy)phenyl)sulfonyl)-4-(tetrahydro-2H-pyran-4-yl)-1-oxa-4,9-diazaspiro[5.5]undecane (7 mg, 10%, 100% purity). LCMS m/z=447.1 (M+H)⁺. ¹H NMR (500 MHz, DMSO-d₆) δ (ppm): 7.81 (br d, J=8.6 Hz, 2H), 7.61-7.28 (m, 3H), 3.98 (br d, J=10.4 Hz, 2H), 3.89-3.72 (m, 1H), 3.71-3.41 (m, 1H), 3.33-3.19 (m, 1H), 3.00-2.78 (m, 1H), 2.48-2.21 (m, 5H), 2.05-1.88 (m, 3H), 1.78-1.67 (m, 1H), 1.67-1.40 (m, 7H), 1.39-1.25 (m, 1H).

### Example 31: 7-((2,4-Dimethylphenyl)sulfonyl)-2-(tetrahydro-2H-pyran-4-yl)-2,7-diazaspiro[3.5]nonane

### 1. Synthesis of tert-butyl 7-((2,4-dimethylphenyl)sulfonyl)-2,7-diazaspiro[3.5]nonane-2-carboxylate

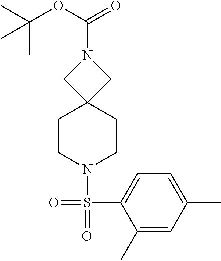 [SMILES: Cc1ccc(S(=O)(=O)N2CCC3(CC2)CN(C(=O)OC(C)(C)C)C3)c(C)c1]

To a mixture of tert-butyl 2,7-diazaspiro[3.5]nonane-2-carboxylate (226 mg, 1.0 mmol) and 2,4-dimethylbenzenesulfonyl chloride (205 mg, 1.0 mmol) in DCM (5 mL) was added DIPEA (260 μL, 1.5 mmol). The reaction mixture was stirred at room temperature overnight, then washed with sat. aq. NaHCO₃ and water. The organic phase was concentrated and purified by column chromatography on silica gel (24 g, EtOAc in heptane 10-50%) to afford tert-butyl 7-((2,4-dimethylphenyl)sulfonyl)-2,7-diazaspiro[3.5]nonane-2-carboxylate as a white foam (294 mg, 75%). LCMS m/z=392.2 (M+H)⁺.

### 2. Synthesis of 7-((2,4-dimethylphenyl)sulfonyl)-2,7-diazaspiro[3.5]nonane

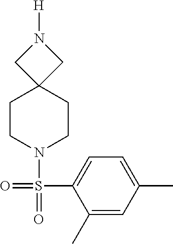 [SMILES: Cc1ccc(S(=O)(=O)N2CCC3(CC2)CNC3)c(C)c1]

To a reaction vial containing tert-butyl 7-((2,4-dimethylphenyl)sulfonyl)-2,7-diazaspiro[3.5]nonane-2-carboxylate (294 mg, 0.8 mmol) in 1,1,1,3,3,3-hexafluoropropan-2-ol (3 mL) was added TFA (140 μL, 1.9 mmol) dropwise at room temperature. The mixture was stirred at room temperature for 5 h, concentrated under reduced pressure and co-evaporated with MeCN (3×) to get the trifluoroacetate salt of 7-((2,4-dimethylphenyl)sulfonyl)-2,7-diazaspiro[3.5]nonane as a white solid (316 mg). The product was used without further purification. LCMS m/z=295.0 (M+H)^{m}.

### 3: Synthesis of 7-((2,4-dimethylphenyl)sulfonyl)-2-(tetrahydro-2H-pyran-4-yl)-2,7-diazaspiro[3.5]nonane

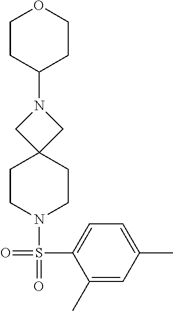 [SMILES: Cc1ccc(S(=O)(=O)N2CCC3(CC2)CN(C2CCOCC2)C3)c(C)c1]

To a mixture of tetrahydropyran-4-one (13 mg, 0.1 mmol) and the trifluoroacetate salt of 7-((2,4-dimethylphenyl)sulfonyl)-2,7-diazaspiro[3.5]nonane (42 mg, 0.1 mmol) in DCM (2 mL) was added DIPEA (50 μL, 0.3 mmol). The reaction mixture was stirred at for 5 min. AcOH (18 mg, 0.3 mmol) was added followed, after stirring for another 5 min, by NaBH(OAc)₃ (64 mg, 0.3 mmol) in one portion. The mixture was stirred at room temperature overnight, then quenched with sat. aq. NaHCO₃. More DCM was added and the mixture was stirred for 5 min before the layers were separated. The organic layer was washed with water, concentrated under reduced pressure and purified by HPLC (basic conditions) to afford 7-((2,4-dimethylphenyl)sulfonyl)-2-(tetrahydro-2H-pyran-4-yl)-2,7-diazaspiro[3.5]nonane (28 mg, 73%). LCMS m/z=379.1 (M+H)⁺. ¹H NMR (500 MHz DMSO-d₆) δ (ppm): 7.67 (d, J=7.9 Hz, 1H), 7.25 (s, 1H), 7.21 (d, J=7.9 Hz, 1H), 3.7-3.8 (m, 2H), 3.33 (s, 5H), 3.23 (dt, J=2.1, 11.1 Hz, 2H), 2.9-3.0 (i, 4H), 2.83 (s, 3H), 2.34 (s, 3H), 1.6-1.8 (m, 4H), 1.53 (br d, J=11.6 Hz, 2H), 1.0-1.1 (in, 2H).

### Examples 32 and 33: 7-((4-(Difluoromethoxy)phenyl)sulfonyl)-2-(tetrahydro-2H-pyran-4-yl)-2,7-diazaspiro[3.5]nonane and 7-((2-methyl-6-(trifluoromethyl)pyridin-3-yl)sulfonyl)-2-(tetrahydro-2H-pyran-4-yl)-2,7-diazaspiro[3.5]nonane

The following Examples 32 and 33 were prepared from tetrahydropyran-4-one using a similar method described for 7-((2,4-dimethylphenyl)sulfonyl)-2,7-diazaspiro[3.5]nonane (Example 31). Data for intermediates in Tables D and E (steps 1 and 2), data for Examples 32 and 33 in Table F (step 3).

TABLE D
Intermediates of Step 1

| Intermediate name/Sulfonyl chloride | | |
|---|---|---|
| (SM) | Structure | Data |
| tert-Butyl 7-((4- (difluoromethoxy)phenyl)sulfonyl)- 2,7-diazaspiro[3.5]nonane-2- carboxylate (552 mg, 2.4 mmol) SM: 4- (difluoromethoxy)benzenesulfonyl chloride (592 mg, 2.4 mmol) | 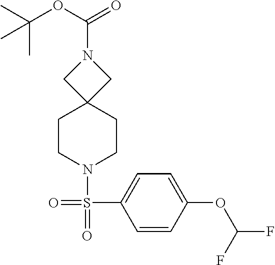 [SMILES: CC(C)(C)OC(=O)N1CC2(CCN(S(=O)(=O)c3ccc(OC(F)F)cc3)CC2)C1] | Yield: 1.06 g. ¹H NMR (400 MHz, CD₃OD): δ (ppm): 7.91-7.74 (m, 2H), 7.39- 7.28 (m, 2H), 7.25- 6.74 (m, 1H), 3.53 (br s, 4H), 2.98 (br s, 4H), 1.87-1.74 (m, 4H), 1.40 (s, 9H). |
| tert-Butyl 7-((2-methyl-6- (trifluoromethyl)pyridin-3- yl)sulfonyl)-2,7- diazaspiro[3.5]nonane-2- carboxylate (452 mg, 2.0 mmol) SM: 2-methyl-6- (trifluoromethyl)pyridine-3- sulfonyl chloride (519 mg, 2.0 mmol) | 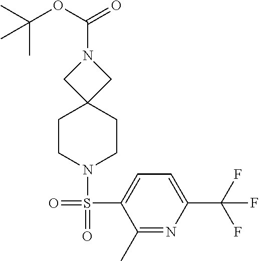 [SMILES: Cc1nc(C(F)(F)F)ccc1S(=O)(=O)N1CCC2(CC1)CN(C(=O)OC(C)(C)C)C2] | Yield: 803 mg. ¹H NMR (400 MHz, CD₃OD): δ (ppm): 8.48-8.35 (m, 1H), 7.82 (d, J = 8.0 Hz, 1H), 3.78-3.56 (m, 4H), 3.27-3.20 (m, 4H), 2.86 (s, 3H), 1.87-1.80 (m, 4H), 1.42 (s, 9H). |

TABLE E
Intermediates of Step 2

| Intermediate name | Structure | Data |
|---|---|---|
| 7-((4- (Difluoromethoxy)phenyl)sulfonyl)- 2,7-diazaspiro[3.5]nonane | 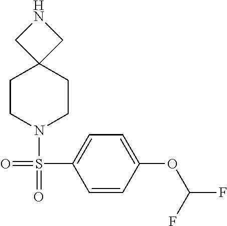 [SMILES: O=S(=O)(c1ccc(OC(F)F)cc1)N1CCC2(CC1)CNC2] | Yield: 1.18 g, TFA salt. LCMS m/z = 333.1 (M + H)⁺. |
| 7-((2-Methyl-6- (trifluoromethyl)pyridin-3- yl)sulfonyl)-2,7- diazaspiro[3.5]nonane | 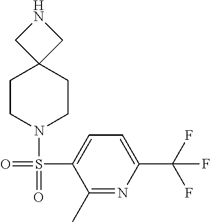 [SMILES: Cc1nc(C(F)(F)F)ccc1S(=O)(=O)N1CCC2(CC1)CNC2] | Yield: 1.24 g, TFA salt. LCMS m/z = 350.1 (M + H)⁺. |

TABLE F
Examples 32 and 33

| Example | Structure | Data |
|---|---|---|
| 32 | 7-((4-(Difluoromethoxy)phenyl)sulfonyl)- 2-(tetrahydro-2H-pyran-4-yl)- 2,7-diazaspiro[3.5]nonane 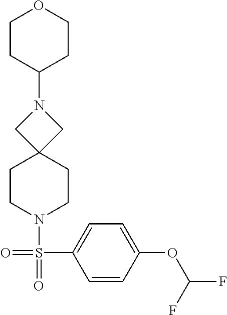 [SMILES: O=S(=O)(c1ccc(OC(F)F)cc1)N1CCC2(CC1)CN(C1CCOCC1)C2] | Yield: 52 mg, 75.8%. LCMS m/z = 417.2 (M + H)⁺. ¹H NMR (400 MHz, CD₃OD): δ (ppm): 7.78-7.90 (m, 2H), 7.32-7.41 (m, 2H), 6.77-7.25 (m, 1H), 3.92 (dt, J = 9.8, 2.3 Hz, 2H), 3.34-3.41 (m, 2H), 2.92-3.07 (m, 8H), 2.25-2.42 (m, 1H), 1.79-1.90 (m, 4H), 1.67 (dt, J = 10.7, 1.9 Hz, 2H), 1.23 (br dd, J = 12.0, 4.5 Hz, 2H). |
| 33 | 7-((2-Methyl-6-(trifluoromethyl)pyridin- 3-yl)sulfonyl)-2-(tetrahydro-2H-pyran-4- yl)- 2,7-diazaspiro[3.5]nonane 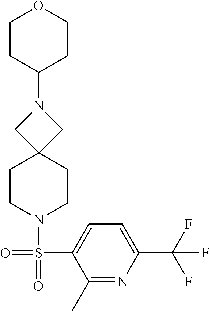 [SMILES: Cc1nc(C(F)(F)F)ccc1S(=O)(=O)N1CCC2(CC1)CN(C1CCOCC1)C2] | Yield: 43 mg, 77.6%. LCMS m/z = 434.2 (M + H)⁺. ¹H NMR (500 MHz, DMSO-d₆) δ (ppm): 7.64 (d, 1H, J = 7.9 Hz), 7.2- 7.3 (m, 2H), 3.7-3.8 (m, 2H), 3.21 (dt, 2H, J = 2.1, 11.1 Hz), 2.9-3.0 (m, 4H), 2.80 (s, 3H), 2.31 (s, 3H), 1.6-1.7 (m, 5H), 1.50 (br d, 2H, J = 11.6 Hz), 0.9-1.1 (m, 2H). |

### Example 34: 4-((7-((2,4-Dimethylphenyl)sulfonyl)-2,7-diazaspiro[3.5]nonan-2-yl)methyl)tetrahydro-2H-pyran-4-ol

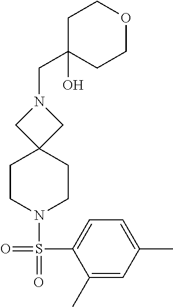 [SMILES: Cc1ccc(S(=O)(=O)N2CCC3(CC2)CN(CC2(O)CCOCC2)C3)c(C)c1]

To a mixture of 1,6-dioxaspiro[2.5]octane (10 mg, 0.1 mmol) and the trifluoroacetate salt of 7-((2,4-dimethylphenyl)sulfonyl)-2,7-diazaspiro[3.5]nonane in EtOH (1 mL) was added DIPEA (40 μL, 0.2 mmol). The mixture was stirred at 60° C. overnight, then partitioned between EtOAc and sat. aq. NaHCO₃. The organic phase was separated, concentrated and purified by column chromatography on silica gel (12 g, EtOAc/EtOH 3:1) to obtain 1-((7-((2,4-dimethylphenyl)sulfonyl)-2,7-diazaspiro[3.5]nonan-2-yl)methyl)cyclohexan-1-ol as a sticky solid after lyophilization (19 mg, 69%). LCMS m/z=409.2 (M+H)⁺. ¹H NMR (400 MHz, CD₃OD) δ (ppm): 7.75 (d, J=8.0 Hz, 1H), 7.33-7.12 (m, 2H), 3.87-3.60 (m, 4H), 3.21 (s, 4H), 3.13-3.04 (m, 4H), 2.58 (s, 3H), 2.55 (s, 2H), 2.39 (s, 3H), 1.88-1.75 (m, 4H), 1.54-1.70 (m, 2H), 1.44 (br d, J=13.3 Hz, 2H).

### Example 35: 7-((2,4-Dimethylphenyl)sulfonyl)-2-((tetrahydro-2H-pyran-4-yl)methyl)-2,7-diazaspiro[3.5]nonane

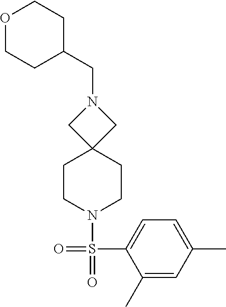 [SMILES: Cc1ccc(S(=O)(=O)N2CCC3(CC2)CN(CC2CCOCC2)C3)c(C)c1]

The title compound 35 was prepared using a similar method described for Example 31 from the trifluoroacetate salt of 7-((2,4-dimethylphenyl)sulfonyl)-2,7-diazaspiro[3.5]nonane (42 mg, 0.1 mmol) and tetrahydro-4-carbaldehyde (15 mg, 0.1 mmol) to afford after column chromatography on silica gel (12 g, EtOAc/EtOH 3:1) 7-((2,4-dimethylphenyl)sulfonyl)-2-((tetrahydro-2H-pyran-4-yl)methyl)-2,7-diazaspiro[3.5]nonane as a colorless oil (29 mg, 74%). LCMS m/z=393.3 (M+H)⁺. ¹H NMR (400 MHz, CD₃OD): δ (ppm): 7.79-7.73 (m, 1H), 7.27-7.15 (m, 2H), 3.91 (br dd, J=11.4, 4.4 Hz, 2H), 3.43-3.35 (m, 2H), 3.05-3.13 (m, 8H), 2.58 (s, 3H), 2.42 (br d, J=6.5 Hz, 2H), 2.40-2.38 (m, 3H), 1.85-1.77 (m, 4H), 1.69-1.57 (m, 3H), 1.33-1.18 (m, 2H).

### Example 36: 8-((2-Chloro-4-methylphenyl)sulfonyl)-2-(tetrahydro-2H-pyran-4-yl)-2,8-diazaspiro[4.5]decane

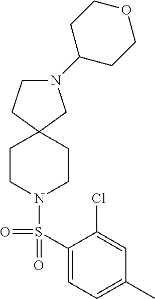 [SMILES: Cc1ccc(S(=O)(=O)N2CCC3(CCN(C4CCOCC4)C3)CC2)c(Cl)c1]

### 1. Synthesis of tert-butyl 2-(tetrahydro-2H-pyran-4-yl)-2,8-diazaspiro[4.5]decane-8-carboxylate

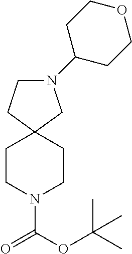 [SMILES: CC(C)(C)OC(=O)N1CCC2(CC1)CCN(C1CCOCC1)C2]

To tetrahydropyran-4-one (610 mg, 6.1 mmol) and tert-butyl 2,8-diazaspiro[4.5]decane-8-carboxylate (1.22 g, 5.1 mmol) in DCM (20 mL) was added AcOH (580 μL, 10.1 mmol) followed by NaBH(OAc)₃ (4.0 g, 18.9 mmol) in 4 portions. The reaction mixture was stirred at room temperature overnight, then quenched with sat. aq. NaHCO₃ and extracted with DCM (3×). The organic layers were dried with MgSO₄, filtered, concentrated and co-evaporated with EtOAc to give tert-butyl 2-(tetrahydro-2H-pyran-4-yl)-2,8-diazaspiro[4.5]decane-8-carboxylate as a pale yellow oil (1.69 g) which was used without further purification. LCMS m/z=309.1 (M+CH₃)⁺.

### 2. Synthesis of 2-(tetrahydro-2H-pyran-4-yl)-2,8-diazaspiro[4.5]decane

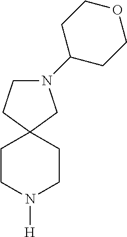 [SMILES: C1CC2(CCN1)CCN(C1CCOCC1)C2]

To a solution of tert-butyl 2-(tetrahydro-2H-pyran-4-yl)-2,8-diazaspiro[4.5]decane-8-carboxylate (1.68 g) in EtOAc (2 mL) was added HCl (1 M in EtOAc, 15.2 mL) at room temperature. After 10 min, a further 5 mL of 1M HCl in EtOAc were added and the reaction mixture was stirred at room temperature overnight. MeOH (2 mL) and 1M HCl in EtOAc (5 mL) were added and the reaction mixture was stirred at room temperature for 3 h. The solvent was evaporated and co-evaporated with MeCN/MeOH to afford the hydrochloride salt of 2-(tetrahydro-2H-pyran-4-yl)-2,8-diazaspiro[4.5]decane as a off white solid (1.52 g). LCMS m/z=225.2 (M+H)⁺.

### 3. Synthesis of 8-((2-chloro-4-methylphenyl)sulfonyl)-2-(tetrahydro-2H-pyran-4-yl)-2,8-diazaspiro[4.5]decane

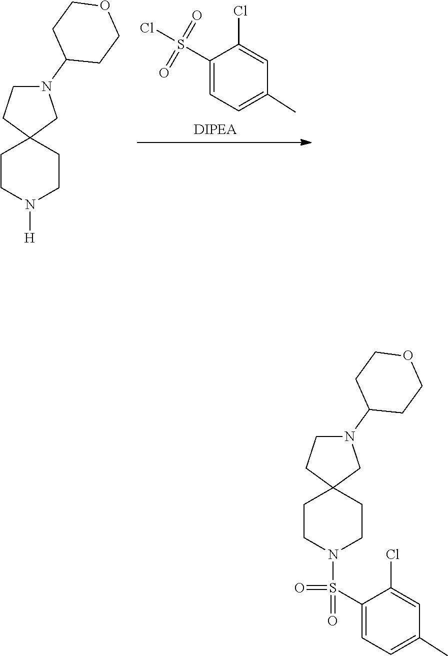

To the hydrochloride salt of 2-tetrahydropyran-4-yl-2,8-diazaspiro[4.5]decane (54 mg) and 2-chloro-4-methyl-benzenesulfonylchloride (34 mg, 0.2 mmol) in DCM (3 mL) was added DIPEA (80 μL, 0.5 mmol). The mixture was stirred at room temperature for 3 d, then quenched with sat. aq. NaHCO₃ and water and stirred at room temperature for 5 min. The organic layer was separated, washed with water and concentrated under reduced pressure. The residue was purified by column chromatography on silica gel (12 g, EtOAc/EtOH 3:1) to get 8-((2-chloro-4-methylphenyl)sulfonyl)-2-(tetrahydro-2H-pyran-4-yl)-2,8-diazaspiro[4.5]decane as a sticky solid after lyophilization (43 mg, 69%). LCMS m/z=413.1 (M+H)⁺. ¹H NMR (400 MHz, CD₃OD) δ (ppm): 7.91 (d, J=8.0 Hz, 1H), 7.47 (s, 1H), 7.35-7.27 (m, 1H), 3.95 (br dd, J=11.3, 3.3 Hz, 2H), 3.48-3.35 (m, 2H), 3.32-3.26 (m, 2H), 3.24-3.15 (m, 2H), 2.73 (t, J=6.9 Hz, 2H), 2.54 (s, 2H), 2.43 (s, 3H), 2.34-2.23 (m, 1H), 1.89-1.78 (m, 2H), 1.73-1.58 (m, 6H), 1.57-1.44 (in, 2H).

### Example 37-43

The Examples 37-43 in the following Table G were prepared using a similar method described for Example 36 from the hydrochloride salt of 2-tetrahydropyran-4-yl-2,8-diazaspiro[4.5]decane (1 equiv.) and the corresponding sulfonyl chloride.

TABLE G

| Example | Structure/Name/Sulfonyl chloride (SM) | Data |
|---|---|---|
| 37 | 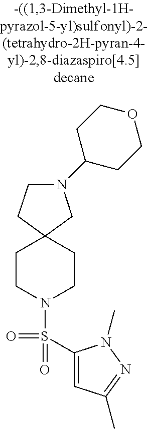 [SMILES: Cc1cc(S(=O)(=O)N2CCC3(CCN(C4CCOCC4)C3)CC2)n(C)n1] | Yield: 26 mg, 54%. LCMS m/z = 383.2 (M + H)⁺. ¹H NMR (400 MHz, CD₃OD) δ (ppm): 6.66-6.51 (m, 1H), 4.01 (d, J = 1.0 Hz, 3H), 3.98-3.89 (m, 2H), 3.45-3.36 (m, 2H), 3.28-3.20 (m, 2H), 3.17-3.08 (m, 2H), 2.76-2.67 (m, 2H), 2.53 (s, 2H), 2.26 (s, 3H), 2.30-2.25 (m, 1H), 1.85 (br d, J = 12.5 Hz, 2H), 1.73-1.62 (m, 6H), 1.59-1.43 (m, 2H). |
| | SM: 2,5-dimethylpyrazole-3- | |
| | sulfonylchloride | |
| | (25 mg, 0.1 mmol) | |
| 38 | 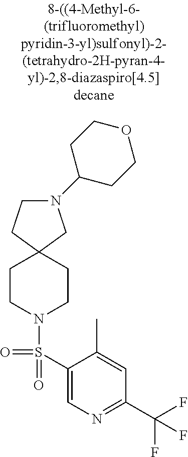 [SMILES: Cc1cc(C(F)(F)F)ncc1S(=O)(=O)N1CCC2(CCN(C3CCOCC3)C2)CC1] | Yield: 19 mg, 47%. LCMS m/z = 448.2 (M + H)⁺. ¹H NMR (400 MHz, CD₃OD) δ (ppm): 9.05 (s, 1H), 7.91 (s, 1H), 4.00-3.89 (m, 2H), 3.46-3.37 (m, 2H), 3.36-3.30 (m, 2H), 3.28-3.19 (m, 2H), 2.76 (s, 3H), 2.71 (t, J = 6.7 Hz, 2H), 2.53 (s, 2H), 2.33-2.21 (m, 1H), 1.85 (br dd, J = 12.2, 1.4 Hz, 2H), 1.70-1.65 (br t, J = 6.4 Hz, 6H), 1.58-1.45 (m, 2H). |
| | SM: 4-methyl-6-(trifluoromethyl)pyridine- | |
| | 3-sulfonyl chloride (24 mg, 0.1 mmol) | |
| 39 | 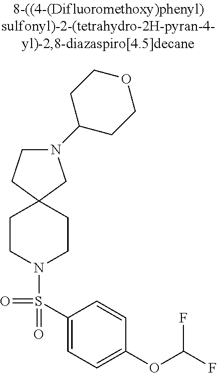 [SMILES: O=S(=O)(c1ccc(OC(F)F)cc1)N1CCC2(CCN(C3CCOCC3)C2)CC1] | Yield: 1.2 g, 45%. LCMS m/z = 431.2 (M + H)⁺. ¹H NMR (400 MHz, CD₃OD) δ (ppm): 8.00-7.74 (m, 2H), 7.45-7.29 (m, 2H), 7.22-6.74 (m, 1H), 3.91 (dd, J = 10.8, 4.3 Hz, 2H), 3.42-3.32 (m, 2H), 3.12-3.01 (m, 2H), 2.97-2.85 (m, 2H), 2.70-2.57 (m, 2H), 2.41 (s, 2H), 2.26-2.12 (m, 1H), 1.85-1.73 (m, 2H), 1.72-1.59 (m, 4H), 1.57-1.38 (m, 4H). |
| | SM: 4-(difluoromethoxy)benzenesulfonyl | |
| | chloride (1.5 g, 6.0 mmol) | |
| 40 | 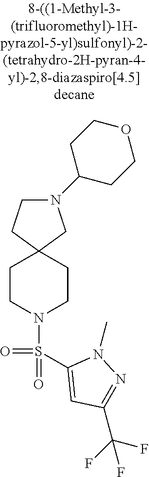 [SMILES: Cn1nc(C(F)(F)F)cc1S(=O)(=O)N1CCC2(CCN(C3CCOCC3)C2)CC1] | Yield: 47 mg, 54%. LCMS m/z = 437.2 (M + H)⁺. ¹H NMR (400 MHz, CD₃OD) δ (ppm): 7.16-7.03 (m, 1H), 4.14 (s, 3H), 4.01-3.83 (m, 2H), 3.38 (td, J = 12.0, 1.9 Hz, 2H), 3.28-3.24 (m, 2H), 3.23-3.14 (m, 2H), 2.68 (t, J = 6.9 Hz, 2H), 2.50 (s, 2H), 2.24 (tt, J = 11.1, 4.0 Hz, 1H), 1.87-1.78 (m, 2H), 1.72-1.61 (m, 6H), 1.56-1.42 (m, 2H). |
| | SM: 2-methyl-5-(trifluoromethyl)pyrazole-3- | |
| | sulfonyl chloride (50 mg, 0.2 mmol) | |
| 41 | 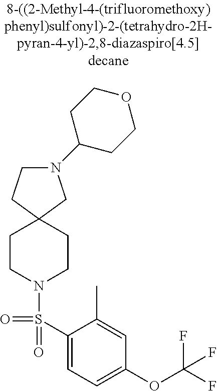 [SMILES: Cc1cc(OC(F)(F)F)ccc1S(=O)(=O)N1CCC2(CCN(C3CCOCC3)C2)CC1] | Yield: 50 mg, 60%. LCMS m/z = 463.2 (M + H)⁺. ¹H NMR (400 MHz, CD₃OD) δ (ppm): 8.06-7.89 (m, 1H), 7.37-7.23 (m, 2H), 3.93 (dd, J = 10.8, 3.8 Hz, 2H), 3.38 (td, J = 11.9, 2.0 Hz, 2H), 3.25-3.18 (m, 2H), 3.17-3.10 (m, 2H), 2.67 (t, J = 6.9 Hz, 2H), 2.64 (s, 3H), 2.49 (s, 2H), 2.24 (s, 1H), 1.87-1.78 (m, 2H), 1.70-1.60 (m, 6H), 1.49 (br dd, J = 11.3, 4.0 Hz, 2H). |
| | SM: 2-methyl-4-(trifluoromethoxy)benzene | |
| | sulfonyl chloride (50 mg, 0.2 mmol) | |
| 42 | 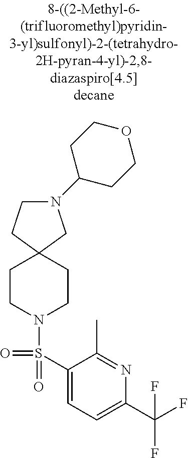 [SMILES: Cc1nc(C(F)(F)F)ccc1S(=O)(=O)N1CCC2(CCN(C3CCOCC3)C2)CC1] | Yield: 46 mg, 86%. LCMS m/z = 448.2 (M + H)⁺. ¹H NMR (400 MHz, CD₃OD) δ (ppm): 8.43 (d, J = 8.0 Hz, 1H), 7.82 (d, J = 8.0 Hz, 1H), 4.00-3.86 (m, 2H), 3.38 (td, J = 11.9, 2.1 Hz, 2H), 3.29-3.18 (m, 4H), 2.87 (s, 3H), 2.68 (t, J = 6.8 Hz, 2H), 2.50 (s, 2H), 2.30-2.14 (m, 1H), 1.87- 1.78 (m, 2H), 1.73-1.59 (m, 6H), 1.49 (br dd, J = 12.2, 4.6 Hz, 2H). |
| | SM: 2-methyl-6-(trifluoromethyl)pyridine- | |
| | 3-sulfonylchloride (31 mg, 0.1 mmol) | |
| 43 | 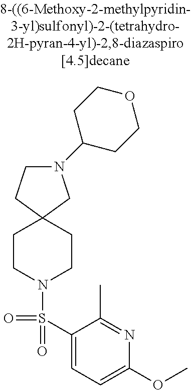 [SMILES: COc1ccc(S(=O)(=O)N2CCC3(CCN(C4CCOCC4)C3)CC2)c(C)n1] | Yield: 38 mg, 59%. LCMS m/z = 410.3 (M + H)⁺. ¹H NMR (400 MHz, CD₃OD) δ (ppm): 8.06 (d, J = 8.8 Hz, 1H), 6.76 (d, J = 8.8 Hz, 1H), 3.99 (s, 3H), 3.98-3.90 (m, 2H), 3.40 (td, J = 11.9, 2.0 Hz, 2H), 3.25-3.04 (m, 4H), 2.74 (s, 3H), 2.69 (t, J = 6.9 Hz, 2H), 2.50 (s, 2H), 2.31-2.19 (m, 1H), 1.89-1.80 (m, 2H), 1.70-1.60 (m, 6H), 1.51 (br s, 2H). |
| | SM: 6-methoxy-2-methylpyridine-3- | |
| | sulfonyl | |
| | chloride (35 mg, 0.2 mmol) | |

### Example 44: 2-((2,4-Dimethylphenyl)sulfonyl)-6-(tetrahydro-2H-pyran-4-yl)-2,6-diazaspiro[3.3]heptane

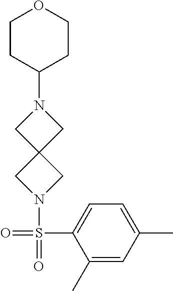 [SMILES: Cc1ccc(S(=O)(=O)N2CC3(CN(C4CCOCC4)C3)C2)c(C)c1]

### 1. Synthesis of 2-((2,4-dimethylphenyl)sulfonyl)-2,6-diazaspiro[3.3]heptane

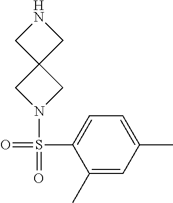 [SMILES: Cc1ccc(S(=O)(=O)N2CC3(CNC3)C2)c(C)c1]

The title compound was prepared using a similar method described in step 1 and 2 for the intermediates of Example 31 from tert-butyl 2,6-diazaspiro[3.3]heptane-2-carboxylate (204 mg, 1.0 mmol) and 2,4-dimethylbenzenesulphonyl chloride (204 mg, 1.0 mmol) to give tert-butyl 6-((2,4-dimethylphenyl)sulfonyl)-2,6-diazaspiro[3.3]heptane-2-carboxylate (315 mg, 86%), purified by column chromatography on silica gel (24 g, EtOAc in heptane 10-20%). ¹H NMR (400 MHz, CD₃OD) δ (ppm) 7.79 (d, J=8.0 Hz, 1H), 7.32-7.13 (m, 2H), 4.00 (s, 4H), 3.93 (s, 4H), 2.59 (s, 3H), 2.41 (s, 3H), 1.43 (s, 9H). TFA deprotection afforded the trifluoroacetate salt of 2-((2,4-dimethylphenyl)sulfonyl)-2,6-diazaspiro[3.3]heptane (365 mg) which was used without further purification. ¹H NMR (400 MHz, CD₃OD) δ (ppm): 7.87-7.76 (m, 1H), 7.33-7.15 (m, 2H), 4.22 (s, 4H), 4.02 (d, J=2.0 Hz, 4H), 2.59 (s, 3H), 2.41 (s, 3H).

### 2. Synthesis of 2-((2,4-dimethylphenyl)sulfonyl)-6-(tetrahydro-2H-pyran-4-yl)-2,6-diazaspiro[3.3]heptane

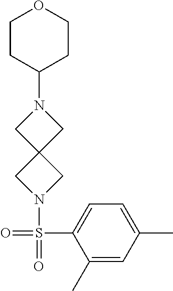 [SMILES: Cc1ccc(S(=O)(=O)N2CC3(CN(C4CCOCC4)C3)C2)c(C)c1]

The title compound was prepared using a similar method described for Example 31 from the trifluoroacetate salt of 2-((2,4-dimethylphenyl)sulfonyl)-2,6-diazaspiro[3.3]heptane (47 mg) and tetrahydropyran-4-one (14 mg, 0.1 mol). The crude was purified by column chromatography on silica gel (12 g, EtOAc/EtOH 3:1) to afford 2-((2,4-dimethylphenyl)sulfonyl)-6-(tetrahydro-2H-pyran-4-yl)-2,6-diazaspiro[3.3]heptane as an colorless oil (32 mg, 84%). LCMS m/z=351.1 (M+H)⁺. ¹H NMR (400 MHz, CD₃OD) δ (ppm): 7.79 (d, J=8.0 Hz, 1H), 7.26 (s, 1H), 7.22 (d, J=8.0 Hz, 1H), 3.98-3.81 (m, 6H), 3.41-3.35 (m, 2H), 3.34-3.33 (m, 4H), 2.59 (s, 3H), 2.40 (s, 3H), 2.32-2.19 (m, 1H), 1.66 (br d, J=12.5 Hz, 2H), 1.34-1.14 (m, 2H).

### Example 45: 4-((6-((2,4-Dimethylphenyl)sulfonyl)-2,6-diazaspiro[3.3]heptan-2-yl)methyl)tetrahydro-2H-pyran-4-ol

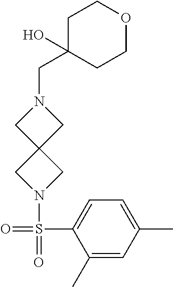 [SMILES: Cc1ccc(S(=O)(=O)N2CC3(CN(CC4(O)CCOCC4)C3)C2)c(C)c1]

The title compound was prepared using a similar method described in step 3 for Example 34 from the trifluoroacetate salt of 2-((2,4-dimethylphenyl)sulfonyl)-2,6-diazaspiro[3.3]heptane (Example 45, step 1, 43 mg, 100 μmol) and 1,6-dioxaspiro[2.5]octane (14.8 mg, 130 μmol) to yield 4-((6-((2,4-dimethylphenyl)sulfonyl)-2,6-diazaspiro[3.3]heptan-2-yl)methyl)tetrahydro-2H-pyran-4-ol after purification by column chromatography on silica gel (12 g, EtOAc/EtOH 3:1) as a white solid (28 mg, 74%). LCMS m/z=381.2 (M+H)⁺. ¹H NMR (400 MHz, CD₃OD) δ (ppm): 7.78 (d, J=8.0 Hz, 1H), 7.25 (s, 1H), 7.22 (dd, J=8.0, 0.8 Hz, 1H), 3.87 (s, 4H), 3.79-3.63 (m, 4H), 3.39 (s, 4H), 2.58 (s, 3H), 2.43 (s, 2H), 2.40 (s, 3H), 1.65-1.55 (m, 2H), 1.49-1.40 (m, 2H).

### Example 46: 2-((1-Methyl-3-(trifluoromethyl)-1H-pyrazol-5-yl)sulfonyl)-6-(tetrahydro-2H-pyran-4-yl)-2,6-diazaspiro[3.3]heptane

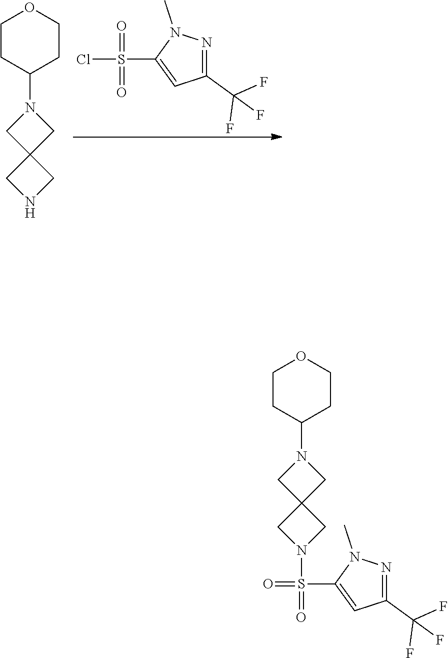

The title compound was prepared using a similar method to step 3 of Example 36 from the hydrochloride salt of 2-(tetrahydro-2H-pyran-4-yl)-2,6-diazaspiro[3.3]heptane (commercial, 26 mg, 0.1 μmol) and 2-methyl-5-(trifluoromethyl)pyrazole-3-sulfonyl chloride (27 mg, 0.1 mmol) to afford the trifluoroacetate salt of 2-((1-methyl-3-(trifluoromethyl)-1H-pyrazol-5-yl)sulfonyl)-6-(tetrahydro-2H-pyran-4-yl)-2,6-diazaspiro[3.3]heptane after HPLC (acidic conditions) (49 mg, 670%). LCMS m/z=395.2 (M+H)⁺. ¹H NMR (400 MHz, CD₃OD) δ (ppm): 7.23 (s, 1H), 4.18 (s, 3H), 4.04 (s, 4H), 4.00-3.90 (m, 2H), 3.40-3.30 (m, 6H), 2.28 (tt, J=4.0, 10.8 Hz, 1H), 1.70-1.60 (m, 2H), 1.30-1.20 (in, 2H).

### Example 47-50

The Examples 47-50 in the following Table H were prepared using a similar method described for Example 36 from the hydrochloride salt of 2-(tetrahydro-2H-pyran-4-yl)-2,6-diazaspiro[3.3]heptane (25.5 mg, 100 μmol) and the corresponding sulfonyl chloride (110 μmol) unless stated otherwise. Purified by HPLC (acidic or basic conditions), unless stated otherwise.

TABLE H

| Example | Name/Structure/Sulfonyl chloride (SM) | Data |
|---|---|---|
| 47 | 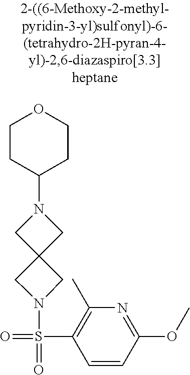 [SMILES: COc1ccc(S(=O)(=O)N2CC3(CN(C4CCOCC4)C3)C2)c(C)n1] | Yield: 39 mg, 79%, TFA salt. LCMS m/z = 368.2 (M + H)⁺. ¹H NMR (600 MHz, DMSO- d₆) δ (ppm) 8.03 = (d, J = 8.7 Hz, 1H), 6.86 (d, J = 8.7 Hz, 1H), 4.27 (br s, 4H), 4.04 (br s, 2H), 4.00-3.90 (m, 9H), 3.30-3.2 (m, 1H), 2.54 (s, 3H), 1.80-1.70 (m, 2H), 1.2- 1.3 (m, 2H). |
| | SM: 6-methoxy-2-methylpyridine-3-sulfonyl | |
| | chloride | |
| 48 | 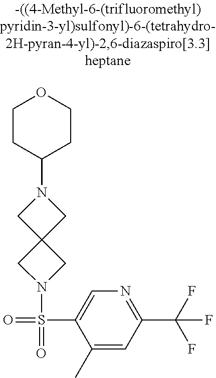 [SMILES: Cc1cc(C(F)(F)F)ncc1S(=O)(=O)N1CC2(CN(C3CCOCC3)C2)C1] | Yield: 37 mg, 86%, TFA salt. LCMS m/z = 406.2 (M + H)⁺. LCMS t_{R} (4 min) = 1.44 min. |
| | SM: 4-methyl-6-(trifluoromethyl)pyridine- | |
| | 3-sulfonyl chloride | |
| 49 | 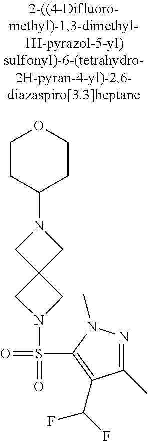 [SMILES: Cc1nn(C)c(S(=O)(=O)N2CC3(CN(C4CCOCC4)C3)C2)c1C(F)F] | Yield: 43 mg, 91%, TFA salt. LCMS m/z = 391.2 (M + H)⁺. LCMS t_{R} (4 min) = 1.39 min. |
| | SM: 4-(difluoromethyl)-2,5-dimethyl-pyrazole- | |
| | 3-sulfonyl chloride | |
| 50 | 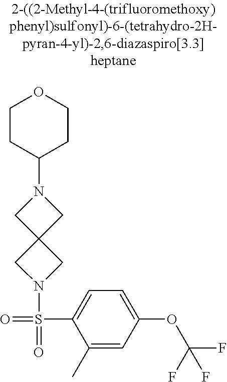 [SMILES: Cc1cc(OC(F)(F)F)ccc1S(=O)(=O)N1CC2(CN(C3CCOCC3)C2)C1] | Yield: 42 mg, %, TFA salt. LCMS m/z = 421.2 (M + H)⁺. LCMS t_{R} (4 min) = 1.71 min. |
| | SM: 2-methyl-4- | |
| | (trifluoromethoxy)benzenesulfonyl chloride | |

### Example 51: 2-((2-Methyl-6-(trifluoromethyl)pyridin-3-yl)sulfonyl)-6-(tetrahydro-2H-pyran-4-yl)-2,6-diazaspiro[3.3]heptane

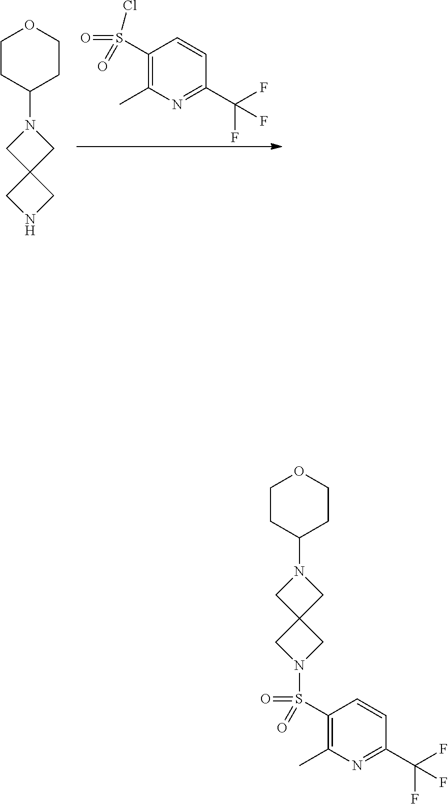

Hunigs base (322.56 mg, 2.50 mmol, 434.72 uL) was added to a mixture of 2-tetrahydropyran-4-yl-2,6-diazaspiro[3.3]heptane (167.19 mg, 655.16 umol, 2HCl) and 2-methyl-6-(trifluoromethyl)pyridine-3-sulfonyl chloride (162 mg, 623.96 umol) in DCM (5 mL). The reaction mixture was stirred at rt for 2.5 h. The reaction was then quenched with satd. NaHCO3 and water, and the solution was stirred at rt for 5 min. The aqueous layer was removed and the organic phase washed with water and concentrated. The residue was purified by normal phase chromatography (24 g, EtOAc/EtOH 3/1 in heptane 20-100%) to afford the product as a colorless oil, which precipitated out as a white solid upon addition of MeCN. Product was lyophilized overnight to afford the desired product (208 mg, 82.22% yield) as a white solid. LCMS m/z=406.2 (M+H)⁺. ¹H NMR (400 MHz, CD₃OD) δ (ppm): 8.46 (d, J=8.3 Hz, 1H), 7.83 (d, J=8.3 Hz, 1H), 4.04 (s, 4H), 3.94-3.86 (m, 2H), 3.39-3.32 (m, 6H), 2.86 (s, 3H), 2.26 (tt, J=10.7, 4.1 Hz, 1H), 1.72-1.60 (m, 2H), 1.30-1.14 (m, 2H).

### Example 52: 2-((2,4-Dimethylphenyl)sulfonyl)-6-(tetrahydro-2H-pyran-4-yl)-2,6-diazaspiro[3.4]octane

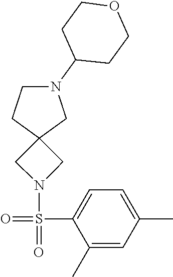 [SMILES: Cc1ccc(S(=O)(=O)N2CC3(CCN(C4CCOCC4)C3)C2)c(C)c1]

### 1. Synthesis of of 6-(tetrahydro-2H-pyran-4-yl)-2,6-diazaspiro[3.4]octane

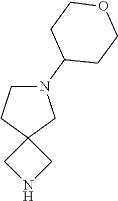 [SMILES: C1CC(N2CCC3(CNC3)C2)CCO1]

The title compound was prepared using a similar method described for step 1 of Example 36 from tetrahydropyran-4-one (300 mg, 3.0 mmol) and tert-butyl 2,6-diazaspiro[3.4]octane-2-carboxylate (530 mg, 2.5 mmol) to yield tert-butyl 6-(tetrahydro-2H-pyran-4-yl)-2,6-diazaspiro[3.4]octane-2-carboxylate (448 mg, 60%) which was used without further purification. ¹H NMR (400 MHz, CD₃OD) δ (ppm): 4.06-3.82 (m, 6H), 3.42 (br t, J=11.9 Hz, 2H), 3.18 (s, 2H), 3.02 (t, J=7.3 Hz, 2H), 2.85-2.65 (m, 1H), 2.21 (t, J=7.3 Hz, 2H), 1.98-1.91 (m, 2H), 1.60 (br dd, J=12.3, 4.8 Hz, 2H), 1.50-1.41 (m, 9H).

Subsequent deprotection of tert-butyl 6-(tetrahydro-2H-pyran-4-yl)-2,6-diazaspiro[3.4]octane-2-carboxylate with HCl (step 2 in Example 36) afforded the hydrochloride salt of 6-(tetrahydro-2H-pyran-4-yl)-2,6-diazaspiro[3.4]octane (222 mg) which was used without further purification. LCMS m/z=365.2 (M+H)⁺.

### 2. Synthesis of 2-((2,4-dimethylphenyl)sulfonyl)-6-(tetrahydro-2H-pyran-4-yl)-2,6-diazaspiro[3.4]octane

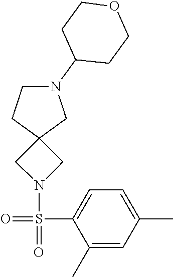 [SMILES: Cc1ccc(S(=O)(=O)N2CC3(CCN(C4CCOCC4)C3)C2)c(C)c1]

The title compound was prepared using a similar method to step 3 of Example 36 from the hydrochloride salt of 6-(tetrahydro-2H-pyran-4-yl)-2,6-diazaspiro[3.4]octane (38 mg, 0.1 mmol) and 2,4-dimethylbenzenesulfonyl chloride (25 mg, 0.1 mmol) to afford, after purification by column chromatography on silica gel (12 g, EtOAc/EtOH 3:1), 2-((2,4-dimethylphenyl)sulfonyl)-6-(tetrahydro-2H-pyran-4-yl)-2,6-diazaspiro[3.4]octane as an oil (13 mg, 29%). LCMS m/z=365.2 (M+H)⁺. ¹H NMR (400 MHz, CD₃OD) δ (ppm): 7.79 (d, J=8.0 Hz, 1H), 7.35-7.13 (m, 2H), 4.06-3.88 (m, 2H), 3.74 (q, J=7.9 Hz, 4H), 3.40 (br t, J=11.8 Hz, 2H), 2.76 (s, 2H), 2.69-2.62 (m, 2H), 2.61 (s, 3H), 2.41 (s, 3H), 2.32-2.22 (m, 1H), 2.02 (t, J=7.2 Hz, 2H), 1.83 (br d, J=12.8 Hz, 2H), 1.48 (qd, J=12.0, 4.8 Hz, 2H).

### Example 53-59

The Examples 53-59 in the following Table I were prepared using a similar method described for Example 52 from the hydrochloride salt of 6-(tetrahydro-2H-pyran-4-yl)-2,6-diazaspiro[3.4]octane (0.1 mmol) and the corresponding sulfonyl- or carbonyl chloride (0.1 mmol), unless stated otherwise. Purified by HPLC (acidic or basic conditions), unless otherwise stated.

TABLE I

| | Name/Structure/Sulfonyl- or carbonyl | |
|---|---|---|
| Example | chloride (SM) | Data |
| 53 | 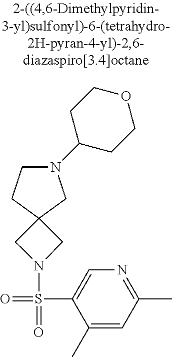 [SMILES: Cc1cc(C)c(S(=O)(=O)N2CC3(CCN(C4CCOCC4)C3)C2)cn1] | Yield: 12 mg, 33%. Purified by column chromatography on silica gel (12 g, EtOAc/MeOH 3:1). LCMS m/z = 366.1 (M + H)⁺. ¹H NMR (400 MHz, CD₃OD) δ (ppm): 8.87-8.74 (m, 1H), 7.40 (s, 1H), 4.07-3.99 (m, 2H), 3.98-3.82 (m, 4H), 3.53- 3.36 (m, 4H), 3.22-2.89 (m, 3H), 2.64 (s, 3H), 2.60 (s, 3H), 2.37-2.17 (m, 2H), 2.03- 1.90 (m, 2H), 1.72-1.51 (m, 2H). |
| | SM: 4,6-dimethylpyridine-3-sulfonyl | |
| | chloride | |
| | (21 mg, 0.1 mmol), amine (54 mg, 0.2 mmol) | |
| 54 | 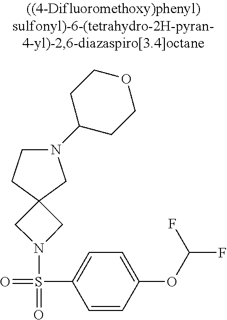 [SMILES: O=S(=O)(c1ccc(OC(F)F)cc1)N1CC2(CCN(C3CCOCC3)C2)C1] | Yield: 26 mg, 61%, TFA salt. LCMS m/z = 403.2 (M + H)⁺. LCMS t_{R} (4 min) = 1.39 min. |
| | SM: (4-difluoromethoxy)benzenesulfonyl chloride | |
| 55 | 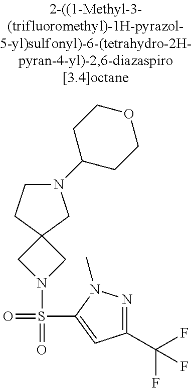 [SMILES: Cn1nc(C(F)(F)F)cc1S(=O)(=O)N1CC2(CCN(C3CCOCC3)C2)C1] | Yield: 27 mg, 45%, TFA salt. LCMS m/z = 409.2 (M + H)⁺. ¹H NMR (400 MHz, CD₃OD) δ (ppm): 7.25-7.18 (m, 1H), 4.17 (s, 3H), 3.97-3.90 (m, 2H), 3.89-3.82 (m, 4H), 3.38 (td, J = 12.0, 2.1 Hz, 2H), 2.73 (s, 2H), 2.64 (t, J = 7.2 Hz, 2H), 2.25 (tt, J = 11.0, 4.0 Hz, 1H), 2.00 (t, J = 7.2 Hz, 2H), 1.85-1.75 (m, 2H), 1.54- 1.39 (m, 2H). |
| | SM: 2-methyl-5- | |
| | (trifluoromethyl)pyrazole-3-sulfonyl chloride | |
| 56 | 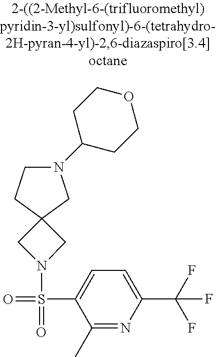 [SMILES: Cc1nc(C(F)(F)F)ccc1S(=O)(=O)N1CC2(CCN(C3CCOCC3)C2)C1] | Yield: 29 mg, 47%. Purified by column chromatography on silica gel (12 g, EtOAc/MeOH 3:1 50- 100%). LCMS m/z = 420.2 (M + H)⁺. ¹H NMR (400 MHz, CD₃OD) δ (ppm): 8.44-8.52 (m, 1H), 7.83 (d, J = 8.0 Hz, 1H), 3.98-3.84 (m, 6H), 3.38 (td, J = 11.9, 2.0 Hz, 2H), 2.88 (s, 3H), 2.81 (s, 2H), 2.66 (t, J = 7.2 Hz, 2H), 2.28 (s, 1H), 2.06 (t, J = 7.3 Hz, 2H), 1.88-1.77 (m, 2H), 1.55-1.41 (m, 2H). |
| | SM: 2-methyl-6-(trifluoromethyl)pyridine- | |
| | 3-sulfonyl chloride (41 mg, 0.2 mmol), | |
| | amine 29 mg, 0.2 mmol) | |
| 57 | 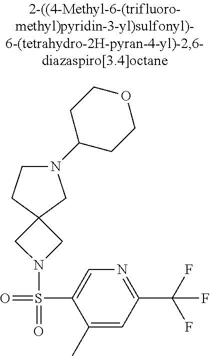 [SMILES: Cc1cc(C(F)(F)F)ncc1S(=O)(=O)N1CC2(CCN(C3CCOCC3)C2)C1] | Yield: 37 mg, 82%, TFA salt. LCMS m/z = 420.2 (M + H)⁺. ¹H NMR (400 MHz, CD₃OD) δ (ppm): 8.52-8.44 (m, 1H), 7.83 (d, J = 8.0 Hz, 1H), 3.98-3.84 (m, 6H), 3.38 (td, J = 11.9, 2.0 Hz, 2H), 2.88 (s, 3H), 2.81 (s, 2H), 2.66 (t, J = 7.2 Hz, 2H), 2.28 (s, 1H), 2.06 (t, J = 7.3 Hz, 2H), 1.88-1.77 (m, 2H), 1.55-1.41 (m, 2H). |
| | SM: 4-methyl-6-(trifluoromethyl)pyridine- | |
| | 3-sulfonyl chloride | |
| 58 | 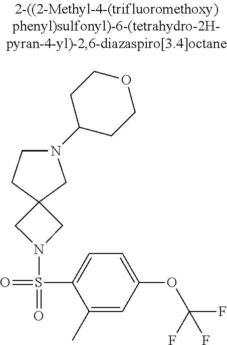 [SMILES: Cc1cc(OC(F)(F)F)ccc1S(=O)(=O)N1CC2(CCN(C3CCOCC3)C2)C1] | Yield: 48 mg, 100%, TFA salt. LCMS m/z = 435.2 (M + H)⁺. LCMS t_{R} (4 min) = 1.20 min. |
| | SM: 2-methyl-4-(trifluoromethoxy)benzenesulfonyl | |
| | chloride | |
| 59 | 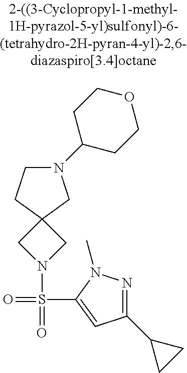 [SMILES: Cn1nc(C2CC2)cc1S(=O)(=O)N1CC2(CCN(C3CCOCC3)C2)C1] | Yield: 43 mg, 95%, TFA salt. LCMS m/z = 381.2 (M + H)⁺. LCMS t_{R} (4 min) = 1.33 min. |
| | SM: 5-cyclopropyl-2-methyl-pyrazole- | |
| | 3-sulfonyl chloride | |

### Example 60: 6-((1-methyl-3-(Trifluoromethyl)-1H-pyrazol-5-yl)sulfonyl)-2-(tetrahydro-2H-pyran-4-yl)-2,6-diazaspiro[3.4]octane

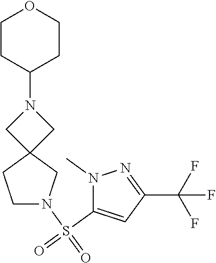 [SMILES: Cn1nc(C(F)(F)F)cc1S(=O)(=O)N1CCC2(CN(C3CCOCC3)C2)C1]

### 1. Synthesis of of 2-(tetrahydro-2H-pyran-4-yl)-2,6-diazaspiro[3.4]octane

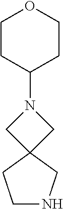 [SMILES: C1CC2(CN1)CN(C1CCOCC1)C2]

The title compound was prepared using a similar method described in step 1 for Example 36 from tetrahydropyran-4-one (251 mg, 2.5 mmol) and tert-butyl 2,6-diazaspiro[3.4]octane-6-carboxylate (commercial, 443 mg, 2.1 mmol) to yield tert-butyl 2-(tetrahydro-2H-pyran-4-yl)-2,6-diazaspiro[3.4]octane-6-carboxylate as a yellow oil (548 mg) which was used without further purification. ¹H NMR (400 MHz, CD₃OD) δ (ppm): 3.94 (br dd, J=10.8, 4.3 Hz, 2H), 3.89-3.78 (m, 4H), 3.39 (td, J=11.9, 2.0 Hz, 2H), 2.83 (s, 2H), 2.68 (t, J=7.2 Hz, 2H), 2.36-2.21 (m, 1H), 2.08 (t, J=7.2 Hz, 2H), 1.88-1.78 (m, 2H), 1.57-1.44 (m, 2H), 1.43 (s, 9H). Subsequent deprotection with HCl yielded the hydrochloride salt of 2-(tetrahydro-2H-pyran-4-yl)-2,6-diazaspiro[3.4]octane (87 mg) which was used without further purification. LCMS m/z=197.2 (M+H)⁺.

### 2. Synthesis of 6-((1-methyl-3-(trifluoromethyl)-1H-pyrazol-5-yl)sulfonyl)-2-(tetrahydro-2H-pyran-4-yl)-2,6-diazaspiro[3.4]octane

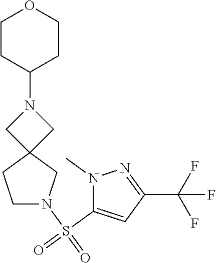 [SMILES: Cn1nc(C(F)(F)F)cc1S(=O)(=O)N1CCC2(CN(C3CCOCC3)C2)C1]

The title compound was prepared using a similar method to step 3 of Example 36 from the hydrochloride salt of 2-(tetrahydro-2H-pyran-4-yl)-2,6-diazaspiro[3.4]octane (24 mg, 0.1 mmol) and 2-methyl-5-(trifluoromethyl)pyrazole-3-sulfonyl chloride (22 mg, 0.1 mmol) to afford, after purification by HPLC (basic conditions), 6-((1-methyl-3-(trifluoromethyl)-1H-pyrazol-5-yl)sulfonyl)-2-(tetrahydro-2H-pyran-4-yl)-2,6-diazaspiro[3.4]octane as an oil (21 mg, 58%). LCMS m/z=409.2 (M+H)⁺. LCMS t_{R} (2 min)=0.60 min

### Example 61: (1′R,5′S)-8′-((4-(Difluoromethoxy)phenyl)sulfonyl)-1-(tetrahydro-2H-pyran-4-yl)-8′-azaspiro[azetidine-3,3′-bicyclo[3.2.1]octane]

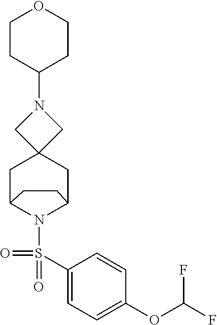 [SMILES: O=S(=O)(c1ccc(OC(F)F)cc1)N1C2CCC1CC1(C2)CN(C2CCOCC2)C1]

### 1. Synthesis of tert-butyl (1′R,5′S)-1-(tetrahydro-2H-pyran-4-yl)-8′-azaspiro[azetidine-3,3′-bicyclo[3.2.1]octane]-8′-carboxylate

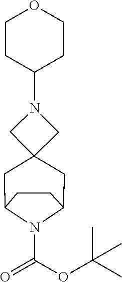 [SMILES: CC(C)(C)OC(=O)N1C2CCC1CC1(C2)CN(C2CCOCC2)C1]

The title compound was prepared using a similar method described in step 1 for Example 36 from tetrahydropyran-4-one (143 mg, 1.43 mmol) and v-butyl (1′R,5′S)-8′-azaspiro[azetidine-3,3′-bicyclo[3.2.1]octane]-8′-carboxylate (300 mg, 1.2 mmol) to yield, after purification by column chromatography on silica gel (DCM/MeOH 9:1), tert-butyl (1′R,5′S)-1-(tetrahydro-2H-pyran-4-yl)-8′-azaspiro[azetidine-3,3′-bicyclo[3.2.1]octane]-8′-carboxylate as colourless oil (426 mg). LCMS m/z=281.1 (M+H-t-Bu)⁺.

### 2. Synthesis of (1′R,5′S)-1-(tetrahydro-2H-pyran-4-yl)-8′-azaspiro[azetidine-3,3′-bicyclo[3.2.1]octane]

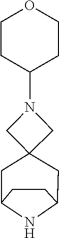 [SMILES: C1CC(N2CC3(CC4CCC(C3)N4)C2)CCO1]

To a solution of tert-butyl (1′R,5′S)-1-(tetrahydro-2H-pyran-4-yl)-8′-azaspiro[azetidine-3,3′-bicyclo[3.2.1]octane]-8′-carboxylate (426 mg) in DCM (10 mL) was added TFA (2.0 mL, 25.3 mmol) and the reaction mixture was stirred at 20° C. for 2 h, then concentrated to give the trifluoroacetate of (1′R,5′S)-1-(tetrahydro-2H-pyran-4-yl)-8′-azaspiro[azetidine-3,3′-bicyclo[3.2.1]octane] as brown oil (800 mg, crude), which was used without further purification. LCMS m/z=237.1 (M+H)⁺.

### 3. Synthesis of (1′R,5′S)-8′-((4-(difluoromethoxy)phenyl)sulfonyl)-1-(tetrahydro-2H-pyran-4-yl)-8′-azaspiro[azetidine-3,3′-bicyclo[3.2.1]octane]

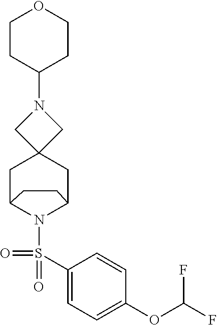 [SMILES: O=S(=O)(c1ccc(OC(F)F)cc1)N1C2CCC1CC1(C2)CN(C2CCOCC2)C1]

The title compound was prepared using a similar method to step 3 of Example 36 from the trifluoroacetate salt of (1′R,5′S)-1-(tetrahydro-2H-pyran-4-yl)-8′-azaspiro[azetidine-3,3′-bicyclo[3.2.1]octane] (350 mg) and 4-(difluoromethoxy)benzenesulfonyl chloride (276 mg, 1.1 mmol) to yield, after column chromatography on silica gel (DCM/MeOH 19:1) (1′R,5′S)-8′-((4-(difluoromethoxy)phenyl)sulfonyl)-1-(tetrahydro-2H-pyran-4-yl)-8′-azaspiro[azetidine-3,3′-bicyclo[3.2.1]octane] as a beige solid (160 mg, 48%). LCMS m/z=443.2 (M+H)⁺. ¹H NMR (400 MHz, CD₃OD) δ (ppm): 7.94-7.87 (m, 2H), 7.30 (d, J=8.8 Hz, 2H), 7.21-6.81 (m, 1H), 4.23 (br d, J=2.8 Hz, 2H), 3.91 (br dd, J=2.4, 11.6 Hz, 2H), 3.38-3.33 (m, 3H), 3.30-3.27 (m, 1H), 3.01 (s, 2H), 2.35-2.23 (m, 1H), 2.14 (dd, J=2.4, 14.0 Hz, 2H), 1.91 (br dd, J=3.2, 13.6 Hz, 2H), 1.71-1.58 (m, 4H), 1.52-1.39 (m, 2H), 1.31-1.15 (m, 2H).

### Example 62: 1-((1′R,5′S)-1-((2,4-Dimethylphenyl)sulfonyl)-8′-azaspiro[azetidine-3,3′-bicyclo[3.2.1]octan]-8′-yl)-2-methylpropan-2-ol

### 1. Synthesis of tert-butyl (1R,5S)-3-cyano-8-azabicyclo[3.2.1]octane-8-carboxylate

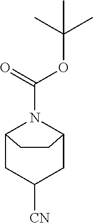 [SMILES: CC(C)(C)OC(=O)N1C2CCC1CC(C#N)C2]

Potassium 2-methylpropan-2-olate (1 M, 110 mmol) was suspended at −10° C. in DME (160 mL) under N₂ atmosphere. TosMIC (13.0 g, 66.6 mmol), dissolved in DME (10 mL), was added at 0° C. The reaction mixture was stirred for 1 h at 0° C. Isopropanol (7.0 mL, 88.8 mmol) was added at −10° C. and the reaction mixture was stirred for additional 30 min prior to addition of tert-butyl (1R,5S)-3-oxo-8-azabicyclo[3.2.1]octane-8-carboxylate (10 g, 44.4 mmol), dissolved in DME (10 mL), at 0° C. The reaction mixture was stirred 1 h at 0° C., then heated at 50° C. for 16 h, then filtered over celite. The residue was washed with ethyl acetate (100 mL, 2×). The organic layers were combined and evaporated and the resulting residue was purified by column chromatography on silica gel (PE/EtOAc 3:1) to give tert-butyl (1R,5S)-3-cyano-8-azabicyclo[3.2.1]octane-8-carboxylate as a white solid (8.0 g, 76%). Rf (TLC, PE/EtOAc 1:1) 0.43.

### 2. Synthesis of 8-(tert-butyl) 3-ethyl (1R,5S)-3-cyano-8-azabicyclo[3.2.1]octane-3,8-dicarboxylate

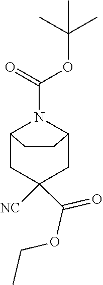 [SMILES: CCOC(=O)C1(C#N)CC2CCC(C1)N2C(=O)OC(C)(C)C]

LiHMDS (50 mL, 1 M in THF) was added dropwise to a solution of tert-butyl (1R,5S)-3-cyano-8-azabicyclo[3.2.1]octane-8-carboxylate (6.0 g, 25.0 mmol) in anhydrous THE (120 mL) at −78° C. The reaction mixture was stirred for 1 h, then ethyl carbonochloridate (5.7 g, 52.3 mmol) was added at −78° C., followed by stirring for 1 h. The mixture was warmed to 0° C. and stirred 1 h, then quenched with aq. NaHCO₃ (1 M, 50 mL) and extracted with EtOAc (50 mL, 3×). The combined organic layers were washed with brine (50 mL), dried over anhydrous Na₂SO₄, filtered and concentrated. The crude was purified by column chromatography on silica gel (PE/EtOAc 1:1) to give 8-(tert-butyl) 3-ethyl (1R,5S)-3-cyano-8-azabicyclo[3.2.1]octane-3,8-dicarboxylate as yellow liquid (7.8 g, 100%). ¹H NMR (400 MHz, CDCl₃) δ (ppm): 4.43-4.22 (m, 4H), 2.36 (br dd, J=3.2, 14.4 Hz, 2H), 2.27 (q, J=7.6 Hz, 2H), 2.16-2.04 (m, 4H), 1.47 (s, 9H), 1.31 (t, J=7.2 Hz, 3H).

### 3. Synthesis of tert-butyl (1R,5S)-3-cyano-3-(hydroxymethyl)-8-azabicyclo[3.2.1]octane-8-carboxylate

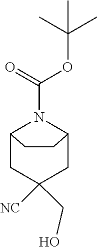 [SMILES: CC(C)(C)OC(=O)N1C2CCC1CC(C#N)(CO)C2]

To a solution of 8-(tert-butyl) 3-ethyl (1R,5S)-3-cyano-8-azabicyclo[3.2.1]octane-3,8-dicarboxylate (7.8 g, 25.3 mmol) in MeOH (120 mL) was added NaBH₄ (1.5 g, 39.6 mmol) at 0° C. The reaction mixture was stirred at 25° C. for 1 h, then quenched with sat. NH₄Cl (20 ml) and extracted with EtOAc (30 mL, 3×). The combined organic phase was washed with brine (20 mL, 2×), dried over anhydrous Na₂SO₄, filtered and concentrated to give a residue which was purified by column chromatography on silica gel (PE/EtOAc 3:1 to 1:1) to give tert-butyl (1R,5S)-3-cyano-3-(hydroxymethyl)-8-azabicyclo[3.2.1]octane-8-carboxylate as a white solid (6.3 g, 94%). ¹H NMR (400 MHz, CDCl₃) δ (ppm): 4.29 (br s, 2H), 3.54 (s, 2H), 2.26-2.18 (m, 2H), 2.13-2.04 (m, 2H), 2.01-1.94 (m, 2H), 1.90-1.83 (m, 2H), 1.45 (s, 9H).

### 4. Synthesis of tert-butyl (1R,5S)-3-cyano-3-((tosyloxy)methyl)-8-azabicyclo[3.2.1]octane-8-carboxylate

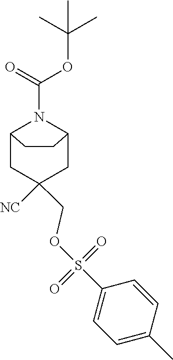 [SMILES: Cc1ccc(S(=O)(=O)OCC2(C#N)CC3CCC(C2)N3C(=O)OC(C)(C)C)cc1]

To solution of tert-butyl (1R,5S)-3-cyano-3-(hydroxymethyl)-8-azabicyclo[3.2.1]octane-8-carboxylate (6.3 g, 23.6 mmol), DMAP (289 mg, 2.4 mmol) and TEA (10 mL, 71.0 mmol) in DCM (150 mL) was added 4-methylbenzenesulfonyl chloride (9.0 g, 47.3 mmol) and the reaction mixture was stirred at 25° C. for 16 h, then concentrated. The residue was purified by column chromatography on silica gel (PE/EtOAc 3:1 to 1:1) to give tert-butyl (1R,5S)-3-cyano-3-((tosyloxy)methyl)-8-azabicyclo[3.2.1]octane-8-carboxylate as colourless oil (9.1 g, 92%). Rf (TLC, PE/EtOAc 1:1) 0.64.

### 5. Synthesis of tert-butyl (1′R,5′S)-8′-azaspiro[azetidine-3,3′-bicyclo[3.2.1]octane]-8′-carboxylate

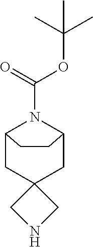 [SMILES: CC(C)(C)OC(=O)N1C2CCC1CC1(CNC1)C2]

To a solution of tert-butyl (1R,5S)-3-cyano-3-((tosyloxy)methyl)-8-azabicyclo[3.2.1]octane-8-carboxylate (7.1 g, 16.9 mmol) in THF (60 mL) at 0° C. was added LiAlH₄ (30 mL, 1 M in THF) and the mixture was stirred at 25° C. for 3 h. The reaction was quenched with Na₂SO₄×10H₂O (1 g), then filtered, washed with THE (20 ml) and concentrated to give tert-butyl (1′R,5′S)-8′-azaspiro[azetidine-3,3′-bicyclo[3.2.1]octane]-8′-carboxylate as a yellow solid (3.1 g, 73%) which was used without further purification. ¹H NMR (400 MHz, CD₃OD) δ (ppm): 4.22-4.14 (m, 2H), 3.69 (s, 2H), 3.25 (br s, 2H), (br d, J=11.8 Hz, 2H), 1.94-1.84 (m, 2H), 1.82-1.70 (m, 4H), 1.45 (s, 9H).

### 6. Synthesis of tert-butyl (1′R,5′S)-1-((2,4-dimethylphenyl)sulfonyl)-8′-azaspiro[azetidine-3,3′-bicyclo[3.2.1]octane]-8′-carboxylate

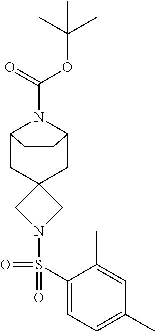 [SMILES: Cc1ccc(S(=O)(=O)N2CC3(CC4CCC(C3)N4C(=O)OC(C)(C)C)C2)c(C)c1]

To a solution of tert-butyl (1′R,5′S)-8′-azaspiro[azetidine-3,3′-bicyclo[3.2.1]octane]-8′-carboxylate (300 mg, 1.2 mmol) in DCM (6.0 mL) was added DIPEA (620 μL, 3.6 mmol) and 2,4-dimethylbenzenesulfonyl chloride (365 mg, 1.8 mmol) at 0° C. The reaction mixture was stirred at 20° C. for 4 h, then extracted with DCM (30 mL, 3×). The combined organic phase was washed with brine (30 mL, 3×), dried over anhydrous Na₂SO₄, filtered and concentrated to give a residue which was purified by column chromatography on silica gel (PE/EtOAC 1:1) to give tert-butyl (1′R,5′S)-1-((2,4-dimethylphenyl)sulfonyl)-8′-azaspiro[azetidine-3,3′-bicyclo[3.2.1]octane]-8′-carboxylate as a white solid (420 mg, 84%). LCMS m/z=365.2 (M+H-Boc)⁺.

### 7. Synthesis of (1′R,5′S)-1-((2,4-dimethylphenyl)sulfonyl)-8′-azaspiro[azetidine-3,3′-bicyclo[3.2.1]octane]

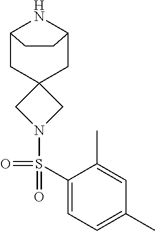 [SMILES: Cc1ccc(S(=O)(=O)N2CC3(CC4CCC(C3)N4)C2)c(C)c1]

A solution of tert-butyl (1′R,5′S)-1-((2,4-dimethylphenyl)sulfonyl)-8′-azaspiro[azetidine-3,3′-bicyclo[3.2.1]octane]-8′-carboxylate (400 mg, 1.0 mmol) in DCM (5.0 mL) and TFA (1.0 mL, 13.1 mmol) was stirred at 20° C. for 2 h. The mixture was concentrated in vacuo to give the trifluoroacetate salt of (1′R,5′S)-1-((2,4-dimethylphenyl)sulfonyl)-8′-azaspiro[azetidine-3,3′-bicyclo[3.2.1]octane] as a white oil (320 mg, crude). LCMS m/z=321.2 (M+H)⁺.

### 8. Synthesis of 1-((1′R,5′S)-1-((2,4-dimethylphenyl)sulfonyl)-8′-azaspiro[azetidine-3,3′-bicyclo[3.2.1]octan]-8′-yl)-2-methylpropan-2-ol

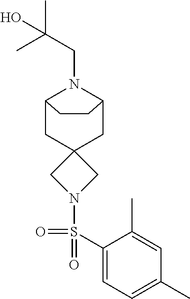 [SMILES: Cc1ccc(S(=O)(=O)N2CC3(CC4CCC(C3)N4CC(C)(C)O)C2)c(C)c1]

The trifluoroacetate salt of (1′R,5′S)-1-((2,4-dimethylphenyl)sulfonyl)-8′-azaspiro[azetidine-3,3′-bicyclo[3.2.1]octane](200 mg, 0.5 mmol), DIPEA (240 μL, 1.4 mmol) and 2,2-dimethyloxirane (1.6 g, 22.5 mmol) were added to a microwave tube in THE (1 mL). The sealed tube was heated at 80° C. for 3 h in a microwave. The mixture was concentrated in vacuo, the residue was purified by prep-HPLC (Column Welch Xtimate C18 150×25 mm×5 μm; Condition water (10 mM NH₄HCO₃)-ACN Begin B 50% End B 80% FlowRate (ml/min) 25) to give 1-((1′R,5′S)-1-((2,4-dimethylphenyl)sulfonyl)-8′-azaspiro[azetidine-3,3′-bicyclo[3.2.1]octan]-8′-yl)-2-methylpropan-2-ol as a white solid (55 mg, 30%, 100% purity). LCMS m/z=393.2 (M+H)⁺. ¹H NMR (400 MHz, CD₃OD) δ (ppm) 7.75 (d, J=8.1 Hz, 1H), 7.23 (s, 1H), 7.19 (d, J=8.1 Hz, 1H), 3.77 (s, 2H), 3.46-3.40 (m, 2H), 3.16 (br s, 2H), 2.57 (s, 3H), 2.38 (s, 3H), 2.20 (br s, 2H), 1.92-1.79 (m, 6H), 1.56 (br d, J=7.9 Hz, 2H), 1.12 (s, 6H).

### Example 63: (1′R,5′S)-8′-((4-(Difluoromethoxy)phenyl)sulfonyl)-1-(2-methoxy-2-methylpropyl)-8′-azaspiro[azetidine-3,3′-bicyclo[3.2.1]octane]

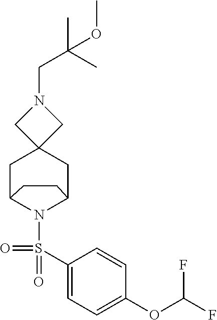 [SMILES: COC(C)(C)CN1CC2(CC3CCC(C2)N3S(=O)(=O)c2ccc(OC(F)F)cc2)C1]

### 1. Synthesis of tert-butyl (1′R,5′S)-1-(2-ethoxy-2-oxoethyl)-8′-azaspiro[azetidine-3,3′-bicyclo[3.2.1]octane]-8′-carboxylate

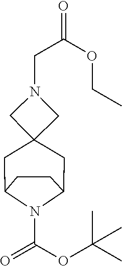 [SMILES: CCOC(=O)CN1CC2(CC3CCC(C2)N3C(=O)OC(C)(C)C)C1]

A solution of tert-butyl (1′R,5′S)-8′-azaspiro[azetidine-3,3′-bicyclo[3.2.1]octane]-8′-carboxylate (Example 63, step 5, 500 mg, 2.0 mmol) and ethyl 2-oxoacetate (809 mg, 4.0 mmol) in DCM (10 mL) was adjusted to pH 5-6 with AcOH and the reaction mixture was stirred at 18° C. for 30 min. NaBH(OAc)₃ (2.1 g, 9.9 mmol) was added. The mixture was stirred at 18° C. for 16 h, then quenched with sat. aq. NaHCO₃ (30 mL) and extracted with DCM (30 mL, 3×). The combined organic phase was dried with anhydrous Na₂SO₄, filtered and concentrated to give a residue which was purified by column chromatography on silica gel (PE/EtOAc 1:1 to EtOAc) to give tert-butyl (1′R,5′S)-1-(2-ethoxy-2-oxoethyl)-8′-azaspiro[azetidine-3,3′-bicyclo[3.2.1]octane]-8′-carboxylate as colorless oil (530 mg, 79%). LCMS m/z=283.1 [M+H-t-Bu]+.

### 2. Synthesis of ethyl 2-((1′R,5′S)-8′-azaspiro[azetidine-3,3′-bicyclo[3.2.1]octan]-1-yl)acetate

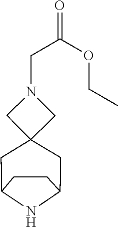 [SMILES: CCOC(=O)CN1CC2(CC3CCC(C2)N3)C1]

To a solution of tert-butyl (1′R,5′S)-1-(2-ethoxy-2-oxoethyl)-8′-azaspiro[azetidine-3,3′-bicyclo[3.2.1]octane]-8′-carboxylate (510 mg, 1.5 mmol) in EtOAc (20 mL) was added HCl (4 M, 11.3 mL) and the reaction mixture was stirred at 20° C. for 6 h, then concentrated to give the hydrochloride salt of ethyl 2-((1′R,5′S)-8′-azaspiro[azetidine-3,3′-bicyclo[3.2.1]octan]-1-yl)acetate as a white solid (460 mg). LCMS m/z=239.2 (M+H)⁺.

### 3. Synthesis of ethyl 2-((1′R,5′S)-8′-((4-(difluoromethoxy)phenyl)sulfonyl)-8′-azaspiro[azetidine-3,3′-bicyclo[3.2.1]octan]-1-yl)acetate

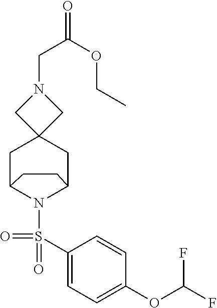 [SMILES: CCOC(=O)CN1CC2(CC3CCC(C2)N3S(=O)(=O)c2ccc(OC(F)F)cc2)C1]

To a solution of the hydrochloride salt of ethyl 2-((1′R,5′S)-8′-azaspiro[azetidine-3,3′-bicyclo[3.2.1]octan]-1-yl)acetate (280 mg, 0.9 mmol) and TEA (380 μL, 2.7 mmol) in DCM (10 mL) was added 4-(difluoromethoxy)benzenesulfonyl chloride (327 mg, 1.4 mmol) and the reaction mixture was stirred at 20° C. for 4 h. The mixture was diluted with water (10 mL) and extracted with DCM (20 mL, 3×). The combined organic phase was washed with brine (10 mL, 3×), dried over anhydrous Na₂SO₄, filtered and concentrated to give a residue which was purified by column chromatography on silica gel (DCM/MeOH 19:1) to afford ethyl 2-((1′R,5′S)-8′-((4-(difluoromethoxy)phenyl)sulfonyl)-8′-azaspiro[azetidine-3,3′-bicyclo[3.2.1]octan]-1-yl)acetate as a white solid (180 mg, 45%). LCMS m/z=445.2 (M+H)⁺.

### 4. Synthesis of 1-((1′R,5′S)-8′-((4-(difluoromethoxy)phenyl)sulfonyl)-8′-azaspiro[azetidine-3,3′-bicyclo[3.2.1]octan]-1-yl)-2-methylpropan-2-ol

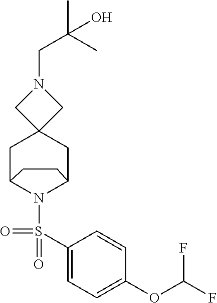 [SMILES: CC(C)(O)CN1CC2(CC3CCC(C2)N3S(=O)(=O)c2ccc(OC(F)F)cc2)C1]

In a Schlenk tube, to a 0° C. stirred solution of ethyl 2-((1′R,5′S)-8′-((4-(difluoromethoxy)phenyl)sulfonyl)-8′-azaspiro[azetidine-3,3′-bicyclo[3.2.1]octan]-1-yl)acetate (180 mg, 0.4 mmol) in THF (5 mL) was added bromo(methyl)magnesium (3 M, 2.8 mmol). The reaction mixture was stirred at 20° C. for 3 h, then quenched with sat. aq. NH₄Cl (10 mL) and extracted with EtOAc (20 mL, 3×). The combined organic layers were washed with brine (10 ml, 3×), dried with anhydrous Na₂SO₄, filtered and concentrated to give 1-((1′R,5′S)-8′-((4-(difluoromethoxy)phenyl)sulfonyl)-8′-azaspiro[azetidine-3,3′-bicyclo[3.2.1]octan]-1-yl)-2-methylpropan-2-ol as yellow oil (150 mg, crude). LCMS m/z=431.1 (M+H)⁺.

### 5. Synthesis of (1′R,5′S)-8′-((4-(difluoromethoxy)phenyl)sulfonyl)-1-(2-methoxy-2-methylpropyl)-8′-azaspiro[azetidine-3,3′-bicyclo[3.2.1]octane]

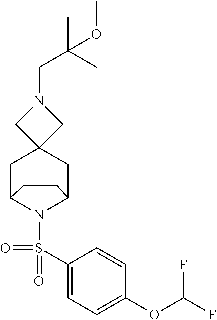 [SMILES: COC(C)(C)CN1CC2(CC3CCC(C2)N3S(=O)(=O)c2ccc(OC(F)F)cc2)C1]

To a mixture of 1-((1′R,5′S)-8′-((4-(difluoromethoxy)phenyl)sulfonyl)-8′-azaspiro[azetidine-3,3′-bicyclo[3.2.1]octan]-1-yl)-2-methylpropan-2-ol (150 mg, 0.4 mmol) in THF (10 mL) was added NaH (70 mg, 1.7 mmol, 60% purity) and Mel (130 μL, 2.2 mmol). The mixture was stirred at 0° C. for 10 min and then warmed to 20° C. for 16 h. The mixture was quenched with H₂O (10 mL, 3×), the combined organic layers was washed with DCM (20 mL), dried with anhydrous Na₂SO₄, filtered and concentrated to give a residue which was purified by prep-HPLC (Column Welch Xtimate C18 150×25 mm×5 μm Condition water (10 mM NH₄HCO₃)-ACN Begin B 41% End B 71% FlowRate (ml/min) 25) to give (1′R,5′i)-8′-((4-(difluoromethoxy)phenyl)sulfonyl)-1-(2-methoxy-2-methylpropyl)-8′-azaspiro[azetidine-3,3′-bicyclo[3.2.1]octane] as a brown solid (20 mg, 13%).

LCMS m/z=445.3 (M+H)⁺. ¹H NMR (400 MHz, CD₃OD) δ (ppm): 7.91 (d, J=8.8 Hz, 2H), 7.29 (d, J=8.8 Hz, 2H), 7.20-6.81 (m, 1H), 4.21 (br s, 2H), 3.38 (br s, 2H), 3.16 (s, 3H), 3.07 (br s, 2H), 2.47 (br s, 2H), 2.18 (br d, J=12.4 Hz, 2H), 1.87 (dd, J=2.4, 13.8 Hz, 2H), 1.67-1.56 (m, 2H), 1.50-1.36 (m, 2H), 1.09 (s, 6H).

### Example 64: (1′R,5′S)-1-((4-(Difluoromethoxy)phenyl)sulfonyl)-8′-(2-methoxy-2-methylpropyl)-8′-azaspiro[azetidine-3,3′-bicyclo[3.2.1]octane]

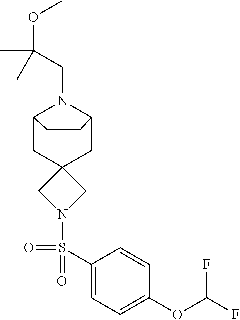 [SMILES: COC(C)(C)CN1C2CCC1CC1(C2)CN(S(=O)(=O)c2ccc(OC(F)F)cc2)C1]

### 1. Synthesis of tert-butyl (1′R,5′S)-1-((4-(difluoromethoxy)phenyl)sulfonyl)-8′-azaspiro[azetidine-3,3′-bicyclo[3.2.1]octane]-8′-carboxylate

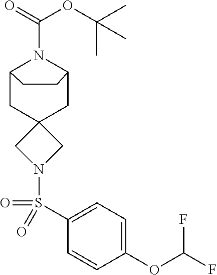 [SMILES: CC(C)(C)OC(=O)N1C2CCC1CC1(C2)CN(S(=O)(=O)c2ccc(OC(F)F)cc2)C1]

To a solution of tert-butyl (1′R,5′S)-8′-azaspiro[azetidine-3,3′-bicyclo[3.2.1]octane]-8′-carboxylate (Example 63, step 5, 200 mg, 0.8 mmol) in DCM (6.0 mL) was added DIPEA (410 μL, 2.4 mmol) and 4-difluoromethoxybenzene sulfonyl chloride (192 mg, 0.8 mmol) at 0° C. The reaction mixture was stirred at 20° C. for 3 h, then extracted with DCM (30 mL, 3×). The combined organic layers were washed with brine (30 mL, 3×), dried with anhydrous Na₂SO₄, filtered and concentrated to give a residue which was purified by column chromatography on silica gel (PE/EtOAc 1:1) to give tert-butyl (1′R,5′S)-1-((4-(difluoromethoxy)phenyl)sulfonyl)-8′-azaspiro[azetidine-3,3′-bicyclo[3.2.1]octane]-8′-carboxylate as a white solid (300 mg, 83%). LCMS m/z=403.1 (M+H-Boc)⁺.

### 2. Synthesis of (1′R,5′S)-1-((4-(difluoromethoxy)phenyl)sulfonyl)-8′-azaspiro[azetidine-3,3′-bicyclo[3.2.1]octane]

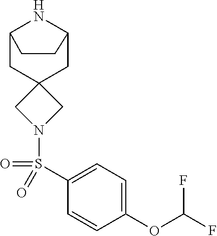 [SMILES: O=S(=O)(c1ccc(OC(F)F)cc1)N1CC2(CC3CCC(C2)N3)C1]

To a solution of tert-butyl (1′R,5′S)-1-((4-(difluoromethoxy)phenyl)sulfonyl)-8′-azaspiro[azetidine-3,3′-bicyclo[3.2.1]octane]-8′-carboxylate (300 mg, 0.7 mmol) in DCM (5.0 mL) was added TFA (1.0 mL, 13.1 mmol). The mixture was stirred at 20° C. for 2 h, then concentrated in vacuo to give the trifluoroacetate salt of (1′R,5′S)-1-((4-(difluoromethoxy)phenyl)sulfonyl)-8′-azaspiro[azetidine-3,3′-bicyclo[3.2.1]octane](250 mg, crude) as a white solid. LCMS m/z=359.1 (M+H)⁺.

### 3. Synthesis of 1-((1′R,5′S)-1-((4-(difluoromethoxy)phenyl)sulfonyl)-8′-azaspiro[azetidine-3,3′-bicyclo[3.2.1]octan]-8′-yl)-2-methylpropan-2-ol

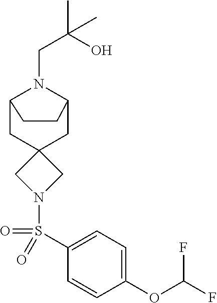 [SMILES: CC(C)(O)CN1C2CCC1CC1(C2)CN(S(=O)(=O)c2ccc(OC(F)F)cc2)C1]

The trifluoroacetic acid salt of (1′R,5′S)-1-((4-(difluoromethoxy)phenyl)sulfonyl)-8′-azaspiro[azetidine-3,3′-bicyclo[3.2.1]octane](150 mg, 0.3 mmol), DIPEA (220 μL, 1.3 mmol) and 2,2-dimethyloxirane (1.2 g, 17.1 mmol) were taken up into a microwave tube in THE (1 mL). The sealed tube was heated at 80° C. for 3 h in a microwave. The mixture was concentrated in vacuo, diluted with water (10 mL), extracted with EtOAc (20 mL, 3×). The combined organic layers were dried with anhydrous Na₂SO₄, filtered and concentrated under reduced pressure to give a residue which was purified by column chromatography on silica gel (DCM/MeOH 1:1) to give 1-((1′R,5′S)-1-((4-(difluoromethoxy)phenyl)sulfonyl)-8′-azaspiro[azetidine-3,3′-bicyclo[3.2.1]octan]-8′-yl)-2-methylpropan-2-ol as a white solid (130 mg, 95%). LCMS m/z=431.1 (M+H)⁺.

### 4. Synthesis of (1′R,5′S)-1-((4-(difluoromethoxy)phenyl)sulfonyl)-8′-(2-methoxy-2-methylpropyl)-8′-azaspiro[azetidine-3,3′-bicyclo[3.2.1]octane]

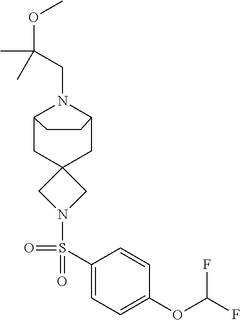 [SMILES: COC(C)(C)CN1C2CCC1CC1(C2)CN(S(=O)(=O)c2ccc(OC(F)F)cc2)C1]

To a solution of 1-((1′R,5′S)-1-((4-(difluoromethoxy)phenyl)sulfonyl)-8′-azaspiro[azetidine-3,3′-bicyclo[3.2.1]octan]-8′-yl)-2-methylpropan-2-ol (130 mg, 0.3 mmol) in THE (10 mL) was added NaH (60 mg, 1.5 mmol, 60% purity) at 0° C. under N₂. The reaction mixture was stirred at 0° C. for 30 min. Iodomethane (60 μL, 0.9 mmol) was added dropwise to the reaction mixture at 0° C. under N₂. The reaction mixture was stirred at 20° C. for 16 h, then extracted with DCM (10 mL, 3×). The combined organic layers were washed with brine (10 mL, 3×), dried over anhydrous Na₂SO₄, filtered and concentrated to give a residue which was purified by prep-HPLC (Column: Welch Xtimate C18 150×25 mm×5 μm; Condition: water (NH₄HCO₃)-CAN; Begin B: 51%; End B: 81%; Gradient Time (min): 10; 100% B Hold Time (min): 2; FlowRate (ml/min): 25) followed by prep-TLC (DCM/MeOH 10:1) to give (1′R,5′S)-1-((4-(difluoromethoxy)phenyl)sulfonyl)-8′-(2-methoxy-2-methylpropyl)-8′-azaspiro[azetidine-3,3′-bicyclo[3.2.1]octane] as a yellow solid (49 mg, 49%). LCMS m/z=445.2 (M+H)⁺. ¹H NMR: (400 MHz, DMSO-d₆) δ (ppm): 7.94-7.83 (m, 2H), 7.69-7.24 (m, 3H), 3.71 (s, 2H), 3.11-2.93 (m, 5H), 2.09 (s, 2H), 1.77-1.55 (m, 4H), 1.53-1.33 (m, 4H), 1.29-1.19 (m, 2H), 1.01 (s, 6H).

### Example 65: 2-((1-(cyclopropylmethyl)-3-(trifluoromethyl)-1H-pyrazol-5-yl)sulfonyl)-6-(tetrahydro-2H-pyran-4-yl)-2,6-diazaspiro[3.3]heptane

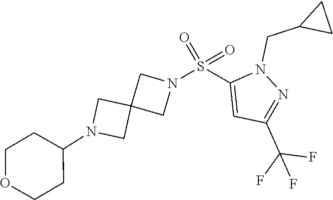 [SMILES: O=S(=O)(c1cc(C(F)(F)F)nn1CC1CC1)N1CC2(CN(C3CCOCC3)C2)C1]

2-((1-(Cyclopropylmethyl)-3-(trifluoromethyl)-1H-pyrazol-5-yl)sulfonyl)-6-(tetrahydro-2H-pyran-4-yl)-2,6-diazaspiro[3.3]heptane was prepared using a similar method described for Example 36 from the hydrochloride salt of 2-(tetrahydro-2H-pyran-4-yl)-2,6-diazaspiro[3.3]heptane (75 mg, 411 μmol) and 2-(cyclopropylmethyl)-5-(trifluoromethyl)pyrazole-3-sulfonyl chloride (91 mg, 316 μmol). The crude product was purified on column chromatography with {20-100%: EtOAc:EtOH (3:1) with 0.2% NH3OH} in EtOH, yielding the title compound as an off white residue (137 mg, 94% yield). LCMS m/z=435.3 (M+H)⁺. ¹H NMR (CHLOROFORM-d, 400 MHz) δ (ppm) 7.00 (s, 1H), 4.30 (d, 2H, J=7.5 Hz), 4.00 (s, 4H), 3.93 (td, 2H, J=3.8, 11.5 Hz), 3.34 (dt, 2H, J=2.3, 11.4 Hz), 3.25 (s, 4H), 2.1-2.2 (m, 1H), 1.6-1.6 (m, 2H), 1.45 (tquin, 1H, J=4.9, 7.7 Hz), 1.2-1.3 (m, 2H), 0.6-0.6 (m, 2H), 0.5-0.5 (m, 2H).

### Example 66: 2-((1-(cyclopropylmethyl)-3-(trifluoromethyl)-1H-pyrazol-5-yl)sulfonyl)-6-((tetrahydro-2H-pyran-4-yl)methyl)-2,6-diazaspiro[3.3]heptane

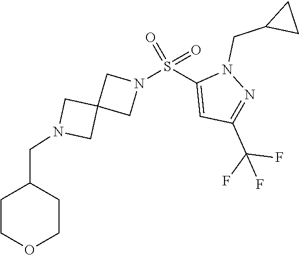 [SMILES: O=S(=O)(c1cc(C(F)(F)F)nn1CC1CC1)N1CC2(CN(CC3CCOCC3)C2)C1]

### 1. Synthesis of tert-butyl 6-((tetrahydro-2H-pyran-4-yl)methyl)-2,6-diazaspiro[3.3]heptane-2-carboxylate

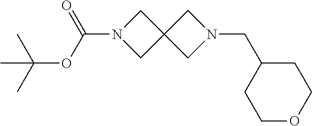 [SMILES: CC(C)(C)OC(=O)N1CC2(CN(CC3CCOCC3)C2)C1]

tetrahydropyran-4-carbaldehyde (2.06 g, 18.09 mmol) was added to a solution of tert-butyl 2,6-diazaspiro[3.3]heptane-2-carboxylate;oxalic acid (4 g, 8.22 mmol) and TEA (2.50 g, 24.66 mmol, 3.44 mL) in MeOH (60 mL). Acetic acid (493.67 mg, 8.22 mmol, 470.61 uL) was added until the pH=5-6 at 0° C. for 30 min, Sodium cyanoborohydride (2.58 g, 41.11 mmol) was added and the mixture was stirred at 20° C. for 2 h. The mixture was filtered and concentrated in vacuo. The crude material was purified on silica gel column chromatography (from MeOH/DCM=10/1) to yield the desired compound (3.36 g, 68.94% yield) as a white oil. LCMS m/z=297.2 (M+H)⁺. ¹H NMR (CHLOROFORM-d, 500 MHz) δ (ppm) 4.07 (s, 4H), 3.97-3.93 (m, 2H), 3.79 (br s, 4H), 3.39-3.32 (m, 2H), 2.68-2.64 (m, 2H), 1.72-1.62 (m, 1H), 1.63-1.58 (m, 2H), 1.42 (s, 9H), 1.33-1.27 (m, 2H).

### 2. Synthesis of 2-((tetrahydro-2H-pyran-4-yl)methyl)-2,6-diazaspiro[3.3]heptane

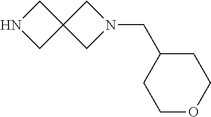 [SMILES: C1CC(CN2CC3(CNC3)C2)CCO1]

To a solution of tert-butyl 6-(tetrahydropyran-4-ylmethyl)-2,6-diazaspiro[3.3]heptane-2-carboxylate (350 mg, 1.18 mmol) in HFiPA (262.24 mg, 1.18 mmol, 197.17 uL) was added TFA (134.64 mg, 1.18 mmol, 90.42 uL) and the reaction mixture was stirred at 15° C. for 2 h. LCMS showed the desired product mass was observed. The mixture was concentrated to give the crude 2-(tetrahydropyran-4-ylmethyl)-2,6-diazaspiro[3.3]heptane (420 mg, crude, TFA) as a colourless oil. LCMS m/z=197.2 (M+H)⁺. ¹H NMR (CHLOROFORM-d, 400 MHz) δ (ppm) 4.52 (s, 4H), 4.34 (s, 4H), 3.96-3.90 (m, 2H), 3.44-3.36 (m, 2H), 3.23-3.18 (m, 1H), 3.12 (d, J=7.2 Hz, 2H), 1.92-1.86 (m, 1H), 1.63-1.58 (m, 2H), 1.37-1.33 (m, 2H).

### 3. Synthesis of 2-((1-(cyclopropylmethyl)-3-(trifluoromethyl)-1H-pyrazol-5-yl)sulfonyl)-6-((tetrahydro-2H-pyran-4-yl)methyl)-2,6-diazaspiro[3.3]heptane

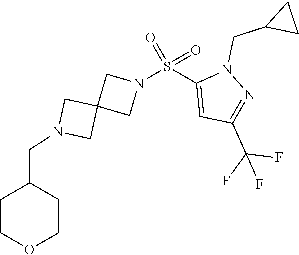 [SMILES: O=S(=O)(c1cc(C(F)(F)F)nn1CC1CC1)N1CC2(CN(CC3CCOCC3)C2)C1]

2-((1-(Cyclopropylmethyl)-3-(trifluoromethyl)-1H-pyrazol-5-yl)sulfonyl)-6-((tetrahydro-2H-pyran-4-yl)methyl)-2,6-diazaspiro[3.3]heptane was prepared using a similar method described for Example 46 from the hydrochloride salt of 2-((tetrahydro-2H-pyran-4-yl)methyl)-2,6-diazaspiro[3.3]heptane and 2-(cyclopropylmethyl)-5-(trifluoromethyl)pyrazole-3-sulfonyl chloride (71 mg, 248 μmol). The crude product was purified on column chromatography with {20-100%: EtOAc:EtOH (3:1) with 0.20% NH3OH} in EtOH, yielding the title compound as an off white residue (69 mg, 62% yield). LCMS m/z=449.3 (M+H)⁺. ¹H NMR (CHLOROFORM-d, 400 MHz) δ (ppm) 6.99 (s, 1H), 4.29 (d, 2H, J=7.5 Hz), 3.99 (s, 4H), 3.9-4.0 (m, 2H), 3.34 (dt, 2H, J=2.0, 11.8 Hz), 3.24 (s, 4H), 2.25 (d, 2H, J=6.5 Hz), 1.4-1.6 (m, 4H), 1.2-1.3 (m, 2H), 0.5-0.6 (m, 4H).

### Example 67: 2-((6-bromo-2-methylpyridin-3-yl)sulfonyl)-6-(tetrahydro-2H-pyran-4-yl)-2,6-diazaspiro[3.3]heptane

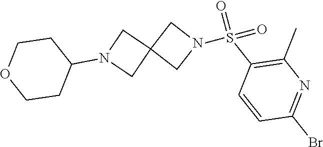 [SMILES: Cc1nc(Br)ccc1S(=O)(=O)N1CC2(CN(C3CCOCC3)C2)C1]

2-((6-Bromo-2-methylpyridin-3-yl)sulfonyl)-6-(tetrahydro-2H-pyran-4-yl)-2,6-diazaspiro[3.3]heptane was prepared using a similar method described for Example 46 from the hydrochloride salt of 2-(tetrahydro-2H-pyran-4-yl)-2,6-diazaspiro[3.3]heptane (350 mg, 1.6 mmol) and 6-bromo-2-methyl-pyridine-3-sulfonyl chloride (333.00 mg, 1.23 mmol). The crude product was purified on column chromatography with {0-100%: EtOAc:EtOH (3:1) with 0.2% NH3OH} in EtOH, yielding the title compound (512 mg, 70% yield). LCMS m/z=418.0 (M+H)⁺. ¹H NMR (CHLOROFORM-d, 500 MHz) δ (ppm) 7.93 (d, 1H, J=8.2 Hz), 7.40 (d, 1H, J=8.2 Hz), 3.85 (s, 2H), 3.74 (s, 2H), 3.60 (br d, 4H, J=4.3 Hz), 2.74 (s, 3H), 2.53 (quin, 1H, J=7.6 Hz), 2.1-2.3 (m, 6H), 1.9-2.0 (m, 2H).

### Example 68: rac-2-((2-methyl-6-(trifluoromethyl)pyridin-3-yl)sulfonyl)-6-(1-oxaspiro[3.3]heptan-6-yl)-2,6-diazaspiro[3.3]heptane

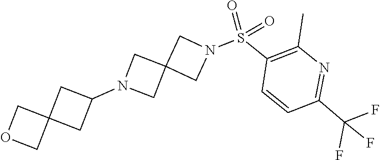 [SMILES: Cc1nc(C(F)(F)F)ccc1S(=O)(=O)N1CC2(CN(C3CC4(COC4)C3)C2)C1]

rac-2-((2-Methyl-6-(trifluoromethyl)pyridin-3-yl)sulfonyl)-6-(1-oxaspiro[3.3]heptan-6-yl)-2,6-diazaspiro[3.3]heptane was prepared using a similar method to Example 31 from the hydrochloride salt of 2-((2-methyl-6-(trifluoromethyl)pyridin-3-yl)sulfonyl)-2,6-diazaspiro[3.3]heptane (75 mg, 172 μmol) and 2-oxaspiro[3.3]heptan-6-one (20.4 mg, 181 μmol) The crude product was purified on column chromatography with {0-100%: EtOAc:EtOH (3:1) with 0.2% NH3OH} in EtOH to yield the title compound (23 mg, 32% yield). LCMS m/z=418.1 (M+H)⁺. ¹H NMR ¹H NMR (CHLOROFORM-d, 500 MHz) δ (ppm) 8.36 (br d, 1H, J=8.2 Hz), 7.65 (br d, 1H, J=8.2 Hz), 4.5-4.8 (m, 4H), 4.03 (s, 4H), 3.21 (s, 4H), 2.89 (s, 3H), 2.81 (br t, 1H, J=7.0 Hz), 2.2-2.4 (m, 2H), 1.9-2.0 (m, 2H).

### Example 69: rac-2-((2-methyl-6-(trifluoromethyl)pyridin-3-yl)sulfonyl)-6-(1-oxaspiro[3.3]heptan-6-yl)-2,6-diazaspiro[3.3]heptane

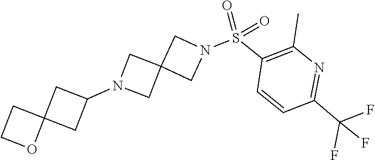 [SMILES: Cc1nc(C(F)(F)F)ccc1S(=O)(=O)N1CC2(CN(C3CC4(CCO4)C3)C2)C1]

rac-2-((2-Methyl-6-(trifluoromethyl)pyridin-3-yl)sulfonyl)-6-(1-oxaspiro[3.3]heptan-6-yl)-2,6-diazaspiro[3.3]heptane was prepared using a similar method to Example 31 from the hydrochloride salt of 2-((2-methyl-6-(trifluoromethyl)pyridin-3-yl)sulfonyl)-2,6-diazaspiro[3.3]heptane (90 mg, 207 μmol) and 1-oxaspiro[3.3]heptan-6-one (24.41 mg, 217.74 umol). The crude product was purified on column chromatography with {0-100%: EtOAc:EtOH (3:1) with 0.2% NH3OH} in EtOH to yield the title compound (58 mg, 67% yield). LCMS m/z=418.1 (M+H)⁺. ¹H NMR (CHLOROFORM-d, 500 MHz) δ (ppm) 8.36 (br d, 1H, J=7.9 Hz), 7.65 (br d, 1H, J=7.9 Hz), 4.4-4.6 (m, 2H), 3.9-4.1 (m, 4H), 3.58 (br d, 1H, J=6.7 Hz), 3.2-3.3 (m, 4H), 2.90 (s, 3H), 2.5-2.7 (m, 2H), 2.2-2.4 (m, 2H), 2.0-2.1 (m, 2H).

### Example 70 and 71: 2-((2-methyl-6-(trifluoromethyl)pyridin-3-yl)sulfonyl)-6-((4s,6s)-1-oxaspiro[3.3]heptan-6-yl)-2,6-diazaspiro[3.3]heptane and 2-((2-methyl-6-(trifluoromethyl)pyridin-3-yl)sulfonyl)-6-((4r,6r)-1-oxaspiro[3.3]heptan-6-yl)-2,6-diazaspiro[3.3]heptane

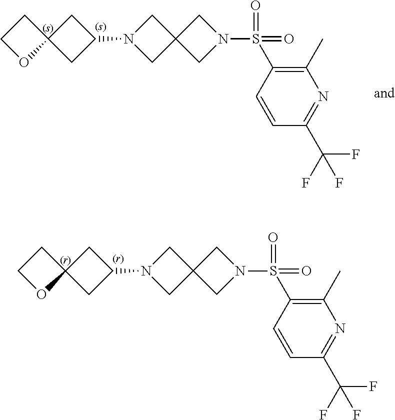

rac-2-((2-Methyl-6-(trifluoromethyl)pyridin-3-yl)sulfonyl)-6-(1-oxaspiro[3.3]heptan-6-yl)-2,6-diazaspiro[3.3]heptane was purified via SFC (CHIRALPAK IG 30×250 mm, 5 um Method: 30% EtOH w/ 0.1% DEA in CO2 (flow rate: 100 mL/min, ABPR 120 bar, MBPR 40 psi, column temp 40 deg C.)) to yield the 2 cis-trans isomers: 2-((2-methyl-6-(trifluoromethyl)pyridin-3-yl)sulfonyl)-6-((4s,6s)-1-oxaspiro[3.3]heptan-6-yl)-2,6-diazaspiro[3.3]heptane (5.8 mg, 100% ee, t_{R}=1.56 min, LCMS m/z=418.1 (M+H)⁺). and 2-((2-methyl-6-(trifluoromethyl)pyridin-3-yl)sulfonyl)-6-((4r,6r)-1-oxaspiro[3.3]heptan-6-yl)-2,6-diazaspiro[3.3]heptane (10.3 mg, 97.5% ee, t_{R}=1.88 min, LCMS m/z=418.1 (M+H)⁺).

### Example 72: 2-((1-methyl-3-(trifluoromethyl)-1H-pyrazol-5-yl)sulfonyl)-6-((4-methyltetrahydro-2H-pyran-4-yl)methyl)-2,6-diazaspiro[3.3]heptane

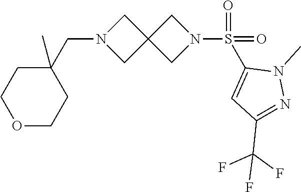 [SMILES: Cn1nc(C(F)(F)F)cc1S(=O)(=O)N1CC2(CN(CC3(C)CCOCC3)C2)C1]

### 1. Synthesis of tert-butyl 6-((4-methyltetrahydro-2H-pyran-4-yl)methyl)-2,6-diazaspiro[3.3]heptane-2-carboxylate

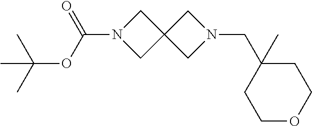 [SMILES: CC1(CN2CC3(C2)CN(C(=O)OC(C)(C)C)C3)CCOCC1]

tert-butyl 3,3-bis(bromomethyl)azetidine-1-carboxylate (350 mg, 1.02 mmol) and (4-methyltetrahydropyran-4-yl)methanamine (131.82 mg, 1.02 mmol) were dissolved in anhydrous DMF (4 mL) and stirred under nitrogen. DBU (341.71 mg, 2.24 mmol, 335.67 uL) was added and the reaction stirred at 60° C. The reaction was cooled to room temp, diluted with EtOAc and washed with water (4×). Solvent was removed in vacuo and the crude material purified by silica gel chromatography (EtOAc-EtOAc/EtOH (0-40%) to yield the title compound (150 mg, 47% yield). LCMS m/z=311.1 (M+H)⁺.

### 2. Synthesis of 2-((4-methyltetrahydro-2H-pyran-4-yl)methyl)-2,6-diazaspiro[3.3]heptane

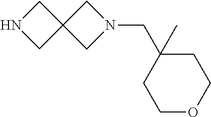

To a flask containing tert-butyl 6-[(4-methyltetrahydropyran-4-yl)methyl]-2,6-diazaspiro[3.3]heptane-2-carboxylate (900 mg, 2.90 mmol) in HFiPA (4 mL) was added TFA (1.30 g, 11.43 mmol, 875.50 uL) carefully dropwise at <5° C. Upon complete addition of TFA, the mixture was warmed to 23° C. and monitored by LCMS. After 19 hours, the mixture was concentrated under reduced pressure to afford a dark yellow residue that was triturated with ethanol and solvent removed to afford an off white solid. No further purification was performed. LCMS m/z=211.1 (M+H)⁺.

### 3. Synthesis of 2-((6-bromo-2-methylpyridin-3-yl)sulfonyl)-6-(tetrahydro-2H-pyran-4-yl)-2,6-diazaspiro[3.3]heptane

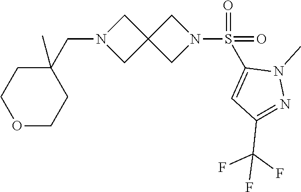 [SMILES: Cn1nc(C(F)(F)F)cc1S(=O)(=O)N1CC2(CN(CC3(C)CCOCC3)C2)C1]

Hunigs base (321.63 mg, 2.49 mmol, 433.47 uL) was added to a vial containing 2-[(4-methyltetrahydropyran-4-yl)methyl]-2,6-diazaspiro[3.3]heptane (128 mg, 395.88 umol, Trifluoroacetate) in anhydrous dichloromethane (2 mL). After 5 minutes, 2-methyl-5-(trifluoromethyl)pyrazole-3-sulfonyl chloride (98.42 mg, 395.88 umol) was added carefully to the solution. Upon complete addition of sulfonyl chloride, the reaction was stirred at room temperature and monitored with LCMS. After 3 hours, the reaction was carefully quenched with water. The solution was extracted three times with dichloromethane. The organic extractions were pooled then washed with NaHCO3 (sat, aq.) and water. Crude material was purified on column chromatography with {0-100%: EtOAc:EtOH (3:1) with 0.20% NH3OH} in EtOH to afford the title compound (78 mg, 47% yield). LCMS m/z=423.1 (M+H)⁺. ¹H NMR (CHLOROFORM-d, 500 MHz) δ (ppm) 6.99 (s, 1H), 4.14 (s, 3H), 3.99 (s, 4H), 3.6-3.7 (m, 2H), 3.5-3.6 (m, 2H), 3.31 (s, 4H), 2.22 (s, 2H), 1.4-1.5 (m, 2H), 1.19 (br d, 2H, J=13.4 Hz), 0.93 (s, 3H).

### Example 73: 2-((1-methyl-2-oxabicyclo[2.1.1]hexan-4-yl)methyl)-6-((2-methyl-6-(trifluoromethyl)pyridin-3-yl)sulfonyl)-2,6-diazaspiro[3.3]heptane

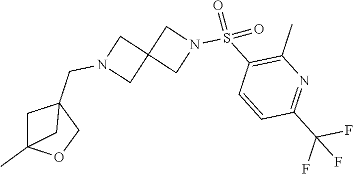 [SMILES: Cc1nc(C(F)(F)F)ccc1S(=O)(=O)N1CC2(CN(CC34COC(C)(C3)C4)C2)C1]

### 1. Synthesis of (6-((2-methyl-6-(trifluoromethyl)pyridin-3-yl)sulfonyl)-2,6-diazaspiro[3.3]heptan-2-yl)(1-methyl-2-oxabicyclo[2.1.1]hexan-4-yl)methanone

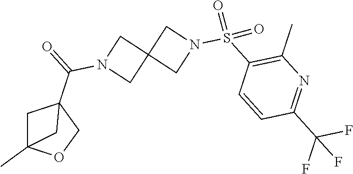 [SMILES: Cc1nc(C(F)(F)F)ccc1S(=O)(=O)N1CC2(CN(C(=O)C34COC(C)(C3)C4)C2)C1]

A mixture of 2-[[2-methyl-6-(trifluoromethyl)-3-pyridyl]sulfonyl]-2,6-diazaspiro[3.3]heptane (200 mg, 622.44 umol) and T3P (594.14 mg, 933.65 umol, 555.79 uL, 50% purity) in DMF (2 mL) and DIPEA (402.22 mg, 3.11 mmol, 542.07 uL) was heated at 80° C. for 10 min. 1-methyl-2-oxabicyclo[2.1.1]hexane-4-carboxylic acid (106.18 mg, 746.92 umol) in DMF (2 mL) was added at 80° C. and the reaction stirred for 3 hrs. The reaction mixture was diluted with DCM (5 mL) and washed with sat. aq. NaHCO₃, water and brine. The organic phase was dried over Na2SO4, filtered and concentrated. Solvent was removed and the crude material purified by silica gel chromatography (12 g SiO2, 50-100% EtOH:EtOAc 1:3 in heptane) to yield the title compound (270 mg, 97% yield). LCMS m/z=446.0 (M+H)⁺. ¹H NMR (CHLOROFORM-d, 500 MHz) δ (ppm) 8.37 (br d, 1H, J=8.2 Hz), 7.67 (br d, 1H, J=7.9 Hz), 4.32 (br s, 2H), 4.1-4.2 (m, 6H), 3.87 (s, 2H), 2.8-2.9 (m, 3H), 1.8-2.0 (m, 4H), 1.44 (s, 3H).

### 2. Synthesis of 2-((1-methyl-2-oxabicyclo[2.1.1]hexan-4-yl)methyl)-6-((2-methyl-6-(trifluoromethyl)pyridin-3-yl)sulfonyl)-2,6-diazaspiro[3.3]heptane

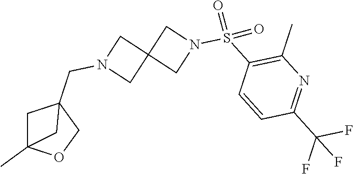 [SMILES: Cc1nc(C(F)(F)F)ccc1S(=O)(=O)N1CC2(CN(CC34COC(C)(C3)C4)C2)C1]

(1-methyl-2-oxabicyclo[2.1.1]hexan-4-yl)-[2-[[2-methyl-6-(trifluoromethyl)-3-pyridyl]sulfonyl]-2,6-diazaspiro[3.3]heptan-6-yl]methanone (270 mg, 606.12 umol) was dissolved in THF (1 mL). 1M Borane-THF (2.4 mL, 2.4 mmol) was added dropwise, and the solution heated to 50° C. overnight. The reaction was cooled to RT and quenched with the addition of 5 ml MeOH. The reaction was once again heated to 50° C. and stirred overnight. The reaction was cooled to RT and solvent removed in vacuo. The crude product was purified by column chromatography with {0-100%: EtOAc:EtOH (3:1) with 0.2% NH₃OH} in EtOH to provide the title compound (68 mg, 26%). LCMS m/z=432.0 (M+H)⁺. ¹H NMR (CHLOROFORM-d, 500 MHz) δ (ppm) 8.36 (br d, 1H, J=7.9 Hz), 7.65 (br d, 1H, J=8.2 Hz), 4.04 (s, 4H), 3.61 (s, 2H), 3.33 (s, 4H), 2.90 (s, 3H), 2.70 (s, 2H), 1.50 (s, 4H), 1.40 (s, 3H).

### Example 74: 2-((1-methyl-2-oxabicyclo[3.1.1]heptan-5-yl)methyl)-6-((2-methyl-6-(trifluoromethyl)pyridin-3-yl)sulfonyl)-2,6-diazaspiro[3.3]heptane

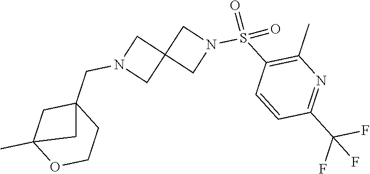 [SMILES: Cc1nc(C(F)(F)F)ccc1S(=O)(=O)N1CC2(CN(CC34CCOC(C)(C3)C4)C2)C1]

### 1. Synthesis of (6-((2-methyl-6-(trifluoromethyl)pyridin-3-yl)sulfonyl)-2,6-diazaspiro[3.3]heptan-2-yl)(1-methyl-2-oxabicyclo[3.1.1]heptan-5-yl)methanone

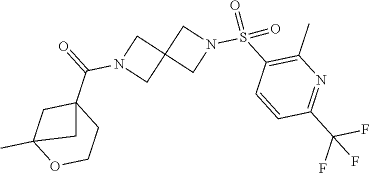 [SMILES: Cc1nc(C(F)(F)F)ccc1S(=O)(=O)N1CC2(CN(C(=O)C34CCOC(C)(C3)C4)C2)C1]

A mixture of 2-[[2-methyl-6-(trifluoromethyl)-3-pyridyl]sulfonyl]-2,6-diazaspiro[3.3]heptane (200 mg, 622.44 umol) and T3P (594.14 mg, 933.65 umol, 555.79 uL, 50% purity) in DMF (2 mL) and DIPEA (402.22 mg, 3.11 mmol, 542.07 uL) was heated at 80° C. for 10 min. A solution of 1-methyl-2-oxabicyclo[3.1.1]heptane-5-carboxylic acid (116.65 mg, 746.92 umol) in DMF (2 mL) was added and the reaction stirred at 80° C. for 4 hours. The reaction mixture was diluted with DCM (5 mL) and washed with sat. aq. NaHCO₃₁, water and brine. The organic phase was dried over Na2SO4, filtered and concentrated. Solvent was removed and the crude material purified by silica gel chromatography (12 g SiO2, 50-100% EtOH:EtOAc 1:3 in heptane) to yield the title compound (195 mg, 68% yield). LCMS m/z=460.1 (M+H)⁺. ¹H NMR (CHLOROFORM-d, 500 MHz) δ (ppm) 8.37 (br d, 1H, J=7.9 Hz), 7.67 (br d, 1H, J=8.2 Hz), 4.30 (br s, 2H), 4.1-4.2 (m, 8H), 2.90 (s, 3H), 2.1-2.2 (m, 4H), 2.0-2.1 (m, 2H), 1.24 (s, 3H).

### 2. Synthesis of 2-((1-methyl-2-oxabicyclo[3.1.1]heptan-5-yl)methyl)-6-((2-methyl-6-(trifluoromethyl)pyridin-3-yl)sulfonyl)-2,6-diazaspiro[3.3]heptane

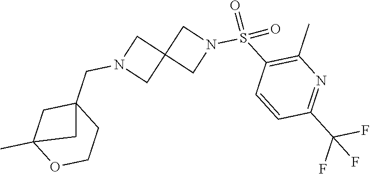 [SMILES: Cc1nc(C(F)(F)F)ccc1S(=O)(=O)N1CC2(CN(CC34CCOC(C)(C3)C4)C2)C1]

(1-methyl-2-oxabicyclo[3.1.1]heptan-5-yl)-[2-[[2-methyl-6-(trifluoromethyl)-3-pyridyl]sulfonyl]-2,6-diazaspiro[3.3]heptan-6-yl]methanone (180 mg, 391.75 umol) was dissolved in THF (1 mL). borane;tetrahydrofuran (1 M, 1.57 mL) was added dropwise and the solution heated to 50° C. overnight. The reaction was cooled to RT and quenched with addition of 5 ml MeoH. The reaction was again heated to 50° C. and stirred overnight. The reaction was cooled to RT and solvent removed. The crude product was purified by column chromatography with {0-100%: EtOAc:EtOH (3:1) with 0.2% NH₃OH} in EtOH to provide the title compound (130 mg, 74%). LCMS m/z=446.1 (M+H)⁺. ¹H NMR (CHLOROFORM-d, 500 MHz) δ (ppm) 8.36 (br d, 1H, J=8.2 Hz), 7.65 (d, 1H, J=7.9 Hz), 3.9-4.1 (m, 6H), 3.29 (s, 4H), 2.90 (s, 3H), 2.33 (s, 2H), 1.87 (br t, 2H, J=6.7 Hz), 1.82 (br dd, 2H, J=2.0, 7.2 Hz), 1.55 (br d, 2H, J=7.9 Hz), 1.21 (s, 3H).

### Example 75: 4-((6-((2-methyl-6-(trifluoromethyl)pyridin-3-yl)sulfonyl)-2,6-diazaspiro[3.3]heptan-2-yl)methyl)tetrahydro-2H-pyran-4-ol

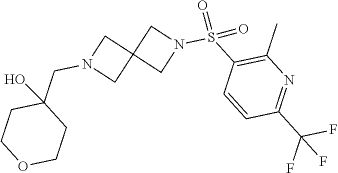 [SMILES: Cc1nc(C(F)(F)F)ccc1S(=O)(=O)N1CC2(CN(CC3(O)CCOCC3)C2)C1]

Hunigs base (122.17 mg, 945.32 umol, 164.65 uL) was added to a mixture of 2-[[6-(1,1-difluoroethyl)-2-methyl-3-pyridyl]sulfonyl]-2,6-diazaspiro[3.3]heptane (150 mg, 472.66 umol, Hydrochloride) and 1,6-dioxaspiro[2.5]octane (80.93 mg, 708.99 umol) in EtOH (4 mL). The mixture was heated at 55 C for 30 min, then RT overnight. The reaction was diluted with EtAOc and washed with satd. NaHCO₃. The organic phase was further washed with water, and concentrated. The residue was purified by column chromatography (12 g, EtOAc/EtOH 3/1 in heptane 50-100%) to afford the title compound (200 mg, 98% yield). LCMS m/z=432.3 (M+H)⁺. ¹H NMR (500 MHz, CHLOROFORM-d) δ (ppm) 8.21 (d, J=8.24 Hz, 1H), 7.54 (d, J=8.24 Hz, 1H), 3.94 (s, 4H), 3.60-3.71 (m, 4H), 3.38 (s, 4H), 2.78 (s, 3H), 2.31 (s, 2H), 1.94 (br t, J=18.77 Hz, 3H), 1.39-1.50 (m, 2H), 1.28 (br d, J=12.82 Hz, 2H).

### Example 76: 2-((1-methyl-3-(trifluoromethyl)-1H-pyrazol-5-yl)sulfonyl)-6-(4-methyltetrahydro-2H-pyran-4-yl)-2,6-diazaspiro[3.3]heptane

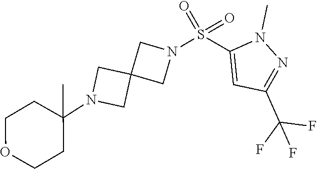 [SMILES: Cn1nc(C(F)(F)F)cc1S(=O)(=O)N1CC2(CN(C3(C)CCOCC3)C2)C1]

### 1. Synthesis of 4-(6-((1-methyl-3-(trifluoromethyl)-1H-pyrazol-5-yl)sulfonyl)-2,6-diazaspiro[3.3]heptan-2-yl)tetrahydro-2H-pyran-4-carbonitrile

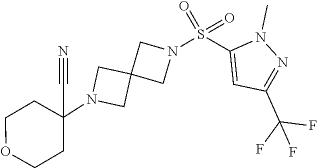 [SMILES: Cn1nc(C(F)(F)F)cc1S(=O)(=O)N1CC2(CN(C3(C#N)CCOCC3)C2)C1]

4-Hydroxytetrahydropyran-4-carbonitrile (90.14 mg, 709.00 umol) and 2-[2-methyl-5-(trifluoromethyl)pyrazol-3-yl]sulfonyl-2,6-diazaspiro[3.3]heptane (220 mg, 709.00 umol) were dissolved in ethanol (3 mL) in a sealed tube and heated to 65° C. Heating was continued for 4 hrs. The reaction was cooled to RT and stirred overnight. Solvent was removed in vacuo and the crude material purified by column chromatography (0-100% EtOAc in Heptane) to afford the desired compound (220 mg, 74%). LCMS m/z=420.1 (M+H)⁺. ¹H NMR (CHLOROFORM-d, 500 MHz) δ (ppm) 7.03 (s, 1H), 4.15 (s, 3H), 4.05 (s, 4H), 3.90 (br d, 2H, J=12.2 Hz), 3.5-3.7 (m, 2H), 3.42 (br s, 4H), 1.77 (br d, 2H, J=13.1 Hz), 1.4-1.6 (m, 2H).

### 2. Synthesis of 2-((1-methyl-3-(trifluoromethyl)-1H-pyrazol-5-yl)sulfonyl)-6-(4-methyltetrahydro-2H-pyran-4-yl)-2,6-diazaspiro[3.3]heptane

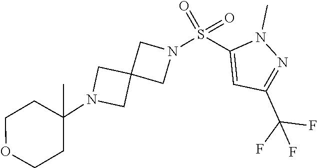 [SMILES: Cn1nc(C(F)(F)F)cc1S(=O)(=O)N1CC2(CN(C3(C)CCOCC3)C2)C1]

4-[2-[2-Methyl-5-(trifluoromethyl)pyrazol-3-yl]sulfonyl-2,6-diazaspiro[3.3]heptan-6-yl]tetrahydropyran-4-carbonitrile (200 mg, 476.85 umol) was dissolved in THF (5 mL) and cooled to 0° C. Methylmagnesium bromide (1.4 M, 1.36 mL) was added dropwise and the reaction stirred. The reaction was allowed to warm to room temp. and stir overnight. The reaction was quenched by addition of NaHCO₃ and extracted with EtOAc. Organics were combined and the solvent removed in vacuo. Crude material was purified by column chromatography with {0-100%: EtOAc:EtOH (3:1) with 0.2% NH₃OH} in EtOH. LCMS m/z=409.1 (M+H)⁺. ¹H NMR (CHLOROFORM-d, 500 MHz) δ (ppm) 6.9-7.1 (m, 1H), 4.15 (s, 3H), 4.01 (s, 4H), 3.7-3.9 (m, 2H), 3.5-3.6 (m, 2H), 3.24 (s, 4H), 1.3-1.5 (m, 2H), 1.2-1.3 (m, 2H), 0.93 (s, 3H).

### Example 77: 2-((2-methyl-6-(trifluoromethyl)pyridin-3-yl)sulfonyl)-6-(4-methyltetrahydro-2H-pyran-4-yl)-2,6-diazaspiro[3.3]heptane

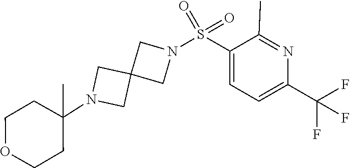 [SMILES: Cc1nc(C(F)(F)F)ccc1S(=O)(=O)N1CC2(CN(C3(C)CCOCC3)C2)C1]

### 1. Synthesis of 4-(6-((2-methyl-6-(trifluoromethyl)pyridin-3-yl)sulfonyl)-2,6-diazaspiro[3.3]heptan-2-yl)tetrahydro-2H-pyran-4-carbonitrile

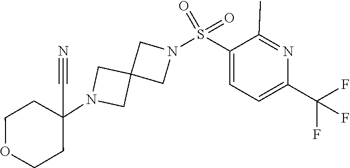 [SMILES: Cc1nc(C(F)(F)F)ccc1S(=O)(=O)N1CC2(CN(C3(C#N)CCOCC3)C2)C1]

4-Hydroxytetrahydropyran-4-carbonitrile (49.46 mg, 389.02 umol) and 2-[[2-methyl-6-(trifluoromethyl)-3-pyridyl]sulfonyl]-2,6-diazaspiro[3.3]heptane (125 mg, 389.02 umol) were dissolved in ethanol (3 mL) in a sealed tube and heated to 65° C. Heating was continued for 4 hrs. The reaction was cooled to RT and stirred overnight. Solvent was removed in vacuo and the crude material was purified by column chromatography (0-100% EtOAc in Heptane) to afford the desired compound (220 mg, 74%). LCMS m/z=431.0 (M+H)⁺. ¹H NMR (CHLOROFORM-d, 500 MHz) δ (ppm) 8.37 (br d, 1H, J=7.9 Hz), 7.66 (br d, 1H, J=7.9 Hz), 4.09 (s, 3H), 4.0-4.0 (m, 1H), 3.91 (br d, 2H, J=12.2 Hz), 3.60 (br t, 2H, J=10.2 Hz), 3.46 (s, 4H), 2.91 (s, 3H), 1.78 (br d, 2H, J=13.4 Hz), 1.5-1.6 (m, 2H).

### 2. Synthesis of 2-((2-methyl-6-(trifluoromethyl)pyridin-3-yl)sulfonyl)-6-(4-methyltetrahydro-2H-pyran-4-yl)-2,6-diazaspiro[3.3]heptane

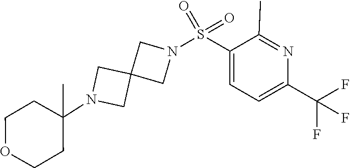 [SMILES: Cc1nc(C(F)(F)F)ccc1S(=O)(=O)N1CC2(CN(C3(C)CCOCC3)C2)C1]

4-[2-[[2-Methyl-6-(trifluoromethyl)-3-pyridyl]sulfonyl]-2,6-diazaspiro[3.3]heptan-6-yl]tetrahydropyran-4-carbonitrile (185 mg, 429.79 umol) was dissolved in THF (5 mL) and cooled to 0° C. Methylmagnesium bromide (1.4 M, 1.23 mL) was added dropwise and the reaction stirred. The reaction was allowed to warm to room temp. and stir overnight. The reaction was quenched by addition of NaHCO₃ and extracted with EtOAc. The organics were combined and solvent removed in vacuo. Crude material was purified by column chromatography with {0-100%: EtOAc:EtOH (3:1) with 0.2% NH3OH} in EtOH to afford the target compound (108 mg, 59%). LCMS m/z=420.0 (M+H)⁺. ¹H NMR (CHLOROFORM-d, 500 MHz) δ (ppm) 8.37 (br d, 1H, J=7.9 Hz), 7.66 (br d, 1H, J=7.9 Hz), 4.05 (s, 4H), 3.7-3.8 (m, 2H), 3.4-3.7 (m, 2H), 3.29 (s, 4H), 2.91 (s, 3H), 1.3-1.5 (m, 2H), 1.2-1.3 (m, 2H), 0.94 (s, 3H).

### Example 78: 2-((6-(1,1-difluoroethyl)-2-methylpyridin-3-yl)sulfonyl)-6-(2-oxaspiro[3.3]heptan-6-yl)-2,6-diazaspiro[3.3]heptane

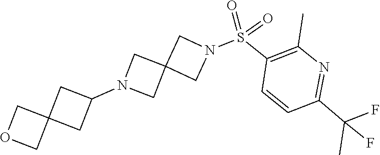 [SMILES: Cc1nc(C(C)(F)F)ccc1S(=O)(=O)N1CC2(CN(C3CC4(COC4)C3)C2)C1]

### 1. Synthesis of 3-Bromo-6-(1,1-difluoroethyl)-2,3-dimethylpyridine

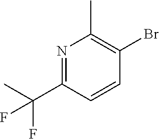

To a solution of 1-(5-bromo-6-methylpyridin-2-yl)ethan-1-one (4 g, 15.94 mmol) in Toluene (100 mL) was added n-BuLi (2.5 M, 6.50 mL) at −78° C. under a nitrogen atmosphere. After stirring for 1 h, DMA (8.38 g, 47.82 mmol, 4.5 mL) was added, the mixture was stirred at −78° C. for 1 h and slowly warmed to 20° C. Thereafter, saturated aqueous ammonium chloride solution was added to quench the reaction, and extraction and liquid separation. The extract was purified by silica gel column chromatography (Petroleum ether/EtOAc=1/0 to 8/1) to give the title compound (2.6 g, 12.15 mmol, 76.19% yield) as a white solid. LCMS m/z=216.1 (M+H)⁺. ¹H NMR (400 MHz, CDCl₃) δ (ppm) 7.93 (d, J=8.0 Hz, 1H), 7.73 (d, J=8.0 Hz, 1H), 2.73 (s, 3H), 2.70 (s, 3H).

### 2. Synthesis of 3-(benzylthio)-6-(1,1-difluoroethyl)-2-methylpyridine

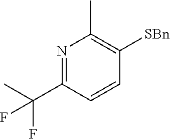

To a solution of 3-Bromo-6-(1,1-difluoroethyl)-2,3-dimethylpyridine (1.9 g, 8.05 mmol) in Dioxane (20 mL) was added DIEA (3.12 g, 24.15 mmol, 4.21 mL), Pd(tBu₃P)₂ (411.34 mg, 804.89 μmol) and BnSH (2.43 g, 19.56 mmol, 2.30 mL). Then the mixture was stirred at 100° C. for 16 h under N₂. The mixture was quenched with H₂O (100 mL). The reaction mixture was extracted with DCM (50 mL×5), and the combined organic phases were washed with brine (50 mL), dried over Na₂SO₄, filtered and concentrated to afford the title compound (3 g, crude) as a yellow oil. LCMS m/z=280.1 [M+H]+. ¹H NMR (500 MHz, CDCl₃) δ (ppm) 7.47 (d, J=8.0 Hz, 1H), 7.31 (d, J=8.5 Hz, 1H), 7.27-7.17 (m, 5H), 4.08 (s, 2H), 2.51 (s, 3H), 1.93 (t, J=19.0 Hz, 3H).

### 3. Synthesis of 6-(1,1-difluoroethyl)-2-methylpyridine-3-sulfonyl chloride

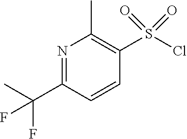

To a solution of 3-(benzylthio)-6-(1,1-difluoroethyl)-2-methylpyridine (1 g, 3.58 mmol) in DCM (10 mL) and water (2 mL) was added SO₂Cl₂ (3.38 g, 25.06 mmol, 2.03 mL at 0° C. The mixture was stirred at 0° C. for 1 h under N₂. The mixture was diluted with water (50 mL) and extracted with DCM (50 mL×5). The combined organic phase was dried over anhydrous sodium sulfate and concentrated in vacuo to give the desired compound (550 mg, crude) as yellow oil. ¹H NMR (400 MHz, CDCl₃) δ (ppm) 8.44 (d, J=8.4 Hz, 1H), 7.74 (d, J=8.0 Hz, 1H), 3.05 (s, 3H), 2.05 (t, J=18.4 Hz, 3H).

### 4. Synthesis of tert-butyl 6-((6-(1,1-difluoroethyl)-2-methylpyridin-3-yl)sulfonyl)-2,6-diazaspiro[3.3]heptane-2-carboxylate

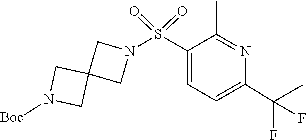 [SMILES: Cc1nc(C(C)(F)F)ccc1S(=O)(=O)N1CC2(CN(C(=O)OC(C)(C)C)C2)C1]

To a solution of tert-butyl 2,6-diazaspiro[3.3]heptane-2-carboxylate (500 mg, 1.75 mmol, Oxalate salt) in DCM (10 mL) was added DIPEA (677.17 mg, 5.24 mmol, 912.63 μL) and 6-(1,1-difluoroethyl)-2-methylpyridine-3-sulfonyl chloride (535.84 mg, 2.10 mmol) at 0° C. The mixture was stirred at 20° C. for 14 h. Water (50 mL) was added and extracted with DCM (50 mL×3). The organic layer was washed with brine (30 mL), dried over Na₂SO₄, filtered and concentrated. The crude product was purified by column chromatography (EtOAc in petroleum ether=30% to 50%) to give the desired compound (720 mg, 1.72 mmol, 98.75% yield) as a white solid. LCMS m/z=418.1 (M+H)⁺. ¹H NMR (400 MHz, MeOD) δ (ppm) 8.37 (d, J=8.4 Hz, 1H), 7.70 (d, J=8.0 Hz, 1H), 4.06 (s, 4H), 4.03 (s, 4H), 2.84 (s, 3H), 2.04-1.94 (m, 3H), 1.41 (s, 9H).

### 5. Synthesis of 2-((6-(1,1-difluoroethyl)-2-methylpyridin-3-yl)sulfonyl)-2,6-diazaspiro[3.3]heptane

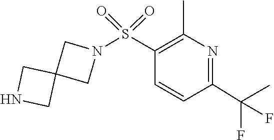 [SMILES: Cc1nc(C(C)(F)F)ccc1S(=O)(=O)N1CC2(CNC2)C1]

To a solution of tert-butyl 6-((6-(1,1-difluoroethyl)-2-methylpyridin-3-yl)sulfonyl)-2,6-diazaspiro[3.3]heptane-2-carboxylate (700 mg, 1.68 mmol) in HFIP (15.96 g, 94.98 mmol, 10 mL) was added TFA (573.55 mg, 5.03 mmol, 385.19 μL) at 20° C. The mixture was stirred at 20° C. for 3 h. The mixture was concentrated to give the desired compound (700 mg, crude, TFA) as a yellow oil. LCMS m/z=318.1 (M+H)⁺. ¹H NMR (400 MHz, MeOD) δ (ppm) 8.37 (d, J=8.0 Hz, 1H), 7.71 (d, J=8.4 Hz, 1H), 4.24 (s, 4H), 4.15 (s, 4H), 2.84 (s, 3H), 1.99 (t, J=18.8 Hz, 3H).

### 6. Synthesis of 2-((6-(1,1-difluoroethyl)-2-methylpyridin-3-yl)sulfonyl)-6-(2-oxaspiro[3.3]heptan-6-yl)-2,6-diazaspiro[3.3]heptane

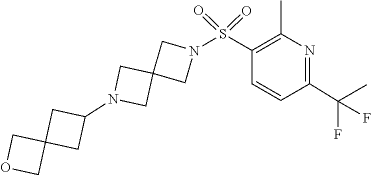 [SMILES: Cc1nc(C(C)(F)F)ccc1S(=O)(=O)N1CC2(CN(C3CC4(COC4)C3)C2)C1]

To a solution of 2-((6-(1,1-difluoroethyl)-2-methylpyridin-3-yl)sulfonyl)-2,6-diazaspiro[3.3]heptane (200 mg, 464.72 μmol, TFA) and 2-oxaspiro[3.3]heptan-6-one (78.16 mg, 697.08 μmol) in MeOH (5 mL) was added TEA (141.07 mg, 1.39 mmol, 194.32 μL) to pH=8. The solution was stirred at 20° C. for 20 minutes then adjusted to pH=6 using acetic acid. The stirring was continued for 30 minutes at 20° C. NaBH₃CN (146.02 mg, 2.32 mmol) was added and the reaction mixture was stirred at 20° C. for 14 h. The mixture was diluted with water (50 mL) and extracted with DCM (50 mL×3). The combined organic phase was dried over anhydrous sodium sulfate, filtered and concentrated in vacuo. The crude material was purified by prep-HPLC ((Column: C18-1 150*30 mm*5 μm, Condition: water (NH₄HCO₃)-ACN, 32%˜62%, Flow Rate (mL/min): 25)) to give the title compound (53.23 mg, 128.74 μmol, 27.70% yield) as a white oil. LCMS m/z=414.1 (M+H)⁺. ¹H NMR (400 MHz, MeOD) δ (ppm) 8.36 (d, J=8.4 Hz, 1H), 7.70 (d, J=8.4 Hz, 1H), 4.67-4.56 (m, 4H), 2.80-3.98 (s, 4H), 3.27 (s, 4H), 2.94-2.87 (m, 1H), 2.83 (s, 3H), 2.33-2.26 (m, 2H), 2.05-1.97 (m, 3H), 1.97-1.93 (m, 2H).

The Examples 79-89 in the following Table were prepared using a similar method described in step 4 for Example 78 from the TFA salt of 2-((6-(1,1-difluoroethyl)-2-methylpyridin-3-yl)sulfonyl)-2,6-diazaspiro[3.3]heptane (1 equiv.) and the corresponding aldehyde or ketone (SM).

| Example | Name/Structure/(SM) | Data |
|---|---|---|
| 79 | 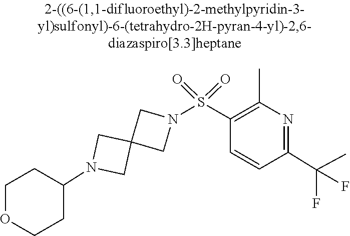 [SMILES: Cc1nc(C(C)(F)F)ccc1S(=O)(=O)N1CC2(CN(C3CCOCC3)C2)C1] | Yield: 65 mg, 50%. LCMS m/z = 402.1 (M + H)⁺. ¹H NMR (400 MHz, MeOD) δ (ppm) 8.37 (d, J = 8.4 Hz, 1H), 7.71 (d, J = 8.4 Hz, 1H), 4.01 (s, 4H), 3.91 (d, J = 10.8 Hz, 2H), 3.38-3.32 (m, 6H), 2.84 (s, 3H), 2.31-2.21 (m, 1H), 2.00 (t, J = 18.8 Hz, 3H), 1.65 (d, J = 12.0 Hz, 2H), 1.26-1.15 (m, 2H). |
| | SM: tetrahydro-4H-pyran-4-one | |
| 80 | 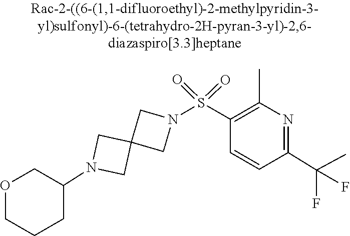 [SMILES: Cc1nc(C(C)(F)F)ccc1S(=O)(=O)N1CC2(CN(C3CCCOC3)C2)C1] | Yield: 17 mg, 18%. LCMS m/z = 402.1 (M + H)⁺. ¹H NMR (400 MHz, MeOD) δ (ppm) 8.36 (d, J = 8.0 Hz, 1H), 7.70 (d, J = 8.0 Hz, 1H), 4.00 (s, 4H), 3.76-3.68 (m, 2H), 3.40-3.33 (m, 5H), 3.10-3.04 (m, 1H), 2.84 (s, 3H), 2.27-2.18 (m, 1H), 1.99 (t, J = 18.8 Hz, 3H), 1.85-1.78 (m, 1H), 1.73-1.64 (m, 1H), 1.56-1.44 (m, 1H), 1.27-1.17 (m, 1H). |
| | SM: dihydro-2H-pyran-3(4H)-one | |
| 81 | 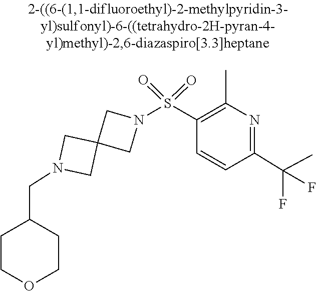 [SMILES: Cc1nc(C(C)(F)F)ccc1S(=O)(=O)N1CC2(CN(CC3CCOCC3)C2)C1] | Yield: 68 mg, 66%. LCMS m/z = 416.2 (M + H)⁺. ¹H NMR (400 MHz, CDCl₃) δ (ppm) 8.28 (d, J = 8.4 Hz, 1H), 7.60 (d, J = 8.4 Hz, 1H), 4.01 (s, 4H), 3.95-3.91 (m, 2H), 3.37- 3.31 (m, 3H), 3.27 (s, 3H), 2.86 (s, 3H), 2.26 (d, J = 6.4 Hz, 2H), 2.01 (t, J = 18.8 Hz, 3H), 1.56 (d, J = 12.8 Hz, 3H), 1.29-1.18 (m, 2H). |
| | SM: tetrahydro-2H-pyran-4-carbaldehyde | |
| 82 | 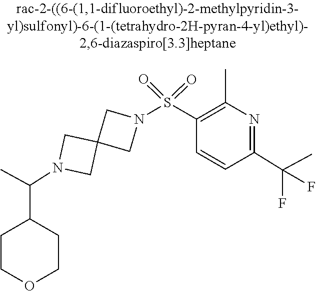 [SMILES: Cc1nc(C(C)(F)F)ccc1S(=O)(=O)N1CC2(CN(C(C)C3CCOCC3)C2)C1] | Yield: 78 mg, 52%. LCMS m/z = 430.1 (M + H)⁺. ¹H NMR (400 MHz, CDCl₃) δ (ppm) 8.28 (d, J = 8.0 Hz, 1H), 7.60 (d, J = 8.0 Hz, 1H), 4.00 (s, 4H), 3.99-3.96 (m, 2H), 3.40- 3.28 (m, 2H), 3.27-3.21 (m, 4H), 2.86 (s, 3H), 2.07-1.96 (m, 4H), 1.54-1.47 (m, 1H), 1.46-1.28 (m, 4H), 0.80 (d, J = 6.4 Hz, 3H). |
| | SM: 1-(tetrahydro-2H-pyran-4-yl)ethan-1-one | |
| 83 | 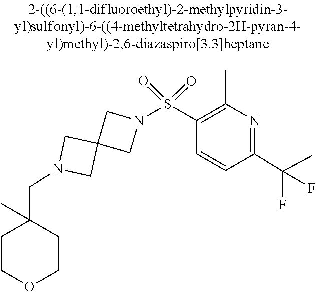 [SMILES: Cc1nc(C(C)(F)F)ccc1S(=O)(=O)N1CC2(CN(CC3(C)CCOCC3)C2)C1] | Yield: 82 mg, 63%. LCMS m/z = 430.1 (M + H)⁺. ¹H NMR (400 MHz, MeOD) δ (ppm) 8.36 (d, J = 8.0 Hz, 1H), 7.70 (d, J = 8.4 Hz, 1H), 3.99 (s, 4H), 3.69-3.56 (m, 4H), 3.39 (s, 4H), 2.83 (s, 3H), 2.31 (s, 2H), 1.99 (t, J = 18.8 Hz, 3H), 1.52- 1.44 (m, 2H), 1.26-1.20 (m, 2H), 0.97 (s, 3H). |
| | SM: 1-(4-methyltetrahydro-2H-pyran-4- | |
| | yl)ethan-1-one | |
| 84 | 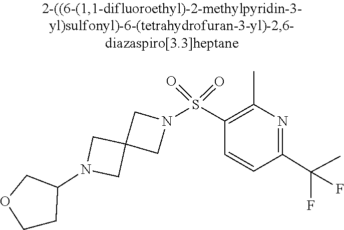 [SMILES: Cc1nc(C(C)(F)F)ccc1S(=O)(=O)N1CC2(CN(C3CCOC3)C2)C1] | Yield: 59 mg, 65%. LCMS m/z = 388.1 (M + H)⁺. ¹H NMR (400 MHz, CDCl₃) δ (ppm) 8.28 (d, J = 8.0 Hz, 1H), 7.60 (d, J = 8.0 Hz, 1H), 4.01 (s, 4H), 3.91-3.85 (m, 1H), 3.76- 3.71 (m, 1H), 3.61-3.54 (m, 2H), 3.32-3.27 (m, 4H), 2.95-2.90 (m, 1H), 2.86 (s, 3H), 2.01 (t, J = 18.8 Hz, 3H), 1.90-1.80 (m, 1H), 1.67- 1.60 (m, 1H). |
| | SM: dihydrofuran-3(2H)-one | |
| 85 | 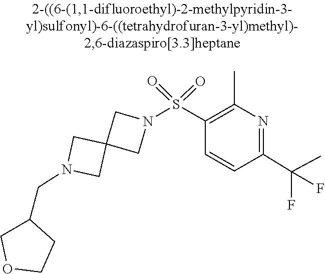 [SMILES: Cc1nc(C(C)(F)F)ccc1S(=O)(=O)N1CC2(CN(CC3CCOC3)C2)C1] | Yield: 70 mg, 47% LCMS m/z = 402.1 (M + H)⁺. ¹H NMR (400 MHz, CDCl₃) δ (ppm) 8.28 (d, J = 8.0 Hz, 1H), 7.60 (d, J = 8.0 Hz, 1H), 4.01 (s, 4H), 3.84-3.78 (m, 2H), 3.74- 3.66 (m, 1H), 3.43-3.37 (m, 1H), 3.28 (s, 4H), 2.86 (s, 3H), 2.38 (br d, J = 7.2 Hz, 2H), 2.20-2.11 (m, 1H), 2.02 (t, J = 18.8 Hz, 4H), 1.54-1.47 (m, 1H). |
| | SM: tetrahydrofuran-3-carbaldehyde | |
| 86 | 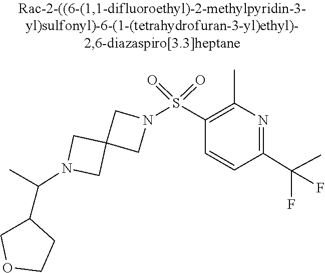 [SMILES: Cc1nc(C(C)(F)F)ccc1S(=O)(=O)N1CC2(CN(C(C)C3CCOC3)C2)C1] | Yield: 68 mg, 54% LCMS m/z = 416.1 (M + H)⁺. ¹H NMR (400 MHz, CDCl₃) δ (ppm) 8.28 (d, J = 8.0 Hz, 1H), 7.60 (d, J = 8.0 Hz, 1H), 4.00 (s, 4H), 3.87-3.64 (m, 3H), 3.53- 3.36 (m, 1H), 3.27-3.25 (m, 4H), 2.86 (s, 3H), 2.18-2.15 (m, 2H), 2.01 (t, J = 18.8 Hz, 3H), 1.69- 1.57 (m, 2H), 0.92-0.82 (m, 3H). |
| | SM: 1-(tetrahydrofuran-3-yl)ethan-1-one | |
| 87 | 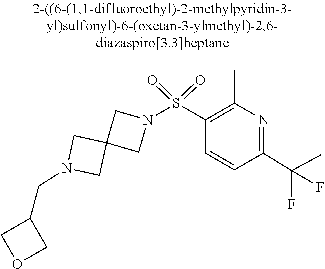 [SMILES: Cc1nc(C(C)(F)F)ccc1S(=O)(=O)N1CC2(CN(CC3COC3)C2)C1] | Yield: 68 mg, 62% LCMS m/z = 388.1 (M + H)⁺. ¹H NMR (400 MHz, CDCl₃) δ (ppm) 8.27 (d, J = 8.4 Hz, 1H), 7.60 (d, J = 8.4 Hz, 1H), 4.77- 4.71 (m, 2H), 4.35 (t, J = 6.0 Hz, 2H), 4.00 (s, 4H), 3.27 (s, 4H), 2.98-2.89 (m, 1H), 2.85 (s, 3H), 2.69 (d, J = 7.2 Hz, 2H), 2.01 (t, J = 18.8 Hz, 3H). |
| | SM: oxetane-3-carbaldehyde | |
| 88 | 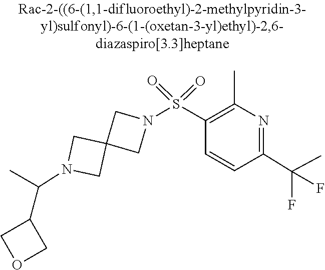 [SMILES: Cc1nc(C(C)(F)F)ccc1S(=O)(=O)N1CC2(CN(C(C)C3COC3)C2)C1] | Yield: 77 mg, 69% LCMS m/z = 402.1 (M + H)⁺. ¹H NMR (400 MHz, CDCl₃) δ (ppm) 8.27 (d, J = 8.0 Hz, 1H), 7.60 (d, J = 8.4 Hz, 1H), 4.70- 4.60 (m, 2H), 4.56-4.45 (m, 2H), 3.99 (s, 4H), 3.30-3.19 (m, 4H), 2.99-2.90 (m, 1H), 2.85 (s, 3H), 2.53-2.44 (m, 1H), 2.01 (t, J = 18.8 Hz, 3H), 0.85 (d, J = 6.4 Hz, 3H). |
| | SM: 1-(oxetan-3-yl)ethan-1-one | |
| 89 | 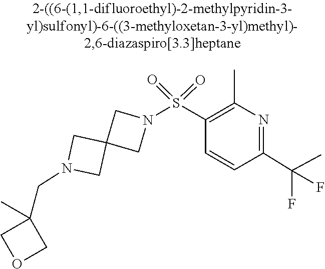 [SMILES: Cc1nc(C(C)(F)F)ccc1S(=O)(=O)N1CC2(CN(CC3(C)COC3)C2)C1] | Yield: 80 mg, 66% LCMS m/z = 402.1 (M + H)⁺. ¹H NMR (400 MHz, CDCl₃) δ (ppm) 8.28 (d, J = 8.4 Hz, 1H), 7.60 (d, J = 8.0 Hz, 1H), 4.39 (d, J = 5.6 Hz, 2H), 4.28 (d, J = 5.6 Hz, 2H), 4.01 (s, 4H), 3.36 (s, 4H), 2.86 (s, 3H), 2.60 (s, 2H), 2.01 (t, J = 18.8 Hz, 3H), 1.24 (s, 3H). |
| | SM: 3-methyloxetane-3-carbaldehyde | |

### Example 90: 2-((6-(difluoromethyl)-2-methylpyridin-3-yl)sulfonyl)-6-(tetrahydro-2H-pyran-4-yl)-2,6-diazaspiro[3.3]heptane

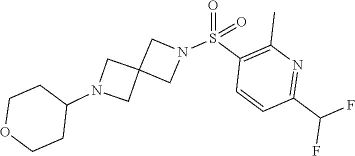 [SMILES: Cc1nc(C(F)F)ccc1S(=O)(=O)N1CC2(CN(C3CCOCC3)C2)C1]

### 1. Synthesis of 6-bromo-2-methylnicotinaldehyde

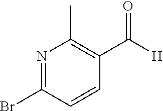

3,6-Dibromo-2-methylpyridine (2 g, 7.97 mmol) was dissolved in toluene (30 mL). The solution was cooled to −78° C. under an argon atmosphere and n-BuLi (2.5 M, 4.78 mL) was added dropwise over 15 minutes. After stirring at −78° C. for 2.5 hours, DMF (1.05 g, 14.35 mmol, 1.11 mL) was added dropwise. The mixture was stirred 1 hour at −78° C. and was then allowed to slowly warm up to 15° C. After stirring for 3 hours at 15° C., NH₄Cl (100 mL) was added dropwise to terminate the reaction. The aqueous phase was extracted with DCM (3×20 mL). The combined organic layers were dried with Na₂SO₄ and the solvent was removed under reduced pressure. The residue was purified by flash column chromatography on silica gel (EtOAc/petroleum ether=1%˜6%) to give the desired compound (300 mg, 1.50 mmol, 18.82% yield) as white solid. ¹H NMR (500 MHz, CDCl₃) δ (ppm) 10.01 (s, 1H), 7.99 (d, J=8.0 Hz, 1H), 7.66 (d, J=8.0 Hz, 1H), 2.77 (s, 3H).

### 2. Synthesis of 6-bromo-3-(difluoromethyl)-2-methylpyridine

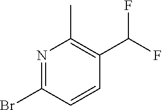

To a solution of 6-bromo-2-methylnicotinaldehyde (2.0 g, 10.00 mmol) in DCM (5 mL) cooled to −78° C. was added DAST (4.83 g, 30.00 mmol, 3.96 mL) in portions under N₂ atmosphere. The mixture was stirred at 25° C. for 4 h. The reaction mixture was quenched by the addition of saturated aqueous NH₄Cl solution (30 mL) at 0° C. The mixture was extracted with DCM (15 mL×3). The combined organic phase was washed with brine (30 mL), dried over anhydrous sodium sulfate, filtered and concentrated to give the desired compound (1.67 g, crude) as yellow oil. ¹H NMR (400 MHz, CDCl₃) δ (ppm) 7.95 (d, J=8.0 Hz, 1H), 7.34 (d, J=8.0 Hz, 1H), 6.56 (t, J=55.2 Hz, 1H), 2.70 (s, 3H).

### 3. Synthesis of 6-(benzylthio)-3-(difluoromethyl)-2-methylpyridine

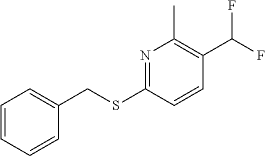 [SMILES: Cc1nc(SCc2ccccc2)ccc1C(F)F]

To a solution of 6-bromo-3-(difluoromethyl)-2-methylpyridine (2.9 g, 10.45 mmol) in Dioxane (40 mL) was added DIEA (4.05 g, 31.35 mmol, 5.46 mL), Pd(tBu₃P)₂ (534.00 mg, 1.04 mmol) and BnSH (2.51 g, 20.21 mmol, 2.37 mL). Then the mixture was stirred at 100° C. for 16 h under N₂. The mixture was quenched with aq. NaClO (200 mL). The reaction mixture was extracted with DCM (40 mL×3). The combined organic phases were washed with brine 100 mL, dried over Na₂SO₄, filtered and concentrated to give the desired compound (4.9 g, crude) as yellow oil. LCMS m/z=266.1 (M+H)⁺. ¹H NMR (500 MHz, CDCl₃) δ (ppm) 7.56 (d, J=8.0 Hz, 1H), 7.36 (d, J=8.0 Hz, 1H), 7.35-7.28 (m, 5H), 6.56 (t, J=55.5 Hz, 1H), 4.15 (s, 2H), 2.57 (s, 3H).

### 4. Synthesis of 5-(difluoromethyl)-6-methylpyridine-2-sulfonyl chloride

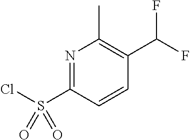

To a solution of 6-(benzylthio)-3-(difluoromethyl)-2-methylpyridine (1.6 g, 6.03 mmol) in DCM (15 mL) and water (3 mL) was added SO₂Cl₂ (5.70 g, 42.21 mmol, 3.42 mL) at −10° C. Then the mixture was stirred at −10° C. for 1 h under N₂. The mixture was diluted with water (20 mL) and extracted with DCM (25 mL×3). The combined organic phase was washed with brine (30 mL×2), dried over anhydrous sodium sulfate, and concentrated in vacuum to give the desired compound (1.56 g, crude) as yellow oil. ¹H NMR (500 MHz, CDCl₃) δ (ppm) 8.48 (d, J=8.0 Hz, 1H), 7.72 (d, J=8.0 Hz, 1H), 6.65 (t, J=54.8 Hz, 1H), 3.05 (s, 3H).

### 5. Synthesis of 2-((6-(difluoromethyl)-2-methylpyridin-3-yl)sulfonyl)-6-(tetrahydro-2H-pyran-4-yl)-2,6-diazaspiro[3.3]heptane

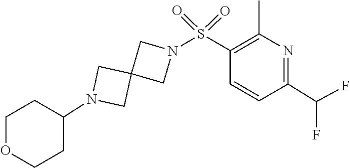 [SMILES: Cc1nc(C(F)F)ccc1S(=O)(=O)N1CC2(CN(C3CCOCC3)C2)C1]

5-(Difluoromethyl)-6-methylpyridine-2-sulfonyl chloride (294.61 mg, 1.22 mmol) was added to a solution of 2-(tetrahydro-2H-pyran-4-yl)-2,6-diazaspiro[3.3]heptane (300 mg, 1.02 mmol, TFA) and DIEA (393.92 mg, 3.05 mmol, 530.89 μL) in DCM (6 mL) at 0-5° C. The mixture was stirred at 20° C. for 2 h. The mixture was diluted with water (20 mL) and extracted with DCM (20 mL×3). The combined organic phase was washed with brine (20 mL), dried over anhydrous sodium sulfate, filtered and concentrated in vacuo. The crude material was purified by prep-HPLC (Column: Welch Xtimate C18 150*25 mm*5 um; Condition: water (NH₄HCO₃)-ACN; Begin B: 26; End B: 56; Flow Rate (ml/min): 25) to give the target compound (170 mg, 438.77 μmol, 43.19% yield) as white solid. LCMS m/z=388.1 (M+H)⁺. ¹H NMR (400 MHz, CDCl₃) δ (ppm) 8.33 (d, J=8.0 Hz, 1H), 7.60 (d, J=8.0 Hz, 1H), 6.62 (t, J=55.2 Hz, 1H), 4.03 (s, 4H), 3.96-3.90 (m, 2H), 3.37-3.30 (m, 2H), 3.28 (s, 4H), 2.86 (s, 3H), 2.19-2.07 (m, 1H), 1.56-1.50 (m, 2H), 1.33-1.23 (m, 2H).

### Example 91: 2-((6-(difluoromethyl)-2-methylpyridin-3-yl)sulfonyl)-6-(2-oxaspiro[3.3]heptan-6-yl)-2,6-diazaspiro[3.3]heptane

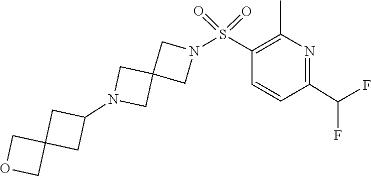 [SMILES: Cc1nc(C(F)F)ccc1S(=O)(=O)N1CC2(CN(C3CC4(COC4)C3)C2)C1]

### 1. Synthesis of tert-butyl 6-((6-(difluoromethyl)-2-methylpyridin-3-yl)sulfonyl)-2,6-diazaspiro[3.3]heptane-2-carboxylate

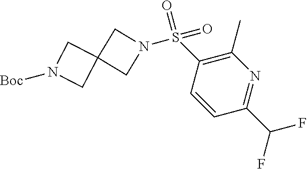 [SMILES: Cc1nc(C(F)F)ccc1S(=O)(=O)N1CC2(CN(C(=O)OC(C)(C)C)C2)C1]

5-(Difluoromethyl)-6-methylpyridine-2-sulfonyl chloride (380 mg, 1.57 mmol) was added to a solution of tert-butyl 2,6-diazaspiro[3.3]heptane-2-carboxylate (260 mg, 1.31 mmol) in DCM (10 mL) and DIEA (508.46 mg, 3.93 mmol, 685.25 μL). The reaction mixture was stirred at 15° C. for 2 h. The mixture was concentrated in vacuum to give crude, which was purified by flash column (EtOAc in petroleum ether=1%˜10%) to give the desired compound (330 mg, 817.96 μmol, 62.37% yield) as a white solid. LCMS m/z=404.1 (M+H)⁺. ¹H NMR (400 MHz, CDCl₃) δ (ppm) 8.33 (d, J=8.0 Hz, 1H), 7.61 (d, J=8.0 Hz, 1H), 6.62 (t, J=54.8 Hz, 1H), 4.06 (s, 4H), 4.03 (s, 4H), 2.86 (s, 3H), 1.42 (s, 9H).

### 2. Synthesis of 2-((6-(difluoromethyl)-2-methylpyridin-3-yl)sulfonyl)-2,6-diazaspiro[3.3]heptane

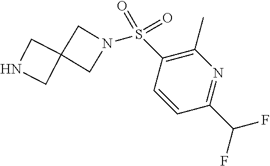 [SMILES: Cc1nc(C(F)F)ccc1S(=O)(=O)N1CC2(CNC2)C1]

To a solution of tert-butyl 6-((6-(difluoromethyl)-2-methylpyridin-3-yl)sulfonyl)-2,6-diazaspiro[3.3]heptane-2-carboxylate (320 mg, 793.17 μmol) in HFIP (10 mL) was added TFA (2 mL) and the reaction mixture was stirred at 15° C. for 2 h. The mixture was concentrated in vacuum to give the desired compound (400 mg, crude, TFA salt) as colorless oil. LCMS m/z=304.1 (M+H)⁺. ¹H NMR (500 MHz, MeOD) δ (ppm) 8.41 (d, J=8.0 Hz, 1H), 7.71 (d, J=8.0 Hz, 1H), 6.74 (t, J=55.0 Hz 1H), 4.24 (s, 4H), 4.16 (s, 4H), 2.84 (s, 3H).

### 3. Synthesis of 2-((6-(difluoromethyl)-2-methylpyridin-3-yl)sulfonyl)-6-(2-oxaspiro[3.3]heptan-6-yl)-2,6-diazaspiro[3.3]heptane

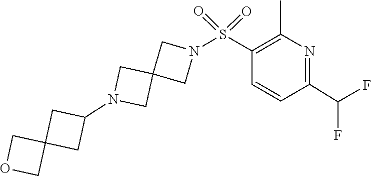 [SMILES: Cc1nc(C(F)F)ccc1S(=O)(=O)N1CC2(CN(C3CC4(COC4)C3)C2)C1]

A solution of 2-((6-(difluoromethyl)-2-methylpyridin-3-yl)sulfonyl)-2,6-diazaspiro[3.3]heptane (400 mg, 960.75 μmol, TFA salt) and TEA (291.65 mg, 2.88 mmol, 401.73 μL) in MeOH (10 mL) was cooled to 0-5° C. 2-Oxaspiro[3.3]heptan-6-one was added in portions. The mixture was stirred at 0-5° C. for 5 min and adjusted pH 5-6 by HOAc in one portion at 0° C. for 30 minutes. NaBH₃CN (181.13 mg, 2.88 mmol) was added and the mixture was stirred at 20° C. for 3 h. The mixture was diluted with water (15 mL) and extracted with DCM (20 mL×3). The combined organic phase was washed with brine (20 mL×1), dried over anhydrous Na₂SO₄, filtered and concentrated in vacuum. The crude which was purified by prep-HPLC (Column: Welch Xtimate C18 150*25 mm*5 um; Condition: water (NH₄HCO₃)-ACN; Begin B: 27; End B: 57; Flow Rate (ml/min): 25) to give the target compound (88 mg, 23% yield) as colorless oil. LCMS m/z=400.1 (M+H)⁺. ¹H NMR (400 MHz, CDCl₃) δ (ppm) 8.31 (d, J=8.0 Hz, 1H), 7.59 (d, J=8.0 Hz, 1H), 6.61 (t, J=54.8 Hz, 1H), 4.64 (s, 2H), 4.58 (s, 2H), 3.99 (s, 4H), 3.20 (s, 4H), 2.85 (s, 3H), 2.84-2.75 (m, 1H), 2.29-2.21 (m, 2H), 1.95-1.86 (m, 2H).

### Example 92: 2-((6-(2-fluoropropan-2-yl)-2-methylpyridin-3-yl)sulfonyl)-6-(2-oxaspiro[3.3]heptan-6-yl)-2,6-diazaspiro[3.3]heptane

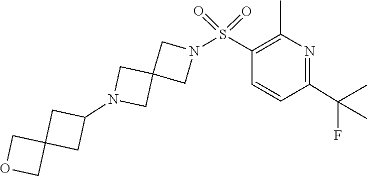 [SMILES: Cc1nc(C(C)(C)F)ccc1S(=O)(=O)N1CC2(CN(C3CC4(COC4)C3)C2)C1]

### 1. Synthesis of 2-(6-bromo-2-methylpyridin-3-yl)propan-2-ol

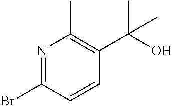

n-BuLi (2.5 M, 8.77 mL) was added dropwise to a stirred solution of 3,6-dibromo-2-methylpyridine (5 g, 19.93 mmol) in toluene (100 mL) under nitrogen at −70° C. The mixture was stirred at −70° C. for 1 h. Acetone (2.31 g, 39.85 mmol, 2.93 mL) was added dropwise and the mixture was stirred at −70° C. for 1 h. TLC (petroleum ether/EtOAc=3/1, R_{f}=0.5) showed the reaction was completed. The mixture was quenched with sat. aqueous NH₄Cl at 0° C. and allowed to warm to 25° C. The organic layer was separated, washed with sat. NaHCO₃ (50 mL) and brine (30 mL). The material was dried over anhydrous sodium sulfate, filtered and concentrated. The residue was purified by flash silica gel chromatography (EtOAc in petroleum ether=from 0% to 30%) to give the desired compound (3.52 g, 77% yield) as a yellow oil. ¹H NMR (500 MHz, CHLOROFORM-d) δ (ppm) 7.79 (d, J=8.5 Hz, 1H), 7.06 (d, J=8.0 Hz, 1H), 2.66 (s, 3H), 1.52 (s, 6H).

### 2. Synthesis of 6-bromo-3-(2-fluoropropan-2-yl)-2-methylpyridine

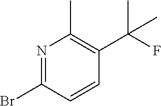

To a stirred solution of 2-(6-bromo-2-methylpyridin-3-yl)propan-2-ol (3.5 g, 15.21 mmol) in DCM (35 mL) was added DAST (9.81 g, 60.84 mmol, 8.04 mL) at −78° C. The reaction mixture was stirred at 25° C. for 16 h. The reaction mixture was quenched by the addition of saturated aqueous NaHCO₃ solution (350 mL) at −5° C., diluted with water (30 mL), extracted with DCM (50 mL×3). The combined organic phase was washed with brine (20 mL×2), dried over anhydrous sodium sulfate, filtered and concentrated. The residue was purified by flash silica gel chromatography (EtOAc in petroleum ether=from 0% to 10%) to give the desired compound (3.3 g, 93% yield) as yellow oil. ¹H NMR (400 MHz, CHLOROFORM-d) δ (ppm) 7.79 (d, J=8.4 Hz, 1H), 7.25 (d, J=8.0 Hz, 1H), 2.64 (s, 3H), 1.72 (s, 3H), 1.66 (s, 3H).

### 3. Synthesis of 6-(benzylthio)-3-(2-fluoropropan-2-yl)-2-methylpyridine

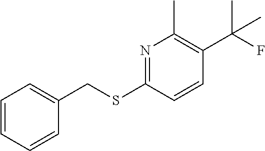 [SMILES: Cc1nc(SCc2ccccc2)ccc1C(C)(C)F]

To a solution of 2-(6-bromo-2-methylpyridin-3-yl)propan-2-ol (3.3 g, 14.22 mmol) in Dioxane (35 mL) was added DIPEA (5.51 g, 42.66 mmol, 7.43 mL), Pd(tBu₃P)₂ (726.64 mg, 1.42 mmol) and BnSH (3.07 g, 24.72 mmol, 2.90 mL). Then the mixture was stirred at 100° C. for 16 h under N₂. LCMS showed the desired product mass was observed. The mixture was quenched with H₂O (130 mL). The reaction mixture was extracted with DCM (50 mL×5). Combined organic phases were washed with brine (50 mL), dried over Na₂SO₄, filtered and concentrated to give the desired compound (5.4 g, crude) as yellow oil. LCMS m/z=276.1 (M+H)⁺. ¹H NMR (400 MHz, CHLOROFORM-d) δ (ppm) 7.47 (d, J=8.0 Hz, 1H), 7.33-7.27 (m, 3H), 7.26-7.18 (m, 3H), 4.05 (s, 2H), 2.51 (s, 3H), 1.70-1.62 (m, 6H).

### 4. Synthesis of 5-(2-fluoropropan-2-yl)-6-methylpyridine-2-sulfonyl chloride

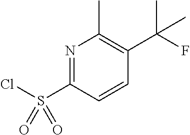

To a solution of 6-(benzylthio)-3-(2-fluoropropan-2-yl)-2-methylpyridine (1.8 g, 6.54 mmol) in DCM (10 mL) and water (2 mL) was added SO₂Cl₂ (6.18 g, 45.75 mmol, 3.71 mL) at −10° C. Then the mixture was stirred at 25° C. for 1 h under N₂. The mixture was diluted with water (20 mL) and extracted with DCM (20 mL×3). The combined organic phase was washed with brine (30 mL×2), dried over anhydrous sodium sulfate, and concentrated in vacuo to give the desired compound (900 mg, crude) as a yellow oil. ¹H NMR (500 MHz, CHLOROFORM-d) δ (ppm) 8.23 (d, J=8.0 Hz, 1H), 7.55-7.51 (m, 1H), 2.89 (s, 3H), 1.65 (s, 3H), 1.61 (s, 3H).

### 5. Synthesis of tert-butyl 6-((6-(2-fluoropropan-2-yl)-2-methylpyridin-3-yl)sulfonyl)-2,6-diazaspiro[3.3]heptane-2-carboxylate

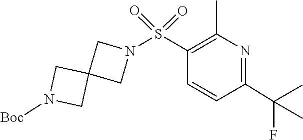 [SMILES: Cc1nc(C(C)(C)F)ccc1S(=O)(=O)N1CC2(CN(C(=O)OC(C)(C)C)C2)C1]

To a solution of tert-butyl 2,6-diazaspiro[3.3]heptane-2-carboxylate (220 mg, 768.48 μmol, Oxalate salt) and DIPEA (496.59 mg, 3.84 mmol, 669.26 μL) in DCM (10 mL) was added 5-(2-fluoropropan-2-yl)-6-methylpyridine-2-sulfonyl chloride (483.57 mg, 1.92 mmol) at 0° C. under N₂. The mixture was stirred at 25° C. for 3 h. LCMS showed the desired product mass was observed. The reaction mixture was diluted with water (30 mL), extracted with DCM (20 mL×3). The combined organic phase was washed with brine (20 mL×2), dried over anhydrous sodium sulfate, filtered and concentrated to give the desired compound (300 mg, crude) as a yellow oil. LCMS m/z=414.1 (M+H)⁺. ¹H NMR (500 MHz, CHLOROFORM-d) δ (ppm) 8.19 (d, J=8.0 Hz, 1H), 7.53-7.50 (m, 1H), 4.05-4.02 (m, 8H), 2.81 (s, 3H), 1.73 (s, 3H), 1.69 (s, 3H), 1.43 (s, 9H).

### 6. Synthesis of 2-((6-(2-fluoropropan-2-yl)-2-methylpyridin-3-yl)sulfonyl)-2,6-diazaspiro[3.3]heptane

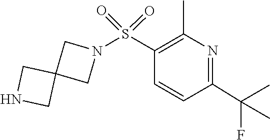 [SMILES: Cc1nc(C(C)(C)F)ccc1S(=O)(=O)N1CC2(CNC2)C1]

TFA (248.17 mg, 2.18 mmol, 166.67 uL) was added to a solution of tert-butyl 6-((6-(2-fluoropropan-2-yl)-2-methylpyridin-3-yl)sulfonyl)-2,6-diazaspiro[3.3]heptane-2-carboxylate (300 mg, 725.50 μmol) in HFIP (31.92 g, 189.95 mmol, 20 mL) at 25° C. under N₂. The mixture was stirred at 25° C. for 16 h. LCMS showed the desired product mass was observed. The mixture was concentrated to give the desired compound (300 mg, crude, TFA salt) as yellow oil. LCMS m/z=314.1 (M+H)⁺. ¹H NMR (500 MHz, CHLOROFORM-d) δ (ppm) 8.16 (d, J=8.0 Hz, 1H), 7.52 (d, J=8.5 Hz, 1H), 4.25 (s, 4H), 4.07 (s, 4H), 2.78 (s, 3H), 1.72 (s, 3H), 1.68 (s, 3H).

### 7. Synthesis of 2-((6-(2-fluoropropan-2-yl)-2-methylpyridin-3-yl)sulfonyl)-6-(2-oxaspiro[3.3]heptan-6-yl)-2,6-diazaspiro[3.3]heptane

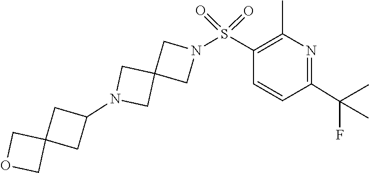 [SMILES: Cc1nc(C(C)(C)F)ccc1S(=O)(=O)N1CC2(CN(C3CC4(COC4)C3)C2)C1]

A solution of 2-((6-(2-fluoropropan-2-yl)-2-methylpyridin-3-yl)sulfonyl)-2,6-diazaspiro[3.3]heptane (300 mg, 703.56 μmol, TFA salt) and 2-oxaspiro[3.3]heptan-6-one (236.66 mg, 2.11 mmol) in MeOH (5 mL) was adjusted to pH=6 using acetic acid. The stirring was continued for 1 h at 25° C. NaBH₃CN (221.06 mg, 3.52 mmol) was added and the reaction mixture was stirred at 25° C. for 6 h. LCMS showed the reaction was completed. The mixture was concentrated in vacuo. The crude was purified by pre-HPLC (Column: Boston Prime C18 150*30 mm*5 μm, Condition: water (NH₃·H₂O+NH₄HCO₃)-ACN, Begin B: 43; End B: 73; Flow Rate (mL/min): 25) to give the title compound (90 mg, 31% yield) as a white solid. LCMS m/z=410.2 (M+H)⁺. ¹H NMR (500 MHz, DMSO-d₆) δ (ppm) 8.25-8.19 (m, 1H), 7.58 (d, J=8.0 Hz, 1H), 4.54-4.37 (m, 4H), 3.90-3.89 (m, 4H), 3.32-3.30 (m, 1H), 3.09-3.06 (m, 4H), 2.73 (s, 3H), 2.16-2.09 (m, 2H), 1.85-1.77 (m, 2H), 1.68 (s, 3H), 1.64 (s, 3H).

### Example 93: 2-((6-(2-fluoropropan-2-yl)-2-methylpyridin-3-yl)sulfonyl)-6-(tetrahydro-2H-pyran-4-yl)-2,6-diazaspiro[3.3]heptane

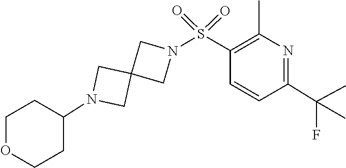 [SMILES: Cc1nc(C(C)(C)F)ccc1S(=O)(=O)N1CC2(CN(C3CCOCC3)C2)C1]

To a solution of 2-(tetrahydro-2H-pyran-4-yl)-2,6-diazaspiro[3.3]heptane (100 mg, 457.20 μmol, HCl salt) in DCM (5 mL) was added DIPEA (295.44 mg, 2.29 mmol, 398.17 μL) and 5-(2-fluoropropan-2-yl)-6-methylpyridine-2-sulfonyl chloride (287.7 mg, 1.14 mmol) at 0° C. under N₂. The mixture was stirred at 25° C. for 4 h. LCMS showed the desired product mass was observed. The mixture was quenched by the addition of water (5 mL), extracted with DCM (20 mL×3). The combined organic phase was washed with brine (20 mL×2), dried over anhydrous sodium sulfate, filtered and concentrated. The residue was purified by prep-HPLC (Column: Boston Prime C18 150*30 mm*5 um; Condition: water (NH₃—H₂O+NH₄HCO₃)-ACN; Begin B: 40; End B: 70; Flow Rate: 25 mL/min) to give the title compound (43 mg, 23.66% yield) as a white solid. LCMS m/z=398.1 (M+H)⁺. ¹H NMR (500 MHz, CHLOROFORM-d) δ (ppm) 8.18 (d, J=8.0 Hz, 1H), 7.50 (d, J=7.5 Hz, 1H), 4.00 (s, 4H), 3.97-3.91 (m, 2H), 3.37-3.31 (m, 2H), 3.28 (s, 4H), 2.81 (s, 3H), 2.19-2.07 (m, 1H), 1.72 (s, 3H), 1.68 (s, 3H), 1.61-1.57 (m, 2H), 1.36-1.23 (m, 2H).

### Example 94: 2-((6-(difluoromethoxy)-2-methylpyridin-3-yl)sulfonyl)-6-(tetrahydro-2H-pyran-4-yl)-2,6-diazaspiro[3.3]heptane

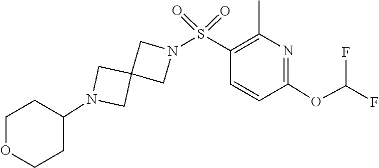 [SMILES: Cc1nc(OC(F)F)ccc1S(=O)(=O)N1CC2(CN(C3CCOCC3)C2)C1]

### 1. Synthesis of 6-(difluoromethoxy)-2-methyl-3-nitropyridine

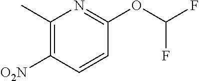

To a solution of 6-methyl-5-nitropyridin-2(1H)-one (5 g, 32.44 mmol) in CH₃CN (150 mL) was added NaH (2.60 g, 64.88 mmol, 60% purity) at 0° C. After stirring at 15° C. for 30 min, 2,2-difluoro-2-(fluorosulfonyl)acetic acid (8.67 g, 48.66 mmol, 5.03 mL) was added to the mixture stirred at 15° C. for 1 h. The mixture was diluted with water (100 ml×2) and extracted with EtOAc (150 mL×2). The combined phased layers were dried over anhydrous Na₂SO₄, filtered and concentrated. The residue was purified by column chromatography on silica gel with petroleum ether and ethyl acetate (EtOAc in Petroleum ether from 0% to 9%) to give the desired compound (3.1 g, 47% yield) as yellow oil. LCMS m/z=205.1 (M+H)⁺. ¹H NMR (400 MHz, CDCl₃) δ (ppm) 8.41 (d, J=8.8 Hz, 1H), 7.55 (t, J=72.0 Hz, 1H), 6.88 (d, J=8.8 Hz, 1H), 2.83 (s, 3H).

### 2. Synthesis of 6-(difluoromethoxy)-2-methylpyridin-3-amine

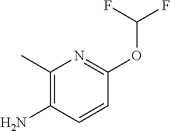

To a solution of 6-(difluoromethoxy)-2-methyl-3-nitropyridine (1 g, 4.90 mmol) was added Pd/C (1.00 g, 939.67 μmol, 10% purity) at 25° C. under N₂. The mixture was purged with H₂ several times and stirred at 95° C. for 16 h under H₂ (15 Psi). The mixture was filtered and concentrated. The residue was purified by column chromatography on silica gel with petroleum ether and ethyl acetate (EtOAc in Petroleum ether from 0% to 50%) to give the desired compound (300 mg, 35% yield) as a yellow oil. LCMS m/z=175.2 (M+H)⁺. ¹H NMR (400 MHz, CDCl₃) δ (ppm) 7.32 (t, J=74.0 Hz, 1H), 7.13 (s, 1H), 7.00 (d, J=8.4 Hz, 1H), 6.61 (d, J=8.4 Hz, 1H), 2.32 (s, 3H).

### 3. Synthesis of 6-(difluoromethoxy)-2-methylpyridine-3-sulfonyl chloride

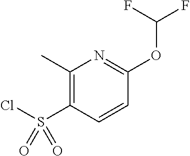

SOCl₂ (2 mL) was added over 10 min to water (10 mL), while the temperature was maintained 0-7° C. The solution was stirred at 15° C. for 16 h. CuCl (3.41 mg, 34.45 μmol) was added and cooled to −3° C. In another flask, a solution of 6-(difluoromethoxy)-2-methylpyridin-3-amine (300 mg, 1.72 mmol) in HCl (12 M, 1.44 mL) at −5° C. was added dropwise to a solution of NaNO₂ (130.74 mg, 1.89 mmol) in water (2 mL) while maintaining temperature −5 to 0° C. When the addition was complete, this solution was then added to the precooled thionyl chloride solution and stirred at −2° C. for 0.5 h, then at 0° C. for 2 h. The mixture was diluted with water (80 ml) and extracted with EtOAc (50 mL×3). The combined organic phase was dried over anhydrous sodium sulfate, filtered and concentrated to give the desired compound (300 mg, crude) as a yellow oil. ¹H NMR (400 MHz, CDCl₃) δ (ppm) 8.34 (d, J=8.4 Hz, 1H), 7.56 (t, J=72.0 Hz, 1H), 6.92 (d, J=8.8 Hz, 1H), 2.93 (s, 3H).

### 4. Synthesis of 2-((6-(difluoromethoxy)-2-methylpyridin-3-yl)sulfonyl)-6-(tetrahydro-2H-pyran-4-yl)-2,6-diazaspiro[3.3]heptane

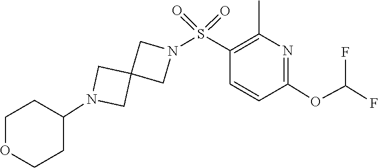 [SMILES: Cc1nc(OC(F)F)ccc1S(=O)(=O)N1CC2(CN(C3CCOCC3)C2)C1]

To a solution of tert-butyl 2,6-diazaspiro[3.3]heptane-2-carboxylate (150 mg, 508.00 μmol, TFA salt) and DIPEA (196.97 mg, 1.52 mmol, 265.45 μL) in DCM (3 mL) was added 6-(difluoromethoxy)-2-methylpyridine-3-sulfonyl chloride (143.97 mg, 558.80 μmol) in DCM (1 mL) at 0° C. for 5 min. Then the mixture was stirred at 20° C. for 2 h. The mixture was diluted with water (50 mL) and extracted with DCM (60 mL×3). The combined organic phase was dried over anhydrous sodium sulfate, filtered and concentrated to give a residue which was purified by prep-HPLC (Column: Welch Xtimate: C18 150*25 mm*5 um; Condition: water (NH₄HCO₃)-ACN; Begin B: 24%; End B: 54%; Flow Rate (ml/min): 25) to afford the title compound (62.2 mg, 30% yield) as a yellow oil. LCMS m/z=404.1 (M+H)⁺. ¹H NMR (400 MHz, CD₃OD) δ (ppm) 8.27 (d, J=8.4 Hz, 1H), 7.65 (t, J=72.0 Hz, 1H), 6.96 (d, J=8.4 Hz, 1H), 3.94 (s, 4H), 3.93-3.88 (m, 2H), 3.38-3.36 (m, 1H), 3.35-3.33 (m, 1H), 3.33 (s, 4H), 2.75 (s, 3H), 2.28-2.22 (m, 1H), 1.68-1.63 (m, 2H), 1.27-1.16 (m, 2H).

### Example 95: 2-((6-(difluoromethoxy)-2-methylpyridin-3-yl)sulfonyl)-6-(2-oxaspiro[3.3]heptan-6-yl)-2,6-diazaspiro[3.3]heptane

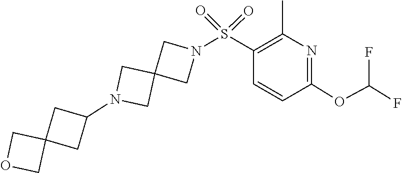 [SMILES: Cc1nc(OC(F)F)ccc1S(=O)(=O)N1CC2(CN(C3CC4(COC4)C3)C2)C1]

### 1. Synthesis of tert-butyl 6-((6-(difluoromethoxy)-2-methylpyridin-3-yl)sulfonyl)-2,6-diazaspiro[3.3]heptane-2-carboxylate

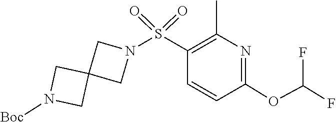 [SMILES: Cc1nc(OC(F)F)ccc1S(=O)(=O)N1CC2(CN(C(=O)OC(C)(C)C)C2)C1]

To a solution of tert-butyl 2,6-diazaspiro[3.3]heptane-2-carboxylate (200 mg, 1.01 mmol) and DIPEA (391.13 mg, 3.03 mmol, 527.13 μL) in DCM (3 mL) was added 6-(difluoromethoxy)-2-methylpyridine-3-sulfonyl chloride (285.89 mg, 1.11 mmol) in DCM (1 mL) at 0° C. for 5 min. The mixture was stirred at 20° C. for 2 h. The mixture was diluted with water (100 mL) and extracted with DCM (40 mL×5). The combined organic phase was dried over anhydrous sodium sulfate, filtered and concentrated to give a residue which was purified by silica gel column chromatography to afford the desired compound (220 mg, 52% yield) as a white solid. LCMS m/z=420.1 (M+H)⁺. ¹H NMR (500 MHz, CD₃OD) δ (ppm) 8.26 (d, J=8.5 Hz, 1H), 7.64 (t, J=72.5 Hz, 1H), 6.96 (d, J=8.5 Hz, 1H), 4.01 (s, 4H), 4.00 (s, 4H), 2.75 (s, 3H), 1.41 (s, 9H).

### 2. Synthesis of 2-((6-(difluoromethoxy)-2-methylpyridin-3-yl)sulfonyl)-2,6-diazaspiro[3.3]heptane

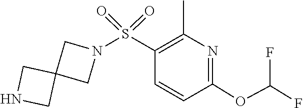 [SMILES: Cc1nc(OC(F)F)ccc1S(=O)(=O)N1CC2(CNC2)C1]

TFA (2.98 g, 26.12 mmol, 2 mL was added to a solution of tert-butyl 6-((6-(difluoromethoxy)-2-methylpyridin-3-yl)sulfonyl)-2,6-diazaspiro[3.3]heptane-2-carboxylate (200 mg, 476.82 μmol) in HFIP (10 mL). The mixture was stirred at 25° C. for 2 h. The residue was concentrated in vacuo to give the desired compound (200 mg, crude, TFA salt) as yellow oil. LCMS m/z=320.1 (M+H)⁺. ¹H NMR (500 MHz, CDCl₃) δ (ppm) 8.19 (d, J=9.0 Hz, 1H), 7.53 (t, J=72.0 Hz, 1H), 6.84 (d, J=8.5 Hz, 1H), 4.33 (br s, 4H), 4.11 (s, 4H), 2.73 (s, 3H).

### 3. Synthesis of 2-((6-(difluoromethoxy)-2-methylpyridin-3-yl)sulfonyl)-6-(2-oxaspiro[3.3]heptan-6-yl)-2,6-diazaspiro[3.3]heptane

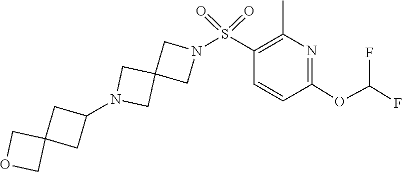 [SMILES: Cc1nc(OC(F)F)ccc1S(=O)(=O)N1CC2(CN(C3CC4(COC4)C3)C2)C1]

A solution of 2-((6-(difluoromethoxy)-2-methylpyridin-3-yl)sulfonyl)-2,6-diazaspiro[3.3]heptane (200 mg, 462.60 μmol, TFA salt) and 2-oxaspiro[3.3]heptan-6-one (155.61 mg, 1.39 mmol) in DCM (3 mL) was stirred at 20° C. for 1 h under N₂. NaBH₃CN (145.35 mg, 2.31 mmol) was added, and the mixture was stirred at 20° C. under N₂ for another 2 h. The mixture was diluted with water (50 mL) and extracted with DCM (50 mL×3). The combined organic phase was dried over anhydrous sodium sulfate, filtered and concentrated. The residue was purified by prep-HPLC (Column: Welch Xtimate: C18 150*25 mm*5 um; Condition: water (NH₄HCO₃)-ACN; Begin B: 33%; End B: 63%; Flow Rate (ml/min): 25) to afford the title compound (23.12 mg, 12% yield) as a yellow oil. LCMS m/z=416.1 (M+H)⁺. ¹H NMR (400 MHz, DMSO-d₆) δ (ppm) 8.23 (d, J=8.8 Hz, 1H), 7.78 (t, J=72.0 Hz, 1H), 7.12 (d, J=8.8 Hz, 1H), 4.48 (s, 2H), 4.42 (s, 2H), 3.86 (s, 4H), 3.07 (s, 4H), 2.80-2.71 (m, 1H), 2.68 (s, 3H), 2.15-2.08 (m, 2H), 1.84-1.77 (m, 2H).

### Example 96: 2-((3-(difluoromethyl)-1-methyl-1H-pyrazol-5-yl)sulfonyl)-6-(tetrahydro-2H-pyran-4-yl)-2,6-diazaspiro[3.3]heptane

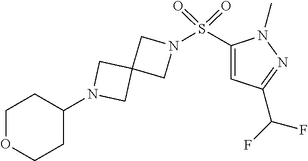 [SMILES: Cn1nc(C(F)F)cc1S(=O)(=O)N1CC2(CN(C3CCOCC3)C2)C1]

### 1. Synthesis of 3-(difluoromethyl)-1-methyl-1H-pyrazole

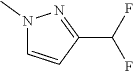

DAST (30.74 g, 190.71 mmol, 25.20 mL) was added dropwise to a −20° C. solution of 1-methyl-1H-pyrazole-3-carbaldehyde (7 g, 63.57 mmol) in DCM (100 mL) and the reaction was mixture stirred for 2 h at 15° C. The mixture was quenched with saturated aqueous NaHCO₃ (40 mL) and extracted with DCM (30 mL×2). The combined organic phase was washed with brine (80 mL×2), dried over anhydrous sodium sulfate, filtered and concentrated in vacuo to give 3-(difluoromethyl)-1-methyl-1H-pyrazole (6.5 g, crude) as brown oil. ¹H NMR (400 MHz, CDCl₃) δ (ppm) 7.38 (d, J=2.0 Hz, 1H), 6.67 (t, J=55.2 Hz, 1H), 6.48-6.42 (m, 1H), 3.92 (s, 3H).

### 2. Synthesis of lithium 3-(difluoromethyl)-1-methyl-1H-pyrazole-5-sulfinate

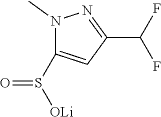

To a −50° C. solution of 3-(difluoromethyl)-1-methyl-1H-pyrazole (3 g, 22.71 mmol) in THE (60 mL) was added n-BuLi (2.5 M, 14.53 mL) dropwise over 10 min under nitrogen. The mixture was stirred at −50° C. for 1 h. Excess SO₂ gas was bubbled into a solution of THF (10 mL) for 10 min which was then added into the above solution at −50° C. The resulting mixture was allowed to warm to 15° C. and stir for 2 h. TLC (petroleum ether/EtOAc=3/1) showed a new spot was formed. The mixture was concentrated to give the desired compound (4.5 g, crude) as brown solid. ¹H NMR (400 MHz, DMSO-d₆) δ (ppm) 6.87 (t, J=55.2 Hz, 1H), 6.29 (s, 1H), 3.91 (s, 3H).

### 3. Synthesis of 3-(difluoromethyl)-1-methyl-1H-pyrazole-5-sulfonyl chloride

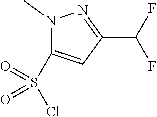

To a 0° C. solution of lithium 3-(difluoromethyl)-1-methyl-1H-pyrazole-5-sulfinate (4.5 g, 22.27 mmol) in DCM (35 mL) and water (35 mL) was added NCS (4.46 g, 33.40 mmol) under nitrogen and the mixture was then stirred at 0˜5° C. for 1 h. TLC (petroleum ether/EtOAc=3/1) showed a new spot was formed. The mixture was diluted with water (10 mL) and extracted with DCM (15 mL×3). The combined organic phase was dried over anhydrous sodium sulfate, filtered and concentrated in vacuo. The crude was purified by flash column (EtOAc in petroleum ether=0%˜10%) to give the desired compound (1.26 g, 24% yield) as a yellow oil. ¹H NMR (400 MHz, CDCl₃) δ (ppm) 7.22 (s, 1H), 6.69 (t, J=54.4 Hz, 1H), 4.24 (s, 3H).

### 4. Synthesis of 2-((3-(difluoromethyl)-1-methyl-1H-pyrazol-5-yl)sulfonyl)-6-(tetrahydro-2H-pyran-4-yl)-2,6-diazaspiro[3.3]heptane

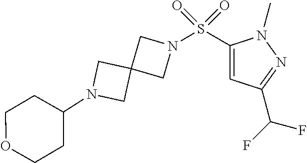 [SMILES: Cn1nc(C(F)F)cc1S(=O)(=O)N1CC2(CN(C3CCOCC3)C2)C1]

To a 0-5° C. solution of 2-(tetrahydro-2H-pyran-4-yl)-2,6-diazaspiro[3.3]heptane (100 mg, 548.66 μmol) and DIPEA (212.73 mg, 1.65 mmol, 286.70 μL) in DCM (4 mL) was added 3-(difluoromethyl)-1-methyl-1H-pyrazole-5-sulfonyl chloride (139.19 mg, 603.53 μmol) and the mixture was stirred at 15° C. for 1 h. The mixture was concentrated in vacuo purified by pre-HPLC (Column: Welch Xtimate C18 150*30 mm*5 um, Condition: water (10 mM NH₄HCO₃)-ACN, 22%˜52%, Flow Rate (mL/min): 25) to give the title compound (24.89 mg, 12% yield) as yellow oil. LCMS m/z=377.1 (M+H)⁺. ¹H NMR (400 MHz, CDCl₃) δ (ppm) 6.95 (s, 1H), 6.66 (t, J=54.8 Hz, 1H), 4.10 (s, 3H), 3.99 (s, 4H), 3.95-3.89 (m, 2H), 3.36-3.29 (m, 2H), 3.25 (s, 4H), 2.22-2.06 (m, 1H), 1.63-1.53 (m, 2H), 1.34-1.22 (m, 2H).

### Example 97: 2-((3-methoxy-1-methyl-1H-pyrazol-5-yl)sulfonyl)-6-(tetrahydro-2H-pyran-4-yl)-2,6-diazaspiro[3.3]heptane

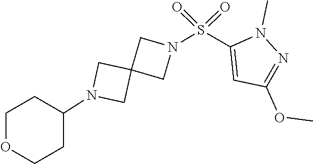 [SMILES: COc1cc(S(=O)(=O)N2CC3(CN(C4CCOCC4)C3)C2)n(C)n1]

### 1. Synthesis of 3-methoxy-1-methyl-1H-pyrazole

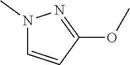

To a solution of 1-methyl-1H-pyrazol-3-ol (8.0 g, 81.55 mmol) in THF (200 mL) was added NaH (4.89 g, 122.32 mmol, 60% purity) at 0° C., after 30 min, Mel (28.94 g, 203.87 mmol, 12.69 mL) was added the mixture was stirred at 25° C. for 16 h under N₂. TLC showed the new spot was detected. The mixture was quenched with H₂O (20 mL) and extracted with DCM (80 mL×2). The combined organic phase was washed with brine (40 mL×1), dried over Na₂SO₄ and filtered. The filtrate was concentrated under reduced pressure and the residue was purified by flash column (EtOAc in petroleum ether from 10% to 30%) to give the desired compound (2.5 g, 27% yield) as colorless oil. ¹H NMR (500 MHz, CDCl₃), δ (ppm): 7.11 (d, J=2.5 Hz, 1H), 5.59 (s, 1H), 3.86 (s, 3H), 3.72 (s, 3H).

### 2. Synthesis of lithium 3-methoxy-1-methyl-1H-pyrazole-5-sulfinate

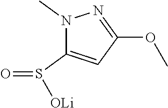

To a solution of 3-methoxy-1-methyl-1H-pyrazole (500 mg, 4.46 mmol) in THE (10 mL) was added n-BuLi (2.5 M, 2.85 mL) dropwise over 5 min at −65° C. and the reaction mixture was stirred at 0° C. for 1 h under N₂. SO₂ was bubbled into THE (10 mL) at −20° C. for 10 min and then added slowly into the solution over 10 min. The resulting mixture was stirred at −65° C. for 1 h and then at 25° C. for 1 h. The mixture was concentrated in vacuo and the residue triturated in petroleum ether (100 mL) and then filtered. The filter cake was collected and dried in vacuo to give the desired compound (1 g, crude) as yellow solid. ¹H NMR (400 MHz, DMSO-d6) δ (ppm) 5.44 (s, 1H), 3.70 (s, 3H), 3.67 (s, 3H).

### 3. Synthesis of 3-methoxy-1-methyl-1H-pyrazole-5-sulfonyl chloride

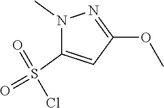

Lithium 3-methoxy-1-methyl-1H-pyrazole-5-sulfinate (1.1 g, 6.04 mmol) was dissolved in DCM (10 mL) and water (12 mL). NCS (1.29 g, 9.66 mmol) was added portion-wise vigorous stirring. The reaction mixture was stirred further for 30 min at 5° C. LCMS showed the reaction was completed. The mixture was diluted with water (30 mL). The mixture was extracted with DCM (30 mL×3). The combined organic phase was washed with brine (20 mL), dried over anhydrous sodium sulfate, filtered and concentrated to give the desired compound (1.0 g, crude) as yellow oil. LCMS m/z=211.0 (M+H)⁺. ¹H NMR (500 MHz, CDCl₃) δ (ppm) 6.37 (s, 1H), 4.05 (s, 3H), 3.90 (s, 3H).

### 4. Synthesis of tert-butyl 6-((3-methoxy-1-methyl-1H-pyrazol-5-yl)sulfonyl)-2,6-diazaspiro[3.3]heptane-2-carboxylate

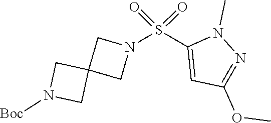 [SMILES: COc1cc(S(=O)(=O)N2CC3(CN(C(=O)OC(C)(C)C)C3)C2)n(C)n1]

To a solution of 3-methoxy-1-methyl-1H-pyrazole-5-sulfonyl chloride (847.12 mg, 4.27 mmol) and DIEA (1.66 g, 12.82 mmol, 2.23 mL) in DCM (10 mL) was added tert-butyl 2,6-diazaspiro[3.3]heptane-2-carboxylate (900 mg, 4.27 mmol) at 0-5° C. The reaction mixture was stirred at 15° C. for 2 h, then diluted with water (15 mL). The reaction was extracted with DCM (15 mL×3). The combined organic phase was washed with brine (15 mL×2), dried over anhydrous sodium sulfate, filtered and concentrated in vacuo. The crude material was purified by flash column (EtOAc in petroleum ether=15%˜50%) to give the desired compound (1.1 g, 69% yield) as white solid. ¹H NMR (400 MHz, CDCl₃) δ (ppm) 6.14 (s, 1H), 3.98 (s, 4H), 3.97 (s, 4H), 3.94 (s, 3H), 3.89 (s, 3H), 1.41 (s, 9H).

### 5. Synthesis of 2-((3-methoxy-1-methyl-1H-pyrazol-5-yl)sulfonyl)-2,6-diazaspiro[3.3]heptane

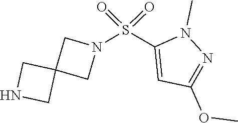 [SMILES: COc1cc(S(=O)(=O)N2CC3(CNC3)C2)n(C)n1]

TFA (2.98 g, 26.12 mmol, 2 mL) was added to a solution of tert-butyl 6-((3-methoxy-1-methyl-1H-pyrazol-5-yl)sulfonyl)-2,6-diazaspiro[3.3]heptane-2-carboxylate (1.1 g, 2.95 mmol) in HFIP (10 mL) and the reaction mixture was stirred at 15° C. for 2 h. The mixture was concentrated in vacuo to give the desired compound (1.2 g, crude, TFA salt) as a colorless oil. LCMS m/z=273.1 (M+H)⁺. ¹H NMR (500 MHz, MeOD) δ (ppm) 6.25 (s, 1H), 4.19 (s, 4H), 4.07 (s, 4H), 3.93 (s, 3H), 3.87 (s, 3H).

### 6. Synthesis of 2-((3-methoxy-1-methyl-1H-pyrazol-5-yl)sulfonyl)-6-(tetrahydro-2H-pyran-4-yl)-2,6-diazaspiro[3.3]heptane

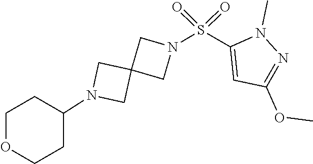 [SMILES: COc1cc(S(=O)(=O)N2CC3(CN(C4CCOCC4)C3)C2)n(C)n1]

Tetrahydro-4H-pyran-4-one (374.13 mg, 3.74 mmol, 345.14 μL) was added to a solution of 2-((3-methoxy-1-methyl-1H-pyrazol-5-yl)sulfonyl)-2,6-diazaspiro[3.3]heptane (1.2 g, 3.11 mmol, TFA) and TEA (945.36 mg, 9.34 mmol, 1.30 mL) in MeOH (12 mL) pH was adjusted to 5-6 by adding acetic acid (187.01 mg, 3.11 mmol, 178.28 μL) in one portion at 0° C. and the solution stirred for 30 min. NaBH₃CN (587.10 mg, 9.34 mmol) was added and the mixture was stirred at 20° C. for 2 h. The mixture was diluted with water (15 mL), extracted with DCM (15 mL×3). The combined organic phase was washed with brine (15 mL×1), dried over anhydrous sodium sulfate, filtered and concentrated. The crude reaction was was purified by prep-HPLC (Column: Welch Xtimate C18 150*25 mm*5 um; Condition: water (NH₄HCO₃)-ACN; Begin B: 16; End B: 46; Flow Rate (ml/min): 25) to give the title compound (1.0 g, 90% yield) as a white solid. LCMS m/z=357.1 (M+H)⁺. ¹H NMR (500 MHz, CDCl₃) δ (ppm) 6.14 (s, 1H), 3.94 (s, 3H), 3.94 (s, 4H), 3.93-3.90 (m, 2H), 3.89 (s, 3H), 3.36-3.30 (m, 2H), 3.22 (s, 4H), 2.15-2.06 (m, 1H), 1.59-1.54 (m, 2H), 1.31-1.22 (m, 2H).

### Example 98: 2-((2-chloro-6-methoxypyridin-3-yl)sulfonyl)-6-(tetrahydro-2H-pyran-4-yl)-2,6-diazaspiro[3.3]heptane

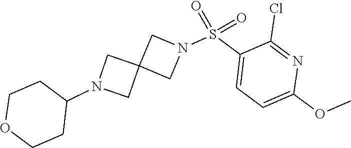 [SMILES: COc1ccc(S(=O)(=O)N2CC3(CN(C4CCOCC4)C3)C2)c(Cl)n1]

2-Chloro-6-methoxypyridine-3-sulfonyl chloride (98.05 mg, 405.02 μmol) was added at 0° C. to a solution of 2-(tetrahydro-2H-pyran-4-yl)-2,6-diazaspiro[3.3]heptane (120 mg, 405.02 μmol, TFA salt) and DIPEA (157.03 mg, 1.22 mmol, 211.63 μL) in DCM (10 mL). The mixture was stirred for 1 h at 20° C. LCMS showed desired mass was observed. The mixture was diluted with water (35 mL) and extracted with DCM (30 mL×3). The combined organic phase was dried over anhydrous sodium sulfate, filtered and concentrated in vacuo. The residue was purified by prep-HPLC (Welch Xtimate C18 150*25 mm*5 μm, Condition: water (NH₄HCO₃)-ACN, Flow Rate (mL/min): 25) to give the title compound (79 mg, 50% yield) as white solid. LCMS m/z=388.1 (M+H)⁺. ¹H NMR (400 MHz, CDCl₃) δ (ppm) 8.14 (d, J=8.4 Hz, 1H), 6.73 (d, J=8.4 Hz, 1H), 4.10 (s, 4H), 4.01 (s, 3H), 3.96-3.91 (m, 2H), 3.37-3.31 (m, 2H), 3.27 (s, 4H), 2.16-2.09 (m, 1H), 1.59-1.57 (m, 2H) 1.33-1.23 (m, 2H).

### Example 99: 2-((5-chloro-2-methoxypyridin-3-yl)sulfonyl)-6-(tetrahydro-2H-pyran-4-yl)-2,6-diazaspiro[3.3]heptane

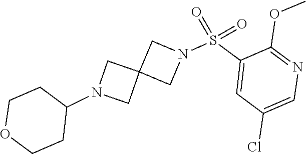 [SMILES: COc1ncc(Cl)cc1S(=O)(=O)N1CC2(CN(C3CCOCC3)C2)C1]

2-((5-Chloro-2-methoxypyridin-3-yl)sulfonyl)-6-(tetrahydro-2H-pyran-4-yl)-2,6-diazaspiro[3.3]heptane was prepared using a similar method described for Example 98 from 2-(tetrahydro-2H-pyran-4-yl)-2,6-diazaspiro[3.3]heptane (120 mg, 405.02 μmol, TFA salt) and 5-chloro-2-methoxypyridine-3-sulfonyl chloride (98.05 mg, 405.02 μmol). The crude product was purified by prep-HPLC (Welch Xtimate C18 150*25 mm*5 μm, Condition: water (NH₄HCO₃)-ACN, Flow Rate (mL/min): 25) to give the title compound (62 mg, 39.47% yield) as white solid.

LCMS m/z=388.1 (M+H)⁺. ¹H NMR (400 MHz, CDCl₃) δ (ppm) 8.26 (d, J=2.8 Hz, 1H), 8.10 (d, J=2.8 Hz, 1H), 4.13 (s, 4H), 4.06 (s, 3H), 3.96-3.93 (m, 2H), 3.37-3.33 (m, 2H), 3.31-3.28 (m, 4H), 2.18-2.13 (m, 1H), 1.61-1.57 (m, 2H), 1.31-1.29 (m, 2H).

### Example 100: 2-((2,4-dimethyl-6-(trifluoromethyl)pyridin-3-yl)sulfonyl)-6-(tetrahydro-2H-pyran-4-yl)-2,6-diazaspiro[3.3]heptane

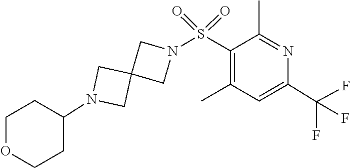 [SMILES: Cc1cc(C(F)(F)F)nc(C)c1S(=O)(=O)N1CC2(CN(C3CCOCC3)C2)C1]

### 1. Synthesis of 2,4-dimethyl-6-(trifluoromethyl)pyridin-3-amine

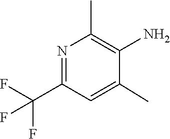

Methylboronic acid (467.80 mg, 7.81 mmol) was added to a solution of 2,4-dibromo-6-(trifluoromethyl)pyridin-3-amine (500 mg, 1.56 mmol) in Dioxane (10 mL) and water (1 mL). K₂CO₃ (864.07 mg, 6.25 mmol) and Pd(dppf)Cl₂-DCM (114.36 mg, 156.30 umol) were added at 25° C. under N₂. The mixture was stirred at 100° C. for 16 h under N₂. LCMS showed the desired product mass was observed. The mixture was filtered and diluted with water (20 mL), extracted with EtOAc (30 mL×2). The combined organic phase was washed with brine (30 mL), dried over anhydrous sodium sulfate, filtered and concentrated. The residue was purified by flash silica gel chromatography (EtOAc in petroleum ether=from 0% to 70%) to give the desired compound (240 mg, 603.79 umol, 91.89% yield) as yellow oil. LCMS m/z=191.1 (M+H)⁺. ¹H NMR (500 MHz, CDCl₃) δ (ppm) 7.27 (s, 1H), 3.87 (br s, 2H), 2.48 (s, 3H), 2.23 (s, 3H).

### 2. Synthesis of 2,4-dimethyl-6-(trifluoromethyl)pyridine-3-sulfonyl chloride

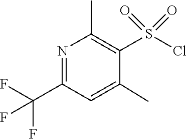 [SMILES: Cc1cc(C(F)(F)F)nc(C)c1S(=O)(=O)Cl]

SOCl₂ (0.5 mL) was added to water (3 mL), while the temperature was maintained 0-7° C., then the solution was stirred at 25° C. for 16 h. CuCl (1.04 mg, 10.52 umol) was added and the solution cooled to −3° C. In another flask, a solution of 2,4-dimethyl-6-(trifluoromethyl)pyridin-3-amine (100 mg, 525.86 umol) in HCl_{aq} (12 M, 625.00 uL) at −5° C. was added dropwise to a solution of NaNO₂ (38.10 mg, 552.15 umol) in water (0.25 mL) while maintaining temperature −5° C. to 0° C. When the addition was completed, this solution was then added to the precooled thionyl chloride solution and stirred at −2° C. for 10 min, then at 0° C. for 2 h. TLC (DCM/MeOH=9/1, R_{f}=0.9) showed a new spot was observed. The mixture was extracted with DCM (10 mL×4). The combined organic phase was dried over anhydrous sodium sulfate, filtered and concentrated to give desired compound (60 mg, crude) as a yellow oil. ¹H NMR (500 MHz, CDCl₃) δ (ppm) 7.57 (s, 1H), 3.08 (s, 3H), 2.89 (s, 3H).

### 3. Synthesis of 2-((2,4-dimethyl-6-(trifluoromethyl)pyridin-3-yl)sulfonyl)-6-(tetrahydro-2H-pyran-4-yl)-2,6-diazaspiro[3.3]heptane

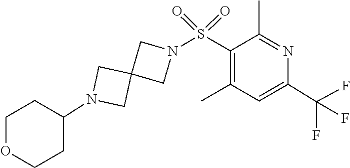 [SMILES: Cc1cc(C(F)(F)F)nc(C)c1S(=O)(=O)N1CC2(CN(C3CCOCC3)C2)C1]

2,4-Dimethyl-6-(trifluoromethyl)pyridine-3-sulfonyl chloride was added at 0° C. to a solution of 2-(tetrahydro-2H-pyran-4-yl)-2,6-diazaspiro[3.3]heptane (50 mg, 274.33 umol) and DIPEA (141.82 mg, 1.10 mmol, 191.13 uL) in DCM (4 mL) under N₂. The mixture was stirred at 25° C. for 3 h. LCMS showed the desired product mass was observed. The mixture was diluted with water (20 mL), extracted with DCM (30 mL×2). The combined organic phase was washed with brine (30 mL), dried over anhydrous sodium sulfate, filtered and concentrated. The residue was purified by prep-HPLC (Column: Welch Xtimate C18 150*25 mm*5 um; Condition: water (NH₄HCO₃)-ACN; Begin B: 35; End B: 65; Flow Rate: 25 mL/min) to give the target compound (13 mg, 11% yield) as a yellow solid. LCMS m/z=420.2 (M+H)⁺. ¹H NMR (500 MHz, CHLOROFORM-d) δ (ppm) 7.45 (s, 1H), 4.05 (s, 4H), 3.95 (br d, J=11.0 Hz, 2H), 3.39-3.27 (m, 6H), 2.93 (s, 3H), 2.74 (s, 3H), 2.14 (br s, 1H), 1.61 (br d, J=12.5 Hz, 2H), 1.33-1.26 (m, 2H).

### Example 101: 2-((2,4-dimethyl-6-(trifluoromethyl)pyridin-3-yl)sulfonyl)-6-(2-oxaspiro[3.3]heptan-6-yl)-2,6-diazaspiro[3.3]heptane

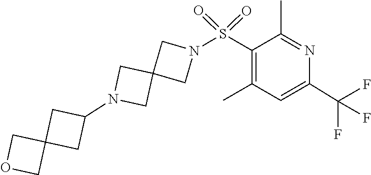 [SMILES: Cc1cc(C(F)(F)F)nc(C)c1S(=O)(=O)N1CC2(CN(C3CC4(COC4)C3)C2)C1]

### 1. Synthesis of tert-butyl 6-((2,4-dimethyl-6-(trifluoromethyl)pyridin-3-yl)sulfonyl)-2,6-diazaspiro[3.3]heptane-2-carboxylate

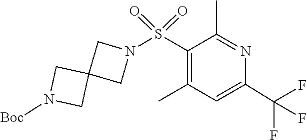 [SMILES: Cc1cc(C(F)(F)F)nc(C)c1S(=O)(=O)N1CC2(CN(C(=O)OC(C)(C)C)C2)C1]

2,4-Dimethyl-6-(trifluoromethyl)pyridine-3-sulfonyl chloride (125.61 mg, 458.99 μmol) in DCM (1 mL) was added to a solution of tert-butyl 2,6-diazaspiro[3.3]heptane-2-carboxylate (70 mg, 353.07 μmol) and DIPEA (182.52 mg, 1.41 mmol, 245.99 μL) at 0° C. under N₂. The mixture was stirred at 25° C. for 3 h. The mixture was diluted with water (20 mL) and extracted with DCM (30 mL×2). The combined organic phase was washed with brine (30 mL), dried over anhydrous sodium sulfate, filtered and concentrated to give the desired compound (140 mg, crude) as a yellow oil. LCMS m/z=436.2 (M+H)⁺. ¹H NMR (400 MHz, CHLOROFORM-d) δ (ppm) 7.45 (s, 1H), 4.07 (s, 4H), 4.05 (s, 4H), 2.91 (s, 3H), 2.73 (s, 3H), 1.42 (s, 9H).

### 2. Synthesis of 2-((2,4-dimethyl-6-(trifluoromethyl)pyridin-3-yl)sulfonyl)-2,6-diazaspiro[3.3]heptane

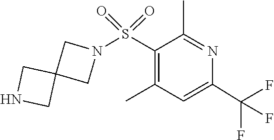 [SMILES: Cc1cc(C(F)(F)F)nc(C)c1S(=O)(=O)N1CC2(CNC2)C1]

TFA (78.55 mg, 688.93 μmol, 52.76 μL) was added to a solution of tert-butyl 6-((2,4-dimethyl-6-(trifluoromethyl)pyridine-3-yl)sulfonyl)-2,6-diazaspiro[3.3]heptane-2-carboxylate (120 mg, 275.57 μmol) in HFIP (57.46 g, 341.92 mmol, 36 mL). The mixture was stirred at 25° C. for 3 h. The mixture was neutralized with DIPEA and concentrated to give the desired compound (110 mg, crude) as a yellow oil. LCMS m/z=336.1 (M+H)⁺. ¹H NMR (400 MHz, CHLOROFORM-d) δ (ppm) 7.45 (s, 1H), 4.11 (s, 4H), 4.08 (s, 4H), 2.90 (s, 3H), 2.71 (s, 3H).

### 3. Synthesis of 2-((2,4-dimethyl-6-(trifluoromethyl)pyridin-3-yl)sulfonyl)-6-(2-azaspiro[3.3]heptan-6-yl)-2,6-diazaspiro[3.3]heptane

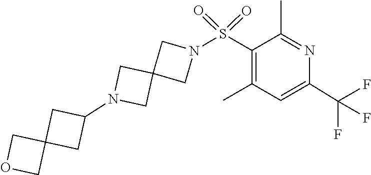 [SMILES: Cc1cc(C(F)(F)F)nc(C)c1S(=O)(=O)N1CC2(CN(C3CC4(COC4)C3)C2)C1]

A solution of 2-oxaspiro[3.3]heptan-6-one (90 mg, 268.38 μmol), 2-((2,4-dimethyl-6-(trifluoromethyl)pyridin-3-yl)sulfonyl)-2,6-diazaspiro[3.3]heptane (180.55 mg, 1.61 mmol) in DCM (5 mL) was added NaBH₃CN (284.40 mg, 1.34 mmol). The reaction was stirred at 25° C. for 16 h. The mixture was diluted with water (50 mL) and extracted with DCM (50 mL×3). The combined organic phase was dried over anhydrous sodium sulfate, filtered and concentrated in vacuo. The crude material was purified by pre-HPLC (Column: Boston Prime C18 150*30 mm*5 μm, Condition: water (NH₃·H₂O+NH₄HCO₃)-ACN, 37%˜67%, Flow Rate (mL/min): 25) to give the title compound (12.20 mg, 10% yield) as a white oil. LCMS m/z=432.1 (M+H)⁺. ¹H NMR (400 MHz, MeOD) δ (ppm) 7.67 (s, 1H), 4.66 (s, 2H), 4.58 (s, 2H), 4.01 (s, 4H), 3.30 (s, 4H), 2.97-2.90 (m, 1H), 2.87 (s, 3H), 2.73 (s, 3H), 2.34-2.27 (m, 2H), 2.00-1.94 (m, 2H).

### Example 102: 2-((2,4-dimethyl-6-(trifluoromethyl)pyridin-3-yl)sulfonyl)-6-(2-((tetrahydro-2H-pyran-4-yl)methyl)-2-azaspiro[3.3]heptan-6-yl)-2,6-diazaspiro[3.3]heptane

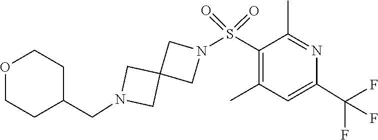 [SMILES: Cc1cc(C(F)(F)F)nc(C)c1S(=O)(=O)N1CC2(CN(CC3CCOCC3)C2)C1]

2-((2,4-dimethyl-6-(trifluoromethyl)pyridin-3-yl)sulfonyl)-6-(2-((tetrahydro-2H-pyran-4-yl)methyl)-2-azaspiro[3.3]heptan-6-yl)-2,6-diazaspiro[3.3]heptane was prepared using a similar method described for Example 101 step 3 from 2-((2,4-dimethyl-6-(trifluoromethyl)pyridin-3-yl)sulfonyl)-2,6-diazaspiro[3.3]heptane (50 mg, 111.52 umol, TFA salt) and tetrahydro-2H-pyran-4-carbaldehyde (25.46 mg, 223.04 umol). The crude product was purified by prep-HPLC (Column: Welch Xtimate C18 150*30 mm*5 um; Condition: water (10 mM NH₄HCO₃)-ACN; Begin B: 38; End B: 68; Gradient Time (min): 10; 100% B Hold Time (min): 2; Flow Rate (ml/min): 25; Detection wavelength: 220 nm) to give the title compound (39 mg, 80% yield) as a white solid. LCMS m/z=434.1 (M+H)⁺. ¹H NMR (400 MHz, CDCl₃) δ (ppm) 7.44 (s, 1H), 4.03 (s, 4H), 3.96-3.91 (m, 2H), 3.38-3.29 (m, 6H), 2.91 (s, 3H), 2.72 (s, 3H), 2.28-2.25 (m, 2H), 1.58-1.55 (m, 3H), 1.29-1.21 (m, 2H).

### Example 103: rac-2-((2,4-dimethyl-6-(trifluoromethyl)pyridin-3-yl)sulfonyl)-6-(2-((tetrahydrofuran-3-yl)methyl)-2-azaspiro[3.3]heptan-6-yl)-2,6-diazaspiro[3.3]heptane

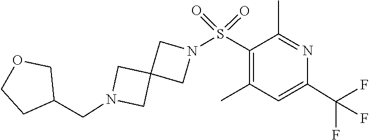 [SMILES: Cc1cc(C(F)(F)F)nc(C)c1S(=O)(=O)N1CC2(CN(CC3CCOC3)C2)C1]

rac-2-((2,4-dimethyl-6-(trifluoromethyl)pyridin-3-yl)sulfonyl)-6-(2-((tetrahydrofuran-3-yl)methyl)-2-azaspiro[3.3]heptan-6-yl)-2,6-diazaspiro[3.3]heptane was prepared using a similar method described for Example 101 step 3 from 2-((2,4-dimethyl-6-(trifluoromethyl)pyridin-3-yl)sulfonyl)-2,6-diazaspiro[3.3]heptane (50 mg, 111.52 umol, TFA salt) and tetrahydrofuran-3-carbaldehyde (22.33 mg, 223.04 umol). The crude product was purified by prep-HPLC (Column: Welch Xtimate C18 150*30 mm*5 um; Condition: water (10 mM NH₄HCO₃)-ACN; Begin B: 35; End B: 65; Gradient Time (min): 10; 100% B Hold Time (min): 2; Flow Rate (ml/min): 25; Detection wavelength: 220 nm) to give the the target compound (35 mg, 75% yield) as a white solid. LCMS m/z=420.1 (M+H)⁺. ¹H NMR (400 MHz, CDCl₃) δ (ppm) 7.44 (s, 1H), 4.03 (s, 4H), 3.84-3.79 (m, 2H), 3.73-3.70 (m, 1H), 3.42-3.39 (m, 1H), 3.30 (s, 4H), 2.91 (s, 3H), 2.73 (s, 3H), 2.40-2.37 (m, 2H), 2.17-2.15 (m, 1H), 1.99-1.96 (m, 1H), 1.53-1.49 (m, 1H).

### Example 104: 2-((2,4-dimethyl-6-(trifluoromethyl)pyridin-3-yl)sulfonyl)-6-(2-(oxetan-3-ylmethyl)-2-azaspiro[3.3]heptan-6-yl)-2,6-diazaspiro[3.3]heptane

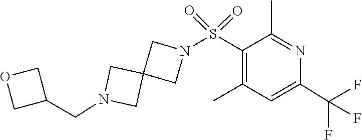 [SMILES: Cc1cc(C(F)(F)F)nc(C)c1S(=O)(=O)N1CC2(CN(CC3COC3)C2)C1]

2-((2,4-dimethyl-6-(trifluoromethyl)pyridin-3-yl)sulfonyl)-6-(2-(oxetan-3-ylmethyl)-2-azaspiro[3.3]heptan-6-yl)-2,6-diazaspiro[3.3]heptane was prepared using a similar method described for Example 101 step 3 from 2-((2,4-dimethyl-6-(trifluoromethyl)pyridin-3-yl)sulfonyl)-2,6-diazaspiro[3.3]heptane (150 mg, 333.80 μmol, TFA salt) and oxetane-3-carbaldehyde (57.47 mg, 667.60 μmol)). The crude product was purified by prep-HPLC (Phenomenex C18 150*40 mm*5 μm, Condition: water (NH₃·H₂O+NH₄HCO₃)-ACN, 20%˜50%, Flow Rate (mL/min): 60) to give the title compound (46 mg, 34% yield) as white solid.

LCMS m/z=406.1 (M+H)⁺. ¹H NMR (400 MHz, CDCl₃) δ (ppm) 7.44 (s, 1H), 4.76-4.72 (m, 2H), 4.35 (t, J=6.0 Hz, 2H), 4.01 (s, 4H), 3.28 (s, 4H), 2.96-2.92 (m, 1H), 2.91 (s, 3H), 2.72 (s, 3H), 2.70-2.68 (m, 2H).

### Example 105: 2-((6-methyl-2-(trifluoromethyl)pyridin-3-yl)sulfonyl)-6-(tetrahydro-2H-pyran-4-yl)-2,6-diazaspiro[3.3]heptane

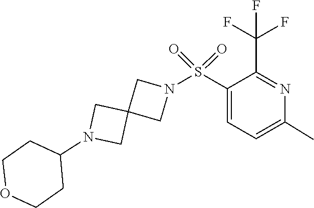 [SMILES: Cc1ccc(S(=O)(=O)N2CC3(CN(C4CCOCC4)C3)C2)c(C(F)(F)F)n1]

### 1. Synthesis of 6-chloro-2-(trifluoromethyl)pyridine-3-sulfonyl chloride

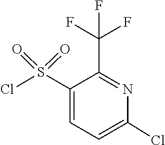 [SMILES: O=S(=O)(Cl)c1ccc(Cl)nc1C(F)(F)F]

While maintaining temperature at 0-7° C., SOCl₂ (5 mL) was added over 10 min to water (25 mL), The solution was stirred at 15° C. for 14 h. CuCl (9.07 mg, 91.58 μmol) was added and and the temperature maintained at −3° C. In another flask, a solution of 6-methyl-2-(trifluoromethyl)pyridin-3-amine (900 mg, 4.58 mmol) in HCl (12 M, 3.36 mL) at −5° C., was added dropwise to a solution of NaNO₂ (338.03 mg, 4.90 mmol) in water (2 mL) while maintaining temperature −5° C. to 0° C. When the addition was complete, this solution was then added to the precooled thionyl chloride solution and stirred at −3° C. for 10 min, then at 0° C. for 75 min. TLC (Petroleum ether/EtOAc=3/1, R_{f}=0.7) showed the reaction was complete. The reaction was added water H₂O (50 mL) and extracted with DCM (50 mL×5), dried over anhydrous Na₂SO₄, filtered and concentrated to give the desired compound (1 g, crude) as a yellow oil. ¹H NMR (400 MHz, CDCl₃) δ (ppm) 8.58 (d, J=8.0 Hz, 1H), 7.82 (d, J=8.4 Hz, 1H).

### 2. Synthesis of 2-((6-chloro-2-(trifluoromethyl)pyridin-3-yl)sulfonyl)-6-(tetrahydro-2H-pyran-4-yl)-2,6-diazaspiro[3.3]heptane

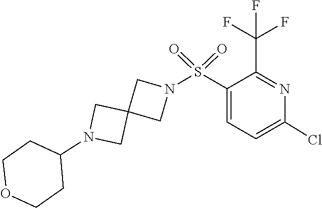 [SMILES: O=S(=O)(c1ccc(Cl)nc1C(F)(F)F)N1CC2(CN(C3CCOCC3)C2)C1]

A solution of 6-chloro-2-(trifluoromethyl)pyridine-3-sulfonyl chloride (210.06 mg, 750.07 μmol), 2-(tetrahydro-2H-pyran-4-yl)-2,6-diazaspiro[3.3]heptane (124.28 mg, 681.89 μmol, TFA salt) and DIEA (264.38 mg, 2.05 mmol, 356.31 μL) in DCM (5 mL) was stirred for 1 h at 0° C. The mixture was concentrated to give the desired compound (250 mg, crude) as a yellow oil. LCMS m/z=426.1 (M+H)⁺.

### Synthesis of 2-((6-methyl-2-(trifluoromethyl)pyridin-3-yl)sulfonyl)-6-(tetrahydro-2H-pyran-4-yl)-2,6-diazaspiro[3.3]heptane

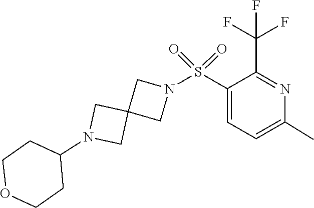 [SMILES: Cc1ccc(S(=O)(=O)N2CC3(CN(C4CCOCC4)C3)C2)c(C(F)(F)F)n1]

To a solution of 2-((6-chloro-2-(trifluoromethyl)pyridin-3-yl)sulfonyl)-6-(tetrahydro-2H-pyran-4-yl)-2,6-diazaspiro[3.3]heptane (100 mg, 234.82 μmol) in Dioxane (5 mL) and water (0.5 mL) was added 2,4,6-trimethyl-1,3,5,2,4,6-trioxatriborinane (147.39 mg, 1.17 mmol, 164.13 μL), K₂CO₃ (97.36 mg, 704.47 μmol) and PEPPSI-IPr catalyst (32.01 mg, 46.96 μmol) under N₂. The mixture was stirred at 100° C. for 3 h. The reaction was filtered and concentrated. The residue was purified by prep-HPLC (Column: Boston Prime C18 150*30 mm*5 μm Condition: water (NH₃·H₂O+NH₄HCO₃)-ACN; from 37% to 67%; Flow Rate: 25 mL/min) to give the title compound (44.80 mg, 47% yield) as a white solid. LCMS m/z=406.1 (M+H)⁺. ¹H NMR (400 MHz, MeOD) δ (ppm) 8.38 (d, J=8.4 Hz, 1H), 7.68 (d, J=8.0 Hz, 1H), 4.04 (s, 4H), 3.93-3.88 (m, 2H), 3.38-3.34 (m, 2H), 3.33 (s, 4H), 2.67 (s, 3H), 2.30-2.22 (m, 1H), 1.68-1.63 (m, 2H), 1.27-1.16 (m, 2H).

### Example 106: 2-((6-methyl-2-(trifluoromethyl)pyridin-3-yl)sulfonyl)-6-((tetrahydro-2H-pyran-4-yl)methyl)-2,6-diazaspiro[3.3]heptane

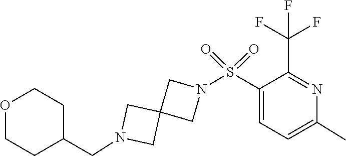 [SMILES: Cc1ccc(S(=O)(=O)N2CC3(CN(CC4CCOCC4)C3)C2)c(C(F)(F)F)n1]

### 1. Synthesis of tert-butyl 6-((6-chloro-2-(trifluoromethyl)pyridin-3-yl)sulfonyl)-2,6-diazaspiro[3.3]heptane-2-carboxylate

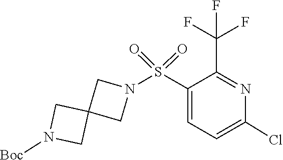 [SMILES: CC(C)(C)OC(=O)N1CC2(C1)CN(S(=O)(=O)c1ccc(Cl)nc1C(F)(F)F)C2]

A solution of 6-chloro-2-(trifluoromethyl)pyridine-3-sulfonyl chloride (621.52 mg, 2.22 mmol), tert-butyl 2,6-diazaspiro[3.3]heptane-2-carboxylate (400 mg, 2.02 mmol) and DIPEA (782.24 mg, 6.05 mmol, 1.05 mL) in DCM (10 mL) was stirred for 1 h at 0° C. Water (50 mL) was added and extracted with DCM (50 mL×3). The organic layer was washed with brine (30 mL), dried over Na₂SO₄, filtered and concentrated. The crude product was purified by column chromatography (EtOAc in petroleum ether=30% to 50%) to give the desired compound (800 mg, 1.81 mmol, 90% yield) as a white solid. LCMS m/z=486.1 (M−t−Bu+H)⁺.

### 2. Synthesis of 2-((6-chloro-2-(trifluoromethyl)pyridin-3-yl)sulfonyl)-2,6-diazaspiro[3.3]heptane

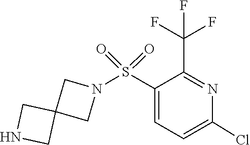 [SMILES: O=S(=O)(c1ccc(Cl)nc1C(F)(F)F)N1CC2(CNC2)C1]

To a solution of tert-butyl 6-((6-chloro-2-(trifluoromethyl)pyridin-3-yl)sulfonyl)-2,6-diazaspiro[3.3]heptane-2-carboxylate (800 mg, 1.81 mmol) in HFIP (10 mL) was added TFA (619.32 mg, 5.43 mmol, 415.93 μL) at 20° C. The mixture was stirred at 20° C. for 14 h. The mixture was concentrated to give the desired compound (800 mg, crude, TFA salt) as a yellow oil. LCMS m/z=342.1 (M+H)⁺. ¹H NMR (400 MHz, MeOD) δ (ppm) 8.51 (d, J=8.8 Hz, 1H), 7.91 (d, J=8.4 Hz, 1H), 4.25-4.22 (m, 8H).

### 3. Synthesis of 2-((6-chloro-2-(trifluoromethyl)pyridin-3-yl)sulfonyl)-6-((tetrahydro-2H-pyran-4-yl)methyl)-2,6-diazaspiro[3.3]heptane

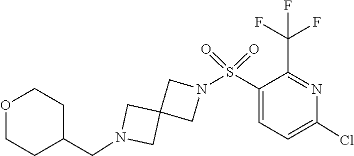 [SMILES: O=S(=O)(c1ccc(Cl)nc1C(F)(F)F)N1CC2(CN(CC3CCOCC3)C2)C1]

To a solution of 2-((6-chloro-2-(trifluoromethyl)pyridin-3-yl)sulfonyl)-2,6-diazaspiro[3.3]heptane (800 mg, 1.76 mmol, TFA salt) and tetrahydro-2H-pyran-4-carbaldehyde (301.20 mg, 2.64 mmol) in MeOH (10 mL) was added TEA (178.01 mg, 1.76 mmol, 245.20 μL) at 20° C., the mixture was stirred at 20° C. for 20 min, then adjusted to pH=6 using acetic acid. The stirring was continued for 30 minutes at 20° C. NaBH₃CN (552.76 mg, 8.80 mmol) was added at 20° C., the mixture was stirred at 20° C. for 14 h. The mixture was concentrated and purified by column chromatography (EtOAc in petroleum ether=30% to 50%) to give the title compound (350 mg, 54% yield) as a clear oil. LCMS m/z=440.1 (M+H)⁺. ¹H NMR (400 MHz, MeOD) δ (ppm) 8.50 (d, J=8.4 Hz, 1H), 7.91 (d, J=8.8 Hz, 1H), 4.19 (s, 3H), 3.98 (s, 3H), 3.92 (dd, J=11.6 Hz, 3.6 Hz, 2H), 3.43-3.35 (m, 2H), 3.25-3.18 (m, 3H), 2.82 (d, J=7.2 Hz, 2H), 1.98 (s, 1H), 1.82-1.72 (m, 1H), 1.61-1.56 (m, 2H).

### 1. Synthesis of 2-((6-methyl-2-(trifluoromethyl)pyridin-3-yl)sulfonyl)-6-((tetrahydro-2H-pyran-4-yl)methyl)-2,6-diazaspiro[3.3]heptane

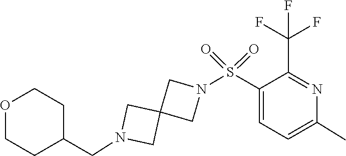 [SMILES: Cc1ccc(S(=O)(=O)N2CC3(CN(CC4CCOCC4)C3)C2)c(C(F)(F)F)n1]

To a solution of 2-((6-chloro-2-(trifluoromethyl)pyridin-3-yl)sulfonyl)-6-((tetrahydro-2H-pyran-4-yl)methyl)-2,6-diazaspiro[3.3]heptane (100 mg, 227.34 μmol) in Dioxane (5 mL) and water (0.5 mL) was added 2,4,6-trimethyl-1,3,5,2,4,6-trioxatriborinane (142.69 mg, 1.14 mmol, 158.90 μL), K₂CO₃ (94.26 mg, 682.01 μmol) and PEPPSI-IPr catalyst (30.98 mg, 45.47 μmol) under N₂. The mixture was stirred at 100° C. for 3 h. The reaction was filtered and concentrated. The residue was purified by prep-HPLC (Column: Boston Prime C18 150*30 mm*5 μm Condition: water (NH₃·H₂O+NH₄HCO₃)-ACN; from 40% to 70%; Flow Rate: 25 mL/min) to give the title compound (43.29 mg, 103.20 μmol, 45.40% yield) as a white solid. LCMS m/z=420.1 (M+H)⁺. (400 MHz, MeOD) δ (ppm) 8.37 (d, J=8.4 Hz, 1H), 7.68 (d, J=8.0 Hz, 1H), 4.04 (s, 4H), 3.89 (dd, J=11.2 Hz, 4.0 Hz, 2H), 3.40-3.36 (m, 2H), 3.34 (s, 4H), 2.67 (s, 3H), 2.32 (d, J=6.4 Hz, 2H), 1.64-1.54 (m, 3H), 1.27-1.16 (m, 2H).

### Example 107: 2-((6-cyclopropyl-2-(trifluoromethyl)pyridin-3-yl)sulfonyl)-6-(tetrahydro-2H-pyran-4-yl)-2,6-diazaspiro[3.3]heptane

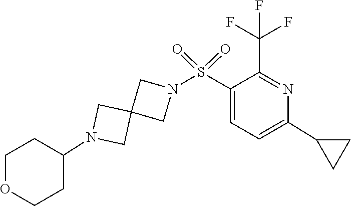 [SMILES: O=S(=O)(c1ccc(C2CC2)nc1C(F)(F)F)N1CC2(CN(C3CCOCC3)C2)C1]

To a solution of 2-((6-chloro-2-(trifluoromethyl)pyridin-3-yl)sulfonyl)-6-(tetrahydro-2H-pyran-4-yl)-2,6-diazaspiro[3.3]heptane (120 mg, 281.79 μmol) and cyclopropylboronic acid (48.41 mg, 563.58 μmol) in Toluene (5 mL) and water (0.5 mL) was added Pd₂(dba)₃ (25.80 mg, 28.18 μmol), SPhos (11.57 mg, 28.18 μmol) and K₃PO₄ (179.45 mg, 845.36 μmol) under N₂. The mixture was stirred at 100° C. for 3 h. The reaction was filtered and concentrated. The residue was purified by prep-HPLC (Column: Boston Prime C18 150*30 mm*5 μm Condition: water (NH₃·H₂O+NH₄HCO₃)-ACN; from 39% to 69%; Flow Rate: 25 mL/min) to give the title compound (106.28 mg, 86% yield) as a clear oil. LCMS m/z=432.1 (M+H)⁺. ¹H NMR (400 MHz, MeOD) δ (ppm) 8.30 (d, J=8.4 Hz, 1H), 7.67 (d, J=8.4 Hz, 1H), 4.02 (s, 4H), 3.90 (d, J=10.8 Hz, 2H), 3.38-3.32 (m, 6H), 2.30-2.21 (m, 2H), 1.65 (d, J=11.6 Hz, 2H), 1.26-1.18 (m, 2H), 1.17-1.13 (m, 4H).

### Example 108: 2-((6-cyclopropyl-2-(trifluoromethyl)pyridin-3-yl)sulfonyl)-6-((tetrahydro-2H-pyran-4-yl)methyl)-2,6-diazaspiro[3.3]heptane

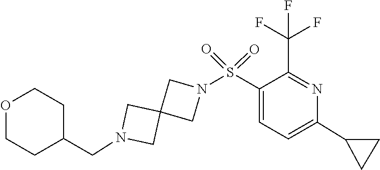 [SMILES: O=S(=O)(c1ccc(C2CC2)nc1C(F)(F)F)N1CC2(CN(CC3CCOCC3)C2)C1]

To a solution of 2-((6-chloro-2-(trifluoromethyl)pyridin-3-yl)sulfonyl)-6-((tetrahydro-2H-pyran-4-yl)methyl)-2,6-diazaspiro[3.3]heptane (100 mg, 227.34 μmol) and cyclopropylboronic acid (39.05 mg, 454.67 μmol) in Toluene (5 mL) and water (0.5 mL) was added Pd₂(dba)₃ (20.82 mg, 22.73 μmol), SPhos (9.33 mg, 22.73 μmol) and K₃PO₄ (144.77 mg, 682.01 μmol) under N₂. The mixture was stirred at 100° C. for 3 h. The reaction was filtered and concentrated. The residue was purified by prep-HPLC (Column: Boston Prime C18 150*30 mm*5 μm; Condition: water (NH₃·H₂O+NH₄HCO₃)-ACN; from 47% to 77%; Flow Rate: 25 mL/min) and further purified by prep-HPLC (Column: Welch Xtimate C18 150*25 mm*5 μm; Condition: water (NH₄HCO₃)-ACN; from 40% to 70%; Flow Rate: 25 mL/min) to give the title compound (22.64 mg, 17% yield) as a clear oil. LCMS m/z=446.1 (M+H)⁺. ¹H NMR (400 MHz, MeOD) δ (ppm) 8.30 (d, J=8.4 Hz, 1H), 7.67 (d, J=8.4 Hz, 1H), 4.01 (s, 4H), 3.89 (dd, J=11.2, 4.0 Hz, 2H), 3.40-3.34 (m, 2H), 3.33 (s, 4H), 2.32 (d, J=6.8 Hz, 2H), 2.29-2.23 (m, 1H), 1.62-1.55 (m, 3H), 1.27-1.19 (m, 2H), 1.18-1.16 (m, 1H), 1.16-1.12 (m, 3H).

### Example 109: 2-((2,6-bis(trifluoromethyl)pyridin-3-yl)sulfonyl)-6-(tetrahydro-2H-pyran-4-yl)-2,6-diazaspiro[3.3]heptane

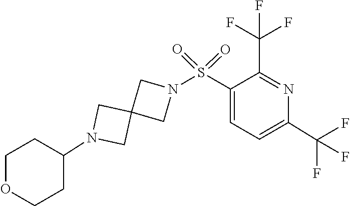 [SMILES: O=S(=O)(c1ccc(C(F)(F)F)nc1C(F)(F)F)N1CC2(CN(C3CCOCC3)C2)C1]

### 1. Synthesis of N-(2,6-bis(trifluoromethyl)pyridin-3-yl)-1,1-diphenylmethanimine

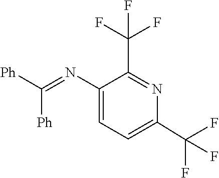 [SMILES: FC(F)(F)c1ccc(N=C(c2ccccc2)c2ccccc2)c(C(F)(F)F)n1]

To a solution of 3-bromo-2,6-bis(trifluoromethyl)pyridine (100 mg, 340.15 μmol) in Toluene (3 mL) was added Xantphos (19.68 mg, 34.01 μmol), Cs₂CO₃ (332.48 mg, 1.02 mmol), diphenylmethanimine (123.29 mg, 680.29 μmol, 114.16 μL) and Pd₂(dba)₃ (15.57 mg, 17.01 μmol) under N₂. The mixture was stirred at 100° C. for 16 h. The reaction mixture was extracted with DCM (30 mL×3). The combined organic phase was washed with brine (20 mL×2), dried over Na₂SO₄, filtered and concentrated to give the desired compound (130 mg, crude) as yellow oil. LCMS m/z=395.1 (M+H)⁺. ¹H NMR (400 MHz, CDCl₃) δ (ppm) 7.60 (d, J=7.6 Hz, 2H), 7.55-7.41 (m, 8H), 7.00 (d, J=8.4 Hz, 1H).

### 2. Synthesis of 2,6-bis(trifluoromethyl)pyridin-3-amine

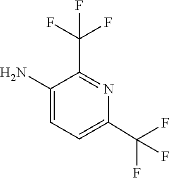 [SMILES: Nc1ccc(C(F)(F)F)nc1C(F)(F)F]

A solution of N-(2,6-bis(trifluoromethyl)pyridin-3-yl)-1,1-diphenylmethanimine (130 mg, 329.69 μmol) in HCl/dioxane (2 mL) was stirred at 20° C. for 16 h. The mixture was concentrated in vacuo to give the residue, which was purified by Combi-Flash (Petroleum ether/EtOAc=1/O to 3/1) to give the desired compound (80 mg, 300.11 μmol, 91.03% yield) as yellow oil. LCMS m/z=231.1 (M+H)⁺. ¹H NMR (400 MHz, CDCl₃) δ (ppm) 7.61 (d, J=8.4 Hz, 1H), 7.19 (d, J=8.8 Hz, 1H), 4.57 (br s, 2H).

### 3. Synthesis of 2,6-bis(trifluoromethyl)pyridine-3-sulfonyl chloride

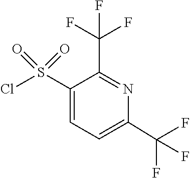 [SMILES: O=S(=O)(Cl)c1ccc(C(F)(F)F)nc1C(F)(F)F]

SOCl₂ (1 mL) was added to water (5 mL), while the temperature was maintained 0-7° C., then the solution was stirred at 15° C. for 14 h. CuCl (1 mg, 10.10 μmol) was added and cooled to −3° C. In another flask, a solution of 2,6-bis(trifluoromethyl)pyridin-3-amine (80 mg, 347.66 μmol) in HCl (12 M, 1 mL) at −5° C., was added dropwise to a solution of NaNO₂ (25.67 mg, 372.00 μmol) in water (0.5 mL) while maintaining temperature −5 to 0° C. When the addition was complete, this solution was then added to the precooled thionyl chloride solution and stirred at −2° C. for 10 min, then at 0° C. for 75 min. TLC (Petroleum ether/EtOAc=3/1, R_{f}=0.7) showed the reaction was complete. The reaction was added H₂O (20 mL) and extracted with DCM (30 mL×5), dried over anhydrous Na₂SO₄, filtered and concentrated to give the desired compound (80 mg, crude) as a yellow oil. ¹H NMR (400 MHz, CDCl₃) δ (ppm) 8.88 (d, J=8.4 Hz, 1H), 8.19 (d, J=8.4 Hz, 1H).

### 4. Synthesis of tert-butyl 6-((2,6-bis(trifluoromethyl)pyridin-3-yl)sulfonyl)-2,6-diazaspiro[3.3]heptane-2-carboxylate

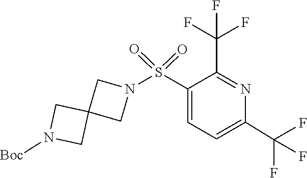 [SMILES: CC(C)(C)OC(=O)N1CC2(C1)CN(S(=O)(=O)c1ccc(C(F)(F)F)nc1C(F)(F)F)C2]

A solution of 2,6-bis(trifluoromethyl)pyridine-3-sulfonyl chloride (80 mg, 255.10 μmol), tert-butyl 2,6-diazaspiro[3.3]heptane-2-carboxylate (50.58 mg, 255.10 μmol) and DIPEA (98.91 mg, 765.29 μmol, 133.30 μL) in DCM (2 mL) was stirred for 1 h at 0° C. The mixture was filtered and concentrated in vacuo. The crude was purified by flash column (Petroleum ether in EtOAc from 0% to 30%) to give the desired compound (260 mg, 33% yield) as a yellow solid. LCMS m/z=420.1 (M-t-Bu+H)⁺. ¹H NMR (400 MHz, CDCl₃) δ (ppm) 8.68 (d, J=8.4 Hz, 1H), 8.03 (d, J=8.0 Hz, 1H), 4.21 (s, 4H), 4.06 (s, 4H), 1.44 (s, 9H).

### 5. Synthesis of 2-((2,6-bis(trifluoromethyl)pyridin-3-yl)sulfonyl)-2,6-diazaspiro[3.3]heptane

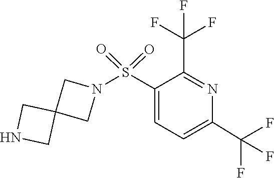 [SMILES: O=S(=O)(c1ccc(C(F)(F)F)nc1C(F)(F)F)N1CC2(CNC2)C1]

TFA (71.95 mg, 631.04 μmol, 48.32 μL) was added to a 20° C. solution of tert-butyl 6-((2,6-bis(trifluoromethyl)pyridin-3-yl)sulfonyl)-2,6-diazaspiro[3.3]heptane-2-carboxylate (100 mg, 210.35 μmol) in HFIP (3 mL). The mixture was stirred at 20° C. for 3 h. The mixture was concentrated to give the desired compound (100 mg, crude, TFA salt) as a yellow solid. LCMS m/z=376.1 (M+H)⁺. ¹H NMR (400 MHz, MeOD) δ (ppm) 8.80 (d, J=8.0 Hz, 1H), 8.29 (d, J=8.4 Hz, 1H), 4.27 (s, 4H), 4.24 (s, 4H).

### 6. Synthesis of 2-((2,6-bis(trifluoromethyl)pyridin-3-yl)sulfonyl)-6-(tetrahydro-2H-pyran-4-yl)-2,6-diazaspiro[3.3]heptane

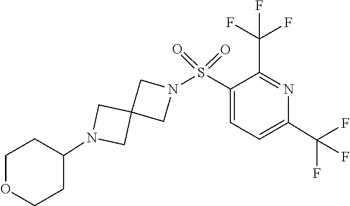 [SMILES: O=S(=O)(c1ccc(C(F)(F)F)nc1C(F)(F)F)N1CC2(CN(C3CCOCC3)C2)C1]

To a solution of 2-((2,6-bis(trifluoromethyl)pyridin-3-yl)sulfonyl)-2,6-diazaspiro[3.3]heptane (90 mg, 184.31 μmol, TFA salt) and tetrahydro-4H-pyran-4-one (36.90 mg, 368.62 μmol, 34.05 μL) in MeOH (5 mL) was added NaBH₃CN (34.75 mg, 552.93 μmol) at 20° C. The mixture was stirred at 20° C. for 3 h. The mixture was concentrated and purified by pre-HPLC (Column: Welch Xtimate C18 150*25 mm*5 μm, Condition: water (NH₄HCO₃)-ACN, 32%˜62%, Flow Rate (mL/min): 25) to give the title compound (50.69 mg, 65% yield) as a white solid. LCMS m/z=460.1 (M+H)⁺. ¹H NR (400 MHz, MeOD) δ (ppm) 8.79 (d, J=8.4 Hz, 1H), 8.28 (d, J=8.0 Hz, 1H), 4.14 (s, 4H), 3.94-3.88 (m, 2H), 3.38-3.34 (m, 6H), 2.31-2.23 (m, 1H), 1.68-1.64 (i, 2H), 1.28-1.16 (i, 2H).

### Examples 110-112

The Examples 110-112 in the following Table were prepared using a similar method described for Example 109 from the TFA salt of 2-((2,6-bis(trifluoromethyl)pyridin-3-yl)sulfonyl)-2,6-diazaspiro[3.3]heptane (1 equiv.) and the corresponding aldehyde or ketone.

| Example | Name/Structure/(SM) | DATA |
|---|---|---|
| 110 | 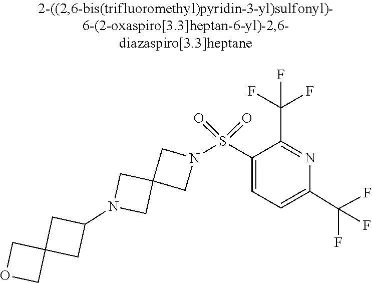 [SMILES: O=S(=O)(c1ccc(C(F)(F)F)nc1C(F)(F)F)N1CC2(CN(C3CC4(COC4)C3)C2)C1] | LCMS m/z = 472.0 (M + H)⁺. ¹H NMR (400 MHz, CDCl₃) δ (ppm) 8.66 (d, J = 8.0 Hz, 1H), 8.01 (d, J = 8.4 Hz, 1H), 4.64 (s, 2H), 4.59 (s, 2H), 4.13 (s, 4H), 3.21 (s, 4H), 2.82-2.77 (m, 1H), 2.28-2.23 (m, 2H), 1.94-1.89 (m, 2H). |
| | SM: 2-oxaspiro[3.3]heptan-6-one | |
| 111 | 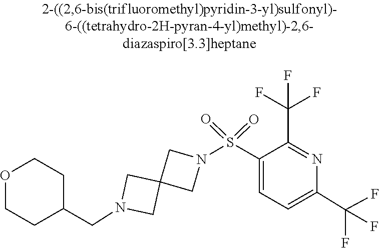 [SMILES: O=S(=O)(c1ccc(C(F)(F)F)nc1C(F)(F)F)N1CC2(CN(CC3CCOCC3)C2)C1] | LCMS m/z = 474.1 (M + H)⁺. ¹H NMR (400 MHz, MeOD) δ (ppm) 8.79 (d, J = 8.4 Hz, 1H), 8.44 (s, 0.5 H), 8.29 (d, J = 8.4 Hz, 1H), 4.21 (s, 4H), 3.95-3.88 (m, 2H), 3.86 (s, 4H), 3.38 (t, J = 11.4 Hz, 2H), 2.72 (d, J = 6.8 Hz, 2H), 1.82-1.67 (m, 1H), 1.61-1.56 (m, 2H), 1.33-1.23 (m, 2H). |
| | SM: tetrahydro-2H-pyran-4-carbaldehyde | |
| 112 | 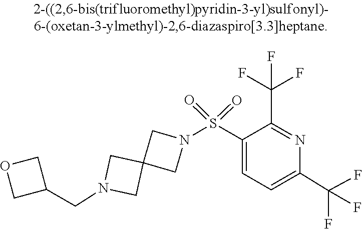 [SMILES: O=S(=O)(c1ccc(C(F)(F)F)nc1C(F)(F)F)N1CC2(CN(CC3COC3)C2)C1] | LCMS m/z = 446.1 (M + H)⁺. ¹H NMR (400 MHz, CDCl₃) δ (ppm) 8.67 (d, J = 8.4 Hz, 1H), 8.02 (d, J = 8.0 Hz, 1H), 4.77-4.73 (m, 2H), 4.36 (t, J = 6.4 Hz, 2H), 4.15 (s, 4H), 3.30 (s, 4H), 2.99-2.91 (m, 1H), 2.70 (d, J = 7.6 Hz, 2H). |
| | SM: oxetane-3-carbaldehyde | |

### Example 113: 2-((2,6-bis(trifluoromethyl)pyridin-3-yl)sulfonyl)-6-(tetrahydro-2H-pyran-4-yl)-2,6-diazaspiro[3.3]heptane

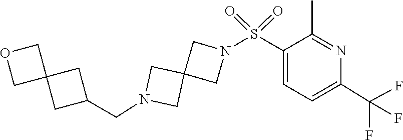 [SMILES: Cc1nc(C(F)(F)F)ccc1S(=O)(=O)N1CC2(CN(CC3CC4(COC4)C3)C2)C1]

### 1. Synthesis of (2-oxaspiro[3.3]heptan-6-yl)methyl 4-methylbenzenesulfonate

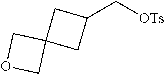 [SMILES: Cc1ccc(S(=O)(=O)OCC2CC3(COC3)C2)cc1]

DMAP (476.59 mg, 3.90 mmol) and DIEA (1.51 g, 11.70 mmol, 2.04 mL) were added to a solution of (2-oxaspiro[3.3]heptan-6-yl)methanol (500 mg, 3.90 mmol) and TsCl (1.12 g, 5.85 mmol) in DCM (10 mL). The mixture was stirred at 25° C. for 12 h. The reaction mixture was diluted with H₂O (50 mL) and extracted with DCM (50 mL×3). The combined organic layers were washed with brine (100 mL), dried over Na₂SO₄, filtered and concentrated under reduced pressure to give a residue, which was purified by flash silica gel chromatography (ISCO®; 12 g SepaFlash® Silica Flash Column, Eluent of 0˜50% Ethyl acetate/Petroleum ether gradient @30 mL/min) to give the desired compound (590 mg, 48% yield) as a yellow oil. ¹H NMR (400 MHz, CDCl₃) δ (ppm) 7.78 (d, J=8.0 Hz, 2H), 7.35 (d, J=8.0 Hz, 2H), 4.64 (s, 2H), 4.52 (s, 2H), 3.91 (d, J=6.0 Hz, 2H), 2.45 (s, 3H), 2.43-2.37 (m, 1H), 2.35-2.27 (m, 2H), 1.99-1.89 (m, 2H).

### 2. Synthesis of 2-((2,6-bis(trifluoromethyl)pyridin-3-yl)sulfonyl)-6-(tetrahydro-2H-pyran-4-yl)-2,6-diazaspiro[3.3]heptane

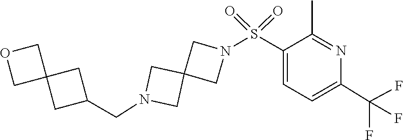 [SMILES: Cc1nc(C(F)(F)F)ccc1S(=O)(=O)N1CC2(CN(CC3CC4(COC4)C3)C2)C1]

To a solution of (2-oxaspiro[3.3]heptan-6-yl)methyl 4-methylbenzenesulfonate (100 mg, 318.75 umol) and 2-[[2-methyl-6-(trifluoromethyl)-3-pyridyl]sulfonyl]-2,6-diazaspiro[3.3]heptane (154.18 mg, 318.75 umol, TFA salt) in CH₃CN (10 mL) was added K₂CO₃ (220.27 mg, 1.59 mmol). The mixture was stirred at 80° C. for 12 h. LCMS showed the reaction was completed. The mixture was filtered and concentrated to give a residue. The residue was purified by preparative HPLC (column: Phenomenex C18 150×25 mm×10 um; mobile phase: [water (NH₄HCO₃)-ACN]; B %: 28%-58%, 8 min) to give the title compound (29.3 mg, 21% yield) as a yellow solid. LCMS m/z=432.1 (M+H)⁺. ¹H NMR (400 MHz, MeOD) δ (ppm) 8.45 (d, J=8.0 Hz, 1H), 7.83 (d, J=8.0 Hz, 1H), 4.69 (s, 2H), 4.54 (s, 2H), 4.02 (s, 4H), 3.32 (s, 4H), 2.86 (s, 3H), 2.40 (d, J=7.2 Hz, 2H), 2.35-2.27 (m, 2H), 2.15-2.01 (m, 1H), 1.91-1.82 (m, 2H).

### Examples 114 and 115

The Examples 114 and 115 in the following Table were prepared using a similar method described for Example 113 from (2-oxaspiro[3.3]heptan-6-yl)methyl 4-methylbenzenesulfonate and the corresponding 2,6-diazaspiro[3.3]heptane sulfonamide.

| Example | Name/Structure/(SM) | DATA |
|---|---|---|
| 114 | 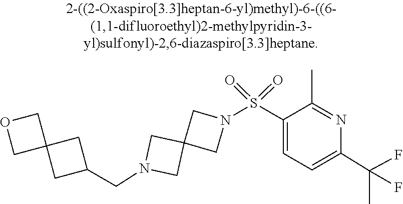 [SMILES: Cc1nc(C(C)(F)F)ccc1S(=O)(=O)N1CC2(CN(CC3CC4(COC4)C3)C2)C1] | LCMS m/z = 428.1 (M + H)⁺. ¹H NMR (500 MHz, CDCl₃) δ (ppm) 8.27 (d, J = 8.0 Hz, 1H), 7.60 (d, J = 8.0 Hz, 1H), 4.68 (s, 2H), 4.55 (s, 2H), 3.99 (s, 4H), 3.25 (s, 4H), 2.85 (s, 3H), 2.34-2.27 (m, 4H), 2.06-1.97 (m, 4H), 1.84-1.78 (m, 2H). |
| | SM: 2-((6-(1,1-difluoroethyl)-2-methylpyridin-3- | |
| | yl)sulfonyl)-2,6-diazaspiro[3.3]heptane | |
| 115 | 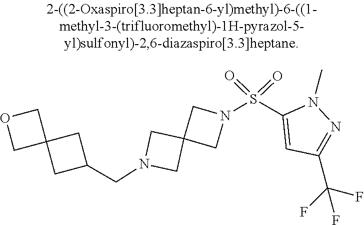 [SMILES: Cn1nc(C(F)(F)F)cc1S(=O)(=O)N1CC2(CN(CC3CC4(COC4)C3)C2)C1] | LCMS m/z = 421.1 (M + H)⁺. ¹H NMR (400 MHz, MeOD) δ (ppm) 7.20 (s, 1H), 4.69 (s, 2H), 4.54 (s, 2H), 4.15 (s, 3H), 4.00 (s, 4H), 3.27 (s, 4H), 2.38 (d, J = 7.2 Hz, 2H), 2.34-2.28 (m, 2H), 2.12-2.03 (m, 1H), 1.88-1.82 (m, 2H) |
| | SM: 2-((1-methyl-3-(trifluoromethyl)-1H- | |
| | pyrazol-5-yl)sulfonyl)-2,6- | |
| | diazaspiro[3.3]heptane | |

### Example 116: 2-((2-cyclopropyl-6-(difluoromethyl)pyridin-3-yl)sulfonyl)-6-(tetrahydro-2H-pyran-4-yl)-2,6-diazaspiro[3.3]heptane

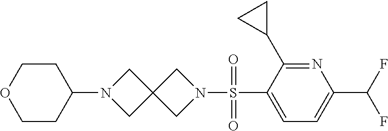 [SMILES: O=S(=O)(c1ccc(C(F)F)nc1C1CC1)N1CC2(CN(C3CCOCC3)C2)C1]

### 1. Synthesis of 2-bromo-6-(difluoromethyl)pyridin-3-amine

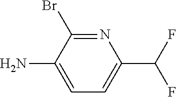 [SMILES: Nc1ccc(C(F)F)nc1Br]

To a solution of 6-(difluoromethyl)pyridin-3-amine (550 mg, 3.82 mmol) in ACN (20 mL) was added NBS (679 mg, 3.82 mmol) at 0° C. The mixture was stirred for 20 min at 0° C. under nitrogen, then the reaction mixture was concentrated in vacuo. The crude was purified by flash column (ethyl acetate in petroleum ether from 10% to 30%) to afford 2-bromo-6-(difluoromethyl)pyridin-3-amine (760 mg, 89%) as a white solid. ¹H NMR (500 MHz, CDCl₃) δ=7.40 (d, J=8.0 Hz, 1H), 7.07 (d, J=8.5 Hz, 1H), 6.52 (t, J=55.5 Hz, 1H), 4.38 (br s, 2H).

### 2. Synthesis of 2-cyclopropyl-6-(difluoromethyl)pyridin-3-amine

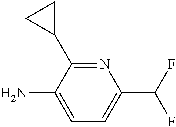 [SMILES: Nc1ccc(C(F)F)nc1C1CC1]

To a solution of 2-bromo-6-(difluoromethyl)pyridin-3-amine (730 mg, 3.27 mmol) and cyclopropylboronic acid (562 mg, 6.55 mmol) in toluene (30 mL) and water (2 mL) was added S-Phos (134 mg, 327 μmol), K₃PO₄ (2.08 g, 9.82 mmol) and Pd₂(dba)₃ (300 mg, 327 μmol) at 20° C. The mixture was stirred for 3 h at 100° C. under nitrogen. The reaction mixture was filtered and concentrated under vacuum. The crude material was purified by flash column (ethyl acetate in petroleum ether from 10% to 20%) to afford 2-cyclopropyl-6-(difluoromethyl)pyridin-3-amine (350 mg, 58%) as a red oil that was used without further purification. LCMS m/z=185.1 (M+H)⁺.

### 3. Synthesis of 2-cyclopropyl-6-(difluoromethyl)pyridine-3-sulfonyl chloride

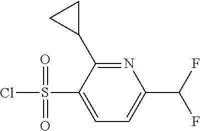 [SMILES: O=S(=O)(Cl)c1ccc(C(F)F)nc1C1CC1]

Thionyl chloride (1 mL, 13.7 mmol) was slowly added over 10 minutes to water (2 mL) at 0° C. During the addition to water, the temperature was maintained between 0-5° C. After addition to water, the solution was warmed to 15° C., then copper chloride (13 mg, 135 μmol) was added. The solution was cooled back to 0° C. A solution of sodium nitrite (100 mg, 1.45 mmol) in water (2 mL) was slowly added to a solution of 2-cyclopropyl-6-(difluoromethyl)pyridin-3-amine (250 mg, 1.36 mmol) in conc. HCl (1 mL) at 0° C. During addition, the temperature was maintained between 0-5° C. This mixture was slowly added to the above prepared solution to maintain a temperature between 0-5° C. The mixture was stirred for an additional 1 hour after addition, then the mixture was extracted three times with dichloromethane. The organic extractions were pooled then dried over anhydrous sodium sulfate, filtered, and concentrated to afford a yellow oil as 2-cyclopropyl-6-(difluoromethyl)pyridine-3-sulfonyl chloride that was used without purification. ¹H NMR (400 MHz, CDCl₃) δ (ppm) 8.41 (d, J=8.0 Hz, 1H), 7.55 (d, J=8.4 Hz, 1H), 6.53 (t, J=54.8 Hz, 1H), 3.03-2.98 (m, 1H), 1.41-1.39 (m, 2H), 1.31-1.28 (m, 2H).

### 4. Synthesis of tert-butyl 6-((2-cyclopropyl-6-(difluoromethyl)pyridin-3-yl)sulfonyl)-2,6-diazaspiro[3.3]heptane-2-carboxylate

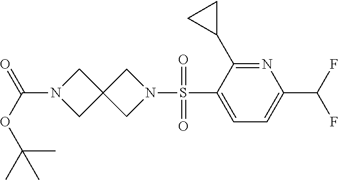 [SMILES: CC(C)(C)OC(=O)N1CC2(C1)CN(S(=O)(=O)c1ccc(C(F)F)nc1C1CC1)C2]

To a solution of tert-butyl 2,6-diazaspiro[3.3]heptane-2-carboxylate (130 mg, 658 μmol) and DIPEA (0.43 mL, 2.5 mmol) in DCM (10 mL) was added 2-cyclopropyl-6-(difluoromethyl)pyridine-3-sulfonyl chloride (220 mg, 822 μmol) at 0° C. under nitrogen. The mixture was stirred at 25° C. for 1 h. The mixture was diluted with water (30 mL) and extracted with DCM (30 mL×2). The combined organic phase was washed with brine (30 mL), dried over anhydrous sodium sulfate, filtered, and concentrated under vacuum. The residue was purified by flash column (ethyl acetate in petroleum ether from 5% to 20%) to afford tert-butyl 6-((2-cyclopropyl-6-(difluoromethyl)pyridin-3-yl)sulfonyl)-2,6-diazaspiro[3.3]heptane-2-carboxylate (260 mg, 52%) as yellow solid. LCMS m/z=430.1 (M+H)⁺. ¹H NMR (400 MHz, CDCl₃) δ (ppm) 8.28 (d, J=8.4 Hz, 1H), 7.47 (d, J=8.4 Hz, 1H), 6.51 (t, J=55.2 Hz, 1H), 4.05 (s, 4H), 4.00 (s, 4H), 2.89-2.83 (m, 1H), 1.42 (s, 9H), 1.31-1.27 (m, 2H), 1.16-1.13 (m, 2H).

### 5. Synthesis of 2-((2-cyclopropyl-6-(difluoromethyl)pyridin-3-yl)sulfonyl)-2,6-diazaspiro[3.3]heptane trifluoroacetate

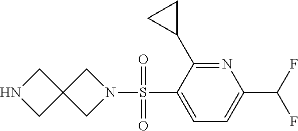 [SMILES: O=S(=O)(c1ccc(C(F)F)nc1C1CC1)N1CC2(CNC2)C1]

To a solution of tert-butyl 6-((2-cyclopropyl-6-(difluoromethyl)pyridin-3-yl)sulfonyl)-2,6-diazaspiro[3.3]heptane-2-carboxylate (250 mg, 582 μmol) in HFiPA (10 mL) was added TFA (0.22 mL, 2.9 mmol) at 15° C. After 3 h, the mixture was concentrated under reduced pressure to afford a yellow oil as 2-((2-cyclopropyl-6-(difluoromethyl)pyridin-3-yl)sulfonyl)-2,6-diazaspiro[3.3]heptane trifluoroacetate that was used without purification. LCMS m/z=330.1 (M+H)⁺. ¹H NMR (400 MHz, CD₃OD) δ (ppm) 8.36 (d, J=8.0 Hz, 1H), 7.57 (d, J=8.0 Hz, 1H), 6.64 (t, J=55.2 Hz, 1H), 4.22 (s, 4H), 4.15 (s, 4H), 2.94-2.88 (m, 1H), 1.28-1.24 (m, 2H), 1.19-1.13 (m, 2H).

### 6. Synthesis of 2-((2-cyclopropyl-6-(difluoromethyl)pyridin-3-yl)sulfonyl)-6-(tetrahydro-2H-pyran-4-yl)-2,6-diazaspiro[3.3]heptane

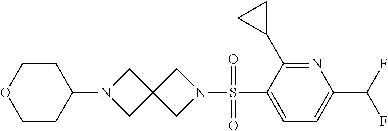 [SMILES: O=S(=O)(c1ccc(C(F)F)nc1C1CC1)N1CC2(CN(C3CCOCC3)C2)C1]

A solution of 2-((2-cyclopropyl-6-(difluoromethyl)pyridin-3-yl)sulfonyl)-2,6-diazaspiro[3.3]heptane trifluoroacetate (130 mg, 293 μmol) and tetrahydropyran-4-one (29 mg, 293 μmol) in MeOH (10 mL) was adjusted to pH 7-8 by dropwise addition of triethylamine. After 10 minutes, the mixture was adjusted to pH 5-6 by acetic acid and stirred for 30 minutes at 20° C. Sodium cyanoborohydride (55 mg, 880 μmol) was added to the mixture and stirred for 1 h at 20° C. The mixture was diluted with water (30 mL) and extracted with DCM (20 mL×3). The combined organic phase was dried over anhydrous sodium sulfate, filtered, and concentrated under vacuum. The residue was purified by prep-HPLC (Welch Xtimate C18 150*25 mm*5 um, Condition: water (NH₄HCO₃)-ACN, Flow Rate (mL/min): 25) to give 2-((2-cyclopropyl-6-(difluoromethyl)pyridin-3-yl)sulfonyl)-6-(tetrahydro-2H-pyran-4-yl)-2,6-diazaspiro[3.3]heptane (51 mg, 42%) as a light-yellow solid. LCMS m/z=414.1 (M+H)⁺. ¹H NMR (400 MHz, CDCl₃) δ (ppm) 8.28 (d, J=8.4 Hz, 1H), 7.47 (d, J=8.0 Hz, 1H), 6.51 (t, J=54.8 Hz, 1H), 4.02 (s, 4H), 3.95-3.90 (m, 2H), 3.36-3.30 (m, 2H), 3.25 (s, 4H), 2.93-2.86 (m, 1H), 2.15-2.08 (m, 1H), 1.60-1.56 (m, 2H), 1.30-1.26 (m, 4H), 1.15-1.12 (m, 2H).

### Examples 117 and 118: 2-((2-cyclopropyl-6-(difluoromethyl)pyridin-3-yl)sulfonyl)-6-(4-methoxycyclohexyl)-2,6-diazaspiro[3.3]heptane and 2-((2-cyclopropyl-6-(difluoromethyl)pyridin-3-yl)sulfonyl)-6-(4-methoxycyclohexyl)-2,6-diazaspiro[3.3]heptane

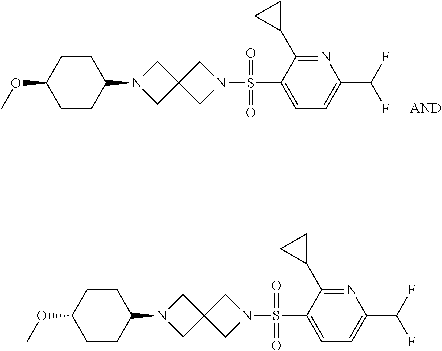

The title compound was prepared using a similar method to step 6 of Example 116 from 2-((2-cyclopropyl-6-(difluoromethyl)pyridin-3-yl)sulfonyl)-2,6-diazaspiro[3.3]heptane trifluoroacetate (200 mg, 451 μmol) and 4-methoxycyclohexanone (58 mg, 451 μmol) to afford the following compounds:

2-((2-cyclopropyl-6-(difluoromethyl)pyridin-3-yl)sulfonyl)-6-(4-methoxycyclohexyl)-2,6-diazaspiro[3.3]heptane (TRANS) after HPLC (basic conditions) (first peak off the prep HPLC) (48 mg, 24%). LCMS m/z=442.2 (M+H)⁺. ¹H NMR (500 MHz, CDCl₃) δ (ppm) 8.27 (d, J=8.0 Hz, 1H), 7.46 (d, J=8.5 Hz, 1H), 6.51 (t, J=55.0 Hz, 1H), 4.01 (s, 4H), 3.32 (s, 3H), 3.25 (s, 4H), 3.10-3.05 (m, 1H), 2.92-2.86 (m, 1H), 2.03-2.01 (m, 2H), 1.90 (br s, 1H), 1.74-1.72 (m, 2H), 1.30-1.27 (m, 2H), 1.16-1.11 (m, 4H), 0.99-0.97 (m, 2H).

2-((2-cyclopropyl-6-(difluoromethyl)pyridin-3-yl)sulfonyl)-6-(4-methoxycyclohexyl)-2,6-diazaspiro[3.3]heptane (CIS) after HPLC (basic conditions) (second peak off the prep HPLC) (46 mg, 23%). LCMS m/z=442.1 (M+H)⁺. ¹H NMR (500 MHz, CDCl₃) δ (ppm) 8.27 (d, J=8.0 Hz, 1H), 7.46 (d, J=8.5 Hz, 1H), 6.51 (t, J=55.0 Hz, 1H), 4.00 (s, 4H), 3.30-3.29 (m, 1H), 3.26 (s, 3H), 3.21 (s, 4H), 2.92-2.87 (m, 1H), 1.94 (br s, 1H), 1.83-1.81 (m, 2H), 1.38-1.35 (m, 6H), 1.30-1.27 (m, 2H), 1.15-1.12 (m, 2H).

### Example 119: 2-((2-cyclopropyl-6-(difluoromethyl)pyridin-3-yl)sulfonyl)-6-(2-oxaspiro[3.3]heptan-6-yl)-2,6-diazaspiro[3.3]heptane

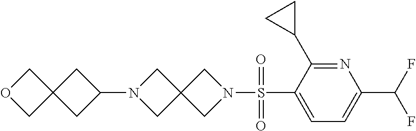 [SMILES: O=S(=O)(c1ccc(C(F)F)nc1C1CC1)N1CC2(CN(C3CC4(COC4)C3)C2)C1]

The title compound was prepared using a similar method to step 6 of Example 116 from 2-((2-cyclopropyl-6-(difluoromethyl)pyridin-3-yl)sulfonyl)-2,6-diazaspiro[3.3]heptane trifluoroacetate (1000 mg, 225 μmol) and oxaspiro[3.3]heptan-6-one (25 mg, 225 μmol) to afford 2-((2-cyclopropyl-6-(difluoromethyl)pyridin-3-yl)sulfonyl)-6-(2-oxaspiro[3.3]heptan-6-yl)-2,6-diazaspiro[3.3]heptane after HPLC (basic conditions) (48 mg, 50%). LCMS m/z=426.2 (M+H)⁺. ¹H NMR (400 MHz, CDCl₃) δ (ppm) 8.27 (d, J=8.0 Hz, 1H), 7.46 (d, J=8.0 Hz, 1H), 6.50 (t, J=55.2 Hz, 1H), 4.64 (s, 2H), 4.58 (s, 2H), 3.99 (s, 4H), 3.19 (s, 4H), 2.91-2.79 (m, 2H), 2.27-2.22 (m, 2H), 1.94-1.89 (m, 2H), 1.30-1.25 (m, 2H), 1.15-1.11 (m, 2H).

### Example 120: 2-((1-cyclopropyl-3-(difluoromethyl)-1H-pyrazol-5-yl)sulfonyl)-6-(tetrahydro-2H-pyran-4-yl)-2,6-diazaspiro[3.3]heptane

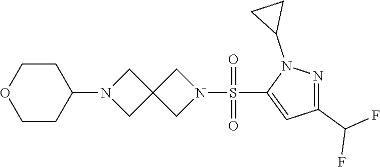 [SMILES: O=S(=O)(c1cc(C(F)F)nn1C1CC1)N1CC2(CN(C3CCOCC3)C2)C1]

### 1. Synthesis of 1-cyclopropyl-3-(difluoromethyl)-1H-pyrazole

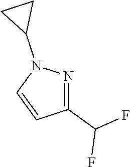 [SMILES: FC(F)c1ccn(C2CC2)n1]

To a solution of 1-cyclopropylpyrazole-3-carbaldehyde (2.2 g, 16 mmol) in DCM (40 mL) was added DAST (5.3 mL, 40 mmol) at −30° C. The mixture was stirred at 20° C. for 12 h. The reaction mixture was carefully quenched by sat. NH₄Cl (20 mL) at 15° C. then diluted with H₂O (20 mL) and extracted with DCM (40 mL×3). The combined organic layers were dried over Na₂SO₄, filtered, and concentrated under reduced pressure. The residue was purified by flash column (ethyl acetate in petroleum ether from 0% to 12%) to give 1-cyclopropyl-3-(difluoromethyl)-1H-pyrazole (1.3 g, 51%) as a colorless oil. LCMS m/z=159.1 (M+H)⁺. ¹H NMR (400 MHz, CDCl₃) δ (ppm) 7.47 (s, 1H), 6.66 (t, J=55.0 Hz, 1H), 6.44 (s, 1H), 3.61-3.60 (m, 1H), 1.15-1.13 (m, 2H), 1.07-1.03 (m, 2H).

### 2. Synthesis of 5-bromo-1-cyclopropyl-3-(difluoromethyl)-1H-pyrazole

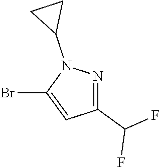 [SMILES: FC(F)c1cc(Br)n(C2CC2)n1]

To a solution of 1-cyclopropyl-3-(difluoromethyl)-1H-pyrazole (1.3 g, 8.2 mmol) in THE (20 mL) was added butyllithium (2.5 M, 3.95 mL) at −65° C. The mixture was stirred at −65° C. for 1 hour, then carbon tetrabromide (3.54 g, 10.7 mmol) in THE (4 mL) was added to the mixture at −65° C. and stirred for 0.5 hour. The mixture was warmed to 25° C. and stirred for 1 hour. The reaction mixture was carefully quenched by sat. NH₄Cl (20 mL) at 0° C. then diluted with H₂O (20 mL) and extracted with ethyl acetate (40 mL×3). The combined organic layers were dried over Na₂SO₄, filtered, and concentrated under reduced pressure. The residue was purified by flash column (ethyl acetate in petroleum ether from 0% to 8%) to give 5-bromo-1-cyclopropyl-3-(difluoromethyl)-1H-pyrazole (1.6 g, 82%) as a colorless oil. LCMS m/z=238.9 (M+H)⁺. ¹H NMR (500 MHz, CDCl₃) δ (ppm) 6.57 (t, J=55.2 Hz, 1H), 6.52 (s, 1H), 3.52-3.47 (m, 1H), 1.24-1.20 (m, 2H), 1.14-1.08 (m, 2H).

### 3. Synthesis of N-(1-cyclopropyl-3-(difluoromethyl)-1H-pyrazol-5-yl)-1,1-diphenylmethanimine

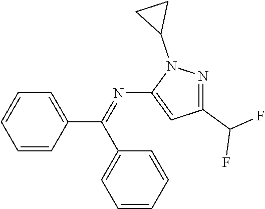 [SMILES: FC(F)c1cc(N=C(c2ccccc2)c2ccccc2)n(C2CC2)n1]

To a solution of 5-bromo-1-cyclopropyl-3-(difluoromethyl)-1H-pyrazole (1.6 g, 6.7 mmol) and diphenylmethanimine (2.3 mL, 13.5 mmol) in dioxane (30 mL) was added Pd₂(dba)₃ (618 mg, 675 μmol), Xantphos (781 mg, 1.35 mmol), and K₂CO₃ (2.80 g, 20.2 mmol), then stirred at 100° C. for 16 h. The mixture was filtered. The organic phase was diluted with water (20 mL) then extracted with ethyl acetate (30 mL×3). The combined organic phase was washed with brine (20 mL×1), dried over anhydrous sodium sulfate, filtered, and concentrated under vacuum. The crude was purified by flash column (ethyl acetate in petroleum ether from 0% to 10%) to give N-(1-cyclopropyl-3-(difluoromethyl)-1H-pyrazol-5-yl)-1,1-diphenylmethanimine as a yellow oil that was used without further purification. LCMS m/z=338.1 (M+H)⁺. ¹H NMR (500 MHz, CDCl₃) δ (ppm) 7.82-7.81 (m, 5H), 7.81-7.80 (m, 5H), 6.43 (t, J=55.0 Hz, 1H), 4.95 (s, 1H), 3.99-3.95 (m, 1H), 1.32-1.28 (m, 2H), 1.09-1.05 (m, 2H).

### 4. Synthesis of 1-cyclopropyl-3-(difluoromethyl)-1H-pyrazol-5-amine

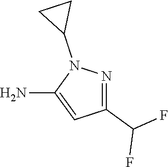 [SMILES: Nc1cc(C(F)F)nn1C1CC1]

To a solution of N-(1-cyclopropyl-3-(difluoromethyl)-1H-pyrazol-5-yl)-1,1-diphenylmethanimine (2.2 g, 3.3 mmol) in THF (40 mL) and water (8 mL) was added HCl (2 M, 11 mL), then the mixture was stirred at 25° C. for 1 h. Aqueous NaHCO₃ (50 mL) solution was added dropwise to quench the reaction, then the mixture was extracted with ethyl acetate (50 mL×3). The combined organic phase was washed with brine, dried over anhydrous sodium sulfate, filtered, and concentrated. The residue was purified by flash column (MeOH in DCM=from 0% to 8%) to give 1-cyclopropyl-3-(difluoromethyl)-1H-pyrazol-5-amine (450 mg, 80%) as a yellow oil. LCMS m/z=174.1 (M+H)⁺.

### 5. Synthesis of 1-cyclopropyl-3-(difluoromethyl)-1H-pyrazole-5-sulfonyl chloride

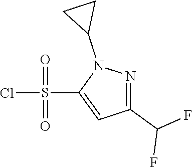 [SMILES: O=S(=O)(Cl)c1cc(C(F)F)nn1C1CC1]

Thionyl chloride (2.5 mL, 34 mmol) was added over 10 min to water (5 mL) while the temperature was maintained 0-5° C., then the solution was stirred at 20° C. for 12 hours. Copper chloride (14 mg, 144 μmol) was added, then the mixture was cooled to −3° C. In another flask, a solution of 1-cyclopropyl-3-(difluoromethyl)-1H-pyrazol-5-amine (250 mg, 1.44 mmol) in HCl (12 M, 3.7 mL) at −5° C. was added dropwise to a solution of sodium nitrite (100 mg, 1.46 mmol) in water (0.5 mL) while maintaining temperature −5 to 0° C. for 1 h. When the addition was complete, this solution was added to a precooled thionyl chloride solution and stirred at −2° C. for 10 min, then at 0° C. for 75 min. The mixture was extracted with DCM (30 mL×3). The combined organic phase was dried over Na₂SO₄, filtered, and concentrated to give 1-cyclopropyl-3-(difluoromethyl)-1H-pyrazole-5-sulfonyl chloride that was used without purification. ¹H NMR (400 MHz, CDCl₃) δ (ppm) 6.55 (t, J=54.8 Hz, 1H), 6.42 (s, 1H), 3.51-3.45 (m, 1H), 1.25-1.20 (m, 2H), 1.13-1.08 (m, 2H).

### 6. Synthesis of 2-((1-cyclopropyl-3-(difluoromethyl)-1H-pyrazol-5-yl)sulfonyl)-6-(tetrahydro-2H-pyran-4-yl)-2,6-diazaspiro[3.3]heptane

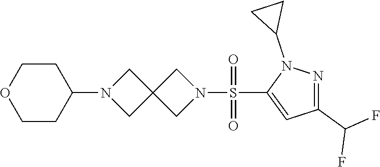 [SMILES: O=S(=O)(c1cc(C(F)F)nn1C1CC1)N1CC2(CN(C3CCOCC3)C2)C1]

To a solution of 2-(tetrahydro-2H-pyran-4-yl)-2,6-diazaspiro[3.3]heptane trifluoroacetate (69 mg, 234 μmol) and DIPEA (0.1 mL, 584 μmol) in DCM (15 mL) was added 1-cyclopropyl-3-(difluoromethyl)-1H-pyrazole-5-sulfonyl chloride (100 mg, 195 μmol) in DCM (3 mL) at 0° C. The mixture was stirred for 1 hour at 20° C. The mixture was diluted with water (30 mL) and extracted with DCM (40 mL×3). The combined organic phase was dried over anhydrous sodium sulfate, filtered, and concentrated in vacuum. The residue was purified by prep-HPLC (Column: Boston Green ODS 150*30 mm*5 um, Condition: water(formic acid)-ACN, 8%˜38%, Flow Rate (mL/min): 25) to give 2-((1-cyclopropyl-3-(difluoromethyl)-1H-pyrazol-5-yl)sulfonyl)-6-(tetrahydro-2H-pyran-4-yl)-2,6-diazaspiro[3.3]heptane (4 mg, 6%) as a white solid. LCMS m/z=403.1 (M+H)⁺. ¹H NMR (500 MHz, CD₃OD) δ (ppm) 7.05 (s, 1H), 6.75 (t, J=54.5 Hz, 1H), 4.22-4.18 (m, 1H), 4.12 (s, 4H), 3.97-3.95 (m, 2H), 3.75 (s, 4H), 3.38-3.35 (m, 2H), 2.75-2.72 (m, 1H), 1.77-1.75 (m, 2H), 1.37-1.32 (m, 2H), 1.30-1.25 (m, 2H), 1.15-1.11 (m, 2H).

### Example 121: 2-((4-cyclopropyl-6-(trifluoromethyl)pyridin-3-yl)sulfonyl)-6-(2-oxaspiro[3.3]heptan-6-yl)-2,6-diazaspiro[3.3]heptane

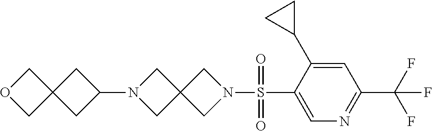 [SMILES: O=S(=O)(c1cnc(C(F)(F)F)cc1C1CC1)N1CC2(CN(C3CC4(COC4)C3)C2)C1]

### 1. Synthesis of 4-cyclopropyl-6-(trifluoromethyl)pyridin-3-amine

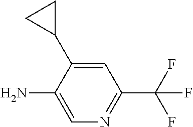 [SMILES: Nc1cnc(C(F)(F)F)cc1C1CC1]

To a solution of 4-bromo-6-(trifluoromethyl)pyridin-3-amine (1.5 g, 6.2 mmol) and cyclopropylboronic acid (1.1 g, 12 mmol) in toluene (20 mL) and water (2 mL) was added S-Phos (255 mg, 622 μmol), Pd₂(dba)₃ (570 mg, 622 μmol) and K₃PO₄ (3.96 g, 18.7 mmol) at 20° C. The mixture was stirred for 3 hours at 100° C. under nitrogen. After cooling to room temperature, the reaction was diluted with water (15 mL) then extracted with DCM (20 mL×3). The combined organic phase was washed with brine (15 mL×1), dried over anhydrous sodium sulfate, filtered, and concentrated under vacuum. The crude material was purified by flash column (ethyl acetate in petroleum ether=10-50%) to give 4-cyclopropyl-6-(trifluoromethyl)pyridin-3-amine (900 mg, 71%) as a brown solid that was used without further purification. LCMS m/z=203.1 (M+H)⁺. ¹H NMR (500 MHz, CDCl₃) δ (ppm) 8.06 (s, 1H), 7.24 (s, 1H), 4.25 (br s, 2H), 1.72-1.61 (m, 1H), 1.06-1.01 (m, 2H), 0.70-0.66 (m, 2H).

### 2. Synthesis of 4-cyclopropyl-6-(trifluoromethyl)pyridine-3-sulfonyl chloride

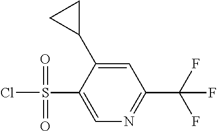 [SMILES: O=S(=O)(Cl)c1cnc(C(F)(F)F)cc1C1CC1]

Thionyl chloride (3 mL) was added over 10 minutes to water (18 mL), while the temperature was maintained 0-5° C., then the solution was stirred at 20° C. for 12 hours. Copper chloride (3 mg, 35 μmol) was added, then the mixture was cooled to −3° C. In another flask, a solution of 4-cyclopropyl-6-(trifluoromethyl)pyridin-3-amine (350 mg, 1.73 mmol) in HCl (12 M, 4.5 mL) at −5° C. was added dropwise to a solution of sodium nitrite (125 mg, 1.82 mmol) in water (1.5 mL) while maintaining temperature −5 to 0° C. for 1 hour. When the addition was complete, this solution was added to the precooled thionyl chloride solution and stirred at −2° C. for 10 minutes, then at 0° C. for 75 minutes. The mixture was concentrated, then water (10 mL) was added. The mixture was extracted with DCM (10 mL×3). The combined organic layers were washed with brine (10 mL), dried over Na₂SO₄, filtered, and concentrated to give 4-cyclopropyl-6-(trifluoromethyl)pyridine-3-sulfonyl chloride as yellow oil that was used without further purification. ¹H NMR (500 MHz, CDCl₃) δ (ppm) 9.25 (s, 1H), 7.28 (s, 1H), 3.00-2.93 (m, 1H), 1.62-1.48 (m, 2H), 1.21-1.13 (m, 2H).

### 3. Synthesis of tert-butyl 6-((4-cyclopropyl-6-(trifluoromethyl)pyridin-3-yl)sulfonyl)-2,6-diazaspiro[3.3]heptane-2-carboxylate

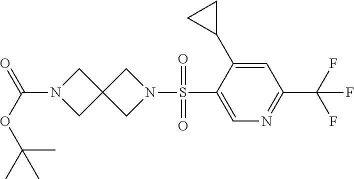 [SMILES: CC(C)(C)OC(=O)N1CC2(C1)CN(S(=O)(=O)c1cnc(C(F)(F)F)cc1C1CC1)C2]

To a solution of 4-cyclopropyl-6-(trifluoromethyl)pyridine-3-sulfonyl chloride (380 mg, 1.33 mmol) and DIPEA (0.7 mL, 4 mmol) in DCM (10 mL) was added tert-butyl 2,6-diazaspiro[3.3]heptane-2-carboxylate (316 mg, 1.60 mmol) at 0° C. The mixture was stirred at 20° C. for 2 hours. The mixture was diluted with water (15 mL) then extracted with DCM (15 mL×3). The combined organic phase was washed with brine (15 mL×1), dried over anhydrous sodium sulfate, filtered, and concentrated under vacuum. The crude material was purified on silica gel column chromatography (petroleum ether/ethyl acetate=5/1 to 1/1) to yield tert-butyl 6-((4-cyclopropyl-6-(trifluoromethyl)pyridin-3-yl)sulfonyl)-2,6-diazaspiro[3.3]heptane-2-carboxylate (370 mg, 62%) as a white solid. ¹H NMR (500 MHz, CDCl₃) δ (ppm) 9.08 (s, 1H), 7.12 (s, 1H), 4.08 (s, 4H), 4.03 (s, 4H), 2.84-2.75 (m, 1H), 1.42 (s, 9H), 1.32 (br s, 2H), 1.05-1.02 (m, 2H).

### 4. Synthesis of 2-((4-cyclopropyl-6-(trifluoromethyl)pyridin-3-yl)sulfonyl)-2,6-diazaspiro[3.3]heptane trifluoroacetate

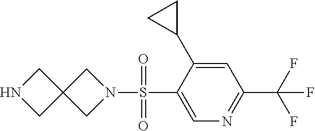 [SMILES: O=S(=O)(c1cnc(C(F)(F)F)cc1C1CC1)N1CC2(CNC2)C1]

To a solution of tert-butyl 6-((4-cyclopropyl-6-(trifluoromethyl)pyridin-3-yl)sulfonyl)-2,6-diazaspiro[3.3]heptane-2-carboxylate (120 mg, 268 μmol) in HFiPA (3 mL) was added TFA (0.5 mL, 6.5 mmol). The reaction mixture was stirred at 15° C. for 2 hours. The mixture was concentrated in vacuum to give 2-((4-cyclopropyl-6-(trifluoromethyl)pyridin-3-yl)sulfonyl)-2,6-diazaspiro[3.3]heptane trifluoroacetate as a yellow oil that was used without purification. ¹H NMR (500 MHz, CD₃OD) δ (ppm) 9.03 (s, 1H), 7.37 (s, 1H), 4.24 (s, 4H), 4.18 (s, 4H), 2.85-2.79 (m, 1H), 1.41-1.36 (m, 2H), 1.15-1.11 (m, 2H).

### 5. Synthesis of 2-((4-cyclopropyl-6-(trifluoromethyl)pyridin-3-yl)sulfonyl)-6-(2-oxaspiro[3.3]heptan-6-yl)-2,6-diazaspiro[3.3]heptane

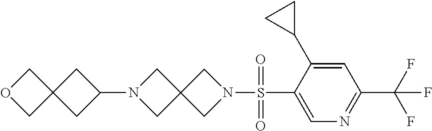 [SMILES: O=S(=O)(c1cnc(C(F)(F)F)cc1C1CC1)N1CC2(CN(C3CC4(COC4)C3)C2)C1]

To a solution of 2-((4-cyclopropyl-6-(trifluoromethyl)pyridin-3-yl)sulfonyl)-2,6-diazaspiro[3.3]heptane trifluoroacetate (130 mg, 282 μmol) and TEA (0.12 mL, 847 μmol) in MeOH (3 mL) was added 2-oxaspiro[3.3]heptan-6-one (38 mg, 339 μmol). The mixture pH was adjusted to pH=5-6 by dropwise addition of acetic acid at 0° C. After 30 minutes, sodium cyanoborohydride (53 mg, 847 μmol) was added, and the mixture was stirred at 20° C. for 2 hours. The mixture was diluted with water (15 mL) and extracted with DCM (15 mL×3). The combined organic phase was washed with brine (15 mL×1), dried over anhydrous sodium sulfate, filtered, and concentrated under vacuum. The crude which was purified by prep-HPLC (Column: Waters Xbridge BEH C18 100*30 mm*10 um; Condition: water (NH₄HCO₃)-ACN; Begin B: 31; End B: 61; Flow Rate (ml/min): 50) to give 2-((4-cyclopropyl-6-(trifluoromethyl)pyridin-3-yl)sulfonyl)-6-(2-oxaspiro[3.3]heptan-6-yl)-2,6-diazaspiro[3.3]heptane (60 mg, 48%) as a white solid. LCMS m/z=444.1 (M+H)⁺. ¹H NMR (500 MHz, CDCl₃) δ (ppm) 9.07 (s, 1H), 7.11 (s, 1H), 4.64 (s, 2H), 4.58 (s, 2H), 4.02 (s, 4H), 3.20 (br s, 4H), 2.88-2.73 (m, 2H), 2.30-2.18 (m, 2H), 1.92 (br s, 2H), 1.42-1.32 (m, 2H), 1.04-0.98 (m, 2H).

### Example 122: 2-((4-cyclopropyl-6-(trifluoromethyl)pyridin-3-yl)sulfonyl)-6-((tetrahydro-2H-pyran-4-yl)methyl)-2,6-diazaspiro[3.3]heptane

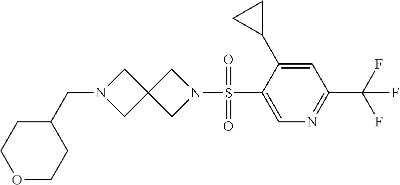 [SMILES: O=S(=O)(c1cnc(C(F)(F)F)cc1C1CC1)N1CC2(CN(CC3CCOCC3)C2)C1]

To a solution of 2-((4-cyclopropyl-6-(trifluoromethyl)pyridin-3-yl)sulfonyl)-2,6-diazaspiro[3.3]heptane trifluoroacetate (100 mg, 287 μmol) and TEA (0.12 mL, 847 μmol) in MeOH (5 mL) was added tetrahydropyran-4-carbaldehyde (33 mg, 287 μmol). The mixture was adjusted to pH=5-6 by dropwise addition of acetic acid at 0° C. After 30 minutes, sodium cyanoborohydride (54 mg, 863 μmol) was added, and the mixture was stirred at 20° C. for 2 hours. The mixture was diluted with water (15 mL) and extracted with DCM (15 mL×3). The combined organic phase was washed with brine (15 mL×1), dried over anhydrous sodium sulfate, filtered, and concentrated under vacuum. The crude material was purified by prep-HPLC (Column: Welch Xtimate C18 150*25 mm*5 um; Condition: water (NH₄HCO₃)-ACN; Begin B: 35; End B: 65; Flow Rate (ml/min): 25) to give 2-((4-cyclopropyl-6-(trifluoromethyl)pyridin-3-yl)sulfonyl)-6-((tetrahydro-2H-pyran-4-yl)methyl)-2,6-diazaspiro[3.3]heptane (25 mg, 19%) as a white solid. LCMS m/z=446.1 (M+H)⁺. ¹H NMR (500 MHz, CDCl₃) δ (ppm) 9.08 (s, 1H), 7.11 (s, 1H), 4.04 (s, 4H), 3.95-3.90 (m, 2H), 3.37-3.30 (m, 2H), 3.27 (s, 4H), 2.88-2.80 (m, 1H), 2.26 (br d, J=6.5 Hz, 2H), 1.57 (br s, 1H), 1.54-1.46 (m, 2H), 1.39-1.35 (m, 2H), 1.28-1.22 (m, 2H), 1.04-1.00 (m, 2H).

### Examples 123 and 124: 4-(6-((4-cyclopropyl-6-(trifluoromethyl)pyridin-3-yl)sulfonyl)-2,6-diazaspiro[3.3]heptan-2-yl)-1-methylcyclohexan-1-ol AND 4-(6-((4-cyclopropyl-6-(trifluoromethyl)pyridin-3-yl)sulfonyl)-2,6-diazaspiro[3.3]heptan-2-yl)-1-methylcyclohexan-1-ol

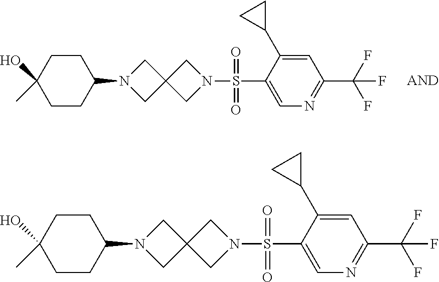

To a solution of 2-((4-cyclopropyl-6-(trifluoromethyl)pyridin-3-yl)sulfonyl)-2,6-diazaspiro[3.3]heptane trifluoroacetate (280 mg, 608 μmol) and TEA (0.25 mL, 1.8 mmol) in MeOH (5 mL) was added 4-hydroxy-4-methyl-cyclohexanone (86 mg, 669 μmol). The mixture was adjusted to pH=5-6 by dropwise addition of acetic acid at 0° C. After 30 minutes, sodium cyanoborohydride (115 mg, 1.8 mmol) was added, and the mixture was stirred at 20° C. for 2 hours. The mixture was diluted with water (15 mL) and extracted with DCM (15 mL×3). The combined organic phase was washed with brine (15 mL×1), dried over anhydrous sodium sulfate, filtered, and concentrated under vacuum. The crude material was purified by prep-HPLC (Column: Waters Xbridge BEH C18 100*30 mm*10 um; Condition: water (NH₄HCO₃)-ACN; Begin B: 32; End B: 62; Flow Rate (ml/min): 50) and by prep-HPLC (Column: Boston Prime C18 150*30 mm*5 um; Condition: water(NH₃H₂O+NH₄HCO₃)-ACN; Begin B: 40; End B: 70; Flow Rate (ml/min): 25) to give the following compounds: 4-(6-((4-cyclopropyl-6-(trifluoromethyl)pyridin-3-yl)sulfonyl)-2,6-diazaspiro[3.3]heptan-2-yl)-1-methylcyclohexan-1-ol (TRANS) after HPLC (basic conditions) (first peak off the prep HPLC) (22 mg, 8%). LCMS m/z=460.1 (M+H)⁺. ¹H NMR (500 MHz, CDCl₃) δ (ppm) 9.07 (s, 1H), 7.11 (s, 1H), 4.04 (s, 4H), 3.29 (s, 4H), 2.88-2.79 (m, 1H), 2.03 (br s, 1H), 1.68-1.64 (m, 4H), 1.40-1.34 (m, 4H), 1.21 (s, 3H), 1.20-1.12 (m, 2H), 1.04-0.99 (m, 2H). 4-(6-((4-cyclopropyl-6-(trifluoromethyl)pyridin-3-yl)sulfonyl)-2,6-diazaspiro[3.3]heptan-2-yl)-1-methylcyclohexan-1-ol (CIS) after HPLC (basic conditions) (second peak off the prep HPLC) (22 mg, 8%). LCMS m/z=460.2 (M+H)⁺. ¹H NMR (500 MHz, CDCl₃) δ (ppm) 9.08 (s, 1H), 7.11 (s, 1H), 4.05 (s, 4H), 3.33 (s, 4H), 2.88-2.79 (m, 1H), 1.92 (br s, 1H), 1.66-1.62 (m, 2H), 1.57-1.47 (m, 2H), 1.42-1.30 (m, 6H), 1.20 (s, 3H), 1.04-0.97 (m, 2H).

### Example 125: 2-((4-ethyl-6-(trifluoromethyl)pyridin-3-yl)sulfonyl)-6-(2-oxaspiro[3.3]heptan-6-yl)-2,6-diazaspiro[3.3]heptane

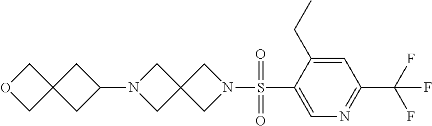 [SMILES: CCc1cc(C(F)(F)F)ncc1S(=O)(=O)N1CC2(CN(C3CC4(COC4)C3)C2)C1]

### 1. Synthesis of 6-(trifluoromethyl)-4-vinylpyridin-3-amine

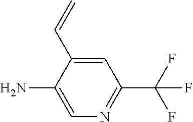

To a solution of 4-bromo-6-(trifluoromethyl)pyridin-3-amine (1.0 g, 4.15 mmol) and 4,4,5,5-tetramethyl-2-vinyl-1,3,2-dioxaborolane (767 mg, 4.98 mmol) in toluene (20 mL) and water (1 mL) was added S-Phos (170 mg, 415 μmol), Pd₂(dba)₃ (380 mg, 415 μmol), and K₃PO₄ (2.64 g, 12.5 mmol) at 20° C. The mixture was stirred for 3 hours at 100° C. under nitrogen. The reaction mixture was cooled then filtered and concentrated under vacuum. The crude material was purified by flash column (ethyl acetate in petroleum ether from 0% to 20%) to afford 6-(trifluoromethyl)-4-vinylpyridin-3-amine (650 mg, 83%) as a red oil that was used without further purification. LCMS m/z=189.1 (M+H)⁺. ¹H NMR (500 MHz, CDCl₃) δ (ppm) 8.11 (s, 1H), 7.50 (s, 1H), 6.70-6.64 (m, 1H), 5.85 (d, J=17.0 Hz, 1H), 5.59 (d, J=11.0 Hz, 1H), 4.11 (br s, 2H).

### 2. Synthesis of 4-ethyl-6-(trifluoromethyl)pyridin-3-amine

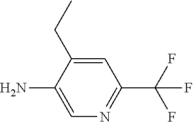

To a solution of 6-(trifluoromethyl)-4-vinylpyridin-3-amine (650 mg, 3.45 mmol) in MeOH (10 mL) was added 10% Pd/C (100 mg, 95 μmol) at 20° C. The mixture was stirred for 16 hours at 20° C. under hydrogen (15 psi). Nitrogen was bubbled through reaction solution for 10 minutes, then the reaction was filtered and concentrated under vacuum to yield 4-ethyl-6-(trifluoromethyl)pyridin-3-amine as a yellow solid that was used without purification. ¹H NMR (400 MHz, CDCl₃) δ (ppm) 8.06 (s, 1H), 7.36 (s, 1H), 2.53 (q, J=7.6 Hz, 2H), 1.30 (t, J=7.6 Hz, 3H).

### 3. Synthesis of 4-ethyl-6-(trifluoromethyl)pyridine-3-sulfonyl chloride

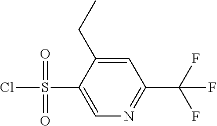 [SMILES: CCc1cc(C(F)(F)F)ncc1S(=O)(=O)Cl]

Thionyl chloride (2.5 mL, 34 mmol) was added over 10 minutes to water (8 mL) while the temperature was maintained 0-5° C., then the solution was stirred at 20° C. for 12 hours. Copper chloride (22 mg, 226 μmol) was added, and the mixture was cooled to −3° C. In another flask, a solution of 4-ethyl-6-(trifluoromethyl)pyridin-3-amine (430 mg, 2.26 mmol) in HCl (12 M, 4 mL) at −5° C. was added dropwise to the solution of sodium nitrite (158 mg, 2.28 mmol) in water (2 mL) while maintaining temperature −5 to 0° C. for 1 hour. When the addition was complete, this solution was added to the precooled thionyl chloride solution and stirred at −2° C. for 10 minutes, then at 0° C. for 75 minutes. The mixture was extracted with DCM (30 mL×3). The combined organic phase was dried over Na₂SO₄, filtered, and concentrated to give 4-ethyl-6-(trifluoromethyl)pyridine-3-sulfonyl chloride as a yellow oil that was used without purification. ¹H NMR (400 MHz, CDCl₃) δ (ppm) 9.27 (s, 1H), 7.80 (s, 1H), 3.30 (q, J=7.6 Hz, 2H), 1.44 (t, J=7.6 Hz, 3H).

### 4. Synthesis of tert-butyl 6-((4-ethyl-6-(trifluoromethyl)pyridin-3-yl)sulfonyl)-2,6-diazaspiro[3.3]heptane-2-carboxylate

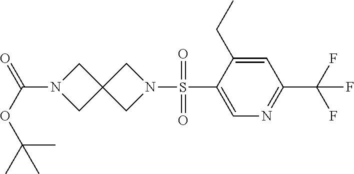 [SMILES: CCc1cc(C(F)(F)F)ncc1S(=O)(=O)N1CC2(CN(C(=O)OC(C)(C)C)C2)C1]

To a solution of tert-butyl 2,6-diazaspiro[3.3]heptane-2-carboxylate (319 mg, 1.61 mmol) and DIPEA (0.77 mL, 4.39 mmol) in DCM (20 mL) was added 4-ethyl-6-(trifluoromethyl)pyridine-3-sulfonyl chloride (400 mg, 1.46 mmol) in DCM (5 mL) at 0° C., then the mixture was stirred for 1 hour at 20° C. The mixture was diluted with water (30 mL) and extracted with DCM (40 mL×3). The combined organic phase was dried over anhydrous sodium sulfate, filtered, and concentrated under vacuum. The residue was purified by flash column (ethyl acetate in petroleum ether from 5% to 35%) to give tert-butyl 6-((4-ethyl-6-(trifluoromethyl)pyridin-3-yl)sulfonyl)-2,6-diazaspiro[3.3]heptane-2-carboxylate (400 mg, 63%) as a yellow solid. ¹H NMR (400 MHz, CDCl₃) δ (ppm) 9.11 (s, 1H), 7.69 (s, 1H), 4.08 (s, 4H), 4.04 (s, 4H), 3.09 (q, J=7.6 Hz, 2H), 1.42 (s, 9H), 1.35 (t, J=7.6 Hz, 3H).

### 5. Synthesis of 2-((4-ethyl-6-(trifluoromethyl)pyridin-3-yl)sulfonyl)-2,6-diazaspiro[3.3]heptane trifluoroacetate

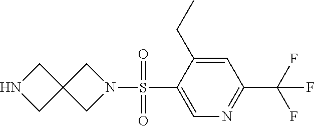 [SMILES: CCc1cc(C(F)(F)F)ncc1S(=O)(=O)N1CC2(CNC2)C1]

To a solution of tert-butyl 6-((4-ethyl-6-(trifluoromethyl)pyridin-3-yl)sulfonyl)-2,6-diazaspiro[3.3]heptane-2-carboxylate (400 mg, 919 μmol) in HFiPA (20 mL) was added TFA (0.35 mL 4.59 mmol). The mixture was stirred for 10 hours at 20° C. The mixture was concentrated under vacuum to give 2-((4-ethyl-6-(trifluoromethyl)pyridin-3-yl)sulfonyl)-2,6-diazaspiro[3.3]heptane trifluoroacetate as a yellow solid that was used without purification. LCMS m/z=336.1 (M+H)⁺.

### 6. Synthesis of 2-((4-ethyl-6-(trifluoromethyl)pyridin-3-yl)sulfonyl)-6-(2-oxaspiro[3.3]heptan-6-yl)-2,6-diazaspiro[3.3]heptane

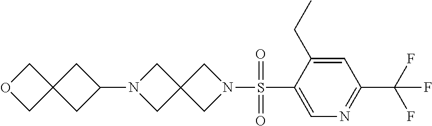 [SMILES: CCc1cc(C(F)(F)F)ncc1S(=O)(=O)N1CC2(CN(C3CC4(COC4)C3)C2)C1]

A solution of 2-((4-ethyl-6-(trifluoromethyl)pyridin-3-yl)sulfonyl)-2,6-diazaspiro[3.3]heptane trifluoroacetate (100 mg, 222 μmol), 2-oxaspiro[3.3]heptan-6-one (25 mg, 222 μmol), and triethylamine in MeOH (10 mL) was adjusted to a pH 5-6 by acetic acid. The mixture was stirred for 0.5 hour at 20° C., then sodium cyanoborohydride (42 mg, 668 μmol) was added to the mixture. The mixture was stirred for 1 hour at 20° C. The mixture was diluted with aqueous NaHCO₃ (15 mL), water (20 mL) then extracted with DCM (30 mL×3). The combined organic phase was dried over anhydrous sodium sulfate, filtered, and concentrated under vacuum. The residue was purified by prep-HPLC (Column: Phenomenex C18 150*40 mm*5 um, Condition: water(NH₄HCO₃)-ACN, 23%˜53%, Flow Rate (mL/min): 60) to give 2-((4-ethyl-6-(trifluoromethyl)pyridin-3-yl)sulfonyl)-6-(2-oxaspiro[3.3]heptan-6-yl)-2,6-diazaspiro[3.3]heptane (39 mg, 41%) as a white solid. LCMS m/z=432.1 (M+H)⁺. ¹H NMR (400 MHz, CDCl₃) δ (ppm) 9.10 (s, 1H), 7.68 (s, 1H), 4.64 (s, 2H), 4.59 (s, 2H), 4.02 (s, 4H), 3.21 (s, 4H), 3.09 (q, J=7.2 Hz, 2H), 2.84-2.78 (m, 1H), 2.28-2.23 (m, 2H), 1.94-1.90 (m, 2H), 1.34 (t, J=7.2 Hz, 3H).

### Example 126: 2-((4-ethyl-6-(trifluoromethyl)pyridin-3-yl)sulfonyl)-6-((tetrahydro-2H-pyran-4-yl)methyl)-2,6-diazaspiro[3.3]heptane

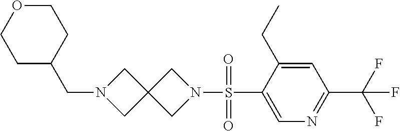 [SMILES: CCc1cc(C(F)(F)F)ncc1S(=O)(=O)N1CC2(CN(CC3CCOCC3)C2)C1]

A solution of 2-((4-ethyl-6-(trifluoromethyl)pyridin-3-yl)sulfonyl)-2,6-diazaspiro[3.3]heptane trifluoroacetate (60 mg, 134 μmol), tetrahydropyran-4-carbaldehyde (15 mg, 134 μmol), and triethylamine in MeOH (10 mL) was adjusted to a pH 5-6 by acetic acid. The mixture was stirred for 0.5 hours at 20° C., then sodium cyanoborohydride (25 mg, 400 μmol) was added to the mixture. The mixture was stirred for 1 hour at 20° C. The mixture was diluted with aqueous NaHCO₃ (15 mL), water (20 mL) then extracted with DCM (30 mL×3). The combined organic phase was dried over anhydrous sodium sulfate, filtered, and concentrated under vacuum. The residue was purified by flash column (MeOH in DCM from 0% to 8%) to give 2-((4-ethyl-6-(trifluoromethyl)pyridin-3-yl)sulfonyl)-6-((tetrahydro-2H-pyran-4-yl)methyl)-2,6-diazaspiro[3.3]heptane (25 mg, 43%) as a white solid. LCMS m/z=434.1 (M+H)⁺. ¹H NMR (400 MHz, CDCl₃) δ (ppm) 9.11 (s, 1H), 7.68 (s, 1H), 4.04 (s, 4H), 3.95-3.91 (m, 2H), 3.37-3.29 (m, 6H), 3.09 (q, J=7.2 Hz, 2H), 2.28-2.27 (m, 2H), 1.62-1.56 (m, 2H), 1.35 (t, J=7.2 Hz, 3H), 1.29-1.19 (m, 3H).

### Examples 127 and 128: 4-(6-((4-ethyl-6-(trifluoromethyl)pyridin-3-yl)sulfonyl)-2,6-diazaspiro[3.3]heptan-2-yl)-1-methylcyclohexan-1-ol (TRANS) AND 4-(6-((4-ethyl-6-(trifluoromethyl)pyridin-3-yl)sulfonyl)-2,6-diazaspiro[3.3]heptan-2-yl)-1-methylcyclohexan-1-ol (CIS)

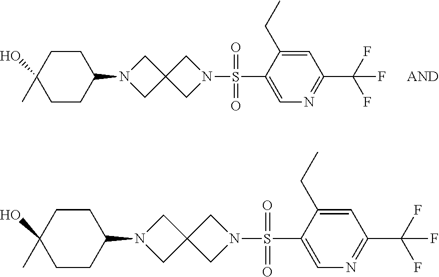

A solution of 2-((4-ethyl-6-(trifluoromethyl)pyridin-3-yl)sulfonyl)-2,6-diazaspiro[3.3]heptane trifluoroacetate (300 mg, 668 μmol), 4-hydroxy-4-methyl-cyclohexanone (94 mg, 734 μmol), and triethylamine in MeOH (15 mL) was adjusted to a pH 5-6 by acetic acid. The mixture was stirred for 0.5 hours at 20° C., then sodium cyanoborohydride (42 mg, 400 μmol) was added to the mixture. The mixture was stirred for 1 hour at 20° C. The mixture was diluted with aqueous NaHCO₃ (15 mL), water (20 mL) then extracted with DCM (30 mL×3). The combined organic phase was dried over anhydrous sodium sulfate, filtered, and concentrated under vacuum. The residue was purified by prep-HPLC (Column: Phenomenex C18 150*40 mm*5 um, Condition: water (NH₄HCO₃)-ACN, 25%-55%, Flow Rate (mL/min): 60) to give the following compounds as white solids:

4-(6-((4-ethyl-6-(trifluoromethyl)pyridin-3-yl)sulfonyl)-2,6-diazaspiro[3.3]heptan-2-yl)-1-methylcyclohexan-1-ol (TRANS) after HPLC (basic conditions) (first peak off the prep HPLC) (43 mg, 14%). LCMS m/z=448.2 (M+H)⁺. ¹H NMR (400 MHz, CDCl₃) δ (ppm) 9.11 (s, 1H), 7.68 (s, 1H), 4.04 (s, 4H), 3.26 (s, 4H), 3.10 (q, J=7.6 Hz, 2H), 2.03-1.99 (m, 1H), 1.68-1.66 (m, 4H), 1.40-1.33 (m, 5H), 1.22 (s, 3H), 1.18-1.12 (m, 2H).

4-(6-((4-ethyl-6-(trifluoromethyl)pyridin-3-yl)sulfonyl)-2,6-diazaspiro[3.3]heptan-2-yl)-1-methylcyclohexan-1-ol (CIS) after HPLC (basic conditions) (second peak off the prep HPLC) (79 mg, 26%). LCMS m/z=448.2 (M+H)⁺. ¹H NMR (400 MHz, CDCl₃) δ (ppm) 9.11 (s, 1H), 7.68 (s, 1H), 4.04 (s, 4H), 3.29 (s, 4H), 3.09 (q, J=7.6 Hz, 2H), 1.89-1.85 (m, 1H), 1.69-1.63 (m, 4H), 1.55-1.51 (m, 2H), 1.36-1.29 (m, 5H), 1.20 (s, 3H).

### Example 129: 2-((2-methyl-6-(trifluoromethyl)pyridin-3-yl)sulfonyl)-6-((4-methyltetrahydro-2H-pyran-4-yl)methyl)-2,6-diazaspiro[3.3]heptane

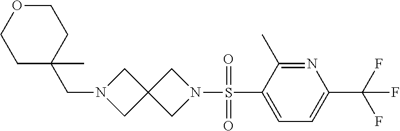 [SMILES: Cc1nc(C(F)(F)F)ccc1S(=O)(=O)N1CC2(CN(CC3(C)CCOCC3)C2)C1]

### 1. Synthesis of tert-butyl 6-((2-methyl-6-(trifluoromethyl)pyridin-3-yl)sulfonyl)-2,6-diazaspiro[3.3]heptane-2-carboxylate

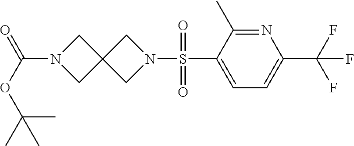 [SMILES: Cc1nc(C(F)(F)F)ccc1S(=O)(=O)N1CC2(CN(C(=O)OC(C)(C)C)C2)C1]

To a vial containing tert-butyl 2,6-diazaspiro[3.3]heptane-2-carboxylate hydrochloride (730 mg, 3.11 mmol) in anhydrous dichloromethane (12 mL) was added Hunigs Base (1.7 mL, 10 mmol) carefully dropwise at <5° C. After 5 minutes, 2-methyl-6-(trifluoromethyl)pyridine-3-sulfonyl chloride (922 mg, 3.55 mmol) was added carefully to the cold solution. Upon complete addition of sulfonyl chloride, the reaction was warmed to 23° C. and monitored with LCMS. After 30 minutes, the reaction was carefully quenched with slow addition of aqueous saturated sodium bicarbonate solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (10-60% ethyl acetate in heptane.) The desired fractions were pooled then concentrated under reduced pressure to afford a white solid as tert-butyl 6-((2-methyl-6-(trifluoromethyl)pyridin-3-yl)sulfonyl)-2,6-diazaspiro[3.3]heptane-2-carboxylate (1.23 g, 94%) that was used without further purification. ¹H NMR (500 MHz, DMSO-d₆) δ (ppm) 8.43 (br d, J=7.9 Hz, 1H), 7.98 (br d, J=7.9 Hz, 1H), 4.05 (s, 4H), 3.94 (br s, 4H), 2.81 (s, 3H), 1.34 (s, 9H).

### 2. Synthesis of 2-((2-methyl-6-(trifluoromethyl)pyridin-3-yl)sulfonyl)-2,6-diazaspiro[3.3]heptane trifluoroacetate

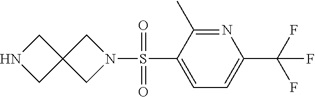 [SMILES: Cc1nc(C(F)(F)F)ccc1S(=O)(=O)N1CC2(CNC2)C1]

To a flask containing tert-butyl 6-((2-methyl-6-(trifluoromethyl)pyridin-3-yl)sulfonyl)-2,6-diazaspiro[3.3]heptane-2-carboxylate (1.23 g, 2.92 mmol) in HFiPA (20 mL) was added TFA (0.67 mL, 8.75 mmol) carefully dropwise at <5° C. Upon complete addition of TFA, the mixture was warmed to 23° C. and monitored with LCMS. After 19 hours, the mixture was concentrated under reduced pressure to afford a dark yellow residue that was triturated with methanol to afford a white solid as 2-((2-methyl-6-(trifluoromethyl)pyridin-3-yl)sulfonyl)-2,6-diazaspiro[3.3]heptane trifluoroacetate (551 mg, 41%) that was used without purification. LCMS m/z=322.0 [M+H]⁺. ¹H NMR (500 MHz, DMSO-d₆) δ (ppm) 8.51 (br s, 1H), 8.45 (br d, J=8.2 Hz, 1H), 7.99 (br d, J=8.2 Hz, 1H), 4.14-4.08 (m, 8H), 2.81 (s, 3H).

### 3. Synthesis of 2-((2-methyl-6-(trifluoromethyl)pyridin-3-yl)sulfonyl)-6-((4-methyltetrahydro-2H-pyran-4-yl)methyl)-2,6-diazaspiro[3.3]heptane

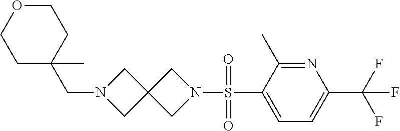 [SMILES: Cc1nc(C(F)(F)F)ccc1S(=O)(=O)N1CC2(CN(CC3(C)CCOCC3)C2)C1]

A solution of 2-((2-methyl-6-(trifluoromethyl)pyridin-3-yl)sulfonyl)-2,6-diazaspiro[3.3]heptane trifluoroacetate (100 mg, 230 μmol), 4-methyltetrahydropyran-4-carbaldehyde (35 mg, 276 μmol), and triethylamine in MeOH (10 mL) was adjusted to a pH 5-6 by acetic acid. The mixture was stirred for 0.5 hour at 20° C., then sodium cyanoborohydride (43 mg, 689 μmol) was added to the mixture. The mixture was stirred for 20 hours at 20° C. The mixture was diluted with aqueous NaHCO₃ (15 mL), water (20 mL) then extracted with DCM (30 mL×3). The combined organic phase was dried over anhydrous sodium sulfate, filtered, and concentrated under vacuum. The residue was purified by prep-HPLC (Welch Xtimate C18 150*25 mm*5 um, Condition: water (NH₄HCO₃)-ACN, Flow Rate (mL/min): 25) to give 2-((2-methyl-6-(trifluoromethyl)pyridin-3-yl)sulfonyl)-6-((4-methyltetrahydro-2H-pyran-4-yl)methyl)-2,6-diazaspiro[3.3]heptane (62 mg, 63%) as a white solid. LCMS m/z=434.1 (M+H)⁺. ¹H NMR (500 MHz, CDCl₃) δ (ppm) 8.35 (d, J=8.0 Hz, 1H), 7.64 (d, J=8.0 Hz, 1H), 4.03 (s, 4H), 3.68-3.65 (m, 2H), 3.59-3.56 (m, 2H), 3.34 (s, 4H), 2.90 (s, 3H), 2.22 (s, 2H), 1.59-1.44 (m, 2H), 1.20-1.18 (m, 2H), 0.94 (s, 3H).

### Examples 130 to 154

The Examples 130 to 154 in the following Table were prepared using a similar method described for Example 129 from 2-((2-methyl-6-(trifluoromethyl)pyridin-3-yl)sulfonyl)-2,6-diazaspiro[3.3]heptane trifluoroacetate (1 equiv.) and the aldehyde or ketone (1.2 equiv.; unless otherwise stated)

| | Name/Structure/ | |
|---|---|---|
| Example | Aldehyde or Ketone (SM) | Data |
| 130 | 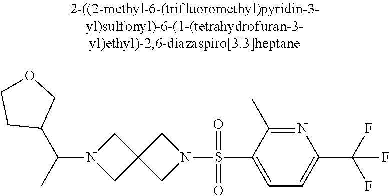 [SMILES: Cc1nc(C(F)(F)F)ccc1S(=O)(=O)N1CC2(CN(C(C)C3CCOC3)C2)C1] | Yield: 22 mg, 52%. LCMS m/z = 420.1 (M + H)⁺. ¹H NMR (400 MHz, DMSO-d6) δ (ppm): 8.44 (d, J = 8.4 Hz, 1H), 7.98 (d, J = 8.0 Hz, 1H), 3.96 (s, 4H), 3.71-3.66 (m, 1H), 3.55-3.50 (m, 1H), 3.39-3.34 (m, 1H), 3.25 (t, J = 8.0 Hz, 1H), 3.18-3.14 (m, 4H), 2.81 (s, 3H), 2.18-1.96 (m, 2H), 1.91-1.70 (m, 1H), 1.57-1.41 (m, 1H), 0.78-0.70 (m, 3H). |
| | Mixture of 4 stereoisomers | |
| | SM: 1-tetrahydrofuran-3-ylethanone (26 mg, | |
| | 0.23 mmol) | |
| 131 | 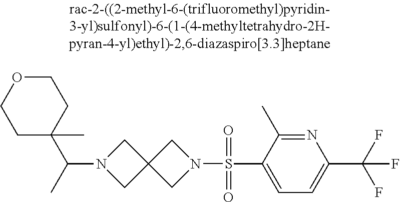 [SMILES: Cc1nc(C(F)(F)F)ccc1S(=O)(=O)N1CC2(CN(C(C)C3(C)CCOCC3)C2)C1] | Yield: 50 mg, 41%. LCMS m/z = 448.2 (M + H)⁺. ¹H NMR (400 MHz, CDCl₃) δ (ppm): 8.36 (d, J = 8.0 Hz, 1H), 7.64 (d, J = 8.0 Hz, 1H), 4.02 (s, 4H), 3.76-3.72 (m, 2H), 3.53 (t, J = 11.6 Hz, 2H), 3.34-3.27 (m, 4H), 2.90 (s, 3H), 2.01-1.98 (m, 1H), 1.56-1.49 (m, 2H), 1.35-1.32 (m, 1H), 1.15-1.12 (m, 1H), 0.91 (s, 3H), 0.79 (d, J = 6.4 Hz, 3H). |
| | SM: 1-(4-methyltetrahydropyran-4- | |
| | yl)ethanone (43 mg, 0.30 mmol) | |
| 132 | 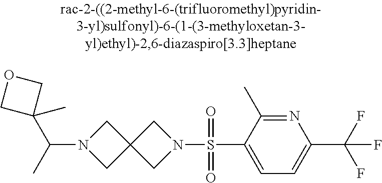 [SMILES: Cc1nc(C(F)(F)F)ccc1S(=O)(=O)N1CC2(CN(C(C)C3(C)COC3)C2)C1] | Yield: 28 mg, 29%. LCMS m/z = 420.1 (M + H)⁺. ¹H NMR (400 MHz, CD₃OD) δ (ppm): 8.45 (d, J = 8.0 Hz, 1H), 7.83 (d, J = 8.0 Hz, 1H), 4.69 (d, J = 5.6 Hz, 1H), 4.50 (d, J = 6.0 Hz, 1H), 4.18 (d, J = 5.6 Hz, 1H), 4.11 (d, J = 6.0, 1H), 4.01 (s, 4H), 3.31-3.27 (m, 2H), 3.25-3.21 (m, 2H), 2.86 (s, 3H), 2.76-2.71 (m, 1H), 1.32 (s, 3H), 0.71 (d, J = 6.4 Hz, 3H). |
| | SM: 1-(3-methyloxetan-3-yl)ethanone (29 mg, | |
| | 0.25 mmol) | |
| 133 | 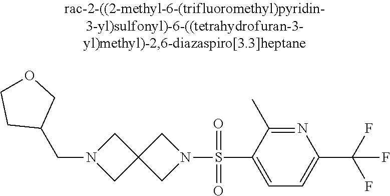 [SMILES: Cc1nc(C(F)(F)F)ccc1S(=O)(=O)N1CC2(CN(CC3CCOC3)C2)C1] | Yield: 73 mg, 78%. LCMS m/z = 406.2 (M + H)⁺. ¹H NMR (500 MHz, CDCl₃) δ (ppm): 8.35 (d, J = 8.0 Hz, 1H), 7.64 (d, J = 8.0 Hz, 1H), 4.04 (s, 4H), 3.83-3.78 (m, 2H), 3.73-3.68 (m, 1H), 3.41- 3.38 (m, 1H), 3.29 (s, 4H), 2.90 (s, 3H), 2.38 (d, J = 7.5 Hz, 2H), 2.19- 2.13 (m, 1H), 2.01-1.95 (m, 1H), 1.55-1.48 (m, 1H). |
| | SM: tetrahydrofuran-3-carbaldehyde (28 mg, | |
| | 0.28 mmol) | |
| 134 | 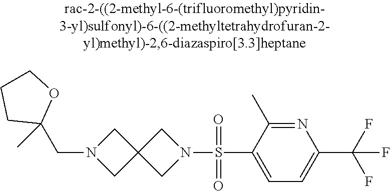 [SMILES: Cc1nc(C(F)(F)F)ccc1S(=O)(=O)N1CC2(CN(CC3(C)CCCO3)C2)C1] | Yield: 70 mg, 73%. LCMS m/z = 420.1 (M + H)⁺. ¹H NMR (500 MHz, CD₃OD) δ (ppm): 8.45 (d, J = 8.0 Hz, 1H), 7.83 (d, J = 8.4 Hz, 1H), 4.02 (s, 4H), 3.82-3.70 (m, 2H), 3.39 (s, 4H), 2.86 (s, 3H), 2.45 (s, 2H), 1.94-1.78 (m, 3H), 1.65-1.56 (m, 1H), 1.12 (s, 3H). |
| | SM: 2-methyltetrahydrofuran-2-carbaldehyde | |
| | (78 mg, 0.69 mmol) | |
| 135 | 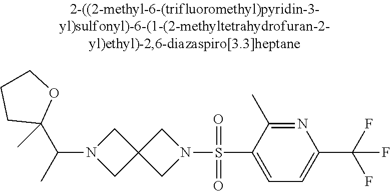 [SMILES: Cc1nc(C(F)(F)F)ccc1S(=O)(=O)N1CC2(CN(C(C)C3(C)CCCO3)C2)C1] | Yield: 48 mg, 44%. LCMS m/z = 434.2 (M + H)⁺. ¹H NMR (500 MHz, CDCl₃) δ (ppm): 8.35 (d, J = 8.0 Hz, 1H), 7.64 (d, J = 8.0 Hz, 1H), 4.02 (br s, 4H), 3.88-3.76 (m, 1H), 3.73-3.66 (m, 1H), 3.40- 3.25 (m, 4H), 2.90 (s, 3H), 2.31- 2.09 (m, 1H), 2.03-1.79 (m, 2.5H), 1.61-1.57 (m, 1.5H), 1.05 (s, 3H), 0.85-0.77 (m, 3H). |
| | Mixture of 4 stereoisomers | |
| | SM: 1-(2-methyltetrahydrofuran-2- | |
| | yl)ethanone (130 mg, 1.0 mmol) | |
| 136 | 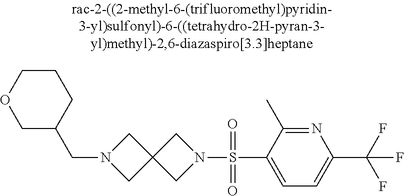 [SMILES: Cc1nc(C(F)(F)F)ccc1S(=O)(=O)N1CC2(CN(CC3CCCOC3)C2)C1] | Yield: 95 mg 89%. LCMS m/z = 420.1 (M + H)⁺. ¹H NMR (500 MHz, CD₃OD) δ (ppm): 8.67 (d, J = 8.0 Hz, 1H), 8.04 (d, J = 8.0 Hz, 1H), 4.25 (s, 4H), 4.07-3.99 (m, 2H), 3.61-3.55 (m, 2H), 3.54 (s, 4H), 3.08 (s, 3H), 2.56-2.44 (m, 2H), 2.02-1.98 (m, 1H), 1.84- 1.74 (m, 3H), 1.46-1.36 (m, 1H). |
| | SM: tetrahydropyran-3-carbaldehyde (43 mg, | |
| | 0.38 mmol) | |
| 137 | 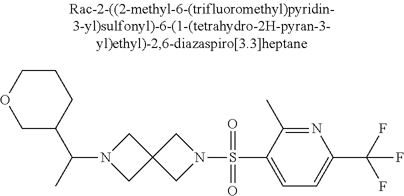 [SMILES: Cc1nc(C(F)(F)F)ccc1S(=O)(=O)N1CC2(CN(C(C)C3CCCOC3)C2)C1] | Yield: 70 mg, 64%. LCMS m/z = 434.1 (M + H)⁺. ¹H NMR (400 MHz, CDCl₃) δ (ppm): 8.36 (d, J = 8.0 Hz, 1H), 7.64 (d, J = 8.0 Hz, 1H), 4.03 (s, 4H), 3.94-3.72 (m, 2H), 3.31-3.20 (m, 5H), 3.19- 3.09 (m, 1H), 2.90 (s, 3H), 2.01 (br s, 1H), 1.74-1.58 (m, 4H), 1.35-1.19 (m, 1H), 0.80 (br d, J = 6.0 Hz, 3H). |
| | SM: 1-(tetrahydro-2H-pyran-3-yl)ethan-1-one | |
| | (130 mg, 1.0 mmol, 4.0 equiv.) | |
| 138 | 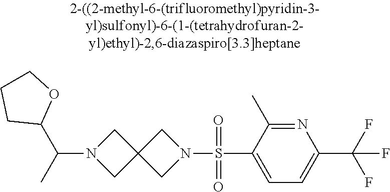 [SMILES: Cc1nc(C(F)(F)F)ccc1S(=O)(=O)N1CC2(CN(C(C)C3CCCO3)C2)C1] | Yield: 50 mg, 52%. LCMS m/z = 420.2 (M + H)⁺. ¹H NMR (400 MHz, CDCl₃) δ (ppm): 8.35 (d, J = 8.0 Hz, 1H), 7.64 (d, J = 8.0 Hz, 1H), 4.03 (s, 4H), 3.82-3.51 (m, 3H), 3.41-3.28 (m, 4H), 2.89 (s, 3H), 2.35-2.13 (m, 1H), 1.92-1.74 (m, 3H), 1.62-1.42 (m, 1H), 0.86- 0.80 (m, 3H). |
| | Mixture of 4 stereoisomers | |
| | SM: 1-tetrahydrofuran-2-ylethanone (32 mg, | |
| | 0.27 mmol) | |
| 139 | 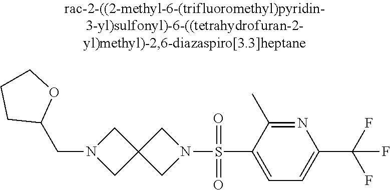 [SMILES: Cc1nc(C(F)(F)F)ccc1S(=O)(=O)N1CC2(CN(CC3CCCO3)C2)C1] | Yield: 45 mg, 48%. LCMS m/z = 406.1 (M + H)⁺. ¹H NMR (500 MHz, CDCl₃) δ (ppm): 8.35 (d, J = 8.0 Hz, 1H), 7.64 (d, J = 8.0 Hz, 1H), 4.04 (s, 4H), 3.84-3.76 (m, 2H), 3.74-3.68 (m, 1H), 3.39- 3.34 (m, 4H), 2.89 (s, 3H), 2.51- 2.41 (m, 2H), 1.95-1.80 (m, 3H), 1.50-1.40 (m, 1H). |
| | SM: tetrahydrofuran-2-carbaldehyde (58 mg, | |
| | 0.58 mmol) | |
| 140 | 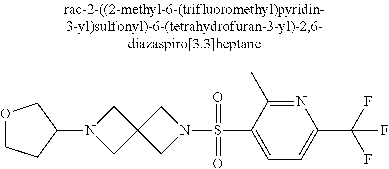 [SMILES: Cc1nc(C(F)(F)F)ccc1S(=O)(=O)N1CC2(CN(C3CCOC3)C2)C1] | Yield: 30 mg, 30%. LCMS m/z = 392.1 (M + H)⁺. ¹H NMR (400 MHz, CDCl₃) δ (ppm): 8.36 (d, J = 8.0 Hz, 1H), 7.65 (d, J = 8.0 Hz, 1H), 4.04 (s, 4H), 3.92-3.84 (m, 1H), 3.77-3.70 (m, 1H), 3.62-3.53 (m, 2H), 3.34-3.26 (m, 4H), 2.97- 2.91 (m, 1H), 2.89 (s, 3H), 1.91- 1.81 (m, 1H), 1.69-1.62 (m, 1H). |
| | SM: tetrahydrofuran-3-one (26 mg, 0.30 | |
| | mmol) | |
| 141 | 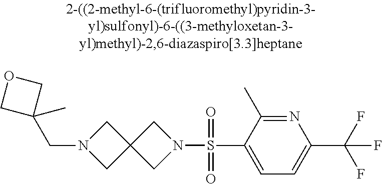 [SMILES: Cc1nc(C(F)(F)F)ccc1S(=O)(=O)N1CC2(CN(CC3(C)COC3)C2)C1] | Yield: 46 mg, 24%. LCMS m/z = 406.1 (M + H)⁺. ¹H NMR (400 MHz, CDCl₃) δ (ppm): 8.35 (d, J = 8.0 Hz, 1H), 7.64 (d, J = 8.4 Hz, 1H), 4.39 (d, J = 5.6 Hz, 2H), 4.29 (d, J = 6.0 Hz, 2H), 4.04 (s, 4H), 3.37 (s, 4H), 2.90 (s, 3H), 2.60 (s, 2H), 1.25 (s, 3H). |
| | SM: tetrahydrofuran-3-one (26 mg, 0.30 | |
| | mmol) | |
| 142 | 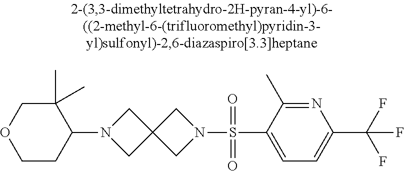 [SMILES: Cc1nc(C(F)(F)F)ccc1S(=O)(=O)N1CC2(CN(C3CCOCC3(C)C)C2)C1] | Yield: 65 mg, 50%. LCMS m/z = 434.2 (M + H)⁺. ¹H NMR (400 MHz, CDCl₃) δ (ppm) 8.36 (d, J = 8.0 Hz, 1H), 7.64 (d, J = 8.0 Hz, 1H), 4.03 (s, 4H), 3.95-3.89 (m, 1H), 3.35-3.29 (m, 5H), 3.28-3.27 (m, 1H), 2.98-2.94 (m, 1H), 2.90 (s, 3H), 1.98-1.89 (m, 1H), 1.42- 1.32 (m, 2H), 0.92 (s, 3H), 0.81 (s, 3H). |
| 143 and 144 | 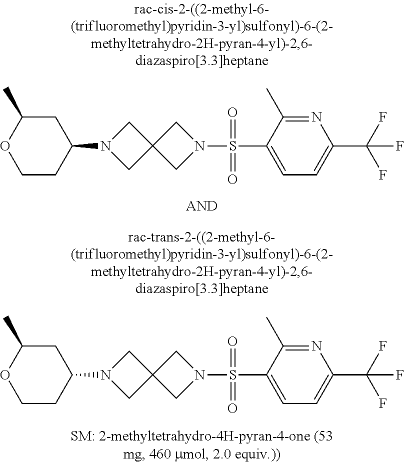 [SMILES: Cc1nc(C(F)(F)F)ccc1S(=O)(=O)N1CC2(CN([C@H]3CCO[C@@H](C)C3)C2)C1 ; Cc1nc(C(F)(F)F)ccc1S(=O)(=O)N1CC2(CN([C@@H]3CCO[C@@H](C)C3)C2)C1] | Peak 1, cis isomer, yield: 23 mg, 24%. LCMS m/z = 420.1 (M + H)⁺. ¹H NMR (400 MHz, CD₃OD) δ (ppm) 8.46 (d, J = 8.4 Hz, 1H), 7.83 (d, J = 8.4 Hz, 1H), 4.04 (s, 4H), 3.96-3.90 (m, 1H), 3.43-3.34 (m, 2H), 3.35 (s, 4H), 2.87 (s, 3H), 2.30-2.20 (m, 1H), 1.76-1.70 (m, 1H), 1.68-1.60 (m, 1H), 1.18- 1.07 (m, 4H), 0.90-0.78 (m, 1H). Peak 2, trans isomer, yield: 18 mg, 19%. LCMS m/z = 420.1 (M + H)⁺. ¹H NMR (400 MHz, CD₃OD) δ (ppm) 8.46 (d, J = 8.4 Hz, 1H), 7.83 (d, J = 8.0 Hz, 1H), 4.04 (s, 4H), 3.81-3.70 (m, 2H), 3.62-3.59 (m, 1H), 3.28 (s, 4H), 2.87 (s, 3H), 2.48-2.44 (m, 1H), 1.60-1.54 (m, 1H), 1.53-1.50 (m, 1H), 1.40-1.38 (m, 1H), 1.32-1.26 (m, 1H), 1.08 (d, J = 6.0 Hz, 3H). |
| 145 | 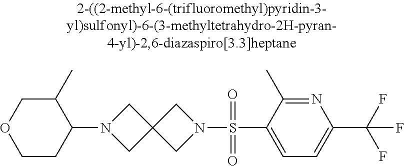 [SMILES: Cc1nc(C(F)(F)F)ccc1S(=O)(=O)N1CC2(CN(C3CCOCC3C)C2)C1] | Yield 45 mg, 52%. LCMS m/z = 420.1 (M + H)⁺. ¹H NMR (400 MHz, CDCl₃) δ (ppm) 8.36 (d, J = 8.0 Hz, 1H), 7.65 (d, J = 8.4 Hz, 1H), 4.05 (s, 4H), 3.94-3.85 (m, 1H), 3.73-3.70 (m, 1H), 3.43-3.23 (m, 6H), 2.90 (s, 3H), 2.19-1.93 (m, 1H), 1.67-1.62 (m, 1H), 1.53- 1.43 (m, 1H), 1.33-1.23 (m, 1H), 0.96-0.90 (m, 3H). |
| | SM: 3-methyltetrahydro-4H-pyran-4-one (26 | |
| | mg, 230 μmol) | |
| 146 and 147 | 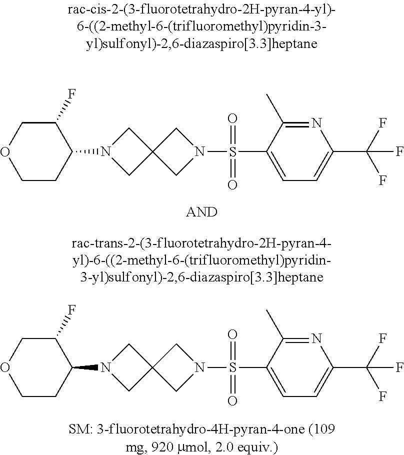 [SMILES: Cc1nc(C(F)(F)F)ccc1S(=O)(=O)N1CC2(CN([C@@H]3CCOC[C@@H]3F)C2)C1 ; Cc1nc(C(F)(F)F)ccc1S(=O)(=O)N1CC2(CN([C@H]3CCOC[C@@H]3F)C2)C1] | Peak 1, cis isomer, yield: 43 mg, 22%. LCMS m/z = 424.1 (M + H)⁺. ¹H NMR (400 MHz, MeOD) δ (ppm) 8.46 (d, J = 8.0 Hz, 1H), 7.83 (d, J = 8.4 Hz, 1H), 4.58-4.44 (m, 1H), 4.05 (s, 4H), 4.03- 3.91 (m, 2H), 3.52-3.43 (m, 1H), 3.41 (s, 4H), 3.40-3.36 (m, 1H), 2.87 (s, 3H), 2.47-2.36 (m, 1H), 1.71-1.59 (m, 1H), 1.57-1.52 (m, 1H). Peak 2, trans isomer, yield: 55 mg, 28%. LCMS m/z = 424.1 (M + H)⁺. ¹H NMR (400 MHz, MeOD) δ (ppm) 8.46 (d, J = 8.4 Hz, 1H), 7.83 (d, J = 8.4 Hz, 1H), 4.34-4.18 (m, 1H), 4.05 (s, 4H), 3.93-3.78 (m, 2H), 3.44 (s, 4H), 3.40-3.35 (m, 1H), 3.30-3.23 (m, 1H), 2.87 (s, 3H), 2.53-2.44 (m, 1H), 1.83-1.78 (m, 1H), 1.34-1.23 (m, 1H). |
| 148 and 148 | 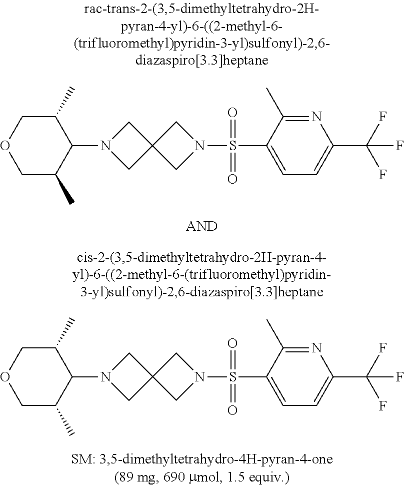 [SMILES: Cc1nc(C(F)(F)F)ccc1S(=O)(=O)N1CC2(CN(C3[C@@H](C)COC[C@@H]3C)C2)C1 ; Cc1nc(C(F)(F)F)ccc1S(=O)(=O)N1CC2(CN(C3[C@H](C)COC[C@@H]3C)C2)C1] | Peak 1, trans isomer: 41 mg, 21%. LCMS m/z = 434.1 (M + H)⁺. ¹H NMR (400 MHz, MeOD) δ (ppm) 8.46 (d, J = 8.4 Hz, 1H), 7.83 (d, J = 8.4 Hz, 1H), 4.04 (s, 4H), 3.68- 3.60 (m, 2H), 3.50-3.38 (m, 5H), 2.93 (t, J = 10.8 Hz, 1H), 2.87 (s, 3H), 2.07-2.04 (m, 1H), 1.83-1.75 (m, 2H), 0.94 (d, J = 6.8 Hz, 3H), 0.82 (d, J = 6.8 Hz, 3H). Peak 2, cis isomer: 55 mg, 27%. LCMS m/z = 434.1 (M + H)⁺. ¹H NMR (400 MHz, MeOD) δ (ppm) 8.46 (d, J = 8.4 Hz, 1H), 7.83 (d, J = 8.0 Hz, 1H), 4.05 (s, 4H), 3.71 (d, J = 11.6 Hz, 2H), 3.46-3.42 (m, 2H), 3.27 (s, 4H), 2.87 (s, 3H), 2.25-2.21 (m, 1H), 1.73-1.63 (m, 2H), 1.01 (d, J = 7.2 Hz, 6H). |
| 150 | 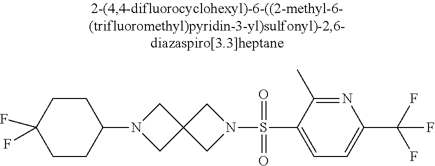 [SMILES: Cc1nc(C(F)(F)F)ccc1S(=O)(=O)N1CC2(CN(C3CCC(F)(F)CC3)C2)C1] | Yield: 55 mg, 45%. LCMS m/z = 440.1 (M + H)⁺. ¹H NMR (400 MHz, CDCl₃) δ (ppm) 8.36 (d, J = 8.4 Hz, 1H), 7.65 (d, J = 8.0 Hz, 1H), 4.04 (s, 4H), 3.27 (s, 4H), 2.90 (s, 3H), 2.09-2.01 (m, 3H), 1.77-1.69 (m, 2H), 1.65-1.60 (m, 2H), 1.46-1.37 (m, 2H). |
| | SM: 4,4-difluorocyclohexan-1-one (41 mg, | |
| | 310 μmol | |
| 151 | 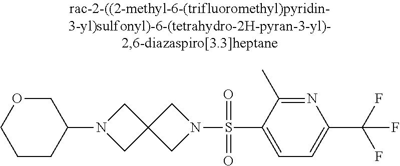 [SMILES: Cc1nc(C(F)(F)F)ccc1S(=O)(=O)N1CC2(CN(C3CCCOC3)C2)C1] | Yield: 34 mg, 30%. LCMS m/z = 406.1 (M + H)⁺. ¹H NMR (400 MHz, MeOD) δ (ppm) 8.45 (d, J = 8.0 Hz, 1H), 7.83 (d, J = 8.0 Hz, 1H), 4.03 (s, 4H), 3.76-3.68 (m, 2H), 3.36-3.33 (m, 5H), 3.07 (t, J = 9.2 Hz, 1H), 2.86 (s, 3H), 2.27- 2.18 (m, 1H), 1.86-1.78 (m, 1H), 1.73-1.62 (m, 1H), 1.57-1.44 (m, 1H), 1.28-1.16 (m, 1H). |
| | SM: dihydro-2H-pyran-3(4H)-one (55 mg, | |
| | 550 μmol, 2.0 equiv.) | |
| 152 | 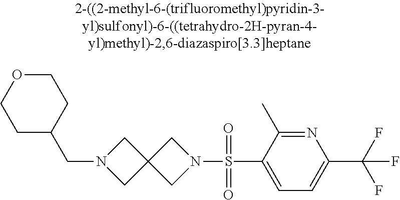 [SMILES: Cc1nc(C(F)(F)F)ccc1S(=O)(=O)N1CC2(CN(CC3CCOCC3)C2)C1] | Yield: 55 mg, 48%. LCMS m/z = 420.1 (M + H)⁺. ¹H NMR (400 MHz, MeOD) δ (ppm) 8.45 (d, J = 8.0 Hz, 1H), 7.83 (d, J = 8.4 Hz, 1H), 4.03 (s, 4H), 3.91-3.86 (m, 2H), 3.30-3.34 (m, 6H), 2.86 (s, 3H), 2.32 (d, J = 6.0 Hz, 2H), 1.62- 1.57 (m, 3H), 1.26-1.16 (m, 2H). |
| | SM: tetrahydro-2H-pyran-4-carbaldehyde (63 | |
| | mg, 550 μmol, 2.0 equiv.) | |
| 153 | 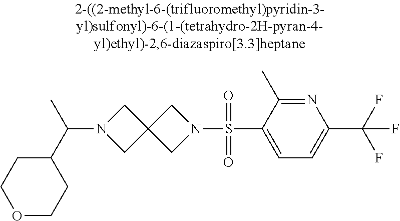 [SMILES: Cc1nc(C(F)(F)F)ccc1S(=O)(=O)N1CC2(CN(C(C)C3CCOCC3)C2)C1] | Yield: 87 mg, 87%. LCMS m/z = 434.1 (M + H)⁺. ¹H NMR (400 MHz, CDCl₃) δ (ppm) 8.36 (d, J = 8.0 Hz, 1H), 7.65 (d, J = 8.0 Hz, 1H), 4.03 (s, 4H), 3.99-3.95 (m, 2H), 3.39-3.29 (m, 2H), 3.28-3.23 (m, 4H), 2.90 (s, 3H), 2.06-1.99 (m, 1H), 1.56-1.49 (m, 1H), 1.47- 1.39 (m, 2H), 1.38-1.29 (m, 2H), 0.80 (d, J = 6.4 Hz, 3H). |
| | SM: -(tetrahydro-2H-pyran-4-yl)ethan-1-one | |
| | (59 mg, 460 μmol, 2.0 equiv.) | |
| 154 | 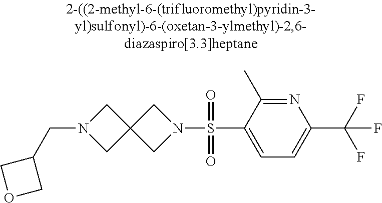 [SMILES: Cc1nc(C(F)(F)F)ccc1S(=O)(=O)N1CC2(CN(CC3COC3)C2)C1] | Yield: 76 mg, 70%. LCMS m/z = 392.1 (M + H)⁺. ¹H NMR (400 MHz, MeOD) δ (ppm) 8.45 (d, J = 8.0 Hz, 1H), 7.83 (d, J = 8.4 Hz, 1H), 4.76-4.71 (m, 2H), 4.36 (t, J = 6.0 Hz, 2H), 4.03 (s, 4H), 3.34 (s, 4H), 3.04-2.95 (m, 1H), 2.86 (s, 3H), 2.74 (d, J = 7.2 Hz, 2H). |
| | SM: oxetane-3-carbaldehyde (48 mg, 550 | |
| | μmol, 2.0 equiv.) | |
| 155 | 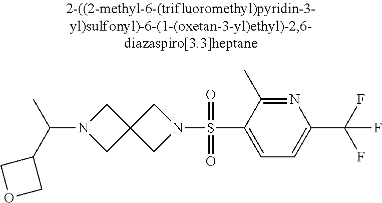 [SMILES: Cc1nc(C(F)(F)F)ccc1S(=O)(=O)N1CC2(CN(C(C)C3COC3)C2)C1] | Yield: 73 mg, 65%. LCMS m/z = 406.1 (M + H)⁺. ¹H NMR (400 MHz, MeOD) δ (ppm) 8.45 (d, J = 8.0 Hz, 1H), 7.83 (d, J = 8.4 Hz, 1H), 4.70-4.48 (m, 4H), 4.02 (s, 4H), 3.36-3.32 (m, 2H), 3.30-3.26 (m, 2H), 3.02-2.95 (m, 1H), 2.86 (s, 3H), 2.62-2.54 (m, 1H), 0.88 (d, J = 6.4 Hz, 3H). |
| | SM: 1-(oxetan-3-yl)ethan-1-one (55 mg, 550 | |
| | μmol, 2.0 equiv.) | |

### Example 156 and 157: (S)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane and (R)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane

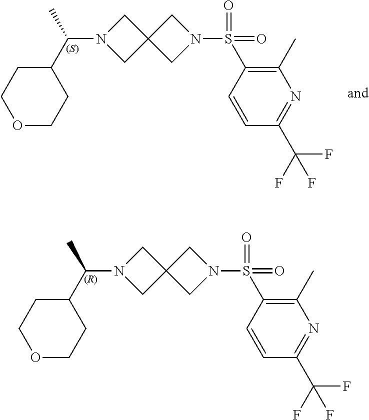

rac-2-((2-methyl-6-(trifluoromethyl)pyridin-3-yl)sulfonyl)-6-(1-(tetrahydro-2H-pyran-4-yl)ethyl)-2,6-diazaspiro[3.3]heptane (Example 153) was purified via SFC (CHIRALPAK AD-H 30×250 mm, 5 um Method: 30% MeOH w/ 0.1% DEA in CO2 (flow rate: 100 mL/min, ABPR 120 bar, MBPR 40 psi, column temp 40 deg C.)), to yield two enantiomers of arbitrarily assigned stereochemistry:

(S)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane (16 mg, 99.4% ee, t_{R}=1.20 min, LCMS m/z=434.1 (M+H)⁺) and

(R)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane (17.5 mg, 97.8% ee, t_{R}=1.36 min, LCMS m/z=434.1 (M+H)⁺).

### Example 158 and 159: (S)-2-((2-methyl-6-(trifluoromethyl)pyridin-3-yl)sulfonyl)-6-(1-(oxetan-3-yl)ethyl)-2,6-diazaspiro[3.3]heptane and (R)-2-((2-methyl-6-(trifluoromethyl)pyridin-3-yl)sulfonyl)-6-(1-(oxetan-3-yl)ethyl)-2,6-diazaspiro[3.3]heptane

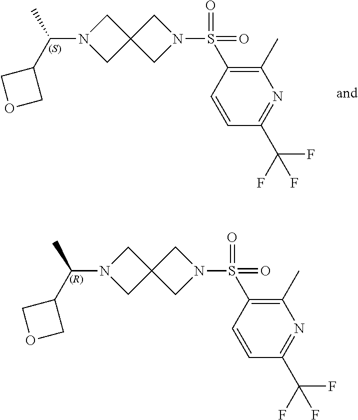

rac-2-((2-methyl-6-(trifluoromethyl)pyridin-3-yl)sulfonyl)-6-(1-(oxetan-3-yl)ethyl)-2,6-diazaspiro[3.3]heptane (Example 155) was purified via SFC (LUX Cellulose-4 LC 30×250 mm, 5 um Method: 30% MeOH w/ 0.1% DEA in CO2 (flow rate: 100 mL/min, ABPR 120 bar, MBPR 40 psi, column temp 40 deg C.)), to yield two enantiomers of arbitrarily assigned stereochemistry: (S)-2-((2-methyl-6-(trifluoromethyl)pyridin-3-yl)sulfonyl)-6-(1-(oxetan-3-yl)ethyl)-2,6-diazaspiro[3.3]heptane (18 mg, 99.6% ee, t_{R}=1.28 min, LCMS m/z=406.1 (M+H)⁺) and

(R)-2-((2-methyl-6-(trifluoromethyl)pyridin-3-yl)sulfonyl)-6-(1-(oxetan-3-yl)ethyl)-2,6-diazaspiro[3.3]heptane (18 mg, 98.7% ee, t_{R}=1.69 min, LCMS m/z=406.1 (M+H)⁺).

### Examples 160 and 161: (R)-2-((2-methyl-6-(trifluoromethyl)pyridin-3-yl)sulfonyl)-6-((tetrahydrofuran-3-yl)methyl)-2,6-diazaspiro[3.3]heptane and (S)-2-((2-methyl-6-(trifluoromethyl)pyridin-3-yl)sulfonyl)-6-((tetrahydrofuran-3-yl)methyl)-2,6-diazaspiro[3.3]heptane

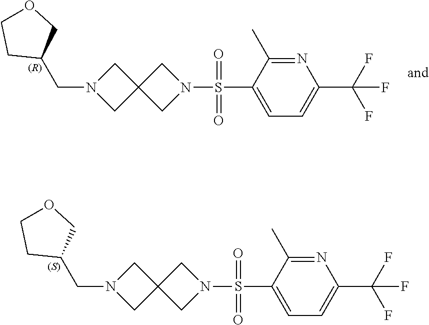

rac-2-((2-methyl-6-(trifluoromethyl)pyridin-3-yl)sulfonyl)-6-((tetrahydrofuran-3-yl)methyl)-2,6-diazaspiro[3.3]heptane (67 mg, 165 umol) (Example 139) was purified via SFC (CHIRALPAK IG 30×250 mm, 5 um Method: 20% MeOH w/ 0.1% DEA in CO2 (flow rate: 100 mL/min, ABPR 120 bar, MBPR 40 psi, column temp 40 C)) to yielding two enantiomers of arbitrarily assigned stereochemistry: first off column (R)-2-((2-methyl-6-(trifluoromethyl)pyridin-3-yl)sulfonyl)-6-((tetrahydrofuran-3-yl)methyl)-2,6-diazaspiro[3.3]heptane (22 mg, 100% ee, 33% yield, t_{R}=2.64 min, LCMS m/z=406.1 (M+H)⁺); Second off column (R)-2-((2-methyl-6-(trifluoromethyl)pyridin-3-yl)sulfonyl)-6-((tetrahydrofuran-3-yl)methyl)-2,6-diazaspiro[3.3]heptane (23 mg, 96.6% ee, 34% yield, t_{R}=2.90 min, LCMS m/z=406.1 (M+H)⁺).

### Example 162: 2-(tetrahydro-2H-pyran-4-yl)-6-((2-(trifluoromethyl)pyridin-3-yl)sulfonyl)-2,6-diazaspiro[3.3]heptane

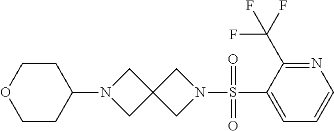 [SMILES: O=S(=O)(c1cccnc1C(F)(F)F)N1CC2(CN(C3CCOCC3)C2)C1]

2-(tetrahydro-2H-pyran-4-yl)-6-((2-(trifluoromethyl)pyridin-3-yl)sulfonyl)-2,6-diazaspiro[3.3]heptane was prepared using a similar method described for Example 36 from the hydrochloride salt of 2-(tetrahydro-2H-pyran-4-yl)-2,6-diazaspiro[3.3]heptane (50 mg, 200 μmol) and 2-(trifluoromethyl)pyridine-3-sulfonyl chloride (77 mg, 247 μmol). The crude product was purified by HPLC (basic conditions, 5-50% acetonitrile) yielding the title compound as an off-white residue (13 mg, 12% yield). LCMS m/z=392.2 (M+H)⁺. ¹H NMR (400 MHz, CD₃OD) δ (ppm): 9.0-8.8 (m, 1H), 8.6-8.5 (m, 1H), 7.87 (dd, J=4.7, 8.1 Hz, 1H), 4.10 (s, 4H), 3.9-4.0 (m, 2H), 3.36 (s, 6H), 2.28 (tt, J=4.1, 10.7 Hz, 1H), 1.68 (br dd, J=1.7, 12.7 Hz, 2H), 1.3-1.2 (m, 2H).

### Example 163: 2-((6-(2,2-difluorocyclopropyl)-2-methylpyridin-3-yl)sulfonyl)-6-(tetrahydro-2H-pyran-4-yl)-2,6-diazaspiro[3.3]heptane

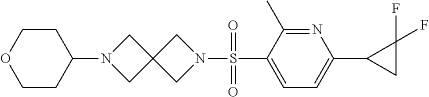 [SMILES: Cc1nc(C2CC2(F)F)ccc1S(=O)(=O)N1CC2(CN(C3CCOCC3)C2)C1]

### 1. Synthesis of of tert-butyl 6-((6-bromo-2-methylpyridin-3-yl)sulfonyl)-2,6-diazaspiro[3.3]heptane-2-carboxylate

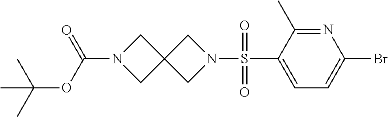 [SMILES: Cc1nc(Br)ccc1S(=O)(=O)N1CC2(CN(C(=O)OC(C)(C)C)C2)C1]

To a solution of tert-butyl 2,6-diazaspiro[3.3]heptane-2-carboxylate (1 g, 5.04 mmol) and DIPEA (717 mg, 5.6 mmol, 970 uL) in DCM (10 mL) was added 6-bromo-2-methyl-pyridine-3-sulfonyl chloride (1.38 g, 5.09 mmol) in batches at rt. Stirring was continued for 15 min. The reaction mixture was diluted with DCM, washed twice with 1N HCl, washed with water and brine, dried over Na2SO4, filtered concentrated. Crude tert-butyl 2-[(6-bromo-2-methyl-3-pyridyl)sulfonyl]-2,6-diazaspiro[3.3]heptane-6-carboxylate (2.05 g, 4.7 mmol, 94% yield) was used without further purification in the next step

### 2. Synthesis of of tert-butyl 6-((2-methyl-6-vinylpyridin-3-yl)sulfonyl)-2,6-diazaspiro[3.3]heptane-2-carboxylate

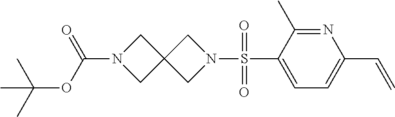 [SMILES: C=Cc1ccc(S(=O)(=O)N2CC3(CN(C(=O)OC(C)(C)C)C3)C2)c(C)n1]

A mixture of 6-((6-bromo-2-methylpyridin-3-yl)sulfonyl)-2,6-diazaspiro[3.3]heptane-2-carboxylate (120 mg, 278 umol) Potassium vinyltrifluoroborate (56 mg, 416 umol), Pd(dppf)Cl2 (40 mg, 56 umol) and potassium carbonate (115 mg, 833 umol) in dioxane (2.25 mL) and water (0.25 mL) was degassed by purging with nitrogen for 5 min. The resulting orange mixture was heated at 80 C for overnight. The reaction mixture was cooled to rt diluted with EtOAc, filtered and directly subjected to column chromatography (24 g SiO2, 0-100% EtOH:EtOAc 1:3 in heptane). The title compound was obtained as an off white solid (73 mg, 192 umol, 69% yield). LCMS m/z=380.2 (M+H)⁺. ¹H NMR (400 MHz, CD₃OD) δ (ppm): 8.08 (d, J=8.5 Hz, 1H), 7.39 (d, J=8.5 Hz, 1H), 6.76 (dd, J=11.0, 17.5 Hz, 1H), 6.28 (dd, J=1.0, 17.5 Hz, 1H), 5.55 (dd, J=1.3, 10.8 Hz, 1H), 3.9-3.9 (m, 8H), 2.69 (s, 3H), 1.31 (s, 9H).

### 3. Synthesis of tert-butyl 6-((6-(2,2-difluorocyclopropyl)-2-methylpyridin-3-yl)sulfonyl)-2,6-diazaspiro[3.3]heptane-2-carboxylate

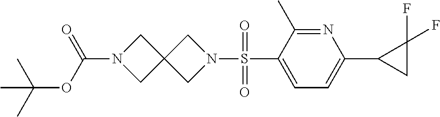 [SMILES: Cc1nc(C2CC2(F)F)ccc1S(=O)(=O)N1CC2(CN(C(=O)OC(C)(C)C)C2)C1]

To a mixture of tert-butyl 6-((2-methyl-6-vinylpyridin-3-yl)sulfonyl)-2,6-diazaspiro[3.3]heptane-2-carboxylate (72 mg, 190 umol) and sodium iodide (28 mg, 190 umol) in THE (1.5 mL) was added trimethyl(trifluoromethyl)silane (216 mg, 1.5 mmol, 220 uL) in a microwave flask at rt. The vial was capped and heated at 75 C for 4 hrs after which another batch of sodium iodide (28 mg, 190 umol) and trimethyl(trifluoromethyl)silane (216 mg, 1.5 mmol, 220 uL) were added and heating was continues for 8 hr. The contents of the microwave vial were cooled to rt and poured onto a mixture of EtOAc (4 mL) and water (4 mL). The aq. phase was extracted twice with EtOAc (4 mL) and the combined organic layers were dried over Na2SO4, filtered and concentrated. Column chromatography (12 g SiO2, 0-100% EtOH:EtOAc 1:3 in heptane) yielded the desired product (49 mg, 114 umol, 60% yield). LCMS m/z=430.2 (M+H)⁺. ¹H NMR (400 MHz, CD₃OD) δ (ppm): 8.06 (d, 1H, J=8.0 Hz), 7.27 (d, 1H, J=8.0 Hz), 3.91 (s, 8H), 2.98 (ddd, 1H, J=8.0, 11.3, 12.8 Hz), 2.6-2.7 (m, 3H), 2.2-2.3 (m, 1H), 1.8-1.9 (m, 1H), 1.31 (s, 9H).

### 4. Synthesis of 2-((6-(2,2-difluorocyclopropyl)-2-methylpyridin-3-yl)sulfonyl)-2,6-diazaspiro[3.3]heptane

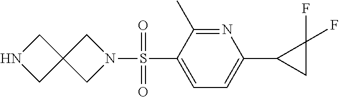 [SMILES: Cc1nc(C2CC2(F)F)ccc1S(=O)(=O)N1CC2(CNC2)C1]

To a solution of tert-butyl 6-((6-(2,2-difluorocyclopropyl)-2-methylpyridin-3-yl)sulfonyl)-2,6-diazaspiro[3.3]heptane-2-carboxylate (49 mg, 114 umol) in DCM (0.5 mL) was added TFA (149 mg, 1.31 mmol, 100 uL) at rt. The resulting mixture was stirred for 60 min after which sodium bicarbonate (125 mg, 1.48 mmol) was added followed by methanol (3 mL). The reaction mixture was sonicated for 5 min and the resulting brown mixture was directly loaded onto a silica. Column chromatography (12 g SiO2, 50-100% EtOH:EtOAc 1:3 in heptane) yielded the title compound (19 mg, 58 umol, 51% yield). LCMS m/z=330.1 (M+H)⁺. LCMS t_{R} (2 min)=0.54 min.

### 5. Synthesis of 2-((6-(2,2-difluorocyclopropyl)-2-methylpyridin-3-yl)sulfonyl)-6-(tetrahydro-2H-pyran-4-yl)-2,6-diazaspiro[3.3]heptane

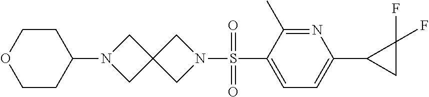 [SMILES: Cc1nc(C2CC2(F)F)ccc1S(=O)(=O)N1CC2(CN(C3CCOCC3)C2)C1]

To a solution of 2-((6-(2,2-difluorocyclopropyl)-2-methylpyridin-3-yl)sulfonyl)-2,6-diazaspiro[3.3]heptane (19 mg, 58 umol), tetrahydropyran-4-one (12 mg, 115 umol, 11 uL) and acetic acid (10 mg, 173. umol, 10 uL) in DCM (1.5 mL) was added sodium triacetoxyborohydride (STAB) (36.68 mg, 173.06 umol). The resulting reaction mixture was stirred for 1 hr diluted with DCM (5 mL) and sat. aq. NaHCO₃ (5 mL) was added. The organic phase was separated, dried over Na2SO4, filtered and concentrated. Purification by basic HPLC (5-45% acetonitrile) yielded the title compound (13 mg, 31 umol, 55% yield) as a colorless residue. LCMS m/z=414.2 (M+H)⁺. ¹H NMR (400 MHz, CD₃OD) δ (ppm): 8.07 (d, 1H, J=8.2 Hz), 7.28 (d, 1H, J=8.2 Hz), 3.87 (s, 4H), 3.81 (br d, 2H, J=10.7 Hz), 3.32 (s, 4H), 3.2-3.3 (m, 2H), 2.99 (dt, 1H, J=8.2, 11.9 Hz), 2.68 (s, 3H), 2.2-2.3 (m, 2H), 1.8-1.9 (m, 1H), 1.57 (br d, 2H, J=12.2 Hz), 1.13 (dq, 2H, J=4.3, 11.8 Hz).

### Example 164: 2-((2-methylpyridin-3-yl)sulfonyl)-6-(tetrahydro-2H-pyran-4-yl)-2,6-diazaspiro[3.3]heptane

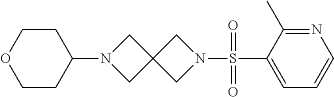 [SMILES: Cc1ncccc1S(=O)(=O)N1CC2(CN(C3CCOCC3)C2)C1]

### 4. Synthesis of 2-((6-bromo-2-methylpyridin-3-yl)sulfonyl)-2,6-diazaspiro[3.3]heptane

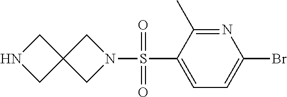 [SMILES: Cc1nc(Br)ccc1S(=O)(=O)N1CC2(CNC2)C1]

2-((6-bromo-2-methylpyridin-3-yl)sulfonyl)-2,6-diazaspiro[3.3]heptane was prepared from tert-butyl 6-((6-bromo-2-methylpyridin-3-yl)sulfonyl)-2,6-diazaspiro[3.3]heptane-2-carboxylate (1 g, 2.31 mmol) using a similar method described in step 4 for Example 163. The crude compound (768, 2.18 mmol, 94% yield) was obtained by filtration and used without further purification in the next step. LCMS m/z=332.0 (M+H)⁺.

### 5. Synthesis of 2-((6-bromo-2-methylpyridin-3-yl)sulfonyl)-6-(tetrahydro-2H-pyran-4-yl)-2,6-diazaspiro[3.3]heptane

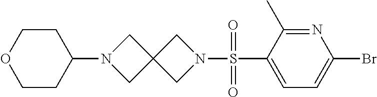 [SMILES: Cc1nc(Br)ccc1S(=O)(=O)N1CC2(CN(C3CCOCC3)C2)C1]

2-((6-bromo-2-methylpyridin-3-yl)sulfonyl)-6-(tetrahydro-2H-pyran-4-yl)-2,6-diazaspiro[3.3]heptane was prepared from 2-((6-bromo-2-methylpyridin-3-yl)sulfonyl)-2,6-diazaspiro[3.3]heptane (71 mg, 214 μmol) and tetrahydropyran-4-one (24 mg, 230 μmol) using a similar method described in step 5 for Example 163. The crude compound (77 mg, 185 umol, 87% yield) was used without further purification in the next step. LCMS m/z=416.1 (M+H)⁺.

### 6. Synthesis of 2-((2-methylpyridin-3-yl)sulfonyl)-6-(tetrahydro-2H-pyran-4-yl)-2,6-diazaspiro[3.3]heptane

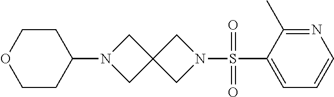 [SMILES: Cc1ncccc1S(=O)(=O)N1CC2(CN(C3CCOCC3)C2)C1]

A mixture of 2-((6-bromo-2-methylpyridin-3-yl)sulfonyl)-6-(tetrahydro-2H-pyran-4-yl)-2,6-diazaspiro[3.3]heptane (200 mg, 480 umol) Pd(dppf)Cl2 (70 mg, 96 umol), potassium carbonate (199 mg, 1.44 mmol) and trifluoro-[1-(trifluoromethyl)vinyl]boron (117 mg, 721 umol) in dioxane (2.25 mL) and water (0.25 mL) was degassed by purging with nitrogen for 5 min. The resulting orange mixture was heated at 80 C for overnight. The mixture was diluted with EtOAc (5 mL), filtered and directly subjected to column chromatography (24 g SiO2, 0-100% EtOH:EtOAc 1:3 in heptane). The obtained material was further purified by HPLC (NH₄OH, 0-60% ACN in water, 60 mL/min system) yielding the title compound (7 mg, 22 umol, 5% yield) as a colorless oil. LCMS m/z=338.3 (M+H)⁺. ¹H NMR (400 MHz, CD₃OD) δ (ppm): 8.67 (dd, 1H, J=1.5, 4.9 Hz), 8.30 (dd, 1H, J=1.5, 7.9 Hz), 7.48 (dd, 1H, J=5.0, 7.8 Hz), 4.00 (s, 4H), 3.93 (br dd, 2H, J=1.5, 11.9 Hz), 3.35 (m, 6H), 2.83 (s, 3H), 2.27 (tt, 1H, J=4.0, 10.6 Hz), 1.67 (br dd, 2H, J=1.7, 12.7 Hz), 1.2-1.3 (m, 2H).

### Example 165: 2-((6-chloro-5-(trifluoromethyl)pyridin-3-yl)sulfonyl)-6-(tetrahydro-2H-pyran-4-yl)-2,6-diazaspiro[3.3]heptane

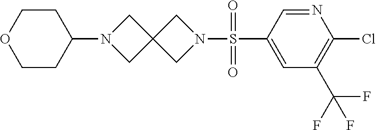 [SMILES: O=S(=O)(c1cnc(Cl)c(C(F)(F)F)c1)N1CC2(CN(C3CCOCC3)C2)C1]

2-((6-chloro-5-(trifluoromethyl)pyridin-3-yl)sulfonyl)-6-(tetrahydro-2H-pyran-4-yl)-2,6-diazaspiro[3.3]heptane was prepared using a similar method described for Example 36 from the hydrochloride salt of 2-(tetrahydro-2H-pyran-4-yl)-2,6-diazaspiro[3.3]heptane (50 mg, 200 μmol) and 6-chloro-5-(trifluoromethyl)pyridine-3-sulfonyl chloride (77 mg, 247 μmol). The crude product was purified by HPLC (basic conditions, 5-50% acetonitrile) yielding the title compound as a colorless oil (16 mg, 14% yield). LCMS m/z=426.2 (M+H)⁺. ¹H NMR (400 MHz, CD₃OD) δ (ppm): 8.93 (d, 1H, J=2.1 Hz), 8.39 (d, 1H, J=2.1 Hz), 3.86 (s, 4H), 3.79 (br dd, 2H, J=1.8, 10.1 Hz), 3.2-3.3 (m, 2H), 3.15 (s, 4H), 2.12 (tt, 1H, J=4.0, 10.8 Hz), 1.52 (br dd, 2H, J=1.8, 12.5 Hz), 1.0-1.1 (m, 2H).

### Example 166: 2-((3-(1,1-difluoroethyl)-1-methyl-1H-pyrazol-5-yl)sulfonyl)-6-(tetrahydro-2H-pyran-4-yl)-2,6-diazaspiro[3.3]heptane

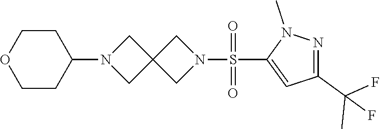 [SMILES: Cn1nc(C(C)(F)F)cc1S(=O)(=O)N1CC2(CN(C3CCOCC3)C2)C1]

### 1. Synthesis of 2-((3-bromo-1-methyl-1H-pyrazol-5-yl)sulfonyl)-6-(tetrahydro-2H-pyran-4-yl)-2,6-diazaspiro[3.3]heptane

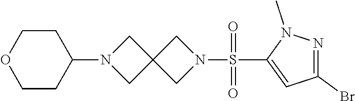 [SMILES: Cn1nc(Br)cc1S(=O)(=O)N1CC2(CN(C3CCOCC3)C2)C1]

2-((3-bromo-1-methyl-1H-pyrazol-5-yl)sulfonyl)-6-(tetrahydro-2H-pyran-4-yl)-2,6-diazaspiro[3.3]heptane was prepared using a similar method described for Example 36 from the hydrochloride salt of 2-(tetrahydro-2H-pyran-4-yl)-2,6-diazaspiro[3.3]heptane (328 mg, 1.5 mmol) and 3-bromo-1-methyl-1H-pyrazole-5-sulfonyl chloride (503 mg, 1.9 mmol). The crude product was purified by column chromatography (24 g SiO2, 40-85% EtOH:EtOAc 1:3 in heptane) yielding the title compound (316 mg, 780 umol, 52% yield). LCMS m/z=405.2 (M+H)⁺. ¹H NMR (500 MHz, DICHLOROMETHANE-d₂) δ (ppm) 6.74 (s, 1H), 4.05 (s, 3H), 3.94 (s, 4H), 3.88-3.82 (m, 2H), 3.33-3.26 (m, 2H), 3.17 (s, 4H), 2.14-2.03 (m, 1H), 1.58-1.53 (m, 2H), 1.24-1.14 (m, 2H).

### 2. Synthesis of 1-(1-methyl-5-((6-(tetrahydro-2H-pyran-4-yl)-2,6-diazaspiro[3.3]heptan-2-yl)sulfonyl)-1H-pyrazol-3-yl)ethan-1-one

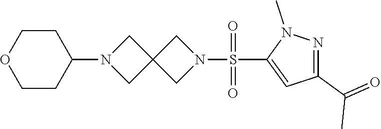 [SMILES: CC(=O)c1cc(S(=O)(=O)N2CC3(CN(C4CCOCC4)C3)C2)n(C)n1]

A mixture of 2-((3-bromo-1-methyl-1H-pyrazol-5-yl)sulfonyl)-6-(tetrahydro-2H-pyran-4-yl)-2,6-diazaspiro[3.3]heptane (82 mg, 202 umol), Tetrakis(triphenylphosphine)palladium(0) (47 mg, 41 umol) and dibutyl-(1-ethoxyvinyl)-propyl-stannane (141 mg, 405 umol, 131 uL) in dioxane (2 mL) was degassed by purging with nitrogen for 15 min. the resulting orange mixture was heated at reflux for overnight. The resulting reaction mixture was cooled to room temperature and 3N HCl (2 mL) was added. After 2 hr, EtOAc (5 mL) was added. the organic phase was separated, dried over Na2SO4, filtered and concentrated under reduced pressure. Column chromatography (12 g SiO2, 30-100% EtOH:EtOAc 1:3 in heptane) of the crude residue yielded the title compound (51 mg, 138 umol, 68% yield) as a pale orange residue as a mixture of unidentified impurities. The material was used without further purification in the next step. LCMS m/z=369.3 (M+H)⁺.

### 3. Synthesis of 2-((3-(1,1-difluoroethyl)-1-methyl-1H-pyrazol-5-yl)sulfonyl)-6-(tetrahydro-2H-pyran-4-yl)-2,6-diazaspiro[3.3]heptane

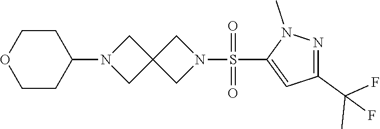 [SMILES: Cn1nc(C(C)(F)F)cc1S(=O)(=O)N1CC2(CN(C3CCOCC3)C2)C1]

1-(1-methyl-5-((6-(tetrahydro-2H-pyran-4-yl)-2,6-diazaspiro[3.3]heptan-2-yl)sulfonyl)-1H-pyrazol-3-yl)ethan-1-one (51 mg, 138 umol) was taken up in a DCM (2 mL) and DAST (134 mg, 831 umol, 110 uL) was added at room temperature. As no product formation was observed the reaction mixture was concentrated under reduced pressure more DAST (134 mg, 831 umol, 110 uL) and triethylamine trihydrofluoride (199 mg, 1.23 mmol, 200 uL) were added and the resulting mixture was stirred at rt for 5 hr. EtOAc (10 mL) and sat. aq. NaHCO₃ (10 mL) were added. The organic phase was separated, dried over Na₂SO₄, filtered and concentrated under reduced pressure. The resulting residue was purified by HPLC under basic conditions (5-55% CH₃CN in water) yielding the title compound (4 mg, 10 umol, 7% yield) as a colorless residue. LCMS m/z=391.3 (M+H)⁺. ¹H NMR (400 MHz, CD₃OD) δ (ppm): 6.92 (s, 1H), 4.00 (d, 7H, J=7.0 Hz), 3.88 (br dd, 2H, J=3.2, 11.8 Hz), 3.83 (br s, 4H), 3.2-3.3 (m, 4H), 2.82 (br d, 1H, J=0.9 Hz), 1.88 (t, 3H, J=18.6 Hz), 1.6-1.8 (m, 2H), 1.2-1.3 (m, 3H).

### Example 167: rac-2-(1-(1-methyl-2-oxabicyclo[2.1.1]hexan-4-yl)ethyl)-6-((2-methyl-6-(trifluoromethyl)pyridin-3-yl)sulfonyl)-2,6-diazaspiro[3.3]heptane

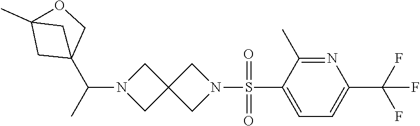 [SMILES: Cc1nc(C(F)(F)F)ccc1S(=O)(=O)N1CC2(CN(C(C)C34COC(C)(C3)C4)C2)C1]

To a mixture of 2-((2-methyl-6-(trifluoromethyl)pyridin-3-yl)sulfonyl)-2,6-diazaspiro[3.3]heptane (50 mg, 115 umol, Trifluoroacetic acid) and 1-(1-methyl-2-oxabicyclo[2.1.1]hexan-4-yl)ethanone (20 mg, 138 umol) in acetic acid (41 mg, 689 umol, 40 uL) and DCM (2 mL) was added STAB (100 mg, 472 umol) at rt. Stirring was continued for 30 min. The reaction mixture was diluted with EtOAc (5 mL) and washed with sat. aq. NaHCO3 (2 mL), water (2 mL) and brine (2 mL). The organic phase was dried over Na2SO4, filtered and concentrated. Column chromatography (12 g SiO2, 50-100% EtOH:EtOAc 1:3 in heptane) of the resulting residue resulted in the title compound as a pale yellow residue (41 mg, 92 umol, 80% yield). LCMS m/z=446.0 (M+H)⁺. ¹H NMR (500 MHz, CD₃OD) δ (ppm): 8.35 (br d, 1H, J=7.9 Hz), 7.73 (br d, 1H, J=8.2 Hz), 3.93 (s, 4H), 3.53 (br s, 2H), 3.2-3.4 (m, 4H), 2.76 (s, 3H), 2.53 (br d, 1H, J=6.4 Hz), 1.4-1.6 (m, 2H), 1.3-1.4 (m, 2H), 1.26 (s, 3H), 0.83 (br d, 3H, J=6.7 Hz).

### Example 168: 2-((6-methoxy-4-methylpyridin-3-yl)sulfonyl)-6-(tetrahydro-2H-pyran-4-yl)-2,6-diazaspiro[3.3]heptane

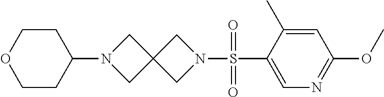 [SMILES: COc1cc(C)c(S(=O)(=O)N2CC3(CN(C4CCOCC4)C3)C2)cn1]

The title compound was prepared using a similar method described for Example 36 from the hydrochloride salt of 2-(tetrahydro-2H-pyran-4-yl)-2,6-diazaspiro[3.3]heptane (26 mg, 100 μmol) and 6-methoxy-4-methylpyridine-3-sulfonyl chloride (24 mg, 110 μmol). The crude product was purified by HPLC (acidic conditions, 5-55% acetonitrile) yielding the title compound as an off-white residue (43 mg, 88% yield, TFA salt). LCMS m/z=368.2 (M+H)⁺. LCMS t_{R} (2 min)=0.49 min.

### Example 169: 2-methyl-3-((6-(tetrahydro-2H-pyran-4-yl)-2,6-diazaspiro[3.3]heptan-2-yl)sulfonyl)-2,4,5,6-tetrahydrocyclopenta[c]pyrazole

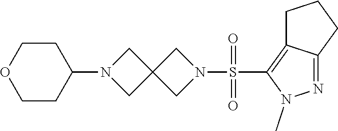 [SMILES: Cn1nc2c(c1S(=O)(=O)N1CC3(CN(C4CCOCC4)C3)C1)CCC2]

The title compound was prepared using a similar method described for Example 36 from the hydrochloride salt of 2-(tetrahydro-2H-pyran-4-yl)-2,6-diazaspiro[3.3]heptane (26 mg, 100 μmol) and 2-methyl-2,4,5,6-tetrahydrocyclopenta[c]pyrazole-3-sulfonyl chloride (24 mg, 110 μmol). The crude product was purified by HPLC (acidic conditions, 5-55% acetonitrile) yielding the title compound as an off-white residue (45 mg, 94% yield, TFA salt). LCMS m/z=367.2 (M+H)⁺. LCMS t_{R} (2 min)=0.49 min.

Examples 170 to 186: Examples 170 to 186, shown in the Table below, were made using the general procedure described below:

### 1. Synthesis of 2-((2-methyl-6-(trifluoromethyl)pyridin-3-yl)sulfonyl)-2,6-diazaspiro[3.3]heptane p-toluenesulfonic acid salt

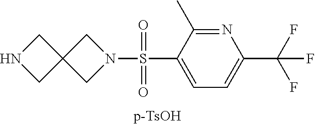 [SMILES: Cc1nc(C(F)(F)F)ccc1S(=O)(=O)N1CC2(CNC2)C1]

tert-Butyl 6-((2-methyl-6-(trifluoromethyl)pyridin-3-yl)sulfonyl)-2,6-diazaspiro[3.3]heptane-2-carboxylate (7.0 g, 16.61 mmol) was dissolved in acetonitrile (50 mL) and p-toluenesulfonic acid monohydrate (7 g, 36.80 mmol) was added. The reaction was stirred at RT for 2 hrs. The resulting thick white solid was filtered and the filter cake was rinsed with acetonitrile to afford the title compound as a TsOH salt. ¹H NMR (500 MHz, DMSO-d6) δ (ppm) 8.39-8.52 (m, 3H), 7.99 (d, J=8.24 Hz, 1H), 7.50 (d, J=7.94 Hz, 2H), 7.13 (d, J=7.94 Hz, 2H), 4.08-4.14 (m, 8H), 2.81 (s, 3H), 2.29 (s, 3H). LCMS m/z=322.0 [M+H]⁺.

### 2. General procedure for the reductive amination using 2-((2-methyl-6-(trifluoromethyl)pyridin-3-yl)sulfonyl)-2,6-diazaspiro[3.3]heptane p-toluenesulfonic acid salt

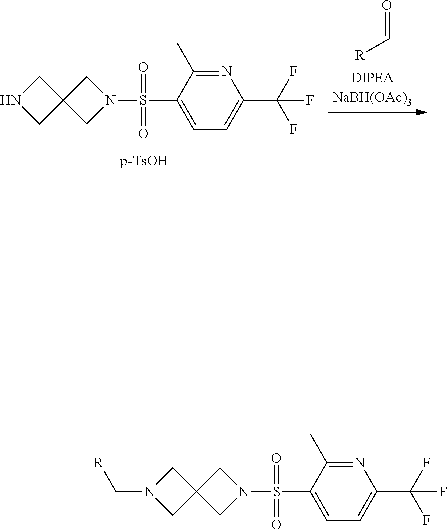

To a mixture of 2-((2-methyl-6-(trifluoromethyl)pyridin-3-yl)sulfonyl)-2,6-diazaspiro[3.3]heptane (35 mg, 70 umol, pTsOH salt, 1 equiv.), aldehyde (1.4 equiv.) and DIPEA (1 equiv.) in 1,2-dichloroethane (1 ml) was added tetramethylammonium triacetoxyborohydride. (5 equiv., In case of using a salt of any reagent, an additional equivalent of DIPEA was added). The reaction mixture was stirred at room temperature for 16 hours. The solvent was evaporated under reduce pressure and the residue was mixed with ammonium hydroxide (1 ml, of 5% solution in MeOH). The resulting mixture was evaporated under reduce pressure and the residue was dissolved in DMSO (appr. 1 ml up to 100 mg of product). The solution was neutralized with acetic acid, filtered, analyzed by LCMS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized Water (phase A) and HPLC-grade Methanol or Acetonitrile (phase B) were used as an eluent. In some cases, ammonia or TFA was used as an additive to improve the separation of the products. In these cases, free bases and TFA salts of the products were formed respectively.

| Example | Name/Structure/Aldehyde (SM) | Data |
|---|---|---|
| 170 | 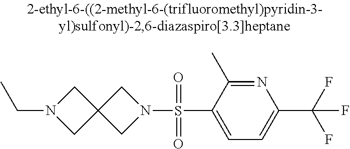 [SMILES: CCN1CC2(C1)CN(S(=O)(=O)c1ccc(C(F)(F)F)nc1C)C2] | Yield: 3.2 mg, 13% LCMS m/z = 350.2 (M + H)⁺ LCMS t_{R} (2 min) = 0.79 min. |
| | SM: acetaldehyde | |
| 171 | 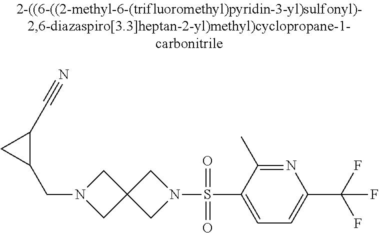 [SMILES: Cc1nc(C(F)(F)F)ccc1S(=O)(=O)N1CC2(CN(CC3CC3C#N)C2)C1] | Yield: 13.8 mg, 49% LCMS m/z = 401.0 (M + H)⁺. LCMS t_{R} (2 min) = 1.05 min. |
| | SM: 2-formylcyclopropane-1-carbonitrile | |
| 172 | 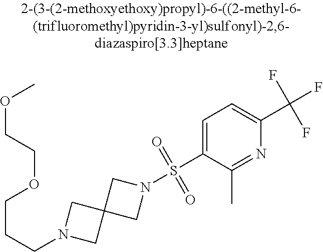 [SMILES: COCCOCCCN1CC2(C1)CN(S(=O)(=O)c1ccc(C(F)(F)F)nc1C)C2] | Yield: 13.4 mg, 44% LCMS m/z = 438.2 (M + H)⁺. LCMS t_{R} (2 min) = 0.75 min. |
| | SM: methoxyethoxy)propanal | |
| 173 | 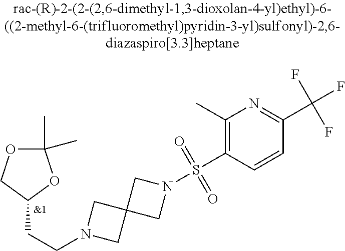 [SMILES: Cc1nc(C(F)(F)F)ccc1S(=O)(=O)N1CC2(CN(CC[C@@H]3COC(C)(C)O3)C2)C1] | Yield: 15.9 mg, 51% LCMS m/z = 450.0 (M + H)⁺. LCMS t_{R} (2 min) = 1.13 min. |
| | SM: rac-(R)-2-(2,2-dimethyl-1,3-dioxolan-4-yl)acetaldehyde | |
| 174 | 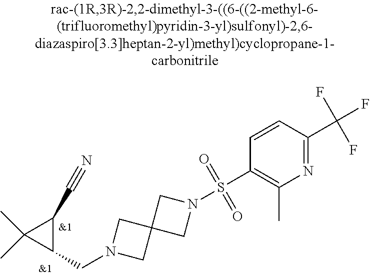 [SMILES: Cc1nc(C(F)(F)F)ccc1S(=O)(=O)N1CC2(CN(C[C@H]3[C@H](C#N)C3(C)C)C2)C1] | Yield: 15.6 mg, 52% LCMS m/z = 429.0 (M + H)⁺. LCMS t_{R} (2 min) = 0.85 min. |
| | SM: rac-(1R,3R)-3-formyl-2,2-dimethylcyclopropane-1- | |
| | carbonitrile | |
| 175 | 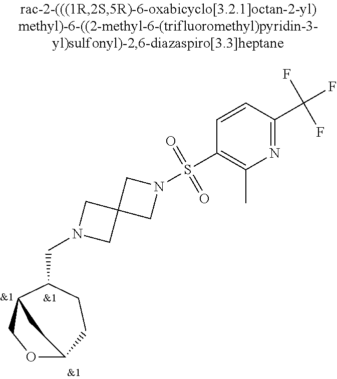 [SMILES: Cc1nc(C(F)(F)F)ccc1S(=O)(=O)N1CC2(CN(C[C@@H]3CC[C@H]4C[C@H]3CO4)C2)C1] | Yield: 18.5 mg, 59% LCMS m/z = 446.0 (M + H)⁺. LCMS t_{R} (2 min) = 1.08 min. |
| | SM: rac-(1R,2S,5R)-6-oxabicyclo[3.2.1]octane-2- | |
| | carbaldehyde | |
| 176 | 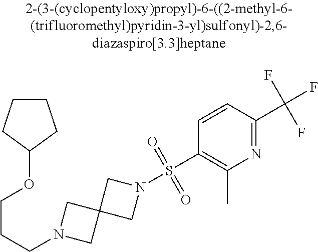 [SMILES: Cc1nc(C(F)(F)F)ccc1S(=O)(=O)N1CC2(CN(CCCOC3CCCC3)C2)C1] | Yield: 13.8 mg, 44% LCMS m/z = 448.2 (M + H)⁺. LCMS t_{R} (2 min) = 1.18 min. |
| | SM: 3-(cyclopentyloxy)propanal | |
| 177 | 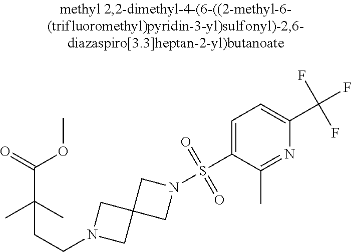 [SMILES: COC(=O)C(C)(C)CCN1CC2(C1)CN(S(=O)(=O)c1ccc(C(F)(F)F)nc1C)C2] | Yield: 18.2 mg, 58%; TFA-salt LCMS m/z = 450.0 (M + H)⁺. LCMS t_{R} (2 min) = 1.14 min. |
| | SM: methyl 2,2-dimethyl-4-oxobutanoate | |
| 178 | 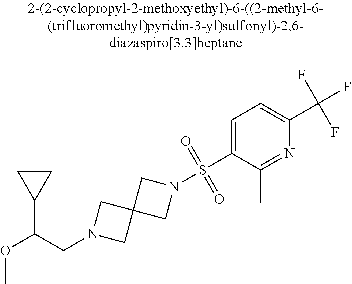 [SMILES: COC(CN1CC2(C1)CN(S(=O)(=O)c1ccc(C(F)(F)F)nc1C)C2)C1CC1] | Yield: 15.1 mg, 51%; LCMS m/z = 420.0 (M + H)⁺. LCMS t_{R} (2 min) = 1.14 min. |
| | SM: 2-cyclopropyl-2-methoxyacetaldehyde | |
| 179 | 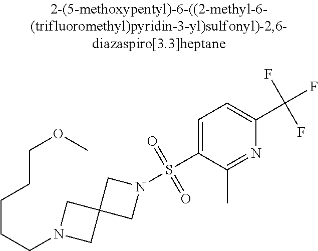 [SMILES: COCCCCCN1CC2(C1)CN(S(=O)(=O)c1ccc(C(F)(F)F)nc1C)C2] | Yield: 15.1 mg, 51%; LCMS m/z = 422.2 (M + H)⁺. LCMS t_{R} (2 min) = 1.12 min. |
| | SM: 5-methoxypentanal | |
| 180 | 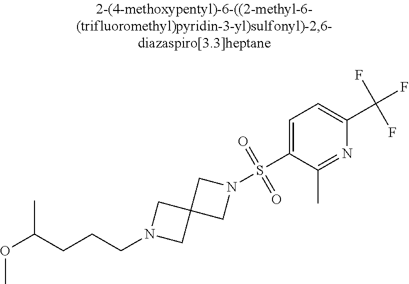 [SMILES: COC(C)CCCN1CC2(C1)CN(S(=O)(=O)c1ccc(C(F)(F)F)nc1C)C2] | Yield: 14.2 mg, 48%; LCMS m/z = 422.2 (M + H)⁺. LCMS t_{R} (2 min) = 1.12 min. |
| | SM: 4-methoxypentanal | |
| 181 | 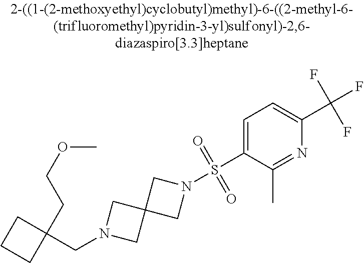 [SMILES: COCCC1(CN2CC3(C2)CN(S(=O)(=O)c2ccc(C(F)(F)F)nc2C)C3)CCC1] | Yield: 13.7 mg, 44%; LCMS m/z = 448.2 (M + H)⁺. LCMS t_{R} (2 min) = 1.09 min. |
| | SM: 1-(2-methoxyethyl)cyclobutane-1-carbaldehyde | |
| 182 | 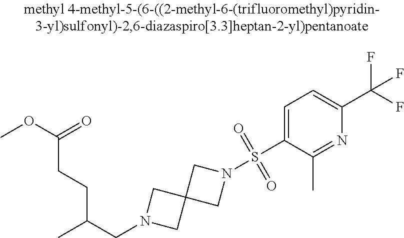 | Yield: 15.5 mg, 49%; TFA salt; LCMS m/z = 422.2 (M + H)⁺. LCMS t_{R} (2 min) = 1.12 min. |
| | SM: methyl 4-methyl-5-oxopentanoate | |
| 183 | 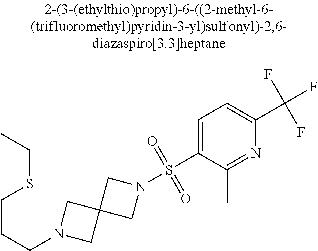 [SMILES: CCSCCCN1CC2(C1)CN(S(=O)(=O)c1ccc(C(F)(F)F)nc1C)C2] | Yield: 12.8 mg, 43%; LCMS m/z = 424.2 (M + H)⁺. LCMS t_{R} (2 min) = 0.79 min. |
| | SM: 3-(ethylthio)propanal | |
| 184 | 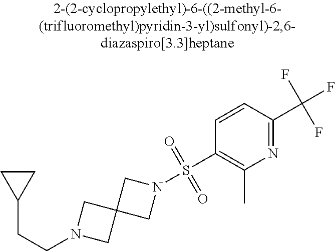 [SMILES: Cc1nc(C(F)(F)F)ccc1S(=O)(=O)N1CC2(CN(CCC3CC3)C2)C1] | Yield: 15.9 mg, 58%; LCMS m/z = 390.0 (M + H)⁺. LCMS t_{R} (2 min) = 0.94 min. |
| | SM: 2-cyclopropylacetaldehyde | |
| 185 | 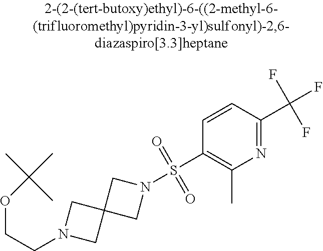 [SMILES: Cc1nc(C(F)(F)F)ccc1S(=O)(=O)N1CC2(CN(CCOC(C)(C)C)C2)C1] | Yield: 19.3 mg, 65%; LCMS m/z = 422.0 (M + H)⁺. LCMS t_{R} (2 min) = 0.99 min. |
| | SM: 2-(tert-butoxy)acetaldehyde | |
| 186 | 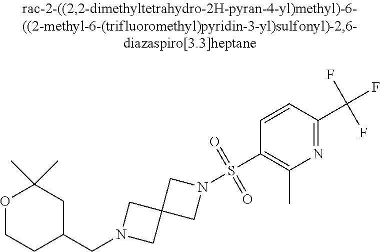 [SMILES: Cc1nc(C(F)(F)F)ccc1S(=O)(=O)N1CC2(CN(CC3CCOC(C)(C)C3)C2)C1] | Yield: 14.2 mg, 45%; LCMS m/z = 448.2 (M + H)⁺. LCMS t_{R} (2 min) = 0.77 min. |
| | SM: rac-2,2-dimethyltetrahydro-2H-pyran-4-carbaldehyde | |

### Example 187: 4-((6-((2-methyl-6-(trifluoromethyl)pyridin-3-yl)sulfonyl)-2,6-diazaspiro[3.3]heptan-2-yl)methyl)tetrahydro-2H-pyran-4-ol

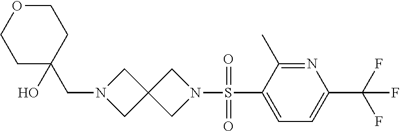 [SMILES: Cc1nc(C(F)(F)F)ccc1S(=O)(=O)N1CC2(CN(CC3(O)CCOCC3)C2)C1]

To a mixture of 1,6-dioxaspiro[2.5]octane (32 mg, 0.28 mmol) and 2-[[2-methyl-6-(trifluoromethyl)-3-pyridyl]sulfonyl]-2,6-diazaspiro[3.3]heptane TFA salt (80 mg, 0.18 mmol) in EtOH (1 mL) was added Hunigs base (64 mL, 0.37 mmol). The reaction solution was heated at 55 C for 30 min, and rt overnight. Added EtOAc and satd. NaHCO₃, the organic phase was washed with water and concentrated. The residue was purified by normal phase column (12 g, EtOAc/EtOH 3/1 in heptane 50-100%) to get 4-((6-((2-methyl-6-(trifluoromethyl)pyridin-3-yl)sulfonyl)-2,6-diazaspiro[3.3]heptan-2-yl)methyl)tetrahydro-2H-pyran-4-ol as a white solid (64 mg, 80% yield). LCMS m/z=436.2 (M+H)⁺. ¹H NMR (400 MHz, CD₃OD) δ (ppm): 8.48 (d, J=8.0 Hz, 1H), 7.85 (d, J=8.0 Hz, 1H), 4.61 (br s, 1H), 4.05 (s, 4H), 3.63-3.82 (m, 4H), 3.44 (s, 4H), 2.88 (s, 3H), 2.44 (s, 2H), 1.55-1.70 (m, 2H), 1.38-1.52 (m, 2H)

### Example 188: 2-((4-fluorotetrahydro-2H-pyran-4-yl)methyl)-6-((2-methyl-6-(trifluoromethyl)pyridin-3-yl)sulfonyl)-2,6-diazaspiro[3.3]heptane

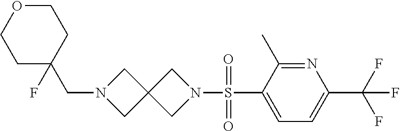 [SMILES: Cc1nc(C(F)(F)F)ccc1S(=O)(=O)N1CC2(CN(CC3(F)CCOCC3)C2)C1]

To a solution of 4-((6-((2-methyl-6-(trifluoromethyl)pyridin-3-yl)sulfonyl)-2,6-diazaspiro[3.3]heptan-2-yl)methyl)tetrahydro-2H-pyran-4-ol (22 mg, 0.051 mmol) in DCM (2 mL) was added DAST (19 mg, 0.12 mmol) at rt. The mixture was stirred at rt for 30 min. Added DCM and satd. NaHCO₃. The organic phase was washed with water and concentrated. The residue was purified by normal phase column chromatography on silica gel (12 g, EtOAc in EtOH 3/1 in heptane 20-100%) to get 2-((4-fluorotetrahydro-2H-pyran-4-yl)methyl)-6-((2-methyl-6-(trifluoromethyl)pyridin-3-yl)sulfonyl)-2,6-diazaspiro[3.3]heptane as a white solid (5.8 mg, 26% yield). LCMS m/z=438.2 (M+H)⁺. ¹H NMR (400 MHz, CD₃OD) δ (ppm): 8.47 (d, J=8.0 Hz, 1H), 7.85 (d, J=8.0 Hz, 1H), 4.05 (s, 4H), 3.61-3.81 (m, 4H), 3.43 (s, 4H), 2.88 (s, 3H), 2.57-2.68 (m, 2H), 1.59-1.82 (m, 4H).

### Example 189: 2-((3-isopropyl-1-methyl-1H-pyrazol-5-yl)sulfonyl)-6-(tetrahydro-2H-pyran-4-yl)-2,6-diazaspiro[3.3]heptane

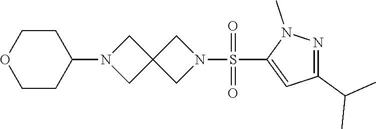 [SMILES: CC(C)c1cc(S(=O)(=O)N2CC3(CN(C4CCOCC4)C3)C2)n(C)n1]

To a mixture of 2-tetrahydropyran-4-yl-2,6-diazaspiro[3.3]heptane, 2HCl (18 mg, 0.071 mmol) and 5-isopropyl-2-methyl-pyrazole-3-sulfonyl chloride (17 mg, 0.074 mmol) in DCM (5 mL) was added Hunigs base (49 mL, 0.28 mmol). The reaction mixture was stirred at rt for 3 h. It was quanched with satd. NaHCO₃, added water, and stirred at rt for 5 min. The organic phase was washed with water and concentrated. The residue was purified by normal phase column chromatography on silica gel (12 g, EtOAc/EtOH 3/1 in heptane 50-100%) to get 2-((3-isopropyl-1-methyl-1H-pyrazol-5-yl)sulfonyl)-6-(tetrahydro-2H-pyran-4-yl)-2,6-diazaspiro[3.3]heptane as a colorless oil after lyophilization. LCMS m/z=369.2 (M+H)⁺. ¹H NMR (400 MHz, CD₃OD) δ (ppm): 6.58 (s, 1H), 3.92 (s, 3H), 3.75-3.88 (m, 7H), 3.23-3.28 (m, 1H), 3.15 (s, 4H), 2.87 (quin, J=7.0 Hz, 1H), 2.13 (tt, J=10.7, 4.1 Hz, 1H), 1.46-1.63 (m, 2H), 1.16 (d, J=7.0 Hz, 6H), 1.03-1.14 (m, 2H).

### Example 190: 2-((2-cyclopropyl-6-(trifluoromethyl)pyridin-3-yl)sulfonyl)-6-((tetrahydro-2H-pyran-4-yl)methyl)-2,6-diazaspiro[3.3]heptane

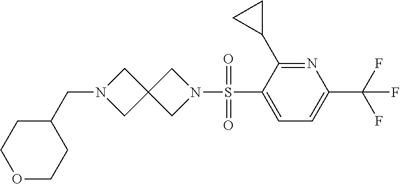 [SMILES: O=S(=O)(c1ccc(C(F)(F)F)nc1C1CC1)N1CC2(CN(CC3CCOCC3)C2)C1]

### 4. Synthesis of 2-cyclopropyl-6-(trifluoromethyl)pyridin-3-amine

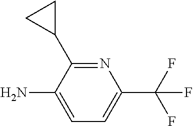 [SMILES: Nc1ccc(C(F)(F)F)nc1C1CC1]

To a solution of 2-iodo-6-(trifluoromethyl)pyridin-3-amine 0.7 g, 51 mmol) in toluene (150 mL) and water (50 mL) was added cyclopropylboronic acid (21.9 g, 255 mmol), K₃PO₄ (32.5 g, 153 mmol), S-Phos (2.1 g, 5.1 mmol), and Pd₂(dba)₃ (2.3 g, 2.5 mmol). The mixture was stirred at 70° C. under nitrogen. After 16 hours, the mixture was cooled to room temperature then filtered. The solution was diluted with brine then extracted with ethyl acetate. After drying over anhydrous sodium sulfate and concentration under vacuum, the residue was purified by a silica gel column eluting with dichloromethane/petroleum ether (1/10) to afford a brown oil as 2-cyclopropyl-6-(trifluoromethyl)pyridin-3-amine (9.5 g, 92%). LCMS m/z=203.0 (M+H)⁺. ¹H NMR (400 MHz, CDCl₃) δ (ppm) 7.35 (d, J=8.8 Hz, 1H), 6.93 (d, J=7.6 Hz, 1H), 4.46 (br s, 2H), 1.80-1.84 (m, 1H), 0.97-1.05 (m, 4H).

### 5. Synthesis of 2-cyclopropyl-6-(trifluoromethyl)pyridine-3-sulfonyl chloride

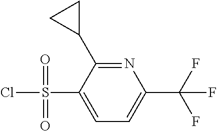 [SMILES: O=S(=O)(Cl)c1ccc(C(F)(F)F)nc1C1CC1]

Thionyl chloride (12 mL, 170 mmol) was slowly added to water (76 mL) at 0° C. During the addition of water, the temperature was maintained between 0-5° C. After addition of water, the solution was warmed to 15° C., then copper chloride (1.2 g, 12 mmol) was added. The solution was diluted with additional water (48 mL) and cooled back to 0° C. A solution of sodium nitrite (2.7 g, 40 mmol) in water (48 mL) was slowly added to a solution of 2-cyclopropyl-6-(trifluoromethyl)pyridin-3-amine (8 g, 40 mmol) in conc. HCl (64 mL) at 0° C. During addition, the temperature was maintained between 0-5° C. This mixture was slowly added to the above prepared solution to maintain a temperature between 0-5° C. A voluminous precipitate formed. The mixture was stirred for an additional 30 min after addition, then the solid was collected by filtration. The filter cake was washed with water (300 mL×2) to afford an orange solid as 2-cyclopropyl-6-(trifluoromethyl)pyridine-3-sulfonyl chloride (5 g, 44%). ¹H NMR (400 MHz, CDCl₃) δ (ppm) 8.43 (d, J=8.4 Hz, 1H), 7.58 (d, J=8.4 Hz, 1H), 2.98-3.04 (m, 1H), 1.44-1.46 (m, 2H), 1.32-1.35 (m, 2H).

### 6. Synthesis of tert-butyl 6-((2-cyclopropyl-6-(trifluoromethyl)pyridin-3-yl)sulfonyl)-2,6-diazaspiro[3.3]heptane-2-carboxylate

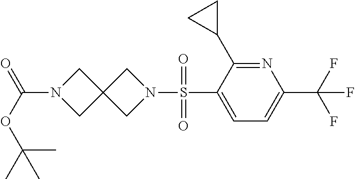 [SMILES: CC(C)(C)OC(=O)N1CC2(C1)CN(S(=O)(=O)c1ccc(C(F)(F)F)nc1C1CC1)C2]

To a vial containing tert-butyl 2,6-diazaspiro[3.3]heptane-2-carboxylate hydrochloride (474 mg, 2.0 mmol) in anhydrous dichloromethane (10 mL) was added Hunigs Base (1.6 mL, 9.2 mmol) carefully dropwise at <5° C. After 5 minutes, 2-cyclopropyl-6-(trifluoromethyl)pyridine-3-sulfonyl chloride (711 mg, 2.5 mmol) was added carefully to the cold solution. Upon complete addition of sulfonyl chloride, the reaction was warmed to 23° C. and monitored with LCMS. After 1 hour, the reaction was carefully quenched with slow addition of aqueous saturated sodium bicarbonate solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (5-45% ethyl acetate in heptane.) The desired fractions were pooled then concentrated under reduced pressure to afford a white solid as tert-butyl 6-((2-cyclopropyl-6-(trifluoromethyl)pyridin-3-yl)sulfonyl)-2,6-diazaspiro[3.3]heptane-2-carboxylate (888 mg, 98%) that was used without further purification. ¹H NMR (500 MHz, DICHLOROMETHANE-d₂) δ (ppm) 8.31 (d, J=8.2 Hz, 1H), 7.53 (d, J=8.2 Hz, 1H), 4.05 (s, 4H), 3.97 (s, 4H), 2.88 (ddd, J=3.2, 4.7, 8.0 Hz, 1H), 1.39 (s, 9H), 1.32-1.29 (m, 2H), 1.20-1.16 (m, 2H).

### 7. Synthesis of 2-((2-cyclopropyl-6-(trifluoromethyl)pyridin-3-yl)sulfonyl)-2,6-diazaspiro[3.3]heptane trifluoroacetate

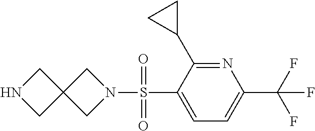 [SMILES: O=S(=O)(c1ccc(C(F)(F)F)nc1C1CC1)N1CC2(CNC2)C1]

To a flask containing tert-butyl 6-((2-cyclopropyl-6-(trifluoromethyl)pyridin-3-yl)sulfonyl)-2,6-diazaspiro[3.3]heptane-2-carboxylate (888 mg, 2 mmol) in HFiPA (6 mL) was added TFA (0.6 mL, 7.8 mmol) carefully dropwise at <5° C. Upon complete addition of TFA, the mixture was warmed to room temperature and monitored with LCMS. After 19 hours, the mixture was concentrated under reduced pressure to afford a dark yellow residue that was triturated with methanol to afford a white solid as 2-((2-cyclopropyl-6-(trifluoromethyl)pyridin-3-yl)sulfonyl)-2,6-diazaspiro[3.3]heptane trifluoroacetate that was used without purification. LCMS m/z=348.2 (M+H)⁺.

### 8. Synthesis of 2-((2-cyclopropyl-6-(trifluoromethyl)pyridin-3-yl)sulfonyl)-6-((tetrahydro-2H-pyran-4-yl)methyl)-2,6-diazaspiro[3.3]heptane

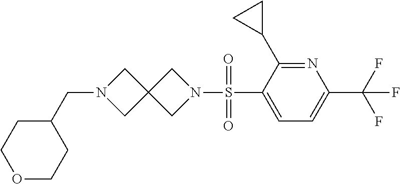 [SMILES: O=S(=O)(c1ccc(C(F)(F)F)nc1C1CC1)N1CC2(CN(CC3CCOCC3)C2)C1]

A vial containing 2-((2-cyclopropyl-6-(trifluoromethyl)pyridin-3-yl)sulfonyl)-2,6-diazaspiro[3.3]heptane trifluoroacetate (80 mg, 174 μmol) in anhydrous methanol (1 mL) was cooled in an ice water bath, then Hunigs Base (0.13 mL, 746 μmol) was added carefully to free base the starting material. After 20 minutes, tetrahydropyran-4-carbaldehyde (46 mg, 401 μmol) and acetic acid (0.05 mL, 873 μmol) were carefully added to the cooled mixture. After 15 minutes, sodium triacetoxyborohydride (214 mg, 1.0 mmol) was added carefully in portions to the cooled reaction solution. Upon complete addition of STAB, the reaction was maintained at <5° C. and monitored with LCMS. After 1.5 hours, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (20-70% 3:1 ethyl acetate:ethanol in heptane.) The desired fractions were pooled then concentrated under reduced pressure to afford a colorless film as 2-((2-cyclopropyl-6-(trifluoromethyl)pyridin-3-yl)sulfonyl)-6-((tetrahydro-2H-pyran-4-yl)methyl)-2,6-diazaspiro[3.3]heptane (36 mg, 44%). LCMS m/z=446.4 (M+H)⁺. ¹H NMR (500 MHz, DMSO-d₆) δ (ppm) 8.39 (d, J=8.2 Hz, 1H), 7.84 (d, J=7.9 Hz, 1H), 3.99 (s, 4H), 3.77 (br dd, J=2.9, 11.1 Hz, 2H), 3.20 (br t, J=11.4 Hz, 2H), 3.13 (s, 4H), 2.91-2.86 (m, 1H), 2.15 (br d, J=6.7 Hz, 2H), 1.48 (br d, J=12.8 Hz, 2H), 1.43-1.36 (m, 1H), 1.24-1.18 (m, 2H), 1.17-1.12 (m, 2H), 1.11-1.02 (m, 2H).

### Example 191: 2-((2-methyl-6-(1-(trifluoromethyl)cyclopropyl)pyridin-3-yl)sulfonyl)-6-((tetrahydro-2H-pyran-4-yl)methyl)-2,6-diazaspiro[3.3]heptane

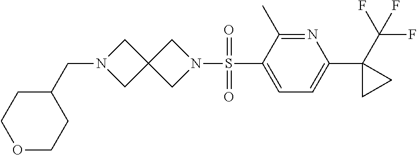 [SMILES: Cc1nc(C2(C(F)(F)F)CC2)ccc1S(=O)(=O)N1CC2(CN(CC3CCOCC3)C2)C1]

### 1. Synthesis of tert-butyl 6-((tetrahydro-2H-pyran-4-yl)methyl)-2,6-diazaspiro[3.3]heptane-2-carboxylate

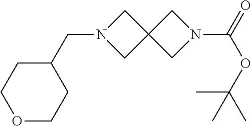 [SMILES: CC(C)(C)OC(=O)N1CC2(CN(CC3CCOCC3)C2)C1]

A flask containing tert-butyl 2,6-diazaspiro[3.3]heptane-2-carboxylate hydrochloride (1.0 g, 4.5 mmol) in anhydrous methanol (25 mL) was cooled in an ice water bath, then Hunigs Base (2.4 mL, 13.8 mmol) was added carefully dropwise. After 10 minutes, tetrahydropyran-4-carbaldehyde (1.1 g, 9.6 mmol) and acetic acid (1.1 mL, 19.2 mmol) were added carefully at <5° C. After 10 minutes, sodium triacetoxyborohydride (4.1 g, 19.5 mmol) was added carefully in portions to the cooled reaction solution. Upon complete addition of STAB, the reaction was warmed to 23° C. and monitored with LCMS. After 2 hours, the reaction was carefully quenched with slow addition of aqueous saturated sodium bicarbonate solution. The mixture was stirred at 23° C. for 30 minutes, then the mixture was extracted three times with ethyl acetate. The organic extractions were pooled then dried over anhydrous sodium sulfate. After filtration and concentration under reduced pressure, the colorless residue was loaded onto a silica gel column and purified with (50-100% 3:1 ethyl acetate:ethanol in heptane.) The desired fractions were pooled then concentrated under reduced pressure to afford a colorless film that solidified into a white solid as tert-butyl 6-((tetrahydro-2H-pyran-4-yl)methyl)-2,6-diazaspiro[3.3]heptane-2-carboxylate (1.1 g, 87%) that was used without further purification. LCMS m/z=297.3 (M+H)⁺. ¹H NMR (500 MHz, DMSO-d₆) δ (ppm) 3.86 (br s, 4H), 3.78 (br dd, J=2.7, 11.3 Hz, 2H), 3.22 (dt, J=1.7, 11.7 Hz, 2H), 3.20-3.12 (m, 4H), 2.17 (d, J=6.7 Hz, 1H), 1.90 (s, 1H), 1.51 (br d, J=13.1 Hz, 2H), 1.43 (dtd, J=3.7, 7.4, 14.6 Hz, 1H), 1.35 (s, 9H), 1.08 (dq, J=4.4, 12.2 Hz, 2H).

### 2. Synthesis of 2-((tetrahydro-2H-pyran-4-yl)methyl)-2,6-diazaspiro[3.3]heptane trifluoroacetate

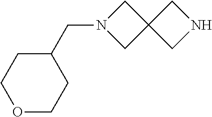 [SMILES: C1CC(CN2CC3(CNC3)C2)CCO1]

To a flask containing tert-butyl 6-((tetrahydro-2H-pyran-4-yl)methyl)-2,6-diazaspiro[3.3]heptane-2-carboxylate (1.1 g, 3.9 mmol) in HFiPA (11 mL) was added TFA (1.1 mL, 14 mmol) carefully dropwise at <5° C. Upon complete addition of TFA, the mixture was warmed to room temperature and monitored with LCMS. After 18 hours, the mixture was concentrated under reduced pressure to afford a yellow film as 2-((tetrahydro-2H-pyran-4-yl)methyl)-2,6-diazaspiro[3.3]heptane trifluoroacetate that was used without purification. LCMS m/z=197.3 (M+H)⁺.

### 3. Synthesis of 2-((2-methylpyridin-3-yl)sulfonyl)-6-((tetrahydro-2H-pyran-4-yl)methyl)-2,6-diazaspiro[3.3]heptane

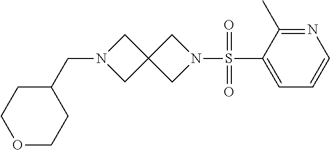 [SMILES: Cc1ncccc1S(=O)(=O)N1CC2(CN(CC3CCOCC3)C2)C1]

To a vial containing 2-((tetrahydro-2H-pyran-4-yl)methyl)-2,6-diazaspiro[3.3]heptane trifluoroacetate (511 mg, 1.6 mmol) in anhydrous dichloromethane (10 mL) was added Hunigs Base (1.3 mL, 7.5 mmol) carefully dropwise at room temperature. After 10 minutes, 2-methylpyridine-3-sulfonyl chloride (406 mg, 2.1 mmol) was added carefully to the homogeneous mixture. Upon complete addition of sulfonyl chloride, the reaction was maintained at 23° C. and monitored with LCMS. After 19 hours, the reaction mixture was cooled to room temperature then carefully quenched with slow addition of aqueous saturated sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (55-100% 3:1 ethyl acetate:ethanol in heptane.) The desired fractions were pooled then concentrated under reduced pressure to afford a faint yellow film as 2-((2-methylpyridin-3-yl)sulfonyl)-6-((tetrahydro-2H-pyran-4-yl)methyl)-2,6-diazaspiro[3.3]heptane (256 mg, 42%) that was used without further purification. LCMS m/z=352.3 (M+H)⁺. ¹H NMR (500 MHz, DMSO-d₆) δ (ppm) 8.72 (br d, J=4.6 Hz, 1H), 8.17 (d, J=7.9 Hz, 1H), 7.49 (dd, J=5.0, 7.8 Hz, 1H), 3.92 (br s, 4H), 3.78 (br dd, J=2.9, 10.8 Hz, 2H), 3.49-3.33 (m, 3H), 3.21 (br t, J=11.4 Hz, 4H), 2.74 (s, 3H), 2.43-2.18 (m, 1H), 1.49 (br d, J=12.5 Hz, 3H), 1.13-1.04 (m, 2H).

### 4. Synthesis of 2-((2-methyl-6-(1-(trifluoromethyl)cyclopropyl)pyridin-3-yl)sulfonyl)-6-((tetrahydro-2H-pyran-4-yl)methyl)-2,6-diazaspiro[3.3]heptane

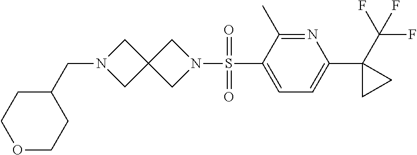 [SMILES: Cc1nc(C2(C(F)(F)F)CC2)ccc1S(=O)(=O)N1CC2(CN(CC3CCOCC3)C2)C1]

A vial containing 2-((2-methylpyridin-3-yl)sulfonyl)-6-((tetrahydro-2H-pyran-4-yl)methyl)-2,6-diazaspiro[3.3]heptane (47 mg, 135 μmol) and sodium 1-(trifluoromethyl)cyclopropanesulfinate (169 mg, 861 μmol) in diethyl carbonate (0.8 mL) and water (0.6 mL) was cooled in an ice water bath, then 2-hydroperoxy-2-methyl-propane, 70 wt % in water (0.2 mL, 1.4 mmol) was added carefully dropwise at <5° C. After 10 minutes, the reaction was carefully heated to 85° C. and monitored with LCMS. After 1.5 hours, the reaction mixture was cooled to room temperature then diluted with dichloromethane. The mixture was carefully quenched with slow addition of saturated, aqueous sodium bicarbonate. The organic layer was dried over anhydrous magnesium sulfate. After filtration and concentration under reduced pressure, the residue was loaded onto a silica gel column and purified with (25-90% 3:1 ethyl acetate:ethanol in heptane.) The desired fractions were pooled then concentrated under reduced pressure to afford a colorless film that was dissolved in DMSO and few drops of water then filtered. The homogeneous solution was submitted for mass directed reverse phase HPLC purification. Liquid chromatography was performed using a Waters XSelect CSH C18, 5 μm, 30 mm×50 mm column with mobile phase H₂O (A) and MeCN (B) and a gradient of 5-60% B (0.2% NH₄OH final v/v % modifier) with flow rate at 60 mL/min. The desired fractions were pooled then concentrated under reduced pressure to afford a colorless film as 2-[[2-methyl-6-[1-(trifluoromethyl)cyclopropyl]-3-pyridyl]sulfonyl]-6-(tetrahydropyran-4-ylmethyl)-2,6-diazaspiro[3.3]heptane (2.8 mg, 4%). LCMS m/z=460.3 (M+H)⁺. ¹H NMR (500 MHz, DICHLOROMETHANE-d₂) δ (ppm) 8.09 (d, J=8.5 Hz, 1H), 7.51 (d, J=8.2 Hz, 1H), 3.92 (s, 4H), 3.86 (br dd, J=3.1, 11.0 Hz, 2H), 3.29 (dt, J=1.5, 11.7 Hz, 2H), 3.20 (s, 4H), 2.74 (s, 3H), 2.20 (br d, J=6.7 Hz, 2H), 1.57-1.52 (m, 4H), 1.49-1.42 (m, 3H), 1.21-1.13 (m, 2H).

### Example 192: 2-((2-chloro-6-(trifluoromethyl)pyridin-3-yl)sulfonyl)-6-(tetrahydro-2H-pyran-4-yl)-2,6-diazaspiro[3.3]heptane

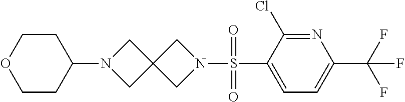 [SMILES: O=S(=O)(c1ccc(C(F)(F)F)nc1Cl)N1CC2(CN(C3CCOCC3)C2)C1]

To a vial containing 2-(tetrahydro-2H-pyran-4-yl)-2,6-diazaspiro[3.3]heptane hydrochloride (529 mg, 2.4 mmol) in anhydrous dichloromethane (10 mL) was added Hunigs Base (1.8 mL, 10 mmol) carefully dropwise at <5° C. After 5 minutes, 2-chloro-6-(trifluoromethyl)pyridine-3-sulfonyl chloride (820 mg, 2.9 mmol) was added carefully to the cold solution. Upon complete addition of sulfonyl chloride, the reaction was warmed to 23° C. and monitored with LCMS and TLC. After 30 minutes, the reaction was carefully quenched with slow addition of saturated, aqueous sodium bicarbonate solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (15-75% 3:1 ethyl acetate:ethanol in heptane.) The desired fractions were pooled then concentrated under reduced pressure to afford a white solid as 2-((2-chloro-6-(trifluoromethyl)pyridin-3-yl)sulfonyl)-6-(tetrahydro-2H-pyran-4-yl)-2,6-diazaspiro[3.3]heptane (624 mg, 58%). LCMS m/z=426.3 (M+H)⁺. ¹H NMR (500 MHz, DICHLOROMETHANE-d₂) δ (ppm) 8.50 (d, J=7.9 Hz, 1H), 7.77 (d, J=7.9 Hz, 1H), 4.19 (s, 4H), 3.85 (td, J=3.5, 11.5 Hz, 2H), 3.33-3.28 (m, 2H), 3.27-3.21 (m, 4H), 2.14-2.07 (m, 1H), 1.56 (br d, J=12.8 Hz, 2H), 1.24-1.16 (m, 2H).

### Example 193: 2-((2-cyclopropyl-6-(trifluoromethyl)pyridin-3-yl)sulfonyl)-6-(tetrahydro-2H-pyran-4-yl)-2,6-diazaspiro[3.3]heptane

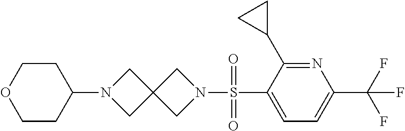 [SMILES: O=S(=O)(c1ccc(C(F)(F)F)nc1C1CC1)N1CC2(CN(C3CCOCC3)C2)C1]

A vial containing 2-((2-chloro-6-(trifluoromethyl)pyridin-3-yl)sulfonyl)-6-(tetrahydro-2H-pyran-4-yl)-2,6-diazaspiro[3.3]heptane (181 mg, 425 μmol), potassium cyclopropyltrifluoroborate (200 mg, 1.5 mmol), tricyclohexylphosphine (42 mg, 151 μmol), Pd₂dba₃ (46 mg, 50 μmol), Pd(dppf)Cl₂ CH₂Cl₂ (73 mg, 89 μmol), and potassium carbonate (277 mg, 2.0 mmol) in Dioxane (4 mL) and water (0.4 mL) was degassed and backfilled with nitrogen. The heterogeneous reaction mixture was carefully heated to 85° C. and monitored with LCMS. After 19 hours, the heterogeneous reaction was cooled to room temperature then filtered through a celite plug. The filtrate was concentrated under reduced pressure, then the residue was loaded onto a silica gel column and purified with (10-65% 3:1 ethyl acetate:ethanol in heptane.) The desired fractions were pooled then concentrated under reduced pressure to afford a brown film that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-55% B (0.2% NH₄OH final v/v % modifier) with flow rate at 50 mL/min. Fractions containing desired product were pooled then concentrated under reduced pressure to afford a brown film as 2-((2-cyclopropyl-6-(trifluoromethyl)pyridin-3-yl)sulfonyl)-6-(tetrahydro-2H-pyran-4-yl)-2,6-diazaspiro[3.3]heptane (46 mg, 24%). LCMS m/z=432.2 (M+H)⁺. ¹H NMR (400 MHz, DICHLOROMETHANE-d₂) δ (ppm) 8.34-8.26 (m, 1H), 7.52 (d, J=8.0 Hz, 1H), 4.01 (s, 4H), 3.88-3.82 (m, 2H), 3.30 (dt, J=2.4, 11.2 Hz, 2H), 3.19 (s, 4H), 2.92 (tt, J=4.8, 8.0 Hz, 1H), 2.09 (tt, J=3.9, 9.8 Hz, 1H), 1.59-1.53 (m, 2H), 1.32-1.27 (m, 2H), 1.23-1.12 (m, 4H).

### Example 194: 2-((2-methyl-4-(trifluoromethyl)phenyl)sulfonyl)-6-((tetrahydro-2H-pyran-4-yl)methyl)-2,6-diazaspiro[3.3]heptane

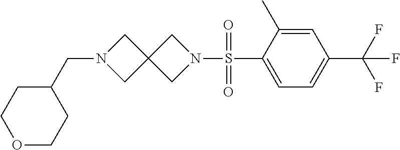 [SMILES: Cc1cc(C(F)(F)F)ccc1S(=O)(=O)N1CC2(CN(CC3CCOCC3)C2)C1]

To a vial containing 2-((tetrahydro-2H-pyran-4-yl)methyl)-2,6-diazaspiro[3.3]heptane trifluoroacetate (65 mg, 210 μmol) in anhydrous dichloromethane (1 mL) was added Hunigs Base (0.16 mL, 919 μmol) carefully dropwise at room temperature. After 10 minutes, 2-methyl-4-(trifluoromethyl)benzenesulfonyl chloride (73 mg, 283 μmol) was added carefully to the homogeneous mixture. Upon complete addition of sulfonyl chloride, the reaction was maintained at 23° C. and monitored with LCMS and TLC. After 1.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (35-95% 3:1 ethyl acetate:ethanol in heptane.) The desired fractions were pooled then concentrated under reduced pressure to afford a colorless film as 2-((2-methyl-4-(trifluoromethyl)phenyl)sulfonyl)-6-((tetrahydro-2H-pyran-4-yl)methyl)-2,6-diazaspiro[3.3]heptane (2.3 mg, 2%). LCMS m/z=419.1 (M+H)⁺. ¹H NMR (500 MHz, DICHLOROMETHANE-d₂) δ (ppm) 8.02 (d, J=8.2 Hz, 1H), 7.62-7.56 (m, 2H), 3.94 (s, 4H), 3.86 (br dd, J=3.1, 11.0 Hz, 2H), 3.36-3.19 (m, 6H), 2.66 (s, 3H), 2.23 (br s, 2H), 1.56-1.39 (m, 3H), 1.21-1.12 (m, 2H).

### Examples 195 and 196: 4-(6-((2-cyclopropyl-6-(trifluoromethyl)pyridin-3-yl)sulfonyl)-2,6-diazaspiro[3.3]heptan-2-yl)-1-methylcyclohexan-1-ol and 4-(6-((2-cyclopropyl-6-(trifluoromethyl)pyridin-3-yl)sulfonyl)-2,6-diazaspiro[3.3]heptan-2-yl)-1-methylcyclohexan-1-ol

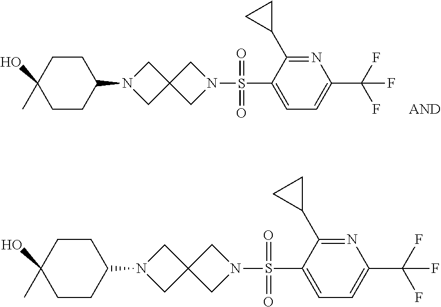

A vial containing 2-((2-cyclopropyl-6-(trifluoromethyl)pyridin-3-yl)sulfonyl)-2,6-diazaspiro[3.3]heptane trifluoroacetate (191 mg, 415 μmol) in anhydrous methanol (3 mL) was cooled in an ice water bath, then Hunigs Base (0.3 mL, 1.7 mmol) was added carefully to free base the starting material. After 20 minutes, 4-hydroxy-4-methyl-cyclohexanone (119 mg, 927 μmol) and acetic acid (0.13 mL, 2.3 mmol) were carefully added to the cooled mixture. After 15 minutes, sodium triacetoxyborohydride (510 mg, 2.4 mmol) was added carefully in portions to the cooled reaction solution. Upon complete addition of STAB, the reaction was maintained at <5° C. and monitored with LCMS. After 1.5 hours, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (20-65% 3:1 ethyl acetate:ethanol in heptane.) Two compounds were obtained as colorless film, stereochemistry was arbitrarily assigned:

First off column: 4-(6-((2-cyclopropyl-6-(trifluoromethyl)pyridin-3-yl)sulfonyl)-2,6-diazaspiro[3.3]heptan-2-yl)-1-methylcyclohexan-1-ol (92 mg, 46%). LCMS m/z=460.3 (M+H)⁺. ¹H NMR (500 MHz, DMSO-d₆) δ (ppm) 8.39 (br d, J=7.9 Hz, 1H), 7.84 (br d, J=8.2 Hz, 1H), 4.09 (br s, 1H), 3.98 (br s, 4H), 3.19-2.95 (m, 4H), 2.92-2.85 (m, 1H), 2.00-1.85 (m, 1H), 1.57-1.48 (m, 2H), 1.47-1.40 (m, 2H), 1.25-1.18 (m, 4H), 1.17-1.13 (m, 2H), 1.05-0.95 (m, 5H).

Second off column: 4-(6-((2-cyclopropyl-6-(trifluoromethyl)pyridin-3-yl)sulfonyl)-2,6-diazaspiro[3.3]heptan-2-yl)-1-methylcyclohexan-1-ol (58 mg, 29%). LCMS m/z=460.3 (M+H)⁺. LCMS m/z=460.3 (M+H)⁺. ¹H NMR (500 MHz, DMSO-d₆) δ (ppm) 8.40 (d, J=8.2 Hz, 1H), 7.85 (d, J=8.2 Hz, 1H), 3.99 (s, 4H), 3.95-3.90 (m, 1H), 3.19-3.16 (m, 1H), 3.14-3.07 (m, 3H), 2.93-2.87 (m, 1H), 1.88-1.67 (m, 1H), 1.47 (br d, J=11.3 Hz, 2H), 1.30 (br s, 2H), 1.25-1.20 (m, 3H), 1.19-1.11 (m, 5H), 1.05 (s, 3H).

### Examples 197: rac-2-((1-Methyl-3-(trifluoromethyl)-1H-pyrazol-5-yl)sulfonyl)-6-((tetrahydrofuran-3-yl)methyl)-2,6-diazaspiro[3.3]heptane AND (S)-2-((1-methyl-3-(trifluoromethyl)-1H-pyrazol-5-yl)sulfonyl)-6-((tetrahydrofuran-3-yl)methyl)-2,6-diazaspiro[3.3]heptane

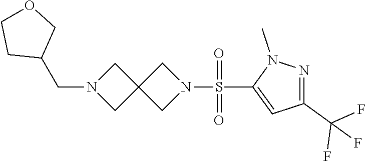 [SMILES: Cn1nc(C(F)(F)F)cc1S(=O)(=O)N1CC2(CN(CC3CCOC3)C2)C1]

### 1. Synthesis of tert-butyl 6-((1-methyl-3-(trifluoromethyl)-1H-pyrazol-5-yl)sulfonyl)-2,6-diazaspiro[3.3]heptane-2-carboxylate

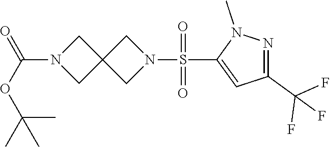 [SMILES: Cn1nc(C(F)(F)F)cc1S(=O)(=O)N1CC2(CN(C(=O)OC(C)(C)C)C2)C1]

To a vial containing tert-butyl 2,6-diazaspiro[3.3]heptane-2-carboxylate hydrochloride (1.2 g, 5.2 mmol) in anhydrous dichloromethane (20 mL) was added Hunigs Base (4.1 mL, 23 mmol) carefully dropwise at <5° C. After 5 minutes, 2-methyl-5-(trifluoromethyl)pyrazole-3-sulfonyl chloride (1.6 g, 6.5 mmol) was added carefully to the cold solution. Upon complete addition of sulfonyl chloride, the reaction was warmed to 23° C. and monitored with LCMS. After 1 hour, the reaction was carefully quenched with slow addition of aqueous 1 M sodium hydroxide solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (10-45% ethyl acetate in heptane.) The desired fractions were pooled then concentrated under reduced pressure to afford a white solid as tert-butyl 6-((1-methyl-3-(trifluoromethyl)-1H-pyrazol-5-yl)sulfonyl)-2,6-diazaspiro[3.3]heptane-2-carboxylate (1.4 g, 65%). ¹H NMR (500 MHz, DICHLOROMETHANE-d₂) δ (ppm) 7.02 (s, 1H), 4.13 (s, 3H), 4.03 (s, 4H), 3.98 (s, 4H), 1.39 (s, 9H).

### 2. Synthesis of 2-((1-methyl-3-(trifluoromethyl)-1H-pyrazol-5-yl)sulfonyl)-2,6-diazaspiro[3.3]heptane trifluoroacetate

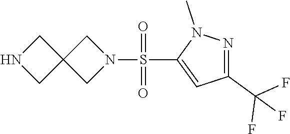 [SMILES: Cn1nc(C(F)(F)F)cc1S(=O)(=O)N1CC2(CNC2)C1]

To a vial containing tert-butyl 6-((1-methyl-3-(trifluoromethyl)-1H-pyrazol-5-yl)sulfonyl)-2,6-diazaspiro[3.3]heptane-2-carboxylate (1.3 g, 3.17 mmol) in anhydrous dichloromethane (11 mL) was added TFA (1 mL, 13 mmol) carefully dropwise at <5° C. Upon complete addition of TFA, the mixture was monitored with LCMS. After 3.5 hours, the mixture was concentrated under reduced pressure to afford a faint yellow oil that solidified into an off white solid as 2-((1-methyl-3-(trifluoromethyl)-1H-pyrazol-5-yl)sulfonyl)-2,6-diazaspiro[3.3]heptane trifluoroacetate that was used without purification. LCMS m/z=311.1 (M+H)⁺.

### 3. Synthesis of rac-2-((1-methyl-3-(trifluoromethyl)-H-pyrazol-5-yl)sulfonyl)-6-((tetrahydrofuran-3-yl)methyl)-2,6-diazaspiro[3.3]heptane

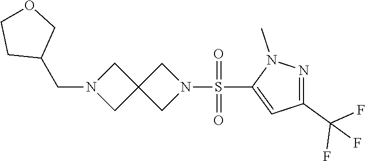 [SMILES: Cn1nc(C(F)(F)F)cc1S(=O)(=O)N1CC2(CN(CC3CCOC3)C2)C1]

A vial containing 2-((1-methyl-3-(trifluoromethyl)-1H-pyrazol-5-yl)sulfonyl)-2,6-diazaspiro[3.3]heptane trifluoroacetate (106 mg, 251 μmol) in anhydrous methanol (2 mL) was cooled in an ice water bath, then Hunigs Base (0.25 mL, 1.4 mmol) was added carefully to free base the starting material. After 20 minutes, tetrahydrofuran-3-carbaldehyde (121 mg, 605 μmol) and acetic acid (1.1 mL, 19 mmol) were carefully added to the cooled mixture. After 15 minutes, sodium triacetoxyborohydride (389 mg, 1.8 mmol) was added carefully in portions to the cooled reaction solution. Upon complete addition of STAB, the reaction was maintained at <5° C. and monitored with LCMS. After 1.5 hours, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a colorless film as rac-2-((1-methyl-3-(trifluoromethyl)-1H-pyrazol-5-yl)sulfonyl)-6-((tetrahydrofuran-3-yl)methyl)-2,6-diazaspiro[3.3]heptane (60 mg, 58%). LCMS m/z=395.3 (M+H)⁺. ¹H NMR (500 MHz, DICHLOROMETHANE-d₂) δ (ppm) 7.01 (s, 1H), 4.13 (s, 3H), 3.98 (br s, 4H), 3.78-3.71 (m, 2H), 3.64 (q, J=7.6 Hz, 1H), 3.34 (br t, J=6.9 Hz, 1H), 3.32-3.05 (m, 4H), 2.44-2.22 (m, 2H), 2.19-1.88 (m, 2H), 1.49 (br d, J=7.0 Hz, 1H).

### Example 198: rac-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane

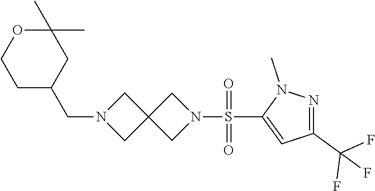 [SMILES: Cn1nc(C(F)(F)F)cc1S(=O)(=O)N1CC2(CN(CC3CCOC(C)(C)C3)C2)C1]

A vial containing 2-((1-methyl-3-(trifluoromethyl)-1H-pyrazol-5-yl)sulfonyl)-2,6-diazaspiro[3.3]heptane trifluoroacetate (122 mg, 393 μmol) in anhydrous methanol (2 mL) was cooled in an ice water bath, then Hunigs Base (0.4 mL, 2.3 mmol) was added carefully to free base the starting material. After 20 minutes, 2,2-dimethyltetrahydropyran-4-carbaldehyde (133 mg, 937 μmol) and acetic acid (0.16 mL, 2.8 mmol) were carefully added to the cooled mixture. After 15 minutes, sodium triacetoxyborohydride (488 mg, 2.3 mmol) was added carefully in portions to the cooled reaction solution. Upon complete addition of STAB, the reaction was maintained at <5° C. and monitored with LCMS. After 2 hours, the milky heterogenous reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (50-85% 3:1 ethyl acetate:ethanol in heptane.) The desired fractions were pooled then concentrated under reduced pressure to afford a colorless film as rac-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane (623 mg, 35%). LCMS m/z=437.4 (M+H)⁺. ¹H NMR (500 MHz, DICHLOROMETHANE-d₂) δ (ppm) 7.01 (s, 1H), 4.13 (s, 3H), 3.97 (s, 4H), 3.67-3.60 (m, 1H), 3.59-3.52 (m, 1H), 3.18 (br s, 4H), 2.15 (br s, 2H), 1.62 (br s, 1H), 1.56-1.47 (m, 2H), 1.13 (d, J=3.7 Hz, 6H), 1.12-0.89 (m, 2H).

### Examples 199 and 200: (R)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane AND (S)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane

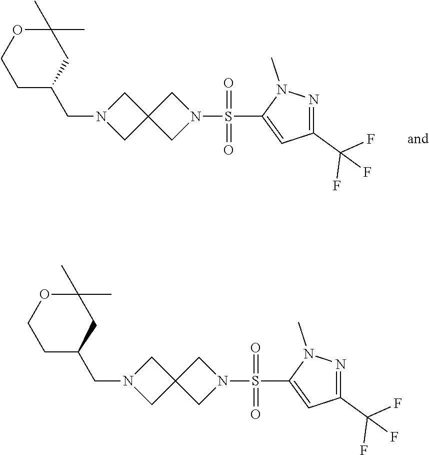 [SMILES: Cn1nc(C(F)(F)F)cc1S(=O)(=O)N1CC2(CN(C[C@@H]3CCOC(C)(C)C3)C2)C1 ; Cn1nc(C(F)(F)F)cc1S(=O)(=O)N1CC2(CN(C[C@H]3CCOC(C)(C)C3)C2)C1]

rac-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane (50 mg, 114 μmol) was dissolved in methanol (5 mL) then purified on a Chiralpak AD-H 30×250 mm, 5 um column using 20% methanol with 0.1% DEA modifier. Flow rate: 100 mL/min; ABPR 120 bar; MBPR 40 psi, column temperature 40° C. to afford the following compounds of arbitrarily assigned stereochemistry:

(R)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane (19 mg, 36%). LCMS m/z=437.4 (M+H)⁺, (E1 with Rf=1.12 min). ¹H NMR (400 MHz, DMSO-d₆) δ (ppm) 7.52 (s, 1H), 4.13 (s, 3H), 3.98 (s, 4H), 3.56-3.51 (m, 1H), 3.49-3.43 (m, 1H), 3.08 (s, 4H), 2.15-2.05 (m, 2H), 1.61-1.51 (m, 1H), 1.44 (dd, J=3.5, 13.0 Hz, 2H), 1.08 (s, 3H), 1.07 (s, 3H), 0.97-0.81 (m, 2H). and

(S)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane (19 mg, 36%). LCMS m/z=437.3 (M+H)⁺, (E2 with Rf=1.25 min). ¹H NMR (400 MHz, DMSO-d₆) δ (ppm) 7.52 (s, 1H), 4.13 (s, 3H), 3.98 (s, 4H), 3.56-3.43 (m, 2H), 3.09 (br s, 4H), 2.17-2.04 (m, 2H), 1.60-1.51 (m, 1H), 1.47-1.41 (m, 2H), 1.08 (s, 3H), 1.07 (s, 3H), 0.97-0.81 (m, 2H).

### Example 201: 2-((tetrahydro-2H-pyran-4-yl)methyl)-6-((2-(trifluoromethyl)pyridin-3-yl)sulfonyl)-2,6-diazaspiro[3.3]heptane

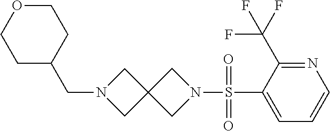 [SMILES: O=S(=O)(c1cccnc1C(F)(F)F)N1CC2(CN(CC3CCOCC3)C2)C1]

To a vial containing 2-((tetrahydro-2H-pyran-4-yl)methyl)-2,6-diazaspiro[3.3]heptane trifluoroacetate (317 mg, 1.02 mmol) in anhydrous dichloromethane (6 mL) was added Hunigs Base (0.72 mL, 4.13 mmol) carefully dropwise at room temperature. After 10 minutes, 2-(trifluoromethyl)pyridine-3-sulfonyl chloride (307 mg, 1.25 mmol) was added carefully to the homogeneous mixture. Upon complete addition of sulfonyl chloride, the reaction was maintained at 23° C. and monitored with LCMS and TLC. After 0.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a white solid as 2-((tetrahydro-2H-pyran-4-yl)methyl)-6-((2-(trifluoromethyl)pyridin-3-yl)sulfonyl)-2,6-diazaspiro[3.3]heptane (348 mg, 84%). LCMS m/z=406.1 [M+H]+. ¹H NMR (500 MHz, DICHLOROMETHANE-d₂) δ (ppm) 8.84 (br d, J=3.7 Hz, 1H), 8.44 (br d, J=7.9 Hz, 1H), 7.69 (br dd, J=4.6, 7.6 Hz, 1H), 4.07 (br s, 4H), 3.86 (br d, J=8.9 Hz, 2H), 3.47-3.02 (m, 6H), 2.47-2.06 (m, 2H), 1.53-1.42 (m, 2H), 1.24-1.13 (m, 3H).

### Example 202: 2-((1-methyl-3-(trifluoromethyl)-1H-pyrazol-5-yl)sulfonyl)-6-((tetrahydro-2H-pyran-4-yl)methyl)-2,6-diazaspiro[3.3]heptane

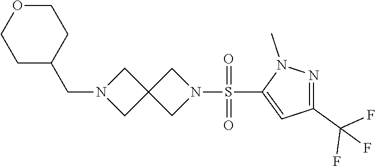 [SMILES: Cn1nc(C(F)(F)F)cc1S(=O)(=O)N1CC2(CN(CC3CCOCC3)C2)C1]

A vial containing 2-((1-methyl-3-(trifluoromethyl)-1H-pyrazol-5-yl)sulfonyl)-2,6-diazaspiro[3.3]heptane trifluoroacetate (122 mg, 393 μmol) in anhydrous methanol (2 mL) was cooled in an ice water bath, then Hunigs Base (0.4 mL, 2.3 mmol) was added carefully to free base the starting material. After 20 minutes, tetrahydropyran-4-carbaldehyde (102 mg, 891 μmol) and acetic acid (0.16 mL, 2.8 mmol) were carefully added to the cooled mixture. After 15 minutes, sodium triacetoxyborohydride (478 mg, 2.25 mmol) was added carefully in portions to the cooled reaction solution. Upon complete addition of STAB, the reaction was maintained at <5° C. and monitored with LCMS. After 2 hours, the milky heterogenous reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (50-100% 3:1 ethyl acetate:ethanol in heptane.) The desired fractions were pooled then concentrated under reduced pressure to afford a colorless film that was dissolved in DMSO and few drops of water then filtered. The homogeneous solution was submitted for mass directed reverse phase HPLC purification. Liquid chromatography was performed using a Waters Xselect CSH C18, 5 μm, 30 mm×100 mm column with mobile phase H₂O (A) and MeCN (B) and a gradient of 5-55% B (0.2% NH₄OH final v/v % modifier) with flow rate at 50 mL/min. The desired fractions were pooled then concentrated under reduced pressure to afford a colorless film as 2-((1-methyl-3-(trifluoromethyl)-1H-pyrazol-5-yl)sulfonyl)-6-((tetrahydro-2H-pyran-4-yl)methyl)-2,6-diazaspiro[3.3]heptane (40 mg, 23%). LCMS m/z=409.3 (M+H)⁺. ¹H NMR (500 MHz, DICHLOROMETHANE-d₂) δ (ppm) 7.01 (s, 1H), 4.13 (s, 3H), 3.97 (br s, 4H), 3.86 (br d, J=9.2 Hz, 2H), 3.29 (br t, J=11.6 Hz, 2H), 3.25-3.14 (m, 4H), 2.20 (br d, J=5.8 Hz, 2H), 1.54-1.40 (m, 3H), 1.23-1.12 (m, 2H).

### Example 203: 2-(tetrahydro-2H-pyran-4-yl)-6-((6-(trifluoromethyl)-2-vinylpyridin-3-yl)sulfonyl)-2,6-diazaspiro[3.3]heptane

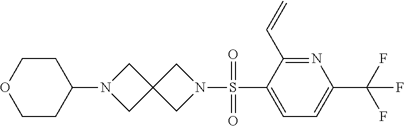 [SMILES: C=Cc1nc(C(F)(F)F)ccc1S(=O)(=O)N1CC2(CN(C3CCOCC3)C2)C1]

A vial containing 2-((2-chloro-6-(trifluoromethyl)pyridin-3-yl)sulfonyl)-6-(tetrahydro-2H-pyran-4-yl)-2,6-diazaspiro[3.3]heptane (152 mg, 357 μmol), potassium vinyltrifluoroborate (100 mg, 745 μmol), Pd(dppf)Cl₂ CH₂Cl₂ (68 mg, 83 μmol), and potassium carbonate (208 mg, 1.5 mmol) in Dioxane (2.5 mL) and water (0.25 mL) was degassed and backfilled with nitrogen. The heterogeneous reaction mixture was carefully heated to 90° C. and monitored with LCMS. After 23 hours, the heterogeneous reaction was cooled to room temperature then carefully partitioned between water and ethyl acetate. The aqueous layer was extracted two additional times with ethyl acetate. The organic extractions were pooled then washed once saturated aqueous sodium chloride solution, then the organic layer was dried over anhydrous sodium sulfate. After filtration and concentration under reduced pressure, the residue was loaded onto a silica gel column and purified with (40-80% 3:1 ethyl acetate:ethanol in heptane.) The desired fractions were pooled then concentrated under reduced pressure to afford a dark yellow film that was dissolved in DMSO and few drops of water then filtered. The homogeneous solution was submitted for mass directed reverse phase HPLC purification. Liquid chromatography was performed using a Waters Xselect CSH C18, 5 μm, 30 mm×100 mm column with mobile phase H₂O (A) and MeCN (B) and a gradient of 5-65% B (0.2% NH₄OH final v/v % modifier) with flow rate at 50 mL/min. The desired fractions were pooled then concentrated under reduced pressure to afford a colorless film as 2-(tetrahydro-2H-pyran-4-yl)-6-((6-(trifluoromethyl)-2-vinylpyridin-3-yl)sulfonyl)-2,6-diazaspiro[3.3]heptane (58 mg, 37%). LCMS m/z=418.3 (M+H)⁺. ¹H NMR (400 MHz, DMSO-d₆) δ (ppm) 8.48 (d, J=7.5 Hz, 1H), 8.02 (d, J=8.0 Hz, 1H), 7.51 (dd, J=10.8, 16.8 Hz, 1H), 6.59 (dd, J=2.0, 17.0 Hz, 1H), 5.86-5.82 (m, 1H), 3.96 (s, 4H), 3.75 (td, J=3.9, 11.3 Hz, 2H), 3.25-3.18 (m, 2H), 3.11 (s, 4H), 2.11-2.03 (m, 1H), 1.49 (br d, J=11.0 Hz, 2H), 1.09-0.99 (m, 2H).

### Example 204: 2-((2-ethyl-6-(trifluoromethyl)pyridin-3-yl)sulfonyl)-6-(tetrahydro-2H-pyran-4-yl)-2,6-diazaspiro[3.3]heptane

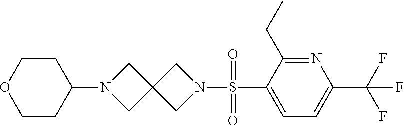 [SMILES: CCc1nc(C(F)(F)F)ccc1S(=O)(=O)N1CC2(CN(C3CCOCC3)C2)C1]

A flask containing 10% palladium on carbon (70 mg, 66 μmol) in ethyl alcohol (2 mL) was evacuated and backfilled with nitrogen. After 5 minutes a homogeneous solution of 2-(tetrahydro-2H-pyran-4-yl)-6-((6-(trifluoromethyl)-2-vinylpyridin-3-yl)sulfonyl)-2,6-diazaspiro[3.3]heptane (106 mg, 254 μmol) in ethyl alcohol (2 mL) was added under nitrogen. Upon complete addition, hydrogen was carefully introduced to the reaction mixture. The reaction was stirred at 23° C. and monitored with LCMS. After 20 hours, nitrogen was carefully bubbled through the reaction solution, then the heterogenous mixture was carefully filtered through a celite plug. The filtrate was concentrated under reduced pressure to afford a dark yellow film that was loaded onto a silica gel column and purified with (40-100% 3:1 ethyl acetate:ethanol in heptane.) The desired fractions were pooled then concentrated under reduced pressure to afford a dark yellow film that was dissolved in DMSO and few drops of water then filtered. The homogeneous solution was submitted for mass directed reverse phase HPLC purification. Liquid chromatography was performed using a Waters Xselect CSH C18, 5 μm, 30 mm×100 mm column with mobile phase H₂O (A) and MeCN (B) and a gradient of 5-65% B (0.2% NH₄OH final v/v % modifier) with flow rate at 50 mL/min. The desired fractions were pooled then concentrated under reduced pressure to afford a colorless film as 2-((2-ethyl-6-(trifluoromethyl)pyridin-3-yl)sulfonyl)-6-(tetrahydro-2H-pyran-4-yl)-2,6-diazaspiro[3.3]heptane (24 mg, 21%). LCMS m/z=420.3 (M+H)⁺. ¹H NMR (500 MHz, DICHLOROMETHANE-d₂) δ (ppm) 8.37 (d, J=8.2 Hz, 1H), 7.66 (d, J=8.2 Hz, 1H), 4.39-4.32 (m, 4H), 4.10-4.03 (m, 4H), 3.87-3.79 (m, 2H), 3.36-3.28 (m, 2H), 3.18 (q, J=7.5 Hz, 2H), 3.09-2.97 (m, 1H), 1.79 (br s, 4H), 1.35 (t, J=7.3 Hz, 3H).

### Example 205: 2-((6-(difluoromethyl)-2-ethylpyridin-3-yl)sulfonyl)-6-(tetrahydro-2H-pyran-4-yl)-2,6-diazaspiro[3.3]heptane

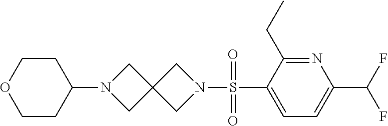 [SMILES: CCc1nc(C(F)F)ccc1S(=O)(=O)N1CC2(CN(C3CCOCC3)C2)C1]

### 1. Synthesis of 6-(difluoromethyl)-2-vinylpyridin-3-amine

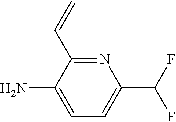

To a solution of compound 3-bromo-6-(difluoromethyl)-2-vinylpyridine (2.5 g, 11.21 mmol) and compound 4,4,5,5-tetramethyl-2-vinyl-1,3,2-dioxaborolane (3.45 g, 22.42 mmol, 3.80 mL) in water (3 mL) and Toluene (50 mL) was added Sphos (460.20 mg, 1.12 mmol), K₃PO₄ (7.14 g, 33.63 mmol) and Pd₂(dba)₃·CHCl₃ (1.03 g, 1.12 mmol) at 20° C. The mixture was stirred for 3 h at 100° C. under N₂. The reaction mixture was filtered and concentrated in vacuum. The crude was purified by flash column (EtOAc in petroleum ether from 0% to 20%) to afford the title compound (1.06 g, 6.23 mmol, 55.57% yield) as red oil. LCMS m/z=171.1 [M+H]+. ¹H NMR (400 MHz, CDCl₃) δ (ppm) 7.35 (d, J=8.4 Hz, 1H), 7.05 (d, J=8.4 Hz, 1H), 6.86-6.79 (m, 1H), 6.57 (t, J=56.0 Hz, 1H), 6.27 (dd, J=17.2 Hz, 2.0 Hz, 1H), 5.58 (dd, J=10.8 Hz, 1.6 Hz, 1H), 3.97 (br s, 2H).

### 2. Synthesis of 6-(difluoromethyl)-2-ethylpyridin-3-amine

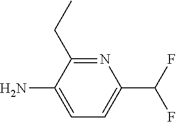

To a solution of compound 6-(difluoromethyl)-2-vinylpyridin-3-amine (1.06 g, 6.23 mmol) in MeOH (30 mL) was added Pd/C (300 mg, 281.90 μmol, 10% purity) at 20° C. The mixture was stirred at 20° C. under H₂ (15 psi) for 16 h. The reaction mixture was filtered and concentrated in vacuum to give the title compound (800 mg, crude) as yellow solid. LCMS m/z=173.1 [M+H]⁺.

### 3. Synthesis of 6-(difluoromethyl)-2-ethylpyridine-3-sulfonyl chloride

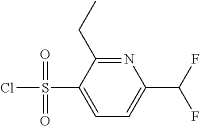

SOCl₂ (1.93 g, 16.26 mmol, 1.19 mL) was added over 10 min to water (1 mL), while the temperature was maintained 0-5° C., then the solution was stirred at 20° C. for 12 h. CuCl (23.00 mg, 232.32 μmol) was added and the mixture was cooled to −3° C. In another flask, a solution of compound 6-(difluoromethyl)-2-ethylpyridin-3-amine (400 mg, 2.32 mmol) in HCl (12 M, 1.70 mL) at −5° C. was added dropwise to the solution of NaNO₂ (160.29 mg, 2.32 mmol) in water (0.5 mL) while maintaining temperature −5 to 0° C. for 1 h. When the addition was complete, this solution was then added to the precooled SOCl₂ solution and stirred at −2° C. for 10 min, then at 0° C. for 75 min. The mixture was extracted with DCM (30 mL×3). The combined organic phase was dried over Na₂SO₄, filtered and concentrated to give the title compound (320 mg, crude) as yellow oil. ¹H NMR (400 MHz, CDCl₃) δ (ppm) 8.48 (d, J=8.5 Hz, 1H), 7.71 (d, J=8.0 Hz, 1H), 6.67 (t, J=55.0 Hz, 1H), 3.38 (q, J=7.5 Hz, 2H), 1.43 (t, J=7.5 Hz, 3H).

### 4. Synthesis of tert-butyl 6-((6-(difluoromethyl)-2-ethylpyridin-3-yl)sulfonyl)-2,6-diazaspiro[3.3]heptane-2-carboxylate

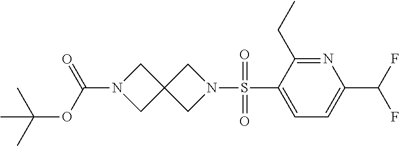 [SMILES: CCc1nc(C(F)F)ccc1S(=O)(=O)N1CC2(CN(C(=O)OC(C)(C)C)C2)C1]

To a solution of compound tert-butyl 2,6-diazaspiro[3.3]heptane-2-carboxylate (293.12 mg, 1.48 mmol) in DCM (20 mL) was added DIEA (955.39 mg, 7.39 mmol, 1.29 mL) and the mixture was stirred at 0° C. 6-(Difluoromethyl)-2-ethylpyridine-3-sulfonyl chloride (630 mg, 2.46 mmol) was added and the mixture was stirred at 20° C. under N₂ for 1 h. The mixture was diluted with water (30 mL) and extracted with DCM (30 mL×3). The combined organic phase was dried over anhydrous sodium sulfate, filtered and concentrated in vacuum. The residue was purified by flash column (EtOAc in petroleum ether from 5% to 20%) to give the title compound (430 mg, 1.03 mmol, 41.80% yield) as yellow solid. LCMS m/z=418.1 [M+H]⁺. ¹H NMR (400 MHz, CDCl₃) δ (ppm) 8.33 (d, J=8.0 Hz, 1H), 7.59 (d, J=8.4 Hz, 1H), 6.63 (t, J=55.2 Hz, 1H), 4.05 (s, 4H), 4.03 (s, 4H), 3.17 (q, J=7.2 Hz, 1H), 1.42 (s, 9H), 1.35 (t, J=7.6 Hz, 3H).

### 5. Synthesis of 2-((6-(difluoromethyl)-2-ethylpyridin-3-yl)sulfonyl)-2,6-diazaspiro[3.3]heptane

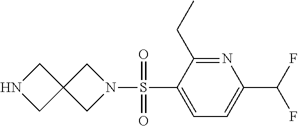 [SMILES: CCc1nc(C(F)F)ccc1S(=O)(=O)N1CC2(CNC2)C1]

To a solution of compound tert-butyl 6-((6-(difluoromethyl)-2-ethylpyridin-3-yl)sulfonyl)-2,6-diazaspiro[3.3]heptane-2-carboxylate (500 mg, 1.20 mmol) in HFIP (15 mL) was added TFA (682.82 mg, 5.99 mmol, 458.58 μL) and the mixture was stirred at 15° C. for 2 h The mixture was concentrated in vacuum to give the title compound (700 mg, crude, TFA salt) as yellow oil. LCMS m/z=318.1 [M+H]+. ¹H NMR (500 MHz, METHANOL-d₄) δ (ppm) 8.42 (d, J=8.0 Hz, 1H), 7.69 (d, J=8.4 Hz, 1H), 6.75 (t, J=55.2 Hz, 1H), 4.24 (s, 4H), 4.14 (s, 4H), 3.18 (q, J=7.2 Hz, 1H), 1.33 (t, J=7.6 Hz, 3H).

### 6. Synthesis of 2-((6-(difluoromethyl)-2-ethylpyridin-3-yl)sulfonyl)-6-(tetrahydro-2H-pyran-4-yl)-2,6-diazaspiro[3.3]heptane

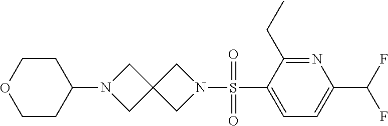 [SMILES: CCc1nc(C(F)F)ccc1S(=O)(=O)N1CC2(CN(C3CCOCC3)C2)C1]

To a solution of compound 2-((6-(difluoromethyl)-2-ethylpyridin-3-yl)sulfonyl)-2,6-diazaspiro[3.3]heptane (120 mg, 378.13 μmol, TFA salt) and compound tetrahydro-4H-pyran-4-one (75.71 mg, 756.25 μmol, 69.85 μL) in MeOH (3 mL) was added TEA (114.79 mg, 1.13 mmol, 158.11 μL) at 20° C. The mixture was stirred at 20° C. for 20 min, then adjusted pH=6 using acetic acid. After 30 minutes NaBH₃CN (118.81 mg, 1.89 mmol) was added at 20° C. and the mixture was stirred for 3 h. The mixture was concentrated and the resulting residue was purified by HPLC (Column: Welch Xtimate C18 150*25 mm*5 μm, Condition: water (NH₄HCO₃)-ACN, 28%˜58%, Flow Rate (mL/min): 25) to give the title compound (35.19 mg, 24% yield) as a white solid. LCMS m/z=402.1 [M+H]+. ¹H NMR (500 MHz, METHANOL-d₄) δ (ppm) 8.41 (d, J=8.4 Hz, 1H), 7.69 (d, J=8.4 Hz, 1H), 6.75 (t, J=55.2 Hz, 1H), 4.00 (s, 4H), 3.91 (d, J=10.4 Hz, 2H), 3.38-3.33 (m, 6H), 3.18 (q, J=7.6 Hz, 2H), 2.30-2.22 (m, 1H), 1.66 (dd, J=12.4, 1.6 Hz, 2H), 1.33 (t, J=7.6 Hz, 3H), 1.27-1.16 (m, 2H).

### Example 206: 2-((6-(difluoromethyl)-2-ethylpyridin-3-yl)sulfonyl)-6-(2-oxaspiro[3.3]heptan-6-yl)-2,6-diazaspiro[3.3]heptane

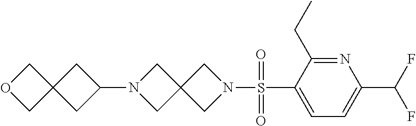 [SMILES: CCc1nc(C(F)F)ccc1S(=O)(=O)N1CC2(CN(C3CC4(COC4)C3)C2)C1]

2-((6-(Difluoromethyl)-2-ethylpyridin-3-yl)sulfonyl)-6-(2-oxaspiro[3.3]heptan-6-yl)-2,6-diazaspiro[3.3]heptane was prepared was prepared using a similar method to step 6 for Example 205 starting from 2-((6-(difluoromethyl)-2-ethylpyridin-3-yl)sulfonyl)-2,6-diazaspiro[3.3]heptane (120 mg, 278.18 μmol, TFA salt) and 2-oxaspiro[3.3]heptan-6-one (31.19 mg, 278.18 μmol). The crude product was purified by prep-HPLC (Welch Xtimate C18 150*25 mm*5 μm, Condition: water (NH₄HCO₃)-ACN, Flow Rate (mL/min): 25) to give the title compound (50 mg, 44% yield) as light yellow oil. LCMS m/z=414.1 [M+H]+. ¹H NMR (400 MHz, CDCl₃) δ (ppm) 8.32 (d, J=8.0 Hz, 1H), 7.58 (d, J=8.0 Hz, 1H), 6.63 (t, J=55.2 Hz, 1H), 4.64 (s, 2H), 4.58 (s, 2H), 3.99 (s, 4H), 3.21-3.14 (m, 6H), 2.84-2.77 (m, 1H), 2.28-2.23 (m, 2H), 1.94-1.89 (m, 2H), 1.34 (t, J=7.6 Hz, 3H).

### Example 207 and 208: (1r,4r)-4-(6-((6-(difluoromethyl)-2-ethylpyridin-3-yl)sulfonyl)-2,6-diazaspiro[3.3]heptan-2-yl)-1-methylcyclohexan-1-ol and (1s,4s)-4-(6-((6-(difluoromethyl)-2-ethylpyridin-3-yl)sulfonyl)-2,6-diazaspiro[3.3]heptan-2-yl)-1-methylcyclohexan-1-ol

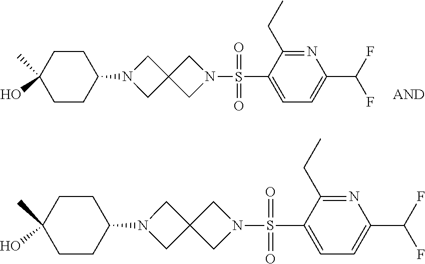

The title compounds were prepared using a similar method to step 6 for Example 205 starting from 2-((6-(difluoromethyl)-2-ethylpyridin-3-yl)sulfonyl)-2,6-diazaspiro[3.3]heptane (300 mg, 695 μmol, TFA salt) and 4-hydroxy-4-methylcyclohexan-1-one (89.13 mg, 695 μmol). The crude residue was purified by prep-HPLC (Welch Xtimate C18 150*25 mm*5 μm, Condition: water (NH₄HCO₃)-ACN, 33%˜60%, Flow Rate (mL/min): 25) to give two compounds of arbitrarily assigned stereochemistry as white solids:

First off column, (1r,4r)-4-(6-((6-(difluoromethyl)-2-ethylpyridin-3-yl)sulfonyl)-2,6-diazaspiro[3.3]heptan-2-yl)-1-methylcyclohexan-1-ol (50 mg, 16% yield, 97.64% purity). LCMS m/z=430.1 [M+H]+. ¹H NMR (400 MHz, CDCl₃) δ (ppm) 8.32 (d, J=8.4 Hz, 1H), 7.58 (d, J=8.4 Hz, 1H), 6.63 (t, J=55.2 Hz, 1H), 4.00 (s, 4H), 3.26 (s, 4H), 3.17 (q, J=7.6 Hz, 2H), 2.02-2.00 (m, 1H), 1.67-1.65 (m, 3H), 1.39-1.32 (m, 5H), 1.25-1.12 (m, 6H).

Second off column, (1s,4s)-4-(6-((6-(difluoromethyl)-2-ethylpyridin-3-yl)sulfonyl)-2,6-diazaspiro[3.3]heptan-2-yl)-1-methylcyclohexan-1-ol (86 mg, 28% yield) LCMS m/z=430.1 [M+H]+. ¹H NMR (400 MHz, CDCl₃) δ (ppm) 8.32 (d, J=8.4 Hz, 1H), 7.58 (d, J=8.4 Hz, 1H), 6.63 (t, J=55.2 Hz, 1H), 4.00 (s, 4H), 3.28 (s, 4H), 3.17 (q, J=7.6 Hz, 2H), 1.89-1.84 (m, 1H), 1.68-1.63 (m, 3H), 1.52-1.51 (m, 2H), 1.36-1.29 (m, 6H), 1.20 (s, 3H).

### Example 209: 2-((2-cyclopropyl-6-(trifluoromethyl)pyridin-3-yl)sulfonyl)-6-(oxetan-3-ylmethyl)-2,6-diazaspiro[3.3]heptane

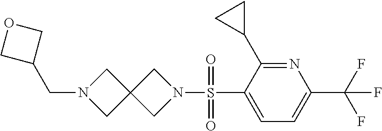 [SMILES: O=S(=O)(c1ccc(C(F)(F)F)nc1C1CC1)N1CC2(CN(CC3COC3)C2)C1]

A vial containing 2-((2-cyclopropyl-6-(trifluoromethyl)pyridin-3-yl)sulfonyl)-2,6-diazaspiro[3.3]heptane trifluoroacetate (92 mg, 199 μmol) in anhydrous methanol (2 mL) was cooled in an ice water bath, then Hunigs Base (0.15 mL, 861 μmol) was added carefully to free base the starting material. After 20 minutes, oxetane-3-carbaldehyde (37 mg, 426 μmol) and acetic acid (0.06 mL, 1.05 mmol) were carefully added to the cooled mixture. After 15 minutes, sodium triacetoxyborohydride (256 mg, 1.2 mmol) was added carefully in portions to the cooled reaction solution. Upon complete addition of STAB, the reaction was maintained at <5° C. and monitored with LCMS. After 1.5 hours, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (35-90% 3:1 ethyl acetate:ethanol in heptane.) The desired fractions were pooled then concentrated under reduced pressure to afford a colorless film as 2-((2-cyclopropyl-6-(trifluoromethyl)pyridin-3-yl)sulfonyl)-6-(oxetan-3-ylmethyl)-2,6-diazaspiro[3.3]heptane (54 mg, 62%). LCMS m/z=418.1 (M+H)⁺. ¹H NMR (500 MHz, DMSO-d₆) δ (ppm) 8.38 (d, J=7.9 Hz, 1H), 7.84 (d, J=7.9 Hz, 1H), 4.54 (br t, J=6.7 Hz, 2H), 4.17 (br t, J=6.0 Hz, 2H), 3.98 (s, 4H), 3.14 (br s, 4H), 2.91-2.81 (m, 2H), 2.56 (br d, J=6.7 Hz, 2H), 1.23-1.18 (m, 2H), 1.16-1.10 (m, 2H).

### Example 210: 2-((3-(1,1-difluoroethyl)-1-methyl-1H-pyrazol-5-yl)sulfonyl)-6-((tetrahydro-2H-pyran-4-yl)methyl)-2,6-diazaspiro[3.3]heptane

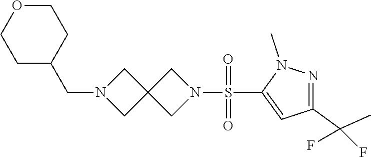 [SMILES: Cn1nc(C(C)(F)F)cc1S(=O)(=O)N1CC2(CN(CC3CCOCC3)C2)C1]

To a vial containing 2-((tetrahydro-2H-pyran-4-yl)methyl)-2,6-diazaspiro[3.3]heptane trifluoroacetate (58 mg, 188 μmol) in anhydrous dichloromethane (1 mL) was added Hunigs Base (0.15 mL, 861 μmol) carefully dropwise at room temperature. After 10 minutes, 5-(1,1-difluoroethyl)-2-methyl-pyrazole-3-sulfonyl chloride (55 mg, 226 μmol) was added carefully to the homogeneous mixture. Upon complete addition of sulfonyl chloride, the reaction was maintained at 23° C. and monitored with LCMS and TLC. After 1.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (40-90% 3:1 ethyl acetate:ethanol in heptane.) The desired fractions were pooled then concentrated under reduced pressure to afford a colorless film that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-50% B (0.2% NH₄OH final v/v % modifier) with flow rate at 50 mL/min. The desired fractions were pooled then concentrated under reduced pressure to afford a colorless film as 2-((3-(1,1-difluoroethyl)-1-methyl-1H-pyrazol-5-yl)sulfonyl)-6-((tetrahydro-2H-pyran-4-yl)methyl)-2,6-diazaspiro[3.3]heptane (5 mg, 6%). LCMS m/z=405.1 (M+H)⁺. ¹H NMR (500 MHz, DICHLOROMETHANE-d₂) δ (ppm) 6.90 (s, 1H), 4.08 (s, 3H), 3.94 (s, 4H), 3.86 (br dd, J=3.2, 11.1 Hz, 2H), 3.32-3.26 (m, 2H), 3.17 (br s, 4H), 2.20 (br d, J=6.7 Hz, 2H), 1.99 (t, J=18.5 Hz, 3H), 1.53 (br d, J=14.0 Hz, 2H), 1.49-1.41 (m, 1H), 1.21-1.12 (m, 2H).

### Example 211: 2-((2,4-bis(trifluoromethyl)phenyl)sulfonyl)-6-((tetrahydro-2H-pyran-4-yl)methyl)-2,6-diazaspiro[3.3]heptane

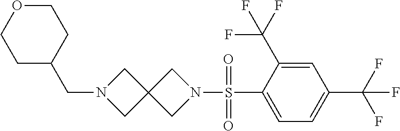 [SMILES: O=S(=O)(c1ccc(C(F)(F)F)cc1C(F)(F)F)N1CC2(CN(CC3CCOCC3)C2)C1]

To a vial 2-((tetrahydro-2H-pyran-4-yl)methyl)-2,6-diazaspiro[3.3]heptane trifluoroacetate (64 mg, 207 μmol) in anhydrous dichloromethane (1.5 mL) was added Hunigs Base (0.16 mL, 919 μmol) carefully dropwise at room temperature. After 10 minutes, 2,4-bis(trifluoromethyl)benzenesulfonyl chloride (69 mg, 220 μmol) was added carefully to the homogeneous mixture. Upon complete addition of sulfonyl chloride, the reaction was maintained at 23° C. and monitored with LCMS and TLC. After 1.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (25-75% 3:1 ethyl acetate:ethanol in heptane.) The desired fractions were pooled then concentrated under reduced pressure to afford a white solid as 2-((2,4-bis(trifluoromethyl)phenyl)sulfonyl)-6-((tetrahydro-2H-pyran-4-yl)methyl)-2,6-diazaspiro[3.3]heptane (8 mg, 8%). LCMS m/z=473.0 [M+H]+. ¹H NMR (500 MHz, DMSO-d₆) δ (ppm) 8.37-8.27 (m, 3H), 3.94 (br d, J=0.9 Hz, 4H), 3.83-3.70 (m, 2H), 3.22-3.14 (m, 6H), 2.19-2.14 (m, 2H), 1.49-1.37 (m, 3H), 1.08-1.02 (m, 2H).

### Example 212: rac-2-((1-methyl-3-(trifluoromethyl)-1H-pyrazol-5-yl)sulfonyl)-6-(1-(tetrahydro-2H-pyran-4-yl)ethyl)-2,6-diazaspiro[3.3]heptane

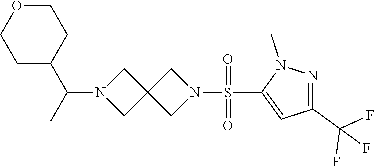 [SMILES: CC(C1CCOCC1)N1CC2(C1)CN(S(=O)(=O)c1cc(C(F)(F)F)nn1C)C2]

A vial containing 2-((1-methyl-3-(trifluoromethyl)-1H-pyrazol-5-yl)sulfonyl)-2,6-diazaspiro[3.3]heptane trifluoroacetate (126 mg, 296 μmol) in anhydrous methanol (3 mL) was cooled in an ice water bath, then Hunigs Base (0.22 mL, 1.3 mmol) was added carefully to free base the starting material. After 20 minutes, 1-tetrahydropyran-4-ylethanone (81 mg, 630 μmol) and acetic acid (0.11 mL, 1.9 mmol) were carefully added to the cooled mixture. After 15 minutes, sodium triacetoxyborohydride (488 mg, 2.3 mmol) was added carefully in portions to the cooled reaction solution. Upon complete addition of STAB, the reaction was maintained at <5° C. and monitored with LCMS. After 1.5 hours, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a colorless film as (R)-2-((1-methyl-3-(trifluoromethyl)-1H-pyrazol-5-yl)sulfonyl)-6-(1-(tetrahydro-2H-pyran-4-yl)ethyl)-2,6-diazaspiro[3.3]heptane AND (S)-2-((1-methyl-3-(trifluoromethyl)-1H-pyrazol-5-yl)sulfonyl)-6-(1-(tetrahydro-2H-pyran-4-yl)ethyl)-2,6-diazaspiro[3.3]heptane (116 mg, 88%). LCMS m/z=423.3 (M+H)⁺. ¹H NMR (500 MHz, DMSO-d₆) δ (ppm) 7.53 (s, 1H), 4.13 (s, 3H), 3.98 (s, 4H), 3.85-3.78 (m, 2H), 3.23 (dt, J=4.0, 10.7 Hz, 1H), 3.17-3.16 (m, 1H), 3.09-3.01 (m, 4H), 1.98-1.92 (m, 1H), 1.49-1.40 (m, 1H), 1.39-1.33 (m, 1H), 1.28-1.20 (m, 2H), 1.19-1.09 (m, 1H), 0.67 (d, J=6.4 Hz, 3H).

### Example 213: 2-((6-chloro-2-(trifluoromethyl)pyridin-3-yl)sulfonyl)-6-(tetrahydro-2H-pyran-4-yl)-2,6-diazaspiro[3.3]heptane

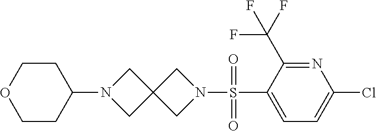 [SMILES: O=S(=O)(c1ccc(Cl)nc1C(F)(F)F)N1CC2(CN(C3CCOCC3)C2)C1]

To a vial containing 2-(tetrahydro-2H-pyran-4-yl)-2,6-diazaspiro[3.3]heptane hydrochloride (81 mg, 371 μmol) in anhydrous dichloromethane (2 mL) was added Hunigs Base (0.26 mL, 1.5 mmol) carefully dropwise at room temperature. After 10 minutes, 6-chloro-2-(trifluoromethyl)pyridine-3-sulfonyl chloride (145 mg, 517 μmol) was added carefully to the homogeneous mixture. Upon complete addition of sulfonyl chloride, the reaction was maintained at 23° C. and monitored with LCMS and TLC. After 1 hour, the reaction mixture was carefully quenched with slow addition of saturated, aqueous sodium bicarbonate. The heterogeneous mixture was extracted three times with dichloromethane. The organic extractions were pooled then dried over anhydrous magnesium sulfate. After filtration and concentration under reduced pressure, the residue was loaded onto a silica gel column and purified with (50-80% 3:1 ethyl acetate:ethanol in heptane.) The desired fractions were pooled then concentrated under reduced pressure to afford a white solid as 2-((6-chloro-2-(trifluoromethyl)pyridin-3-yl)sulfonyl)-6-(tetrahydro-2H-pyran-4-yl)-2,6-diazaspiro[3.3]heptane (32 mg, 19%). LCMS m/z=426.0 (M+H)⁺. ¹H NMR (500 MHz, DMSO-d₆) δ (ppm) 8.50 (d, J=8.5 Hz, 1H), 8.10 (d, J=8.2 Hz, 1H), 4.01 (s, 4H), 3.76 (br d, J=11.3 Hz, 2H), 3.22 (br t, J=10.7 Hz, 2H), 3.15-3.13 (m, 4H), 2.12-2.05 (m, 1H), 1.50 (br d, J=11.9 Hz, 2H), 1.10-1.01 (m, 2H).

### Example 214: 2-((2-fluoro-4-(trifluoromethoxy)phenyl)sulfonyl)-6-(tetrahydro-2H-pyran-4-yl)-2,6-diazaspiro[3.3]heptane

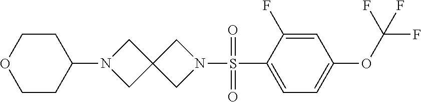 [SMILES: O=S(=O)(c1ccc(OC(F)(F)F)cc1F)N1CC2(CN(C3CCOCC3)C2)C1]

To a vial containing 2-(tetrahydro-2H-pyran-4-yl)-2,6-diazaspiro[3.3]heptane hydrochloride (60 mg, 273 μmol) in anhydrous dichloromethane (2 mL) was added Hunigs base (0.3 mL, 1.7 mmol) carefully dropwise at <5° C. After 5 minutes, 2-fluoro-4-(trifluoromethoxy)benzenesulfonyl chloride (101 mg, 364 μmol) was added carefully to the cold solution. Upon complete addition of sulfonyl chloride, the reaction was allowed to warm to 23° C. and monitored with LCMS and TLC. After 3 hours, the reaction was carefully quenched with slow addition of aqueous 1 M sodium hydroxide solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (40-80% 3:1 ethyl acetate:ethanol in heptane.) The desired fractions were pooled then concentrated under reduced pressure to afford a colorless film as 2-((2-fluoro-4-(trifluoromethoxy)phenyl)sulfonyl)-6-(tetrahydro-2H-pyran-4-yl)-2,6-diazaspiro[3.3]heptane (44 mg, 36%). LCMS m/z=425.3 (M+H)⁺. ¹H NMR (500 MHz, DICHLOROMETHANE-d₂) δ (ppm) 7.90 (t, J=8.1 Hz, 1H), 7.17 (br t, J=10.4 Hz, 2H), 3.98 (s, 4H), 3.84 (td, J=3.7, 11.5 Hz, 2H), 3.29 (dt, J=2.3, 11.2 Hz, 2H), 3.15 (s, 4H), 2.09-2.03 (m, 1H), 1.53 (br d, J=12.8 Hz, 2H), 1.22-1.14 (m, 2H).

### Example 215: rac-2-((1,4-dioxan-2-yl)methyl)-6-((1-methyl-3-(trifluoromethyl)-1H-pyrazol-5-yl)sulfonyl)-2,6-diazaspiro[3.3]heptane

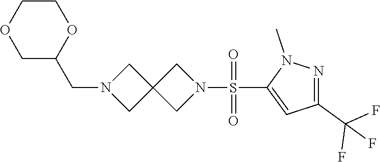 [SMILES: Cn1nc(C(F)(F)F)cc1S(=O)(=O)N1CC2(CN(CC3COCCO3)C2)C1]

A vial containing 2-((1-methyl-3-(trifluoromethyl)-1H-pyrazol-5-yl)sulfonyl)-2,6-diazaspiro[3.3]heptane trifluoroacetate (92 mg, 298 μmol) in anhydrous methanol (2 mL) was cooled in an ice water bath, then Hunigs Base (0.24 mL, 1.4 mmol) was added carefully to free base the starting material. After 20 minutes, 1,4-dioxane-2-carbaldehyde (74 mg, 608 μmol) and acetic acid (0.1 mL, 1.75 mmol) were carefully added to the cooled mixture. After 15 minutes, sodium triacetoxyborohydride (323 mg, 1.5 mmol) was added carefully in portions to the cooled reaction solution. Upon complete addition of STAB, the reaction was maintained at <5° C. and monitored with LCMS. After 1 hour, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (35-85% 3:1 ethyl acetate:ethanol in heptane.) The desired fractions were pooled then concentrated under reduced pressure to afford rac-2-((1,4-dioxan-2-yl)methyl)-6-((1-methyl-3-(trifluoromethyl)-1H-pyrazol-5-yl)sulfonyl)-2,6-diazaspiro[3.3]heptane (46 mg, 36%) as a white solid. LCMS m/z=411.0 [M+H]+. ¹H NMR (500 MHz, DMSO-d₆) δ (ppm) 7.53 (br s, 1H), 4.13 (br s, 3H), 3.98 (br s, 4H), 3.66-3.57 (m, 3H), 3.49 (br t, J=11.3 Hz, 1H), 3.43-3.34 (m, 2H), 3.20-3.10 (m, 5H), 2.29 (br s, 2H).

### Example 216: rac-2-((1,4-dioxan-2-yl)methyl)-6-((2-methyl-6-(trifluoromethyl)pyridin-3-yl)sulfonyl)-2,6-diazaspiro[3.3]heptane

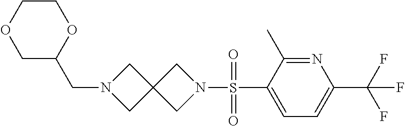 [SMILES: Cc1nc(C(F)(F)F)ccc1S(=O)(=O)N1CC2(CN(CC3COCCO3)C2)C1]

A vial containing 2-((2-methyl-6-(trifluoromethyl)pyridin-3-yl)sulfonyl)-2,6-diazaspiro[3.3]heptane trifluoroacetate (85 mg, 195 μmol) in anhydrous methanol (1 mL) was cooled in an ice water bath, then Hunigs Base (0.16 mL, 919 μmol) was added carefully to free base the starting material. After 20 minutes, 1,4-dioxane-2-carbaldehyde (50 mg, 409 μmol) and acetic acid (0.07 mL, 1.22 mmol) were carefully added to the cooled mixture. After 15 minutes, sodium triacetoxyborohydride (211 mg, 996 μmol) was added carefully in portions to the cooled reaction solution. Upon complete addition of STAB, the reaction was maintained at <5° C. and monitored with LCMS. After 1 hour, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (25-85% 3:1 ethyl acetate:ethanol in heptane.) The desired fractions were pooled then concentrated under reduced pressure to afford a colorless film as rac-2-((1,4-dioxan-2-yl)methyl)-6-((2-methyl-6-(trifluoromethyl)pyridin-3-yl)sulfonyl)-2,6-diazaspiro[3.3]heptane (41 mg, 48%). LCMS m/z=422.0 [M+H]⁺. ¹H NMR (500 MHz, DMSO-d₆) δ (ppm) 8.43 (br d, J=8.5 Hz, 1H), 7.98 (br d, J=7.6 Hz, 1H), 3.97 (br s, 4H), 3.67-3.57 (m, 3H), 3.49 (br t, J=11.1 Hz, 1H), 3.42-3.36 (m, 2H), 3.32-3.28 (m, 2H), 3.28-3.16 (m, 4H), 3.15-3.10 (m, 1H), 2.81 (s, 3H).

### Example 217: 2-((4-methyl-6-(trifluoromethyl)pyridin-3-yl)sulfonyl)-6-((tetrahydro-2H-pyran-4-yl)methyl)-2,6-diazaspiro[3.3]heptane

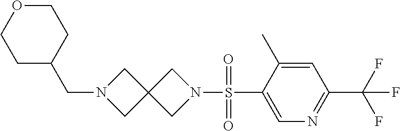 [SMILES: Cc1cc(C(F)(F)F)ncc1S(=O)(=O)N1CC2(CN(CC3CCOCC3)C2)C1]

To a vial containing 2-((tetrahydro-2H-pyran-4-yl)methyl)-2,6-diazaspiro[3.3]heptane trifluoroacetate (104 mg, 334 μmol) in anhydrous dichloromethane (2 mL) was added Hunigs Base (0.35 mL, 2.01 mmol) carefully dropwise at <5° C. After 5 minutes, 4-methyl-6-(trifluoromethyl)pyridine-3-sulfonyl chloride (122 mg, 471 μmol) was added carefully to the cold solution. Upon complete addition of sulfonyl chloride, the reaction was warmed to 23° C. and monitored with LCMS. After 3 hours, the reaction was carefully quenched with slow addition of aqueous saturated sodium bicarbonate solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a colorless film as 2-((4-methyl-6-(trifluoromethyl)pyridin-3-yl)sulfonyl)-6-((tetrahydro-2H-pyran-4-yl)methyl)-2,6-diazaspiro[3.3]heptane (20 mg, 13%). LCMS m/z=420.3 (M+H)⁺. ¹H NMR (500 MHz, DICHLOROMETHANE-d₂) δ (ppm) 9.07 (s, 1H), 7.67 (s, 1H), 4.59-4.50 (m, 2H), 4.40-4.36 (m, 2H), 4.06 (s, 2H), 3.93 (br dd, J=3.8, 11.4 Hz, 2H), 3.87-3.81 (m, 2H), 3.35 (t, J=11.3 Hz, 2H), 2.93-2.86 (m, 2H), 2.69 (s, 3H), 1.96-1.87 (m, 1H), 1.77 (br d, J=12.8 Hz, 2H), 1.37-1.28 (m, 2H).

### Example 218: 2-((1-methyl-3-(trifluoromethyl)-1H-pyrazol-5-yl)sulfonyl)-6-(oxetan-3-ylmethyl)-2,6-diazaspiro[3.3]heptane

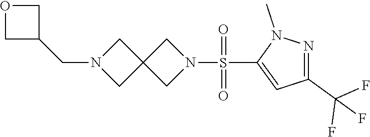 [SMILES: Cn1nc(C(F)(F)F)cc1S(=O)(=O)N1CC2(CN(CC3COC3)C2)C1]

A vial containing 2-((1-methyl-3-(trifluoromethyl)-1H-pyrazol-5-yl)sulfonyl)-2,6-diazaspiro[3.3]heptane trifluoroacetate (105 mg, 338 μmol) in anhydrous methanol (3 mL) was cooled in an ice water bath, then Hunigs Base (0.26 mL, 1.5 mmol) was added carefully to free base the starting material. After 20 minutes, oxetane-3-carbaldehyde (61 mg, 705 μmol) and acetic acid (0.1 mL, 1.7 mmol) were carefully added to the cooled mixture. After 15 minutes, sodium triacetoxyborohydride (422 mg, 1.99 mmol) was added carefully in portions to the cooled reaction solution. Upon complete addition of STAB, the reaction was maintained at <5° C. and monitored with LCMS. After 1.5 hours, the reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous magnesium sulfate. After filtration and concentration under reduced pressure, the residue was loaded onto a silica gel column and purified with (40-90% 3:1 ethyl acetate:ethanol in heptane.) The desired fractions were pooled then concentrated under reduced pressure to afford a white solid as 2-((1-methyl-3-(trifluoromethyl)-1H-pyrazol-5-yl)sulfonyl)-6-(oxetan-3-ylmethyl)-2,6-diazaspiro[3.3]heptane (44 mg, 33%). LCMS m/z=381.0 (M+H)⁺. ¹H NMR (500 MHz, DMSO-d₆) δ (ppm) 7.52 (br s, 1H), 4.56-4.50 (m, 2H), 4.19-4.15 (m, 2H), 4.14 (br s, 3H), 3.97 (br s, 4H), 3.09 (br s, 4H), 2.87-2.78 (m, 1H), 2.56-2.53 (m, 2H).

### Example 219: 2-((3-cyclopropyl-1-methyl-1H-pyrazol-5-yl)sulfonyl)-6-((tetrahydro-2H-pyran-4-yl)methyl)-2,6-diazaspiro[3.3]heptane

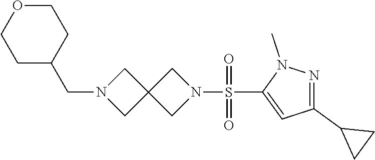 [SMILES: Cn1nc(C2CC2)cc1S(=O)(=O)N1CC2(CN(CC3CCOCC3)C2)C1]

To a vial containing 2-((tetrahydro-2H-pyran-4-yl)methyl)-2,6-diazaspiro[3.3]heptane trifluoroacetate (98 mg, 317 μmol) in anhydrous dichloromethane (2 mL) was added Hunigs Base (0.35 mL, 2.01 mmol) carefully dropwise at <5° C. After 5 minutes, 5-cyclopropyl-2-methyl-pyrazole-3-sulfonyl chloride (91 mg, 411 μmol) was added carefully to the cold solution. Upon complete addition of sulfonyl chloride, the reaction was warmed to 23° C. and monitored with LCMS. After 3 hours, the reaction was carefully quenched with slow addition of aqueous saturated sodium bicarbonate solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a colorless film as 2-((3-cyclopropyl-1-methyl-1H-pyrazol-5-yl)sulfonyl)-6-((tetrahydro-2H-pyran-4-yl)methyl)-2,6-diazaspiro[3.3]heptane (18 mg, 14%). LCMS m/z=381.3 (M+H)⁺. ¹H NMR (500 MHz, DICHLOROMETHANE-d₂) δ (ppm) 6.45 (s, 1H), 4.53-4.45 (m, 2H), 4.24 (s, 2H), 3.96 (s, 3H), 3.95-3.90 (m, 4H), 3.78 (br d, J=6.7 Hz, 2H), 3.34 (br t, J=11.6 Hz, 2H), 2.90-2.85 (m, 2H), 1.92-1.86 (m, 2H), 1.78-1.74 (m, 2H), 1.35-1.28 (m, 2H), 0.95-0.91 (m, 2H), 0.74-0.70 (m, 2H).

### Example 220: 2-((2-fluoro-4-(trifluoromethoxy)phenyl)sulfonyl)-6-((tetrahydro-2H-pyran-4-yl)methyl)-2,6-diazaspiro[3.3]heptane

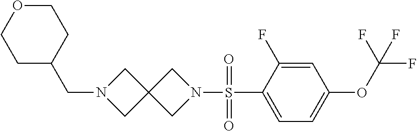 [SMILES: O=S(=O)(c1ccc(OC(F)(F)F)cc1F)N1CC2(CN(CC3CCOCC3)C2)C1]

To a vial containing 2-((tetrahydro-2H-pyran-4-yl)methyl)-2,6-diazaspiro[3.3]heptane trifluoroacetate (107 mg, 345 μmol) in anhydrous dichloromethane (2 mL) was added Hunigs Base (0.35 mL, 2.01 mmol) carefully dropwise at <5° C. After 5 minutes, 2-fluoro-4-(trifluoromethoxy)benzenesulfonyl chloride (156 mg, 560 μmol) was added carefully to the cold solution. Upon complete addition of sulfonyl chloride, the reaction was warmed to 23° C. and monitored with LCMS. After 1.5 hours, the reaction was carefully quenched with slow addition of aqueous saturate sodium bicarbonate solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (35-75% 3:1 ethyl acetate:ethanol in heptane.) The desired fractions were pooled then concentrated under reduced pressure to afford a white solid as 2-((2-fluoro-4-(trifluoromethoxy)phenyl)sulfonyl)-6-((tetrahydro-2H-pyran-4-yl)methyl)-2,6-diazaspiro[3.3]heptane (19 mg, 12%). LCMS m/z=439.2 (M+H)⁺. ¹H NMR (500 MHz, DMSO-d₆) δ (ppm) 7.94 (t, J=8.4 Hz, 1H), 7.77 (br d, J=9.8 Hz, 1H), 7.48 (br d, J=8.9 Hz, 1H), 3.91 (s, 4H), 3.76 (br dd, J=2.6, 11.1 Hz, 2H), 3.23-3.16 (m, 2H), 3.06 (s, 4H), 2.12 (br d, J=6.7 Hz, 2H), 1.47 (br d, J=13.1 Hz, 2H), 1.42-1.34 (m, 1H), 1.09-1.00 (m, 2H).

### Example 221: 2-((3-methyl-5-(trifluoromethyl)pyridin-2-yl)sulfonyl)-6-(tetrahydro-2H-pyran-4-yl)-2,6-diazaspiro[3.3]heptane

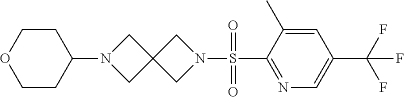 [SMILES: Cc1cc(C(F)(F)F)cnc1S(=O)(=O)N1CC2(CN(C3CCOCC3)C2)C1]

### 1. Synthesis of 2-((2-chloro-6-(trifluoromethyl)pyridin-3-yl)sulfonyl)-6-(tetrahydro-2H-pyran-4-yl)-2,6-diazaspiro[3.3]heptane

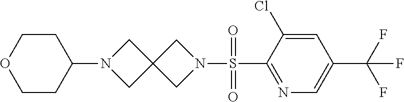 [SMILES: O=S(=O)(c1ncc(C(F)(F)F)cc1Cl)N1CC2(CN(C3CCOCC3)C2)C1]

To a vial containing 2-(tetrahydro-2H-pyran-4-yl)-2,6-diazaspiro[3.3]heptane hydrochloride (206 mg, 942 μmol) in anhydrous methanol (1.5 mL) was added Hunigs Base (0.7 mL, 4.0 mmol) carefully dropwise at room temperature. After 10 minutes, the homogeneous mixture was concentrated to afford an off white solid. 2-Methyl-2-butanol (2 mL), 3-chloro-5-(trifluoromethyl)pyridine-2-sulfonyl fluoride (311 mg, 1.18 mmol), and Ca(NTf₂)₂ (617 mg, 1.03 mmol) were added carefully in portions. Upon complete addition of calcium triflimide, the heterogeneous reaction was heated to 60° C. and monitored with LCMS. After 19 hours, the reaction mixture was cooled to room temperature then carefully quenched with slow addition of aqueous 2 M sodium hydroxide. The heterogeneous mixture was extracted three times with dichloromethane. The organic extractions were pooled then dried over anhydrous magnesium sulfate. After filtration and concentration under reduced pressure, the residue was loaded onto a silica gel column and purified with (35-85% 3:1 ethyl acetate:ethanol in heptane.) The desired fractions were pooled then concentrated under reduced pressure to afford a colorless film as 2-((2-chloro-6-(trifluoromethyl)pyridin-3-yl)sulfonyl)-6-(tetrahydro-2H-pyran-4-yl)-2,6-diazaspiro[3.3]heptane that was used without further purification. LCMS m/z=426.3 (M+H)⁺.

### 2. Synthesis of 2-((3-methyl-5-(trifluoromethyl)pyridin-2-yl)sulfonyl)-6-(tetrahydro-2H-pyran-4-yl)-2,6-diazaspiro[3.3]heptane

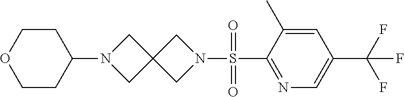 [SMILES: Cc1cc(C(F)(F)F)cnc1S(=O)(=O)N1CC2(CN(C3CCOCC3)C2)C1]

A vial containing 2-((2-chloro-6-(trifluoromethyl)pyridin-3-yl)sulfonyl)-6-(tetrahydro-2H-pyran-4-yl)-2,6-diazaspiro[3.3]heptane (1723 mg, 406 μmol), potassium methyl trifluoroborate (155 mg, 1.27 mmol), Pd(dppf)Cl₂ CH₂Cl₂ (109 mg, 133 μmol), and potassium carbonate (289 mg, 2.09 mmol) in dioxane (2.5 mL) and water (0.25 mL) was degassed and backfilled with nitrogen. The heterogeneous reaction mixture was carefully heated to 90° C. and monitored with LCMS. After 23 hours, the heterogeneous reaction was cooled to room temperature then carefully partitioned between water and dichloromethane. The aqueous layer was extracted two additional times with dichloromethane. The organic extractions were pooled then washed once saturated aqueous sodium chloride solution, then the organic layer was dried over anhydrous magnesium sulfate. After filtration and concentration under reduced pressure, the residue was loaded onto a silica gel column and purified with (40-80% 3:1 ethyl acetate:ethanol in heptane.) The desired fractions were pooled then concentrated under reduced pressure to afford a dark yellow film that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-55% B (0.2% NH₄OH final v/v % modifier) with flow rate at 50 mL/min. The desired fractions were pooled then concentrated under reduced pressure to afford an off-white solid as 2-((3-methyl-5-(trifluoromethyl)pyridin-2-yl)sulfonyl)-6-(tetrahydro-2H-pyran-4-yl)-2,6-diazaspiro[3.3]heptane (31 mg, 19%). LCMS m/z=406.3 (M+H)⁺. ¹H NMR (400 MHz, DMSO-d₆) δ (ppm) 8.93 (s, 1H), 8.46 (d, J=1.5 Hz, 1H), 4.30 (s, 4H), 3.82-3.76 (m, 2H), 3.32-3.29 (m, 4H), 3.28-3.21 (m, 2H), 2.60 (s, 3H), 2.17-2.08 (m, 1H), 1.58 (br d, J=10.5 Hz, 2H), 1.15-1.06 (m, 2H).

### Example 222 and 223: 1-methyl-4-(6-((2-methyl-6-(trifluoromethyl)pyridin-3-yl)sulfonyl)-2,6-diazaspiro[3.3]heptan-2-yl)cyclohexan-1-ol AND 1-methyl-4-(6-((2-methyl-6-(trifluoromethyl)pyridin-3-yl)sulfonyl)-2,6-diazaspiro[3.3]heptan-2-yl)cyclohexan-1-ol

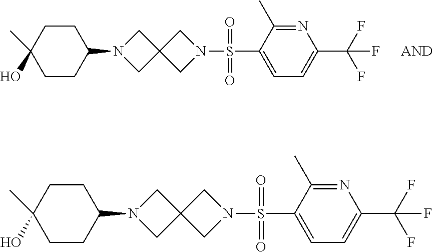

A vial containing 2-((2-methyl-6-(trifluoromethyl)pyridin-3-yl)sulfonyl)-2,6-diazaspiro[3.3]heptane trifluoroacetate (200 mg, 459 μmol) in anhydrous methanol (2 mL) was cooled in an ice water bath, then Hunigs Base (0.3 mL, 1.7 mmol) was added carefully to free base the starting material. After 20 minutes, 4-hydroxy-4-methyl-cyclohexanone (126 mg, 985 μmol) and acetic acid (0.15 mL, 2.6 mmol) were carefully added to the cooled mixture. After 15 minutes, sodium triacetoxyborohydride (513 mg, 2.42 mmol) was added carefully in portions to the cooled reaction solution. Upon complete addition of STAB, the reaction was maintained at <5° C. and monitored with LCMS. After 2 hours, the milky heterogenous reaction was carefully quenched with slow addition of aqueous saturated sodium bicarbonate solution. The mixture was stirred at 23° C. for 30 minutes, then the mixture was extracted three times with dichloromethane. The organic extractions were pooled then dried over anhydrous sodium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a colorless film. The film was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-55% B (0.2% NH₄OH final v/v % modifier) with flow rate at 50 mL/min. The following compounds of arbitrarily assigned stereochemistry were isolated:

First off the column, 1-methyl-4-(6-((2-methyl-6-(trifluoromethyl)pyridin-3-yl)sulfonyl)-2,6-diazaspiro[3.3]heptan-2-yl)cyclohexan-1-ol (17 mg, 8%). LCMS m/z=434.4 (M+H)⁺. ¹H NMR (500 MHz, DMSO-d6) δ (ppm) 8.44 (d, J=8.2 Hz, 1H), 7.98 (d, J=8.2 Hz, 1H), 4.09 (s, 1H), 3.96 (s, 4H), 3.18-3.09 (m, 4H), 2.81 (s, 3H), 1.91 (br s, 1H), 1.57-1.49 (m, 2H), 1.45 (br d, J=9.2 Hz, 2H), 1.21 (br t, J=9.2 Hz, 2H), 1.06-0.96 (m, 5H).

Second off the column, 1-methyl-4-(6-((2-methyl-6-(trifluoromethyl)pyridin-3-yl)sulfonyl)-2,6-diazaspiro[3.3]heptan-2-yl)cyclohexan-1-ol (18 mg, 8%). LCMS m/z=434.4 (M+H)⁺. ¹H NMR (500 MHz, DMSO-d₆) δ (ppm) 8.44 (d, J=8.2 Hz, 1H), 7.98 (d, J=8.2 Hz, 1H), 3.97 (s, 4H), 3.94-3.88 (m, 1H), 3.18-3.07 (m, 4H), 2.81 (s, 3H), 1.82-1.70 (m, 1H), 1.47 (br d, J=11.6 Hz, 2H), 1.35-1.26 (m, 2H), 1.25-1.11 (m, 4H), 1.04 (s, 3H).

### Example 224: 2-((3-methyl-5-(trifluoromethyl)pyridin-2-yl)sulfonyl)-6-((tetrahydro-2H-pyran-4-yl)methyl)-2,6-diazaspiro[3.3]heptane

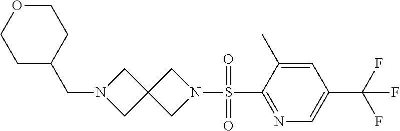 [SMILES: Cc1cc(C(F)(F)F)cnc1S(=O)(=O)N1CC2(CN(CC3CCOCC3)C2)C1]

### 1. Synthesis of 2-((3-chloro-5-(trifluoromethyl)pyridin-2-yl)sulfonyl)-6-((tetrahydro-2H-pyran-4-yl)methyl)-2,6-diazaspiro[3.3]heptane

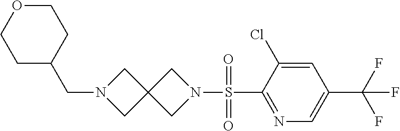 [SMILES: O=S(=O)(c1ncc(C(F)(F)F)cc1Cl)N1CC2(CN(CC3CCOCC3)C2)C1]

To a vial containing 2-((tetrahydro-2H-pyran-4-yl)methyl)-2,6-diazaspiro[3.3]heptane trifluoroacetate (255 mg, 823 μmol) in anhydrous methanol (1.5 mL) was added Hunigs Base (0.7 mL, 4.02 mmol) carefully dropwise at room temperature. After 10 minutes, the homogeneous mixture was concentrated to afford an off-white solid. 2-Methyl-2-butanol (2 mL), 3-chloro-5-(trifluoromethyl)pyridine-2-sulfonyl fluoride (289 mg, 1.10 mmol), and Ca(NTf₂)₂ (544 mg, 907 μmol) were added carefully in portions. Upon complete addition of calcium triflimide, the heterogeneous reaction was heated to 60° C. and monitored with LCMS. After 19 hours, the reaction mixture was cooled to room temperature then carefully quenched with slow addition of aqueous 2 M sodium hydroxide. The heterogeneous mixture was extracted three times with dichloromethane. The organic extractions were pooled then dried over anhydrous magnesium sulfate. After filtration and concentration under reduced pressure, the residue was loaded onto a silica gel column and purified with (35-85% 3:1 ethyl acetate:ethanol in heptane.) The desired fractions were pooled then concentrated under reduced pressure to afford a colorless film as 2-((3-chloro-5-(trifluoromethyl)pyridin-2-yl)sulfonyl)-6-((tetrahydro-2H-pyran-4-yl)methyl)-2,6-diazaspiro[3.3]heptane (105 mg, 29%) that was used without further purification. LCMS m/z=440.3 (M+H)⁺.

### 2. Synthesis of 2-((3-methyl-5-(trifluoromethyl)pyridin-2-yl)sulfonyl)-6-((tetrahydro-2H-pyran-4-yl)methyl)-2,6-diazaspiro[3.3]heptane

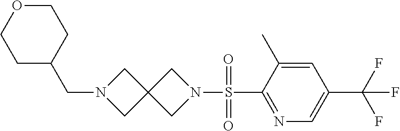 [SMILES: Cc1cc(C(F)(F)F)cnc1S(=O)(=O)N1CC2(CN(CC3CCOCC3)C2)C1]

A vial containing 2-((3-chloro-5-(trifluoromethyl)pyridin-2-yl)sulfonyl)-6-((tetrahydro-2H-pyran-4-yl)methyl)-2,6-diazaspiro[3.3]heptane (105 mg, 240 μmol), potassium methyl trifluoroborate (940 mg, 7.71 mmol), Pd(dppf)Cl₂ CH₂Cl₂ (63 mg, 77 μmol), and potassium carbonate (178 mg, 1.29 mmol) in Dioxane (1.5 mL) and water (0.15 mL) was degassed and backfilled with nitrogen. The heterogeneous reaction mixture was carefully heated to 90° C. and monitored with LCMS. After 19 hours, the heterogeneous reaction was cooled to room temperature then carefully partitioned between water and dichloromethane. The aqueous layer was extracted two additional times with dichloromethane. The organic extractions were pooled then washed once saturated aqueous sodium chloride solution, then the organic layer was dried over anhydrous magnesium sulfate. After filtration and concentration under reduced pressure, the residue was loaded onto a silica gel column and purified with (40-80% 3:1 ethyl acetate:ethanol in heptane.) The desired fractions were pooled then concentrated under reduced pressure to afford a dark yellow film that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-60% B (0.2% NH₄OH final v/v % modifier) with flow rate at 50 mL/min. The desired fractions were pooled then concentrated under reduced pressure to afford an off-white solid as 2-((3-methyl-5-(trifluoromethyl)pyridin-2-yl)sulfonyl)-6-((tetrahydro-2H-pyran-4-yl)methyl)-2,6-diazaspiro[3.3]heptane (19 mg, 18%). LCMS m/z=420.3 (M+H)⁺. ¹H NMR (400 MHz, DMSO-d₆) δ (ppm) 8.91 (s, 1H), 8.46 (s, 1H), 4.29 (s, 4H), 3.80 (br dd, J=3.3, 11.3 Hz, 2H), 3.36-3.30 (m, 4H), 3.29-3.20 (m, 3H), 2.59 (s, 3H), 2.26-2.17 (m, 2H), 1.57-1.51 (m, 2H), 1.16-1.06 (m, 2H).

### Example 225: 2-((4-(difluoromethoxy)-2,6-difluorophenyl)sulfonyl)-6-((tetrahydro-2H-pyran-4-yl)methyl)-2,6-diazaspiro[3.3]heptane

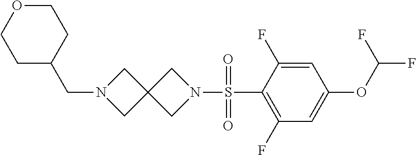 [SMILES: O=S(=O)(c1c(F)cc(OC(F)F)cc1F)N1CC2(CN(CC3CCOCC3)C2)C1]

To a vial containing 2-((tetrahydro-2H-pyran-4-yl)methyl)-2,6-diazaspiro[3.3]heptane trifluoroacetate (63 mg, 203 μmol) in anhydrous dichloromethane (1.5 mL) was added Hunigs Base (0.16 mL, 919 μmol) carefully dropwise at room temperature. After 10 minutes, 4-(difluoromethoxy)-2,6-difluoro-benzenesulfonyl chloride (73 mg, 263 μmol) was added carefully to the homogeneous mixture. Upon complete addition of sulfonyl chloride, the reaction was maintained at 23° C. and monitored with LCMS. After 1.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (35-95% 3:1 ethyl acetate:ethanol in heptane.) The desired fractions were pooled then concentrated under reduced pressure to afford an off-white solid that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-60% B (0.2% NH₄OH final v/v % modifier) with flow rate at 50 mL/min. The desired fractions were pooled then concentrated under reduced pressure to afford a white solid as 2-((4-(difluoromethoxy)-2,6-difluorophenyl)sulfonyl)-6-((tetrahydro-2H-pyran-4-yl)methyl)-2,6-diazaspiro[3.3]heptane (10 mg, 11%). LCMS m/z=439.1 (M+H)⁺. ¹H NMR (500 MHz, DICHLOROMETHANE-d₂) δ (ppm) 6.86 (s, 1H), 6.84 (s, 1H), 6.65 (t, J=71.9 Hz, 1H), 4.56-4.35 (m, 1H), 4.02 (br s, 4H), 3.87-3.76 (m, 2H), 3.30 (br d, J=8.9 Hz, 2H), 3.26-2.85 (m, 4H), 2.19 (br s, 1H), 1.95-1.63 (m, 1H), 1.45-1.11 (m, 4H).

### Example 226: 2-((4-(difluoromethoxy)-2-fluorophenyl)sulfonyl)-6-((tetrahydro-2H-pyran-4-yl)methyl)-2,6-diazaspiro[3.3]heptane

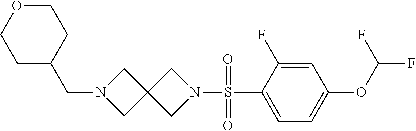 [SMILES: O=S(=O)(c1ccc(OC(F)F)cc1F)N1CC2(CN(CC3CCOCC3)C2)C1]

To a vial containing 2-((tetrahydro-2H-pyran-4-yl)methyl)-2,6-diazaspiro[3.3]heptane trifluoroacetate (100 mg, 324 μmol) in anhydrous dichloromethane (2 mL) was added Hunigs base (0.4 mL, 2.3 mmol) carefully dropwise at <5° C. After 5 minutes, 4-(difluoromethoxy)-2-fluoro-benzenesulfonyl chloride (130 mg, 501 μmol) was added carefully to the cold solution. Upon complete addition of sulfonyl chloride, the reaction was warmed to 23° C. and monitored with LCMS. After 1.5 hours, the reaction was carefully quenched with slow addition of aqueous 1 M sodium hydroxide solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a colorless film as 2-((4-(difluoromethoxy)-2-fluorophenyl)sulfonyl)-6-((tetrahydro-2H-pyran-4-yl)methyl)-2,6-diazaspiro[3.3]heptane (96 mg, 67%). LCMS m/z=421.3 (M+H)⁺. ¹H NMR (500 MHz, DICHLOROMETHANE-d₂) δ (ppm) 7.88-7.82 (m, 1H), 7.15-7.02 (m, 2H), 6.66 (t, J=72.3 Hz, 1H), 3.95 (br s, 4H), 3.85 (br dd, J=3.1, 11.0 Hz, 2H), 3.28 (br t, J=11.6 Hz, 2H), 3.25-3.06 (m, 4H), 2.28-2.11 (m, 2H), 1.62-1.42 (m, 3H), 1.22-1.12 (m, 2H).

### Example 227: 2-((4-(difluoromethoxy)-2-fluorophenyl)sulfonyl)-6-(tetrahydro-2H-pyran-4-yl)-2,6-diazaspiro[3.3]heptane

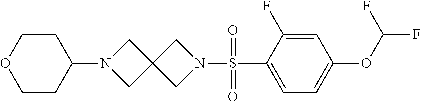 [SMILES: O=S(=O)(c1ccc(OC(F)F)cc1F)N1CC2(CN(C3CCOCC3)C2)C1]

To a vial containing 2-(tetrahydro-2H-pyran-4-yl)-2,6-diazaspiro[3.3]heptane hydrochloride (88 mg, 401 μmol) in anhydrous dichloromethane (2 mL) was added Hunigs Base (0.4 mL 2.3 mmol) carefully dropwise at <5° C. After 5 minutes, 4-(difluoromethoxy)-2-fluoro-benzenesulfonyl chloride (135 mg, 519 μmol) was added carefully to the cold solution. Upon complete addition of sulfonyl chloride, the reaction was warmed to 23° C. and monitored with LCMS and TLC. After 30 minutes, the reaction was carefully quenched with slow addition of aqueous 1 M sodium hydroxide solution. The mixture was stirred at 23° C. for 20 minutes, then the biphasic mixture was extracted three times with dichloromethane. The organic extractions were pooled then dried over anhydrous magnesium sulfate. After filtration and concentration under reduced pressure, the residue was loaded onto a silica gel column and purified with (40-80% 3:1 ethyl acetate:ethanol in heptane.) The desired fractions were pooled then concentrated under reduced pressure to afford a white solid that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-45% B (0.2% NH₄OH final v/v % modifier) with flow rate at 50 mL/min. The desired fractions were pooled then concentrated under reduced pressure to afford a colorless film as 2-((4-(difluoromethoxy)-2-fluorophenyl)sulfonyl)-6-(tetrahydro-2H-pyran-4-yl)-2,6-diazaspiro[3.3]heptane (38 mg, 22%). LCMS m/z=407.3 (M+H)⁺. ¹H NMR (500 MHz, DICHLOROMETHANE-d₂) δ (ppm) 7.85 (t, J=8.2 Hz, 1H), 7.08-7.03 (m, 2H), 6.66 (t, J=72.3 Hz, 1H), 3.96 (s, 4H), 3.84 (br d, J=11.0 Hz, 2H), 3.32-3.26 (m, 2H), 3.14 (br s, 4H), 2.07 (br s, 1H), 1.53 (br d, J=11.9 Hz, 2H), 1.18 (br d, J=9.8 Hz, 2H).

### Example 228: 2-((6-((tetrahydro-2H-pyran-4-yl)methyl)-2,6-diazaspiro[3.3]heptan-2-yl)sulfonyl)-5-(trifluoromethyl)benzonitrile

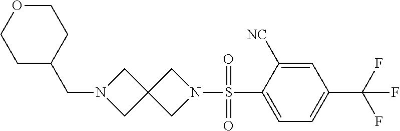 [SMILES: N#Cc1cc(C(F)(F)F)ccc1S(=O)(=O)N1CC2(CN(CC3CCOCC3)C2)C1]

To a vial containing 2-((tetrahydro-2H-pyran-4-yl)methyl)-2,6-diazaspiro[3.3]heptane trifluoroacetate (66 mg, 212 μmol) in anhydrous dichloromethane (1 mL) was added Hunigs Base (0.16 mL, 919 μmol) carefully dropwise at room temperature. After 10 minutes, 2-cyano-4-(trifluoromethyl)benzenesulfonyl chloride (75 mg, 279 μmol) was added carefully to the homogeneous mixture. Upon complete addition of sulfonyl chloride, the reaction was maintained at 23° C. and monitored with LCMS and TLC. After 1.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (35-95% 3:1 ethyl acetate:ethanol in heptane.) The desired fractions were pooled then concentrated under reduced pressure to afford an off-white solid that was dissolved in DMSO and few drops of water then filtered. The homogeneous solution was submitted for mass directed reverse phase HPLC purification. Liquid chromatography was performed using a Waters XSelect CSH C18, 5 μm, 30 mm×100 mm column with mobile phase H₂O (A) and MeCN (B) and a gradient of 5-60% B (0.2% NH₄OH final v/v % modifier) with flow rate at 50 mL/min. The desired fractions were pooled then concentrated under reduced pressure to afford a white solid as 2-((6-((tetrahydro-2H-pyran-4-yl)methyl)-2,6-diazaspiro[3.3]heptan-2-yl)sulfonyl)-5-(trifluoromethyl)benzonitrile (23 mg, 24%). LCMS m/z=430.1 (M+H)⁺. ¹H NMR (500 MHz, DICHLOROMETHANE-d₂) δ (ppm) 8.17 (br d, J=8.2 Hz, 1H), 8.14 (s, 1H), 8.01 (br d, J=8.2 Hz, 1H), 4.12 (br s, 4H), 3.91-3.83 (m, 2H), 3.45-3.14 (m, 6H), 2.21 (br s, 2H), 1.57-1.41 (m, 3H), 1.27-1.14 (m, 2H).

### Example 229: 2-((tetrahydro-2H-pyran-4-yl)methyl)-6-((6-(trifluoromethyl)pyridin-3-yl)sulfonyl)-2,6-diazaspiro[3.3]heptane

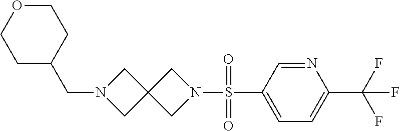 [SMILES: O=S(=O)(c1ccc(C(F)(F)F)nc1)N1CC2(CN(CC3CCOCC3)C2)C1]

To a vial containing 2-((tetrahydro-2H-pyran-4-yl)methyl)-2,6-diazaspiro[3.3]heptane trifluoroacetate (310 mg, 999 μmol) in anhydrous dichloromethane (6 mL) was added Hunigs Base (0.7 mL, 4.02 mmol) carefully dropwise at room temperature. After 10 minutes, 6-(trifluoromethyl)pyridine-3-sulfonyl chloride (307 mg, 1.25 mmol) was added carefully to the homogeneous mixture. Upon complete addition of sulfonyl chloride, the reaction was maintained at 23° C. and monitored with LCMS. After 0.5 hours, the reaction mixture was carefully quenched with slow addition of saturated aqueous sodium bicarbonate solution. The heterogeneous mixture was extracted three times with dichloromethane. The organic extractions were pooled then dried over anhydrous magnesium sulfate. After filtration and concentration under reduced pressure, the residue was loaded onto a silica gel column and purified with (40-100% 3:1 ethyl acetate:ethanol in heptane.) The desired fractions were pooled then concentrated under reduced pressure to afford a white solid as 2-((tetrahydro-2H-pyran-4-yl)methyl)-6-((6-(trifluoromethyl)pyridin-3-yl)sulfonyl)-2,6-diazaspiro[3.3]heptane (139 mg, 33%). LCMS m/z=406.1 [M+H]+. ¹H NMR (500 MHz, DICHLOROMETHANE-d₂) δ (ppm) 9.11 (s, 1H), 8.30 (br d, J=7.3 Hz, 1H), 7.91 (br d, J=8.2 Hz, 1H), 3.91 (br s, 4H), 3.87-3.81 (m, 2H), 3.28 (br t, J=11.4 Hz, 2H), 3.24-2.94 (m, 4H), 2.17 (br s, 2H), 1.53-1.33 (m, 3H), 1.20-1.08 (m, 2H).

### Assays

### EBP Functional Assay

The EBP immunoaffinity (IA) LC-MS assay measures the potency of small molecule inhibitors of EBP by quantifying their concentration-dependent changes in the enzyme's substrate and product using liquid chromatography atmospheric pressure chemical ionization multiple reaction monitoring mass spectrometry (LC-APCI MRM MS). HEK293T cells were utilized as the source of EBP enzyme. The enzyme was incubated with the small molecule inhibitors at variable concentrations for 30 min. Deuterated form of EBP substrate, zymosterol-d5 (Avanti Polar Lipids, Cat #700068P-1 mg), was then added and the plate was incubated at 37° C. for 4 h. Finally, the sterol isomers were extracted and injected to LC-APCI MRM MS. MRM transition used for the quantification for both zymosterol and dihydrolathosterol (substrate and product of EBP enzymatic reaction, respectively) is 372.3-203.2, CE 30 and DP 80 in positive ion mode. Percent conversion of the zymosterol-d5 to dehydrolathosterol-d5 was used to derive IC₅₀ curves. Tasin-1 (1′-[(4-Methoxyphenyl)sulfonyl]-4-methyl-1,4′-bipiperidine, CAS 792927-06-1) was used as the reference small molecule inhibitor.

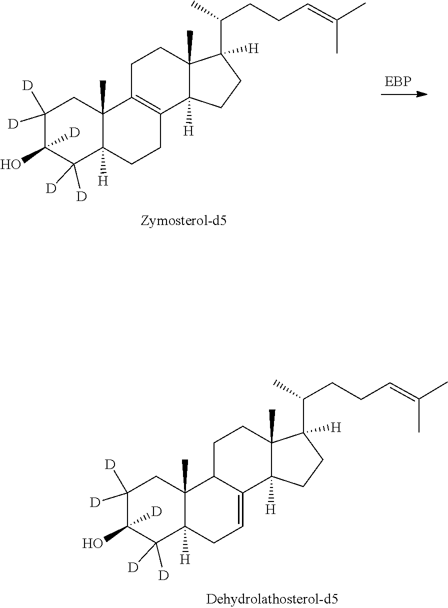

Percent conversion versus the compound concentration data were fit to the following 4-parameter logistic model to generate IC₅₀ curves:

$$y = LA + \frac{UA + LA}{1 + {10}^{( log {IC}_{50} - x ) \cdot Slope}}$$

### Data for Examples

| Example No. | EBP Functional Assay |
|---|---|
| 1 | + |
| 2 | ++ |
| 3 | ++ |
| 4 | +++ |
| 5 | ++ |
| 6 | + |
| 7 | + |
| 8 | + |
| 9 | + |
| 10 | + |
| 11 | ++ |
| 12 | + |
| 13 | +++ |
| 14 | +++ |
| 15 | ++ |
| 16 | + |
| 17 | + |
| 18 | + |
| 19 | + |
| 20 | ++ |
| 21 | + |
| 22 | + |
| 23 | ++ |
| 24 | ++ |
| 25 | +++ |
| 26 | ++ |
| 27 | ++ |
| 28 | ++ |
| 29 | ++ |
| 30 | ++ |
| 31 | + |
| 32 | + |
| 33 | +++ |
| 34 | +++ |
| 35 | +++ |
| 36 | +++ |
| 37 | ++ |
| 38 | +++ |
| 39 | +++ |
| 40 | +++ |
| 41 | +++ |
| 42 | +++ |
| 43 | +++ |
| 44 | +++ |
| 45 | ++ |
| 46 | ++ |
| 47 | ++ |
| 48 | ++ |
| 49 | ++ |
| 50 | +++ |
| 51 | +++ |
| 52 | +++ |
| 53 | +++ |
| 54 | ++ |
| 55 | +++ |
| 56 | +++ |
| 57 | ++ |
| 58 | +++ |
| 59 | ++ |
| 60 | +++ |
| 61 | ++ |
| 62 | +++ |
| 63 | ++ |
| 64 | + |
| 65 | + |
| 66 | + |
| 67 | + |
| 68 | ++ |
| 69 | + |
| 70 | +++ |
| 71 | ++ |
| 72 | ++ |
| 73 | ++ |
| 74 | +++ |
| 75 | + |
| 76 | +++ |
| 77 | +++ |
| 78 | ++ |
| 79 | +++ |
| 80 | + |
| 81 | +++ |
| 82 | + |
| 83 | ++ |
| 84 | + |
| 85 | +++ |
| 86 | +++ |
| 87 | +++ |
| 88 | ++ |
| 89 | +++ |
| 90 | ++ |
| 91 | + |
| 92 | +++ |
| 93 | +++ |
| 94 | +++ |
| 95 | ++ |
| 96 | ++ |
| 97 | + |
| 98 | +++ |
| 99 | +++ |
| 100 | +++ |
| 101 | ++ |
| 102 | +++ |
| 103 | ++ |
| 104 | ++ |
| 105 | ++ |
| 106 | ++ |
| 107 | +++ |
| 108 | +++ |
| 109 | + |
| 110 | + |
| 111 | +++ |
| 112 | + |
| 113 | ++ |
| 114 | +++ |
| 115 | +++ |
| 116 | +++ |
| 117 | + |
| 118 | ++ |
| 119 | ++ |
| 120 | ++ |
| 121 | + |
| 122 | +++ |
| 123 | ++ |
| 124 | ++ |
| 125 | + |
| 126 | + |
| 127 | +++ |
| 128 | +++ |
| 129 | +++ |
| 130 | +++ |
| 131 | ++ |
| 132 | ++ |
| 133 | + |
| 134 | ++ |
| 135 | +++ |
| 136 | ++ |
| 137 | +++ |
| 138 | +++ |
| 139 | + |
| 140 | + |
| 141 | +++ |
| 142 | +++ |
| 143 | +++ |
| 144 | +++ |
| 145 | +++ |
| 146 | ++ |
| 147 | + |
| 148 | +++ |
| 149 | +++ |
| 150 | +++ |
| 151 | + |
| 152 | +++ |
| 153 | +++ |
| 154 | ++ |
| 155 | +++ |
| 156 | +++ |
| 157 | +++ |
| 158 | + |
| 159 | +++ |
| 160 | ++ |
| 161 | + |
| 162 | + |
| 163 | +++ |
| 164 | + |
| 165 | + |
| 166 | ++ |
| 167 | +++ |
| 168 | + |
| 169 | + |
| 170 | + |
| 171 | + |
| 172 | + |
| 173 | ++ |
| 174 | ++ |
| 175 | +++ |
| 176 | +++ |
| 177 | + |
| 178 | + |
| 179 | ++ |
| 180 | ++ |
| 181 | ++ |
| 182 | + |
| 183 | +++ |
| 184 | +++ |
| 185 | +++ |
| 186 | ++ |
| 187 | + |
| 188 | +++ |
| 189 | +++ |
| 190 | +++ |
| 191 | +++ |
| 192 | +++ |
| 193 | +++ |
| 194 | +++ |
| 195 | +++ |
| 196 | ++ |
| 197 | +++ |
| 198 | +++ |
| 199 | ++ |
| 200 | ++ |
| 201 | + |
| 202 | +++ |
| 203 | +++ |
| 204 | +++ |
| 205 | +++ |
| 206 | ++ |
| 207 | + |
| 208 | ++ |
| 209 | ++ |
| 210 | ++ |
| 211 | ++ |
| 212 | +++ |
| 213 | ++ |
| 214 | + |
| 215 | + |
| 216 | + |
| 217 | +++ |
| 218 | ++ |
| 219 | ++ |
| 220 | + |
| 221 | + |
| 222 | ++ |
| 223 | + |
| 224 | + |
| 225 | + |
| 226 | + |
| 227 | ++ |
| 228 | + |
| 229 | + |

*N/A means >4000 nM; + means equal to or greater than 250 nM and equal to or less than 4000 nM; ++ means equal to or greater than 100 nM and less than 250 nM; +++ means <100 nM; NT = Not Tested

What is claimed is:

1. A compound represented by Formula (I): 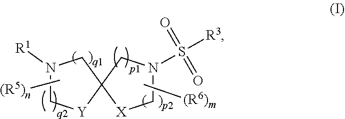
or a pharmaceutically acceptable salt thereof, wherein:
X is CH₂ or O;
Y is CH₂ or O;
provided only one of X and Y is O;
q1 is 1 or 2;
q2 is 0 or 1 when Y is CH₂, or q2 is 2 when Y is O;
p1 is 1 or 2;
p2 is 0 or 1 when X is CH₂, or p2 is 2 when X is O;
R¹ is C₂₋₆alkyl, Het, or —Z-Het, wherein the C₂₋₆alkyl is optionally substituted with one or more R^{A} and Het is optionally substituted with one or more R²;
Z is C₁₋₄alkyl optionally substituted with one or more halo or C₁₋₃alkoxy;
Het is C₃₋₆cycloalkyl, a 4 to 6 membered monocyclic heterocyclyl, or a 6 to 8-membered bicyclic heterocyclyl, each of which is optionally substituted with R²;
R^{A}, for each occurrence, is independently OR^{2a}, SR^{2a}, or C(O)OR^{2a};
R², for each occurrence, is independently C₁₋₆alkyl, halo, —CN or OR^{2a}, wherein the C₁₋₆alkyl is optionally substituted with one or more halo or C₁₋₃alkoxy;
R^{2a} is H C₁₋₆alkyl, or C₃₋₆cycloalkyl wherein the C₁₋₆alkyl is optionally substituted with one or more halo or C₁₋₃alkoxy;
R³ is C₁₋₄alkyl-phenyl, phenyl, 5 or 6-membered monocyclic heteroaryl, or 9 to 10-membered bicyclic heteroaryl, wherein the phenyl, 5 or 6-membered monocyclic heteroaryl and 9 to 10-membered bicyclic heteroaryl are each optionally substituted with one or more substituent R⁴;
R⁴, for each occurrence, is independently halo, —OR^{4a}, —CN, C₁₋₆alkyl, C₃₋₆ cycloalkyl, C₂₋₆alkenyl, phenyl, or 5 or 6-membered monocyclic heteroaryl, wherein the C₁₋₆alkyl or C₃₋₆cycloalkyl are each optionally substituted with one or more C₁₋₆alkyl, C₁₋₆haloalkyl, C₃₋₆cycloalkyl, or halo, and wherein the or 5 or 6-membered monocyclic heteroaryl is optionally substituted with one or more C₁₋₆alkyl; or
two R⁴ together with their intervening atoms form a 5 to 7-membered heterocyclyl;
R^{4a} is H or C₁₋₆alkyl, wherein the C₁₋₆alkyl is optionally substituted with one or more halo;
n is 1 or 2;
m is 1 or 2;
R⁵, for each occurrence, is independently H, halo, C₁₋₃alkyl, or C₁₋₃haloalkyl; or two R⁵ together form a C₁₋₃alkylene;
R⁶, for each occurrence, is independently H, halo, C₁₋₃alkyl, or C₁₋₃haloalkyl; or two R⁶ together form a C₁₋₃alkylene;
provided that the compound is not:

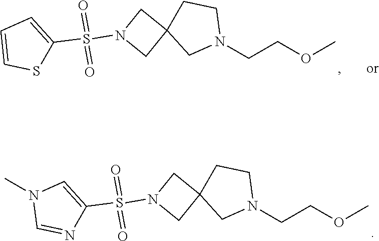

2. The compound of claim 1, wherein the compound is represented by Formula (I): 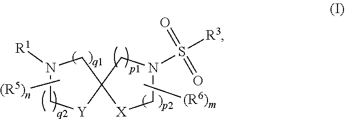
or a pharmaceutically acceptable salt thereof, wherein:
X is CH₂ or O;
Y is CH₂ or O;
provided only one of X and Y is O;
q1 is 1 or 2;
q2 is 0 or 1 when Y is CH₂, or q2 is 2 when Y is O;
p1 is 1 or 2;
p2 is 0 or 1 when X is CH₂, or p2 is 2 when X is O;
R¹ is C₂₋₆alkyl, Het, or —Z-Het, wherein the C₂₋₆alkyl is substituted with one or more OR^{2a} and Het is optionally substituted with one or more R²;
Z is C₁₋₄alkyl optionally substituted with one or more halo;
Het is a 4 to 6 membered monocyclic heterocyclyl, or a 6 to 8-membered bicyclic heterocyclyl, each of which is optionally substituted with R²;
R², for each occurrence, is independently C₁₋₆alkyl or OR^{2a};
R^{2a} is H or C₁₋₆alkyl, wherein the C₁₋₆alkyl is optionally substituted with one or more halo;
R³ is phenyl, 5 or 6-membered monocyclic heteroaryl, or 9 to 10-membered bicyclic heteroaryl, wherein the phenyl, 5 or 6-membered monocyclic heteroaryl and 9 to 10-membered bicyclic heteroaryl are each optionally substituted with one or more substituent R⁴;
R⁴, for each occurrence, is independently halo, —OR^{4a}, —CN, C₁₋₆alkyl, C₃₋₆ cycloalkyl, or 5 or 6-membered monocyclic heteroaryl, wherein the C₁₋₆alkyl is optionally substituted with one or more halo, and wherein the or 5 or 6-membered monocyclic heteroaryl is optionally substituted with one or more C₁₋₆alkyl; or
two R⁴ together with their intervening atoms form a 5 to 7-membered heterocyclyl;
R^{4a} is H or C₁₋₆alkyl, wherein the C₁₋₆alkyl is optionally substituted with one or more halo;
n is 1 or 2;
m is 1 or 2;
R⁵, for each occurrence, is independently H, halo, C₁₋₃alkyl, or C₁₋₃haloalkyl; or two R⁵ together form a C₁₋₃alkylene;
R⁶, for each occurrence, is independently H, halo, C₁₋₃alkyl, or C₁₋₃haloalkyl; or two R⁶ together form a C₁₋₃alkylene;
provided that the compound is not:

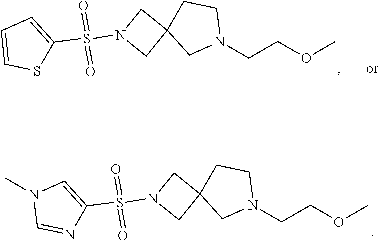

3. The compound of claim 1 or 2, or a pharmaceutically acceptable salt thereof, wherein Het is a 4 to 6 membered oxygen-containing monocyclic saturated heterocyclyl, or a 6 to 8-membered oxygen-containing bicyclic saturated heterocyclyl.

4. The compound of claim 1, or a pharmaceutically acceptable salt thereof, wherein Het is C₃₋₆cycloalkyl, a 4 to 6 membered oxygen-containing monocyclic saturated heterocyclyl, or a 6 to 8-membered oxygen-containing bicyclic saturated heterocyclyl, each of which is optionally substituted with one to three R².

5. The compound of claim 1, or a pharmaceutically acceptable salt thereof, wherein Het is cyclohexyl, oxetanyl, tetrahydrofuranyl, tetrahydropyranyl, dioxolanyl, dioxanyl, 2-oxaspiro[3.3]heptanyl, 2-oxaspiro[3.3]heptanyl, 2-oxabicyclo[2.1.1]hexanyl, 6-oxabicyclo[3.2.1]octanyl, or 2-oxabicyclo[3.1.1]heptanyl, each of which is optionally substituted with one to three R².

6. The compound of any one of claims 1 to 5, wherein the compound is represented by Formula II: 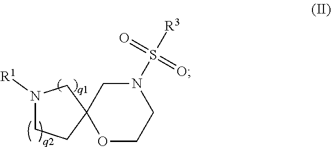
or a pharmaceutically acceptable salt thereof.

7. The compound of any one of claims 1 to 6, or a pharmaceutically acceptable salt thereof, wherein R³ is selected from the group consisting of phenyl, pyridyl, thiazoyl, and pyrazolyl, each of which is optionally substituted with one to three R⁴.

8. The compound of claim 7, or a pharmaceutically acceptable salt thereof, wherein R³ is represented by the following formula: 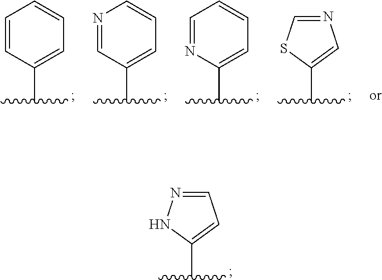
wherein each of the formula depicted above is optionally substituted with one to three R⁴.

9. The compound of claim 8, or a pharmaceutically acceptable salt thereof, wherein R³ is represented by the following formula:

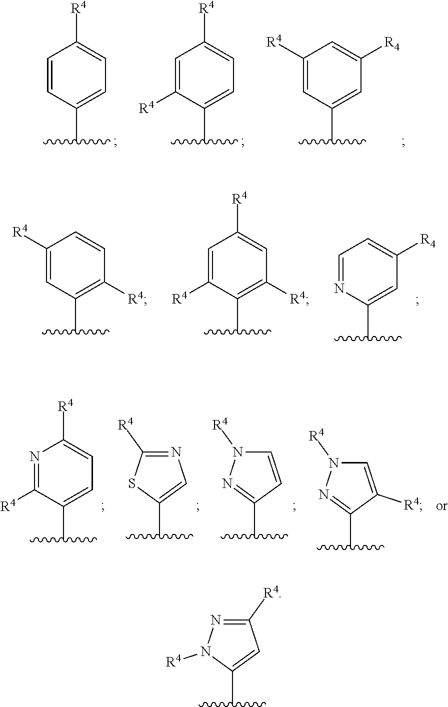

10. The compound of any one of claims 1 to 9, or a pharmaceutically acceptable salt thereof, wherein R⁴, for each occurrence, is independently selected from halo, —CN, —OR^{4a}, C₁₋₄alkyl, C₁₋₄haloalkyl, C₃₋₆cycloalkyl and 5 or 6-membered heteroaryl optionally substituted with C₁₋₃alkyl; and R^{4a} is C₁₋₃alkyl or C₁₋₃haloalkyl.

11. The compound of any one of claims 1 to 10, or a pharmaceutically acceptable salt thereof, wherein R⁴, for each occurrence, is independently selected from —CH₃, —CF₃, —OCHF₂, —OCH₃, —CN, —F, —Cl, isopropyl, cyclopropyl, and 4-methylpyridin-2-yl.

12. The compound of any one of claims 1 to 11, or a pharmaceutically acceptable salt thereof, wherein R¹ is C₂₋₄alkyl substituted with OR^{2a} or is represented by the following formula: 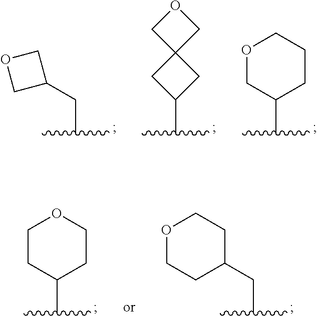 [SMILES: *CC1COC1 ; *C1CC2(COC2)C1 ; *C1CCCOC1 ; *C1CCOCC1 ; *CC1CCOCC1]
wherein each of the formula depicted above is optionally substituted with one to three R²; wherein R², for each occurrence, is independently C₁₋₃alkyl or —OR^{2a}; and R^{2a}, for each occurrence, is independently H or C₁₋₃alkyl.

13. The compound of claim 12, or a pharmaceutically acceptable salt thereof, wherein R¹ is represented by the following formula:

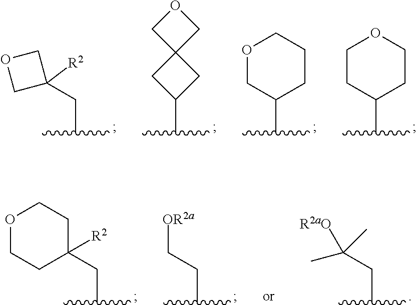

14. The compound of any one of claims 1 to 13, or a pharmaceutically acceptable salt thereof, wherein R², for each occurrence, is independently selected from —CH₃ and —OH; and R^{2a}, for each occurrence, is independently H or —CH₃.

15. The compound of any one of claims 1 to 14, wherein the compound is represented by Formula III: 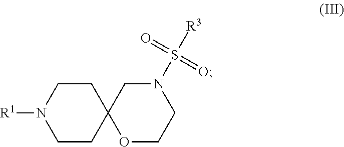
or a pharmaceutically acceptable salt thereof.

16. The compound of formula 15, wherein:
R¹ is represented by the following formula: 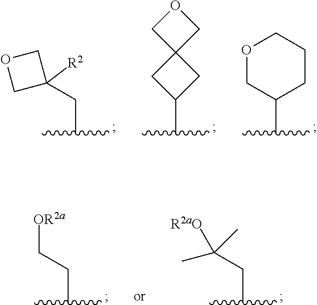
R², for each occurrence, is independently selected from —CH₃ and OH;
R^{2a}, for each occurrence, is independently selected from H or —CH₃;
R³ is represented by the following formula: 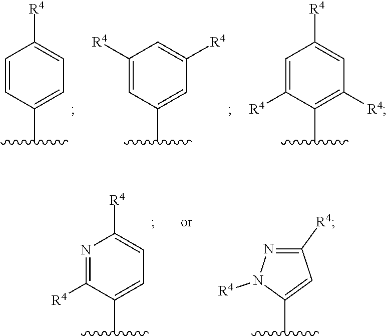
and
R⁴, for each occurrence, is independently selected from —CH₃, —CF₃, —OCHF₂, —OCH₃, and —F.

17. The compound of any one of claims 1 to 14, wherein the compound is represented by Formula IV: 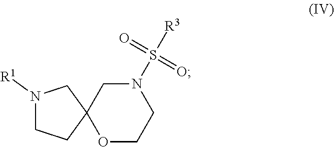
or a pharmaceutically acceptable salt thereof.

18. The compound of claim 17, wherein the compound is represented by Formulas (IVA) or (IVB): 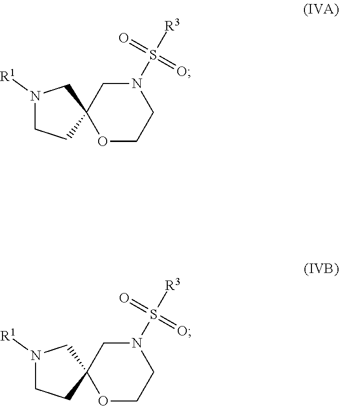
or a pharmaceutically acceptable salt thereof.

19. The compound of claim 17 or 18, or a pharmaceutically acceptable salt thereof, wherein:
R¹ is represented by the following formula: 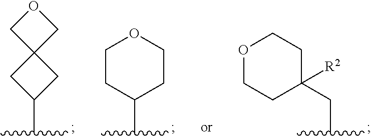
R² is OH;
R³ is represented by the following formula: 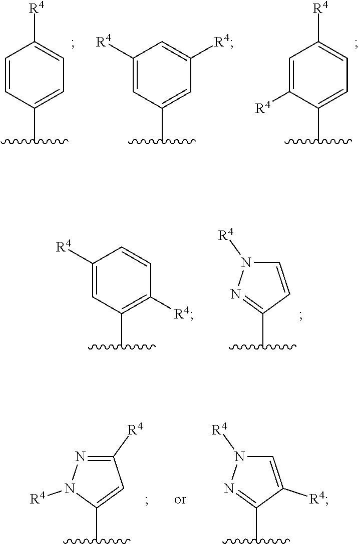
and
R⁴, for each occurrence, is independently selected from —CH₃, —CF₃, —OCH₃, —OCHF₂, —CN, isopropyl, —F, —Cl, and 4-methylpyridin-2-yl.

20. The compound of any one of claims 1 to 14, wherein the compound is represented by Formula V: 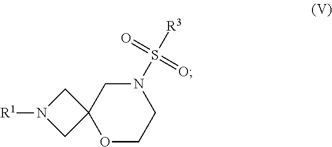
or a pharmaceutically acceptable salt thereof.

21. The compound of claim 20, wherein:
R¹ is represented by the following formula: 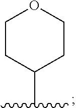 [SMILES: *C1CCOCC1]
R³ is represented by the following formula: 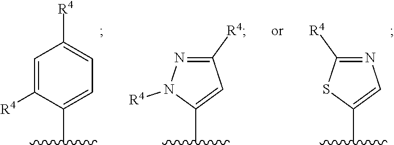
and
R⁴, for each occurrence, is independently selected from —CH₃, —F, and cyclopropyl.

22. The compound of claim 1 or 2, wherein the compound is represented by Formula VI: 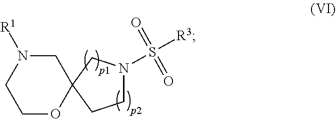
or a pharmaceutically acceptable salt thereof.

23. The compound of claim 22, wherein the compound is represented by Formula VII or VIII: 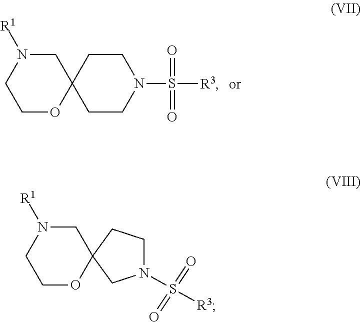
or a pharmaceutically acceptable salt thereof.

24. The compound of claim 22 or 23, or a pharmaceutically acceptable salt thereof, wherein R³ is phenyl optionally substituted with one to three R⁴.

25. The compound of claim 24, or a pharmaceutically acceptable salt thereof, wherein R³ is represented by the following formula:

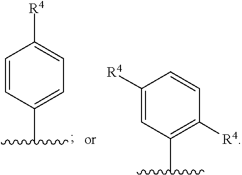

26. The compound of any one of claims 22 to 25, or a pharmaceutically acceptable salt thereof, wherein R⁴, for each occurrence, is independently selected from halo, CN, and OR^{4a}; and R^{4a} is C₁₋₃alkyl or C₁₋₃haloalkyl.

27. The compound of any one of claims 22 to 26, or a pharmaceutically acceptable salt thereof, wherein R⁴, for each occurrence, is independently selected from —OCHF₂, —F, and —CN.

28. The compound of any one of claims 22 to 27, or a pharmaceutically acceptable salt thereof, wherein R¹ is C₂₋₄allkyl substituted with —OR^{2a} or is represented by the following formula: 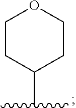 [SMILES: *C1CCOCC1]
optionally substituted with one to three R².

29. The compound of claim 28, or a pharmaceutically acceptable salt thereof, wherein R¹ is represented by the following formula:

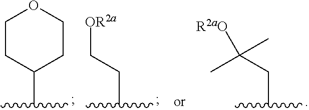

30. The compound of any one of claims 22 to 29, or a pharmaceutically acceptable salt thereof, wherein R², for each occurrence, is independently selected from —CH₃ and —OH; and R^{2a}, for each occurrence, is independently selected from H and —CH₃.

31. The compound of claim 1 or 2, wherein the compound is represented by Formula IX: 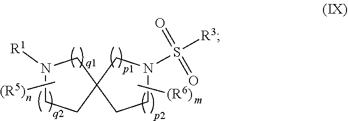
or a pharmaceutically acceptable salt thereof, wherein R⁵ is H, or two R⁵ together form a C₁₋₃alkylene; and R⁶ is H, or two R⁶ together form a C₁₋₃alkylene.

32. The compound of claim 31, or a pharmaceutically acceptable salt thereof, wherein p1 is 1 and p2 is 0; or p1 is 1 and p2 is 1; or p1 is 2 andp 2 is 1.

33. The compound of claim 31 or 32, or a pharmaceutically acceptable salt thereof, wherein R³ is selected from the group consisting of phenyl, pyridyl, and pyrazolyl, each of which is optionally substituted with one to three R⁴.

34. The compound of claim 31 or 32, or a pharmaceutically acceptable salt thereof, wherein R³ is selected from the group consisting of C₁₋₄alkyl-phenyl, phenyl, pyridyl, thiazoyl, and pyrazolyl, each of which is optionally substituted with one to three R⁴.

35. The compound of claim 33, or a pharmaceutically acceptable salt thereof, wherein R³ is represented by the following formula: 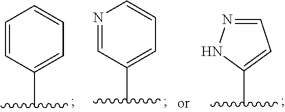
each of which is optionally substituted with one to three R⁴;
or R³ is represented by the following formula: 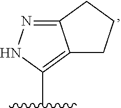 [SMILES: *c1[nH]nc2c1CCC2]
optionally substituted with one or two R⁴.

36. The compound of claim 34, or a pharmaceutically acceptable salt thereof, wherein R³ is represented by the following formula: 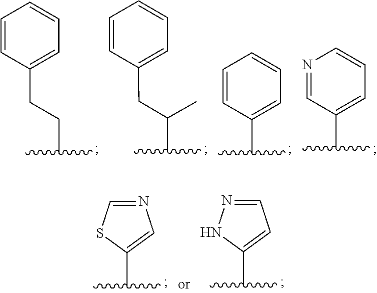
each of which is optionally substituted with one to three R⁴;
or R³ is represented by the following formula: 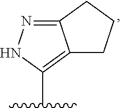 [SMILES: *c1[nH]nc2c1CCC2]
optionally substituted with one or two R⁴.

37. The compound of claim 35, or a pharmaceutically acceptable salt thereof, wherein R³ is represented by the following formula:

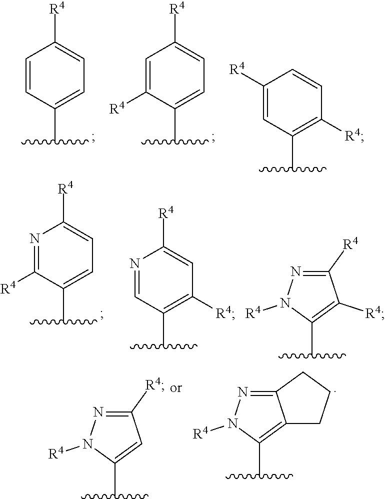

38. The compound of claim 36, or a pharmaceutically acceptable salt thereof, wherein R³ is represented by the following formula:

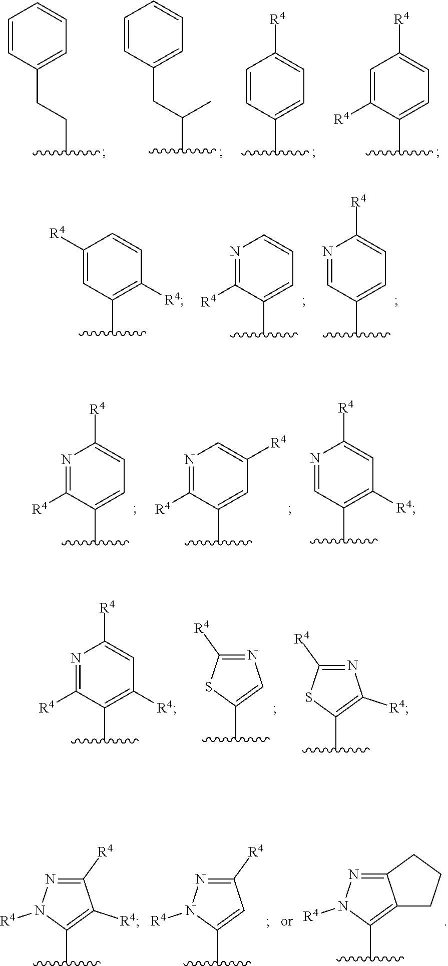

39. The compound of any one of claims 31 to 38, or a pharmaceutically acceptable salt thereof, wherein R⁴, for each occurrence, is independently selected from halo, CN, C₁₋₃alkyl, C₁₋₃haloalkyl, C₂₋₄alkenyl, C₃₋₆cycloalkyl, phenyl, and —OR^{4a}; wherein the C₃₋₆ cycloalkyl is optionally substituted with one to three halo or C₁₋₄haloalkyl and the C₁₋₃ alkyl is optionally substituted with one or two C₃₋₄cycloalkyl; and R^{4a} is C₁₋₃alkyl or C₁₋₃haloalkyl.

40. The compound of any one of claims 31 to 38, or a pharmaceutically acceptable salt thereof, wherein R⁴, for each occurrence, is independently selected from halo, CN, C₁₋₃ alkyl, C₁₋₃haloalkyl, C₃₋₆cycloalkyl, and —OR^{4a}; and R^{4a} is C₁₋₃alkyl or C₁₋₃haloalkyl.

41. The compound of any one of claims 31 to 40, or a pharmaceutically acceptable salt thereof, wherein R⁴, for each occurrence, is independently selected from —CH₃, —CH₂CH₃, —CF₂CH₃, —CF(CH₃)₂, —CH═CH₂, —CHF₂, —CH(CH₃)₂, —CF₃, —OCH₃, —OCHF₂, —OCF₃, cyclopropyl, 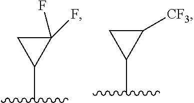
—CH₂-cyclopropyl, cyclobutyl, phenyl, —CN, —Cl, —Br, and —F.

42. The compound of any one of claims 31 to 40, or a pharmaceutically acceptable salt thereof, wherein R⁴, for each occurrence, is independently selected from —CH₃, —CHF₂, —CF₃, —OCH₃, —OCHF₂, —OCF₃, cyclopropyl, —CN, and —F.

43. The compound of any one of claims 31 to 42, or a pharmaceutically acceptable salt thereof, wherein R¹ is represented by the following formula: 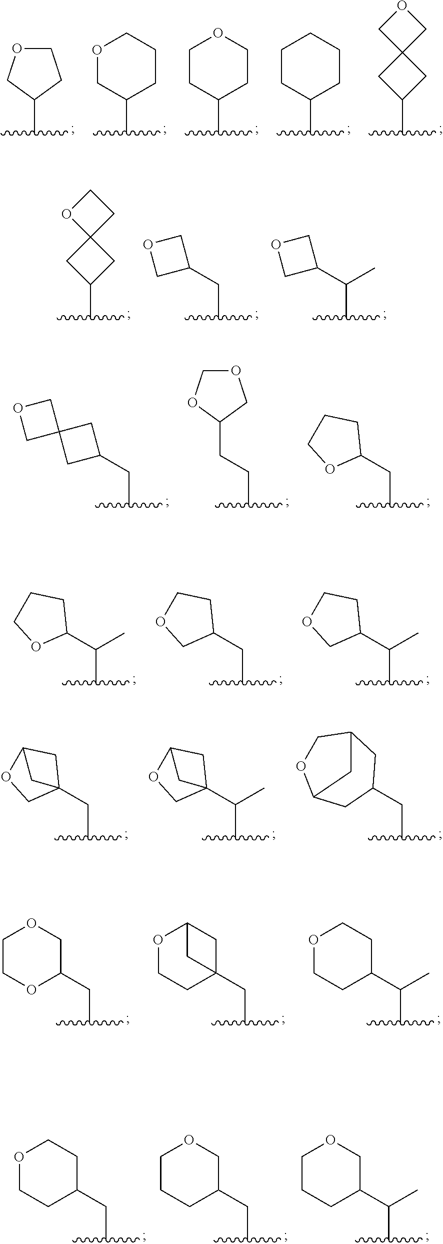 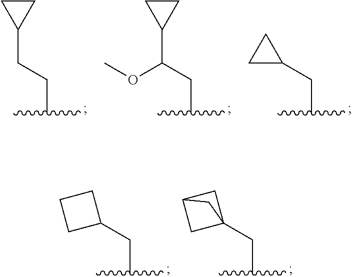
wherein each of the formula depicted above is optionally substituted with one to three R².

44. The compound of any one of claims 31 to 42, or a pharmaceutically acceptable salt thereof, wherein R¹ is represented by the following formula: 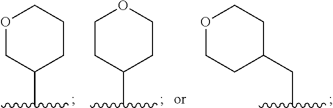
wherein each of the formula depicted above is optionally substituted with one to three R².

45. The compound of claim 43, or a pharmaceutically acceptable salt thereof, wherein R¹ is represented by the following formula:

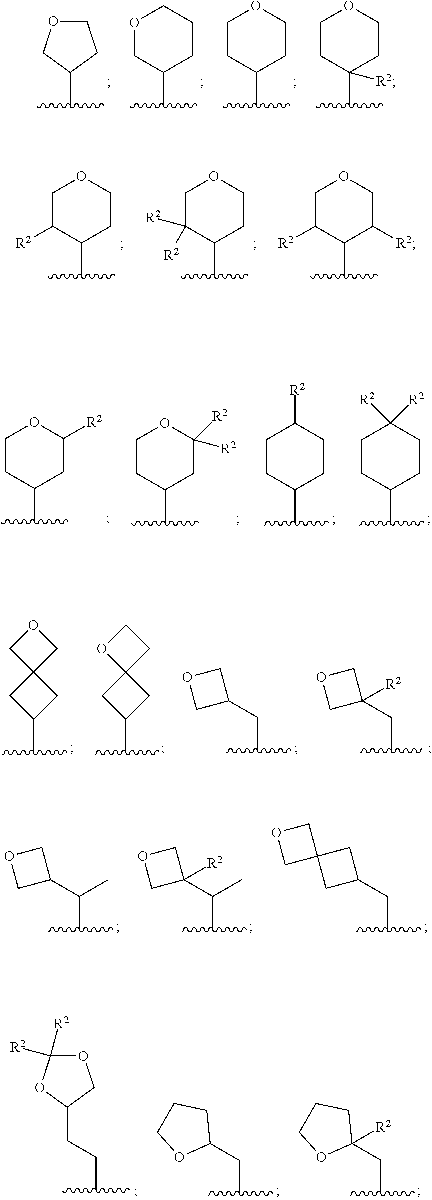

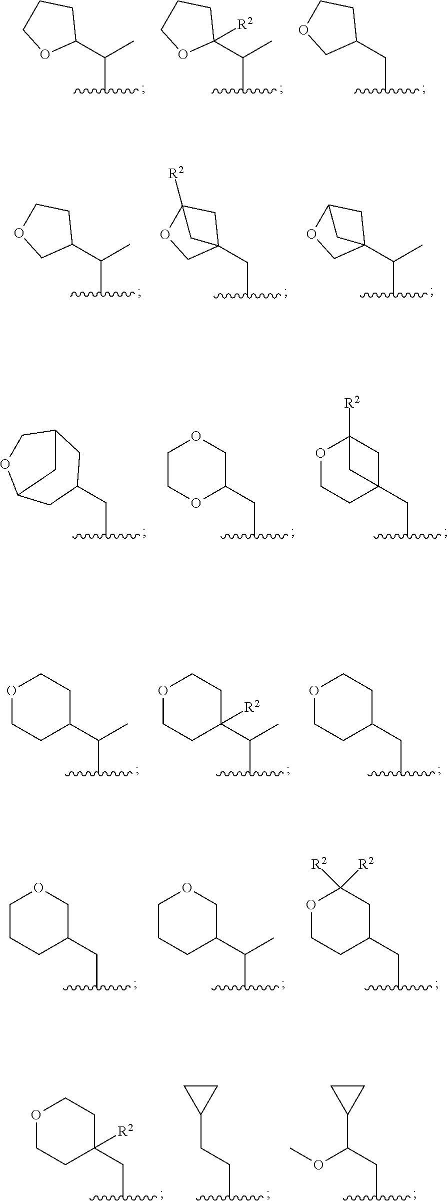

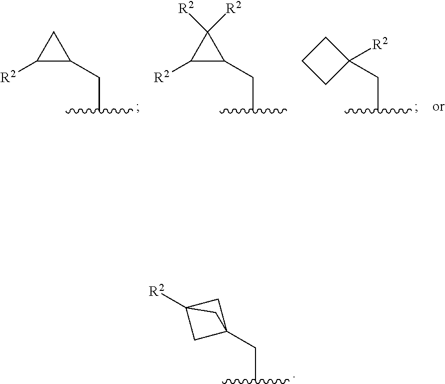

46. The compound of claim 44, or a pharmaceutically acceptable salt thereof, wherein R¹ is represented by the following formula:

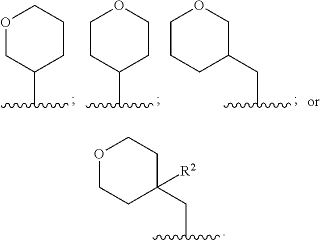

47. The compound of any one of claims 31 to 46, or a pharmaceutically acceptable salt thereof, wherein R² is —CH₃, —F, —OCH₃—CN, —CH₂CH₂OCH₃ or —OH.

48. The compound of any one of claims 31 to 46, or a pharmaceutically acceptable salt thereof, wherein R² is OH.

49. The compound of any one of claims 31 to 42, or a pharmaceutically acceptable salt thereof, wherein:
R¹ is C₂₋₆alkyl optionally substituted with one or two R^{A};
R^{A}, for each occurrence, is independently OR^{2a}, SR^{2a}, or C(O)OR^{2a}; and
R^{2a} is H, C₁₋₄alkyl, or C₃₋₆cycloalkyl wherein the C₁₋₄alkyl is optionally substituted with one or two C₁₋₃alkoxy.

50. The compound of claim 49, or a pharmaceutically acceptable salt thereof, wherein R¹ is
—CH₂CH₃, —CH₂CH₂CH₂SCH₂CH₃, —CH₂CH₂CH₂O-cyclopentyl, —CH₂CH₂CH₂CH₂CH₂OCH₃, —CH₂CH₂OC(CH₃)3, —CH₂CH₂CH₂CH(CH₃)OCH₃, —CH₂CH(CH₃)CH₂CH₂C(O)OCH₃, —CH₂CH₂C(CH₃)₂C(O)OCH₃, or —CH₂CH₂CH₂OCH₂CH₂OCH₃.

51. The compound of any one of claims 31 to 48, wherein the compound is represented by Formula X: 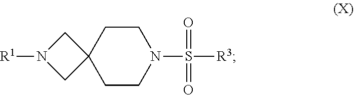
or a pharmaceutically acceptable salt thereof.

52. The compound of claim 51, wherein:
R¹ is represented by the following formula: 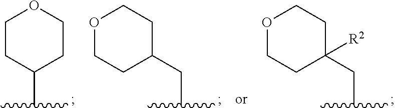
R² is OH;
R³ is represented by the following formula: 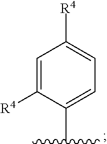
and
R⁴ is —CH₃.

53. The compound of any one of claims 31 to 48, wherein the compound is represented by Formula XI: 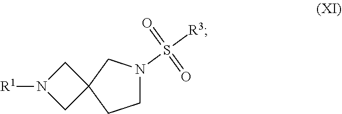
or a pharmaceutically acceptable salt thereof.

54. The compound of claim 53, wherein the compound is represented by Formulas (XIA) or (XIB): 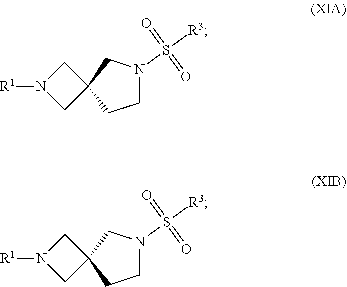
or a pharmaceutically acceptable salt thereof.

55. The compound of claim 53 of 54, wherein:
R¹ is represented by the following formula: 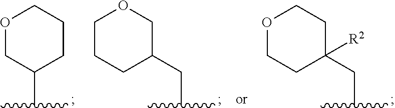
R² is OH;
R³ is represented by the following formula: 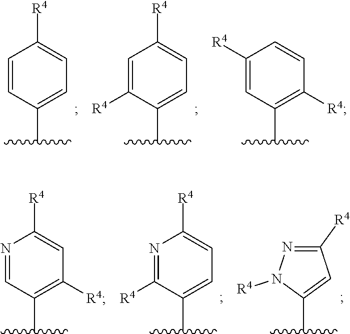
and
R⁴, for each occurrence, is independently selected from —CH₃, —CF₃, —OCH₃, —OCHF₂, —OCF₃, —CN, —F, and cyclopropyl.

56. The compound of any one of claims 31 to 50, wherein the compound is represented by Formula XII: 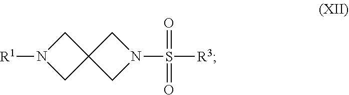
or a pharmaceutically acceptable salt thereof.

57. The compound of claim 56, or a pharmaceutically acceptable salt thereof, wherein:
R¹ is represented by the following formula: 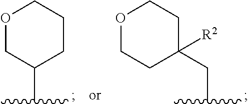
R² is OH;
R³ is represented by the following formula: 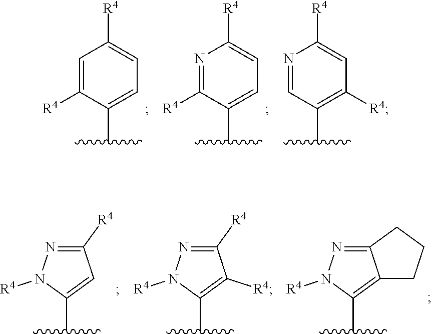
and
R⁴, for each occurrence, is independently selected from —CH₃, —CHF₂, —CF₃, —OCH₃, —OCHF₂, and —OCF₃.

58. The compound of any one of claims 31 to 48, wherein the compound is represented by Formula XII: 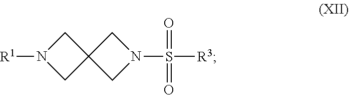
or a pharmaceutically acceptable salt thereof, wherein:
R¹ is Het or Z-Het;
Z is CH₂;
Het is represented by the following formula: 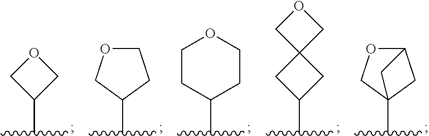
wherein each of the formula depicted above is optionally substituted with one to two R²;
R², for each occurrence, is independently C₁₋₄alkyl, —OH, or halo;
R³ is pyridinyl or pyrazolyl, each of which is optionally substituted with one to two R⁴;
R⁴, for each occurrence, is independently C₁₋₄alkyl or C₁₋₄haloalkyl.

59. The compound of claim 58, or a pharmaceutically acceptable salt thereof, wherein R³ is represented by the following formula: 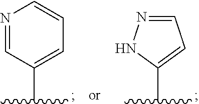 [SMILES: *c1cccnc1 ; *c1ccn[nH]1]
each of which is optionally substituted with one to two R⁴.

60. The compound of claim 59, or a pharmaceutically acceptable salt thereof, wherein R³ is represented by the following formula:

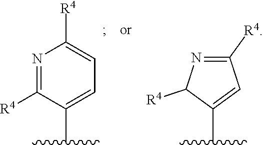

61. The compound of any one of claims 58 to 60, or a pharmaceutically acceptably salt thereof, wherein R⁴, for each occurrence, is independently —CH₃, —CF₃, or —CF₂CH₃.

62. The compound of claim 60, wherein the compound is represented by Formula XIIA: 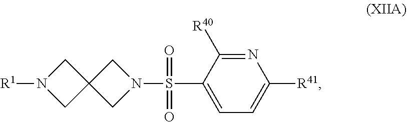
or a pharmaceutically acceptable salt thereof, wherein R⁴⁰ is C₁₋₃alkyl and R⁴¹ is C₁₋₃haloalkyl.

63. The compound of claim 62, or a pharmaceutically acceptable salt thereof, wherein R⁴⁰ is —CH₃ and R⁴¹ is —CF₃ or —CF₂CH₃.

64. The compound of any one of claims 58 to 63, or a pharmaceutically acceptably salt thereof, wherein R¹ is represented by the following formula: 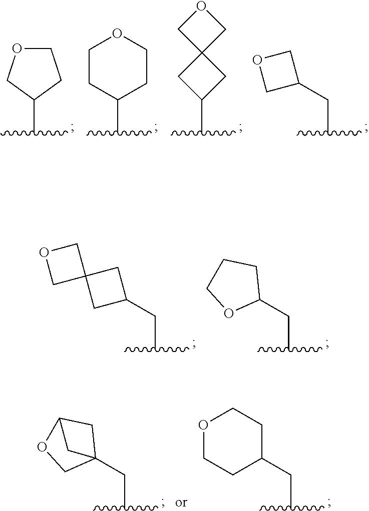
wherein each of the formula depicted above is optionally substituted with one to two R².

65. The compound of claim 64, or a pharmaceutically acceptably salt thereof, wherein R¹ is represented by the following formula:

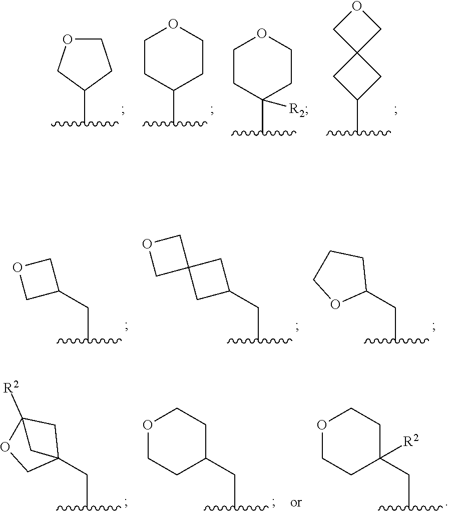

66. The compound of any one of claims 58 to 65, or a pharmaceutically acceptably salt thereof, wherein R², for each occurrence, is independently —CH₃, OH, or —F.

67. The compound of claim 1, or a pharmaceutically acceptable salt thereof, wherein the compound is selected from:
8-((4-(Difluoromethoxy)phenyl)sulfonyl)-2-(tetrahydro-2H-pyran-4-yl)-5-oxa-2,8-diazaspiro[3.5]nonane;
9-((4-(Difluoromethoxy)phenyl)sulfonyl)-2-(tetrahydro-2H-pyran-4-yl)-6-oxa-2,9-diazaspiro[4.5]decane;
(S)-9-((4-(Difluoromethoxy)phenyl)sulfonyl)-2-(tetrahydro-2H-pyran-4-yl)-6-oxa-2,9-diazaspiro[4.5]decane;
(R)-9-((4-(Difluoromethoxy)phenyl)sulfonyl)-2-(tetrahydro-2H-pyran-4-yl)-6-oxa-2,9-diazaspiro[4.5]decane;
4-((4-(Difluoromethoxy)phenyl)sulfonyl)-9-(tetrahydro-2H-pyran-4-yl)-1-oxa-4,9-diazaspiro[5.5]undecane;
4-((4-(Difluoromethoxy)phenyl)sulfonyl)-9-(tetrahydrofuran-3-yl)-1-oxa-4,9-diazaspiro[5.5]undecane;
4-((4-(Difluoromethoxy)phenyl)sulfonyl)-9-(2-methoxyethyl)-1-oxa-4,9-diazaspiro[5.5]undecane;
9-((4-(Difluoromethoxy)phenyl)sulfonyl)-2-((S)-tetrahydro-2H-pyran-3-yl)-6-oxa-2,9-diazaspiro[4.5]decane;
(±)—(R)-9-((4-(Difluoromethoxy)phenyl)sulfonyl)-2-((S)-tetrahydro-2H-pyran-3-yl)-6-oxa-2,9-diazaspiro[4.5]decane;
(±)—(R)-9-((4-(Difluoromethoxy)phenyl)sulfonyl)-2-((R)-tetrahydro-2H-pyran-3-yl)-6-oxa-2,9-diazaspiro[4.5]decane;
1-(4-((4-(Difluoromethoxy)phenyl)sulfonyl)-1-oxa-4,9-diazaspiro[5.5]undecan-9-yl)-2-methylpropan-2-ol;
4-((4-(Difluoromethoxy)phenyl)sulfonyl)-9-(2-methoxy-2-methylpropyl)-1-oxa-4,9-diazaspiro[5.5]undecane;
9-((2,4-Difluorophenyl)sulfonyl)-2-(tetrahydro-2H-pyran-4-yl)-6-oxa-2,9-diazaspiro[4.5]decane;
9-((3,5-Difluorophenyl)sulfonyl)-2-(tetrahydro-2H-pyran-4-yl)-6-oxa-2,9-diazaspiro[4.5]decane;
(S)-9-((3,5-Difluorophenyl)sulfonyl)-2-(tetrahydro-2H-pyran-4-yl)-6-oxa-2,9-diazaspiro[4.5]decane;
(R)-9-((3,5-Difluorophenyl)sulfonyl)-2-(tetrahydro-2H-pyran-4-yl)-6-oxa-2,9-diazaspiro[4.5]decane;
9-((2-Methoxy-5-methylpyridin-3-yl)sulfonyl)-2-(tetrahydro-2H-pyran-4-yl)-6-oxa-2,9-diazaspiro[4.5]decane;
9-((4-(Difluoromethoxy)phenyl)sulfonyl)-2-(oxetan-3-ylmethyl)-6-oxa-2,9-diazaspiro[4.5]decane;
9-((4-(Difluoromethoxy)phenyl)sulfonyl)-2-((tetrahydro-2H-pyran-4-yl)methyl)-6-oxa-2,9-diazaspiro[4.5]decane;
4-((4-(Difluoromethoxy)phenyl)sulfonyl)-9-((tetrahydro-2H-pyran-4-yl)methyl)-1-oxa-4,9-diazaspiro[5.5]undecane;
4-((4-(Difluoromethoxy)phenyl)sulfonyl)-9-((3-methyloxetan-3-yl)methyl)-1-oxa-4,9-diazaspiro[5.5]undecane;
4-((4-(Difluoromethoxy)phenyl)sulfonyl)-9-(2,2-dimethyltetrahydro-2H-pyran-4-yl)-1-oxa-4,9-diazaspiro[5.5]undecane;
8-((2-Cyclopropylthiazol-5-yl)sulfonyl)-2-(tetrahydro-2H-pyran-4-yl)-5-oxa-2,8-diazaspiro[3.5]nonane;
9-((2-Cyclopropylthiazol-5-yl)sulfonyl)-2-(tetrahydro-2H-pyran-4-yl)-6-oxa-2,9-diazaspiro[4.5]decane;
9-((2-Cyclopropylthiazol-5-yl)sulfonyl)-2-(2-oxaspiro[3.3]heptan-6-yl)-6-oxa-2,9-diazaspiro[4.5]decane;
9-((4-(Difluoromethoxy)phenyl)sulfonyl)-2-(2-oxaspiro[3.3]heptan-6-yl)-6-oxa-2,9-diazaspiro[4.5]decane;
(R)-9-((4-(Difluoromethoxy)phenyl)sulfonyl)-2-(2-oxaspiro[3.3]heptan-6-yl)-6-oxa-2,9-diazaspiro[4.5]decane;
(S)-9-((4-(Difluoromethoxy)phenyl)sulfonyl)-2-(2-oxaspiro[3.3]heptan-6-yl)-6-oxa-2,9-diazaspiro[4.5]decane;
9-((3,5-Difluorophenyl)sulfonyl)-2-(2-oxaspiro[3.3]heptan-6-yl)-6-oxa-2,9-diazaspiro[4.5]decane;
4-((3,5-Difluorophenyl)sulfonyl)-9-((3-methyloxetan-3-yl)methyl)-1-oxa-4,9-diazaspiro[5.5]undecane;
9-((4-(Difluoromethoxy)phenyl)sulfonyl)-4-(tetrahydro-2H-pyran-4-yl)-1-oxa-4,9-diazaspiro[5.5]undecane;
7-((2,4-Dimethylphenyl)sulfonyl)-2-(tetrahydro-2H-pyran-4-yl)-2,7-diazaspiro[3.5]nonane;
7-((4-(Difluoromethoxy)phenyl)sulfonyl)-2-(tetrahydro-2H-pyran-4-yl)-2,7-diazaspiro[3.5]nonane;
7-((2-methyl-6-(trifluoromethyl)pyridin-3-yl)sulfonyl)-2-(tetrahydro-2H-pyran-4-yl)-2,7-diazaspiro[3.5]nonane;
1-((7-((2,4-Dimethylphenyl)sulfonyl)-2,7-diazaspiro[3.5]nonan-2-yl)methyl)tetrahydro-2H-pyran-4-ol;
7-((2,4-Dimethylphenyl)sulfonyl)-2-((tetrahydro-2H-pyran-4-yl)methyl)-2,7-diazaspiro[3.5]nonane;
8-((2-Chloro-4-methylphenyl)sulfonyl)-2-(tetrahydro-2H-pyran-4-yl)-2,8-diazaspiro[4.5]decane;
8-((1,3-Dimethyl-1H-pyrazol-5-yl)sulfonyl)-2-(tetrahydro-2H-pyran-4-yl)-2,8-diazaspiro[4.5]decane;
8-((4-Methyl-6-(trifluoromethyl)pyridin-3-yl)sulfonyl)-2-(tetrahydro-2H-pyran-4-yl)-2,8-diazaspiro[4.5]decane;
8-((4-(Difluoromethoxy)phenyl)sulfonyl)-2-(tetrahydro-2H-pyran-4-yl)-2,8-diazaspiro[4.5]decane;
8-((1-Methyl-3-(trifluoromethyl)-1H-pyrazol-5-yl)sulfonyl)-2-(tetrahydro-2H-pyran-4-yl)-2,8-diazaspiro[4.5]decane;
8-((2-Methyl-4-(trifluoromethoxy)phenyl)sulfonyl)-2-(tetrahydro-2H-pyran-4-yl)-2,8-diazaspiro[4.5]decane;
8-((2-Methyl-6-(trifluoromethyl)pyridin-3-yl)sulfonyl)-2-(tetrahydro-2H-pyran-4-yl)-2,8-diazaspiro[4.5]decane;
8-((6-Methoxy-2-methylpyridin-3-yl)sulfonyl)-2-(tetrahydro-2H-pyran-4-yl)-2,8-diazaspiro[4.5]decane;
2-((2,4-Dimethylphenyl)sulfonyl)-6-(tetrahydro-2H-pyran-4-yl)-2,6-diazaspiro[3.3]heptane;
4-((6-((2,4-Dimethylphenyl)sulfonyl)-2,6-diazaspiro[3.3]heptan-2-yl)methyl)tetrahydro-2H-pyran-4-ol;
2-((1-Methyl-3-(trifluoromethyl)-1H-pyrazol-5-yl)sulfonyl)-6-(tetrahydro-2H-pyran-4-yl)-2,6-diazaspiro[3.3]heptane;
2-((6-Methoxy-2-methylpyridin-3-yl)sulfonyl)-6-(tetrahydro-2H-pyran-4-yl)-2,6-diazaspiro[3.3]heptane;
2-((4-Methyl-6-(trifluoromethyl)pyridin-3-yl)sulfonyl)-6-(tetrahydro-2H-pyran-4-yl)-2,6-diazaspiro[3.3]heptane;
2-((4-(Difluoromethyl)-1,3-dimethyl-1H-pyrazol-5-yl)sulfonyl)-6-(tetrahydro-2H-pyran-4-yl)-2,6-diazaspiro[3.3]heptane;
2-((2-Methyl-4-(trifluoromethoxy)phenyl)sulfonyl)-6-(tetrahydro-2H-pyran-4-yl)-2,6-diazaspiro[3.3]heptane;
2-((2-Methyl-6-(trifluoromethyl)pyridin-3-yl)sulfonyl)-6-(tetrahydro-2H-pyran-4-yl)-2,6-diazaspiro[3.3]heptane;
2-((2,4-Dimethylphenyl)sulfonyl)-6-(tetrahydro-2H-pyran-4-yl)-2,6-diazaspiro[3.4]octane;
2-((4,6-Dimethylpyridin-3-yl)sulfonyl)-6-(tetrahydro-2H-pyran-4-yl)-2,6-diazaspiro[3.4]octane;
2-((4-(Difluoromethoxy)phenyl)sulfonyl)-6-(tetrahydro-2H-pyran-4-yl)-2,6-diazaspiro[3.4]octane;
2-((1-Methyl-3-(trifluoromethyl)-1H-pyrazol-5-yl)sulfonyl)-6-(tetrahydro-2H-pyran-4-yl)-2,6-diazaspiro[3.4]octane;
2-((2-Methyl-6-(trifluoromethyl)pyridin-3-yl)sulfonyl)-6-(tetrahydro-2H-pyran-4-yl)-2,6-diazaspiro[3.4]octane;
2-((4-Methyl-6-(trifluoromethyl)pyridin-3-yl)sulfonyl)-6-(tetrahydro-2H-pyran-4-yl)-2,6-diazaspiro[3.4]octane;
2-((2-Methyl-4-(trifluoromethoxy)phenyl)sulfonyl)-6-(tetrahydro-2H-pyran-4-yl)-2,6-diazaspiro[3.4]octane;
2-((3-Cyclopropyl-1-methyl-1H-pyrazol-5-yl)sulfonyl)-6-(tetrahydro-2H-pyran-4-yl)-2,6-diazaspiro[3.4]octane;
6-((1-methyl-3-(Trifluoromethyl)-1H-pyrazol-5-yl)sulfonyl)-2-(tetrahydro-2H-pyran-4-yl)-2,6-diazaspiro[3.4]octane;
(1′R,5′S)-8′-((4-(Difluoromethoxy)phenyl)sulfonyl)-1-(tetrahydro-2H-pyran-4-yl)-8′-azaspiro[azetidine-3,3′-bicyclo[3.2.1]octane];
1-((1′R,5′S)-1-((2,4-Dimethylphenyl)sulfonyl)-8′-azaspiro[azetidine-3,3′-bicyclo[3.2.1]octan]-8′-yl)-2-methylpropan-2-ol;
(1′R,5′S)-8′-((4-(Difluoromethoxy)phenyl)sulfonyl)-1-(2-methoxy-2-methylpropyl)-8′-azaspiro[azetidine-3,3′-bicyclo[3.2.1]octane];
(1′R,5′S)-1-((4-(Difluoromethoxy)phenyl)sulfonyl)-8′-(2-methoxy-2-methylpropyl)-8′-azaspiro[azetidine-3,3′-bicyclo[3.2.1]octane];
2-((1-(cyclopropylmethyl)-3-(trifluoromethyl)-1H-pyrazol-5-yl)sulfonyl)-6-(tetrahydro-2H-pyran-4-yl)-2,6-diazaspiro[3.3]heptane;
2-((1-(cyclopropylmethyl)-3-(trifluoromethyl)-1H-pyrazol-5-yl)sulfonyl)-6-((tetrahydro-2H-pyran-4-yl)methyl)-2,6-diazaspiro[3.3]heptane;
2-((6-bromo-2-methylpyridin-3-yl)sulfonyl)-6-(tetrahydro-2H-pyran-4-yl)-2,6-diazaspiro[3.3]heptane;
2-((2-methyl-6-(trifluoromethyl)pyridin-3-yl)sulfonyl)-6-(2-oxaspiro[3.3]heptan-6-yl)-2,6-diazaspiro[3.3]heptane;
2-((2-methyl-6-(trifluoromethyl)pyridin-3-yl)sulfonyl)-6-(1-oxaspiro[3.3]heptan-6-yl)-2,6-diazaspiro[3.3]heptane;
2-((2-methyl-6-(trifluoromethyl)-1,4-dihydropyridin-3-yl)sulfonyl)-6-(1-oxaspiro[3.3]heptan-6-yl)-2,6-diazaspiro[3.3]heptane;
2-((2-methyl-6-(trifluoromethyl)pyridin-3-yl)sulfonyl)-6-(1-oxaspiro[3.3]heptan-6-yl)-2,6-diazaspiro[3.3]heptane;
2-((1-methyl-3-(trifluoromethyl)-1H-pyrazol-5-yl)sulfonyl)-6-((4-methyltetrahydro-2H-pyran-4-yl)methyl)-2,6-diazaspiro[3.3]heptane;
2-((1-methyl-2-oxabicyclo[2.1.1]hexan-4-yl)methyl)-6-((2-methyl-6-(trifluoromethyl)pyridin-3-yl)sulfonyl)-2,6-diazaspiro[3.3]heptane;
2-((1-methyl-2-oxabicyclo[3.1.1]heptan-5-yl)methyl)-6-((2-methyl-6-(trifluoromethyl)-1,6-dihydropyridin-3-yl)sulfonyl)-2,6-diazaspiro[3.3]heptane;
4-((6-((6-(1,1-difluoroethyl)-2-methylpyridin-3-yl)sulfonyl)-2,6-diazaspiro[3.3]heptan-2-yl)methyl)tetrahydro-2H-pyran-4-ol;
2-((1-methyl-3-(trifluoromethyl)-1H-pyrazol-5-yl)sulfonyl)-6-(4-methyltetrahydro-2H-pyran-4-yl)-2,6-diazaspiro[3.3]heptane;
2-((2-methyl-6-(trifluoromethyl)-1,4-dihydropyridin-3-yl)sulfonyl)-6-(4-methyltetrahydro-2H-pyran-4-yl)-2,6-diazaspiro[3.3]heptane;
2-((6-(1,1-difluoroethyl)-2-methylpyridin-3-yl)sulfonyl)-6-(2-oxaspiro[3.3]heptan-6-yl)-2,6-diazaspiro[3.3]heptane;
2-((6-(1,1-difluoroethyl)-2-methylpyridin-3-yl)sulfonyl)-6-(tetrahydro-2H-pyran-4-yl)-2,6-diazaspiro[3.3]heptane;
(R)-2-((6-(1,1-difluoroethyl)-2-methylpyridin-3-yl)sulfonyl)-6-(tetrahydro-2H-pyran-3-yl)-2,6-diazaspiro[3.3]heptane;
2-((6-(1,1-difluoroethyl)-2-methyl-3,4-dihydropyridin-3-yl)sulfonyl)-6-((tetrahydro-2H-pyran-4-yl)methyl)-2,6-diazaspiro[3.3]heptane;
(S)-2-((6-(1,1-difluoroethyl)-2-methylpyridin-3-yl)sulfonyl)-6-(1-(tetrahydro-2H-pyran-4-yl)ethyl)-2,6-diazaspiro[3.3]heptane;
2-((6-(1,1-difluoroethyl)-2-methylpyridin-3-yl)sulfonyl)-6-((4-methyltetrahydro-2H-pyran-4-yl)methyl)-2,6-diazaspiro[3.3]heptane;
(R)-2-((6-(1,1-difluoroethyl)-2-methylpyridin-3-yl)sulfonyl)-6-(tetrahydrofuran-3-yl)-2,6-diazaspiro[3.3]heptane;
(S)-2-((6-(1,1-difluoroethyl)-2-methylpyridin-3-yl)sulfonyl)-6-((tetrahydrofuran-3-yl)methyl)-2,6-diazaspiro[3.3]heptane;
2-((6-(1,1-difluoroethyl)-2-methylpyridin-3-yl)sulfonyl)-6-((S)-1-((S)-tetrahydrofuran-3-yl)ethyl)-2,6-diazaspiro[3.3]heptane;
2-((6-(1,1-difluoroethyl)-2-methylpyridin-3-yl)sulfonyl)-6-(oxetan-3-ylmethyl)-2,6-diazaspiro[3.3]heptane;
(S)-2-((6-(1,1-difluoroethyl)-2-methylpyridin-3-yl)sulfonyl)-6-(1-(oxetan-3-yl)ethyl)-2,6-diazaspiro[3.3]heptane;
2-((6-(1,1-difluoroethyl)-2-methylpyridin-3-yl)sulfonyl)-6-((3-methyloxetan-3-yl)methyl)-2,6-diazaspiro[3.3]heptane;
2-((6-(difluoromethyl)-2-methylpyridin-3-yl)sulfonyl)-6-(tetrahydro-2H-pyran-4-yl)-2,6-diazaspiro[3.3]heptane;
2-((6-(difluoromethyl)-2-methylpyridin-3-yl)sulfonyl)-6-(2-oxaspiro[3.3]heptan-6-yl)-2,6-diazaspiro[3.3]heptane;
2-((6-(2-fluoropropan-2-yl)-2-methyl-1,4-dihydropyridin-3-yl)sulfonyl)-6-(2-oxaspiro[3.3]heptan-6-yl)-2,6-diazaspiro[3.3]heptane;
2-((6-(2-fluoropropan-2-yl)-2-methylpyridin-3-yl)sulfonyl)-6-(tetrahydro-2H-pyran-4-yl)-2,6-diazaspiro[3.3]heptane;
2-((6-(difluoromethoxy)-2-methyl-3,4-dihydropyridin-3-yl)sulfonyl)-6-(tetrahydro-2H-pyran-4-yl)-2,6-diazaspiro[3.3]heptane;
2-((6-(difluoromethoxy)-2-methylpyridin-3-yl)sulfonyl)-6-(2-oxaspiro[3.3]heptan-6-yl)-2,6-diazaspiro[3.3]heptane;
2-((5-(difluoromethyl)-2-methyl-1H-2l4-pyrazol-3-yl)sulfonyl)-6-(tetrahydro-2H-pyran-4-yl)-2,6-diazaspiro[3.3]heptane;
2-((3-methoxy-1-methyl-1H-pyrazol-5-yl)sulfonyl)-6-(tetrahydro-2H-pyran-4-yl)-2,6-diazaspiro[3.3]heptane;
2-((2-chloro-6-methoxypyridin-3-yl)sulfonyl)-6-(tetrahydro-2H-pyran-4-yl)-2,6-diazaspiro[3.3]heptane;
2-((5-chloro-2-methoxypyridin-3-yl)sulfonyl)-6-(tetrahydro-2H-pyran-4-yl)-2,6-diazaspiro[3.3]heptane;
2-((2,4-dimethyl-6-(trifluoromethyl)pyridin-3-yl)sulfonyl)-6-(tetrahydro-2H-pyran-4-yl)-2,6-diazaspiro[3.3]heptane;
2-((2,4-dimethyl-6-(trifluoromethyl)pyridin-3-yl)sulfonyl)-6-(2-oxaspiro[3.3]heptan-6-yl)-2,6-diazaspiro[3.3]heptane;
2-((2,4-dimethyl-6-(trifluoromethyl)pyridin-3-yl)sulfonyl)-6-((tetrahydro-2H-pyran-4-yl)methyl)-2,6-diazaspiro[3.3]heptane;
(S)-2-((2,4-dimethyl-6-(trifluoromethyl)pyridin-3-yl)sulfonyl)-6-((tetrahydrofuran-3-yl)methyl)-2,6-diazaspiro[3.3]heptane;
2-((2,4-dimethyl-6-(trifluoromethyl)pyridin-3-yl)sulfonyl)-6-(oxetan-3-ylmethyl)-2,6-diazaspiro[3.3]heptane;
2-((6-methyl-2-(trifluoromethyl)-1,4-dihydropyridin-3-yl)sulfonyl)-6-(tetrahydro-2H-pyran-4-yl)-2,6-diazaspiro[3.3]heptane;
2-((6-methyl-2-(trifluoromethyl)pyridin-3-yl)sulfonyl)-6-((tetrahydro-2H-pyran-4-yl)methyl)-2,6-diazaspiro[3.3]heptane;
2-((6-cyclopropyl-2-(trifluoromethyl)pyridin-3-yl)sulfonyl)-6-(tetrahydro-2H-pyran-4-yl)-2,6-diazaspiro[3.3]heptane;
2-((6-cyclopropyl-2-(trifluoromethyl)pyridin-3-yl)sulfonyl)-6-((tetrahydro-2H-pyran-4-yl)methyl)-2,6-diazaspiro[3.3]heptane;
2-((2,6-bis(trifluoromethyl)pyridin-3-yl)sulfonyl)-6-(tetrahydro-2H-pyran-4-yl)-2,6-diazaspiro[3.3]heptane;
2-((2,6-bis(trifluoromethyl)-1,4-dihydropyridin-3-yl)sulfonyl)-6-(2-oxaspiro[3.3]heptan-6-yl)-2,6-diazaspiro[3.3]heptane;
2-((2,6-bis(trifluoromethyl)pyridin-3-yl)sulfonyl)-6-((tetrahydro-2H-pyran-4-yl)methyl)-2,6-diazaspiro[3.3]heptane;
2-((2,6-bis(trifluoromethyl)pyridin-3-yl)sulfonyl)-6-(oxetan-3-ylmethyl)-2,6-diazaspiro[3.3]heptane;
2-((2-oxaspiro[3.3]heptan-6-yl)methyl)-6-((2-methyl-6-(trifluoromethyl)pyridin-3-yl)sulfonyl)-2,6-diazaspiro[3.3]heptane;
2-((2-oxaspiro[3.3]heptan-6-yl)methyl)-6-((6-(1,1-difluoroethyl)-2-methylpyridin-3-yl)sulfonyl)-2,6-diazaspiro[3.3]heptane;
2-((2-oxaspiro[3.3]heptan-6-yl)methyl)-6-((2-methyl-5-(trifluoromethyl)-2,5-dihydro-1H-pyrazol-3-yl)sulfonyl)-2,6-diazaspiro[3.3]heptane;
2-((2-cyclopropyl-6-(difluoromethyl)pyridin-3-yl)sulfonyl)-6-(tetrahydro-2H-pyran-4-yl)-2,6-diazaspiro[3.3]heptane;
2-((2-cyclopropyl-6-(difluoromethyl)-1,4-dihydropyridin-3-yl)sulfonyl)-6-((1s,4s)-4-methoxycyclohexyl)-2,6-diazaspiro[3.3]heptane;
2-((2-cyclopropyl-6-(difluoromethyl)pyridin-3-yl)sulfonyl)-6-(4-methoxycyclohexyl)-2,6-diazaspiro[3.3]heptane;
2-((2-cyclopropyl-6-(difluoromethyl)pyridin-3-yl)sulfonyl)-6-(2-oxaspiro[3.3]heptan-6-yl)-2,6-diazaspiro[3.3]heptane;
2-((1-cyclopropyl-3-(difluoromethyl)-1H-pyrazol-5-yl)sulfonyl)-6-(tetrahydro-2H-pyran-4-yl)-2,6-diazaspiro[3.3]heptane;
2-((4-cyclopropyl-6-(trifluoromethyl)pyridin-3-yl)sulfonyl)-6-(2-oxaspiro[3.3]heptan-6-yl)-2,6-diazaspiro[3.3]heptane;
2-((4-cyclopropyl-6-(trifluoromethyl)pyridin-3-yl)sulfonyl)-6-((tetrahydro-2H-pyran-4-yl)methyl)-2,6-diazaspiro[3.3]heptane;
(1r,4r)-4-(6-((4-cyclopropyl-6-(trifluoromethyl)-2,3-dihydropyridin-3-yl)sulfonyl)-2,6-diazaspiro[3.3]heptan-2-yl)-1-methylcyclohexan-1-ol;
4-(6-((4-cyclopropyl-6-(trifluoromethyl)pyridin-3-yl)sulfonyl)-2,6-diazaspiro[3.3]heptan-2-yl)-1-methylcyclohexan-1-ol;
2-((4-ethyl-6-(trifluoromethyl)pyridin-3-yl)sulfonyl)-6-(2-oxaspiro[3.3]heptan-6-yl)-2,6-diazaspiro[3.3]heptane;
2-((4-ethyl-6-(trifluoromethyl)pyridin-3-yl)sulfonyl)-6-((tetrahydro-2H-pyran-4-yl)methyl)-2,6-diazaspiro[3.3]heptane;
(1r,4r)-4-(6-((4-ethyl-6-(trifluoromethyl)pyridin-3-yl)sulfonyl)-2,6-diazaspiro[3.3]heptan-2-yl)-1-methylcyclohexan-1-ol;
(1s,4s)-4-(6-((4-ethyl-6-(trifluoromethyl)pyridin-3-yl)sulfonyl)-2,6-diazaspiro[3.3]heptan-2-yl)-1-methylcyclohexan-1-ol;
2-((2-methyl-6-(trifluoromethyl)pyridin-3-yl)sulfonyl)-6-((4-methyltetrahydro-2H-pyran-4-yl)methyl)-2,6-diazaspiro[3.3]heptane;
2-((2-methyl-6-(trifluoromethyl)-1,4-dihydropyridin-3-yl)sulfonyl)-6-((1R)-1-(tetrahydrofuran-3-yl)ethyl)-2,6-diazaspiro[3.3]heptane;
(S)-2-((2-methyl-6-(trifluoromethyl)-1,4-dihydropyridin-3-yl)sulfonyl)-6-(1-(4-methyltetrahydro-2H-pyran-4-yl)ethyl)-2,6-diazaspiro[3.3]heptane;
(S)-2-((2-methyl-6-(trifluoromethyl)pyridin-3-yl)sulfonyl)-6-(1-(3-methyloxetan-3-yl)ethyl)-2,6-diazaspiro[3.3]heptane;
(S)-2-((2-methyl-6-(trifluoromethyl)-1,4-dihydropyridin-3-yl)sulfonyl)-6-((tetrahydrofuran-3-yl)methyl)-2,6-diazaspiro[3.3]heptane;
(R)-2-((2-methyl-6-(trifluoromethyl)pyridin-3-yl)sulfonyl)-6-((2-methyltetrahydrofuran-2-yl)methyl)-2,6-diazaspiro[3.3]heptane;
2-((2-methyl-6-(trifluoromethyl)pyridin-3-yl)sulfonyl)-6-((S)-1-((S)-2-methyltetrahydrofuran-2-yl)ethyl)-2,6-diazaspiro[3.3]heptane;
(S)-2-((2-methyl-6-(trifluoromethyl)pyridin-3-yl)sulfonyl)-6-((tetrahydro-2H-pyran-3-yl)methyl)-2,6-diazaspiro[3.3]heptane;
2-((2-methyl-6-(trifluoromethyl)pyridin-3-yl)sulfonyl)-6-((S)-1-((S)-tetrahydro-2H-pyran-3-yl)ethyl)-2,6-diazaspiro[3.3]heptane;
2-((2-methyl-6-(trifluoromethyl)pyridin-3-yl)sulfonyl)-6-((R)-1-((S)-tetrahydrofuran-2-yl)ethyl)-2,6-diazaspiro[3.3]heptane;
(S)-2-((2-methyl-6-(trifluoromethyl)pyridin-3-yl)sulfonyl)-6-((tetrahydrofuran-2-yl)methyl)-2,6-diazaspiro[3.3]heptane;
(R)-2-((2-methyl-6-(trifluoromethyl)pyridin-3-yl)sulfonyl)-6-(tetrahydrofuran-3-yl)-2,6-diazaspiro[3.3]heptane;
2-((2-methyl-6-(trifluoromethyl)pyridin-3-yl)sulfonyl)-6-((3-methyloxetan-3-yl)methyl)-2,6-diazaspiro[3.3]heptane;
(R)-2-(3,3-dimethyltetrahydro-2H-pyran-4-yl)-6-((2-methyl-6-(trifluoromethyl)pyridin-3-yl)sulfonyl)-2,6-diazaspiro[3.3]heptane;
2-((2-methyl-6-(trifluoromethyl)pyridin-3-yl)sulfonyl)-6-((2S,4S)-2-methyltetrahydro-2H-pyran-4-yl)-2,6-diazaspiro[3.3]heptane;
2-((2-methyl-6-(trifluoromethyl)pyridin-3-yl)sulfonyl)-6-((2S,4R)-2-methyltetrahydro-2H-pyran-4-yl)-2,6-diazaspiro[3.3]heptane;
2-((2-methyl-6-(trifluoromethyl)pyridin-3-yl)sulfonyl)-6-((3R,4R)-3-methyltetrahydro-2H-pyran-4-yl)-2,6-diazaspiro[3.3]heptane;
2-((3R,4R)-3-fluorotetrahydro-2H-pyran-4-yl)-6-((2-methyl-6-(trifluoromethyl)pyridin-3-yl)sulfonyl)-2,6-diazaspiro[3.3]heptane;
2-((3R,4S)-3-fluorotetrahydro-2H-pyran-4-yl)-6-((2-methyl-6-(trifluoromethyl)pyridin-3-yl)sulfonyl)-2,6-diazaspiro[3.3]heptane;
2-((3R,5R)-3,5-dimethyltetrahydro-2H-pyran-4-yl)-6-((2-methyl-6-(trifluoromethyl)pyridin-3-yl)sulfonyl)-2,6-diazaspiro[3.3]heptane;
2-((3R,5S)-3,5-dimethyltetrahydro-2H-pyran-4-yl)-6-((2-methyl-6-(trifluoromethyl)-1,4-dihydropyridin-3-yl)sulfonyl)-2,6-diazaspiro[3.3]heptane;
2-(4,4-difluorocyclohexyl)-6-((2-methyl-6-(trifluoromethyl)pyridin-3-yl)sulfonyl)-2,6-diazaspiro[3.3]heptane;
(R)-2-((2-methyl-6-(trifluoromethyl)-1,4-dihydropyridin-3-yl)sulfonyl)-6-(tetrahydro-2H-pyran-3-yl)-2,6-diazaspiro[3.3]heptane;
2-((2-methyl-6-(trifluoromethyl)-1,4-dihydropyridin-3-yl)sulfonyl)-6-((tetrahydro-2H-pyran-4-yl)methyl)-2,6-diazaspiro[3.3]heptane;
(S)-2-((2-methyl-6-(trifluoromethyl)-1,4-dihydropyridin-3-yl)sulfonyl)-6-(1-(tetrahydro-2H-pyran-4-yl)ethyl)-2,6-diazaspiro[3.3]heptane;
2-((2-methyl-6-(trifluoromethyl)pyridin-3-yl)sulfonyl)-6-(oxetan-3-ylmethyl)-2,6-diazaspiro[3.3]heptane;
(S)-2-((2-methyl-6-(trifluoromethyl)pyridin-3-yl)sulfonyl)-6-(1-(oxetan-3-yl)ethyl)-2,6-diazaspiro[3.3]heptane;
(R)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane;
(R)-2-((2-methyl-6-(trifluoromethyl)pyridin-3-yl)sulfonyl)-6-(1-(tetrahydro-2H-pyran-4-yl)ethyl)-2,6-diazaspiro[3.3]heptane;
(R)-2-((2-methyl-6-(trifluoromethyl)pyridin-3-yl)sulfonyl)-6-(1-(oxetan-3-yl)ethyl)-2,6-diazaspiro[3.3]heptane;
(R)-2-((2-methyl-6-(trifluoromethyl)pyridin-3-yl)sulfonyl)-6-(1-(oxetan-3-yl)ethyl)-2,6-diazaspiro[3.3]heptane;
(R)-2-((2-methyl-6-(trifluoromethyl)pyridin-3-yl)sulfonyl)-6-((tetrahydrofuran-3-yl)methyl)-2,6-diazaspiro[3.3]heptane;
(R)-2-((2-methyl-6-(trifluoromethyl)pyridin-3-yl)sulfonyl)-6-((tetrahydrofuran-3-yl)methyl)-2,6-diazaspiro[3.3]heptane;
2-(tetrahydro-2H-pyran-4-yl)-6-((2-(trifluoromethyl)pyridin-3-yl)sulfonyl)-2,6-diazaspiro[3.3]heptane;
2-((6-(2,2-difluorocyclopropyl)-2-methylpyridin-3-yl)sulfonyl)-6-(tetrahydro-2H-pyran-4-yl)-2,6-diazaspiro[3.3]heptane;
2-((2-methylpyridin-3-yl)sulfonyl)-6-(tetrahydro-2H-pyran-4-yl)-2,6-diazaspiro[3.3]heptane;
2-((6-chloro-5-(trifluoromethyl)pyridin-3-yl)sulfonyl)-6-(tetrahydro-2H-pyran-4-yl)-2,6-diazaspiro[3.3]heptane;
2-((3-(1,1-difluoroethyl)-1-methyl-1H-pyrazol-5-yl)sulfonyl)-6-(tetrahydro-2H-pyran-4-yl)-2,6-diazaspiro[3.3]heptane;
2-(1-(1-methyl-2-oxabicyclo[2.1.1]hexan-4-yl)ethyl)-6-((2-methyl-6-(trifluoromethyl)pyridin-3-yl)sulfonyl)-2,6-diazaspiro[3.3]heptane;
2-((6-methoxy-4-methylpyridin-3-yl)sulfonyl)-6-(tetrahydro-2H-pyran-4-yl)-2,6-diazaspiro[3.3]heptane;
2-methyl-3-((6-(tetrahydro-2H-pyran-4-yl)-2,6-diazaspiro[3.3]heptan-2-yl)sulfonyl)-2,4,5,6-tetrahydrocyclopenta[c]pyrazole;
2-ethyl-6-((2-methyl-6-(trifluoromethyl)pyridin-3-yl)sulfonyl)-2,6-diazaspiro[3.3]heptane;
2-((6-((2-methyl-6-(trifluoromethyl)pyridin-3-yl)sulfonyl)-2,6-diazaspiro[3.3]heptan-2-yl)methyl)cyclopropane-1-carbonitrile;
2-(3-(2-methoxyethoxy)propyl)-6-((2-methyl-6-(trifluoromethyl)-1,2-dihydropyridin-3-yl)sulfonyl)-2,6-diazaspiro[3.3]heptane;
(R)-2-(2-(2,2-dimethyl-1,3-dioxolan-4-yl)ethyl)-6-((2-methyl-6-(trifluoromethyl)pyridin-3-yl)sulfonyl)-2,6-diazaspiro[3.3]heptane;
(1R,3R)-2,2-dimethyl-3-((6-((2-methyl-6-(trifluoromethyl)pyridin-3-yl)sulfonyl)-2,6-diazaspiro[3.3]heptan-2-yl)methyl)cyclopropane-1-carbonitrile;
2-(((1S,2R,5S)-6-oxabicyclo[3.2.1]octan-2-yl)methyl)-6-((2-methyl-6-(trifluoromethyl)pyridin-3-yl)sulfonyl)-2,6-diazaspiro[3.3]heptane;
2-(3-(cyclopentyloxy)propyl)-6-((2-methyl-6-(trifluoromethyl)pyridin-3-yl)sulfonyl)-2,6-diazaspiro[3.3]heptane;
methyl 2,2-dimethyl-4-(6-((2-methyl-6-(trifluoromethyl)-1,4-dihydropyridin-3-yl)sulfonyl)-2,6-diazaspiro[3.3]heptan-2-yl)butanoate;
2-(2-cyclopropyl-2-methoxyethyl)-6-((2-methyl-6-(trifluoromethyl)pyridin-3-yl)sulfonyl)-2,6-diazaspiro[3.3]heptane;
2-(5-methoxypentyl)-6-((2-methyl-6-(trifluoromethyl)pyridin-3-yl)sulfonyl)-2,6-diazaspiro[3.3]heptane;
2-(4-methoxypentyl)-6-((2-methyl-6-(trifluoromethyl)pyridin-3-yl)sulfonyl)-2,6-diazaspiro[3.3]heptane;
2-((1-(2-methoxyethyl)cyclobutyl)methyl)-6-((2-methyl-6-(trifluoromethyl)pyridin-3-yl)sulfonyl)-2,6-diazaspiro[3.3]heptane;
methyl 4-methyl-5-(6-((2-methyl-6-(trifluoromethyl)pyridin-3-yl)sulfonyl)-2,6-diazaspiro[3.3]heptan-2-yl)pentanoate;
2-(3-(ethylthio)propyl)-6-((2-methyl-6-(trifluoromethyl)pyridin-3-yl)sulfonyl)-2,6-diazaspiro[3.3]heptane;
2-(2-cyclopropylethyl)-6-((2-methyl-6-(trifluoromethyl)pyridin-3-yl)sulfonyl)-2,6-diazaspiro[3.3]heptane;
2-(2-(tert-butoxy)ethyl)-6-((2-methyl-6-(trifluoromethyl)-1,2-dihydropyridin-3-yl)sulfonyl)-2,6-diazaspiro[3.3]heptane;
2-((2,2-dimethyltetrahydro-2H-pyran-4-yl)methyl)-6-((2-methyl-6-(trifluoromethyl)pyridin-3-yl)sulfonyl)-2,6-diazaspiro[3.3]heptane;
4-((6-((2-methyl-6-(trifluoromethyl)pyridin-3-yl)sulfonyl)-2,6-diazaspiro[3.3]heptan-2-yl)methyl)tetrahydro-2H-pyran-4-ol;
2-((4-fluorotetrahydro-2H-pyran-4-yl)methyl)-6-((2-methyl-6-(trifluoromethyl)pyridin-3-yl)sulfonyl)-2,6-diazaspiro[3.3]heptane;
2-((3-isopropyl-1-methyl-1H-pyrazol-5-yl)sulfonyl)-6-(tetrahydro-2H-pyran-4-yl)-2,6-diazaspiro[3.3]heptane;
2-((2-cyclopropyl-6-(trifluoromethyl)pyridin-3-yl)sulfonyl)-6-((tetrahydro-2H-pyran-4-yl)methyl)-2,6-diazaspiro[3.3]heptane;
2-((2-methyl-6-(1-(trifluoromethyl)cyclopropyl)-1,4-dihydropyridin-3-yl)sulfonyl)-6-((tetrahydro-2H-pyran-4-yl)methyl)-2,6-diazaspiro[3.3]heptane;
2-((2-chloro-6-(trifluoromethyl)pyridin-3-yl)sulfonyl)-6-(tetrahydro-2H-pyran-4-yl)-2,6-diazaspiro[3.3]heptane;
2-((2-cyclopropyl-6-(trifluoromethyl)pyridin-3-yl)sulfonyl)-6-(tetrahydro-2H-pyran-4-yl)-2,6-diazaspiro[3.3]heptane;
2-((2-methyl-4-(trifluoromethyl)phenyl)sulfonyl)-6-((tetrahydro-2H-pyran-4-yl)methyl)-2,6-diazaspiro[3.3]heptane;
(1s,4s)-4-(6-((2-cyclopropyl-6-(trifluoromethyl)pyridin-3-yl)sulfonyl)-2,6-diazaspiro[3.3]heptan-2-yl)-1-methylcyclohexan-1-ol;
(1s,4s)-4-(6-((2-cyclopropyl-6-(trifluoromethyl)pyridin-3-yl)sulfonyl)-2,6-diazaspiro[3.3]heptan-2-yl)-1-methylcyclohexan-1-ol;
2-((2-methyl-5-(trifluoromethyl)-2,5-dihydro-1H-pyrazol-3-yl)sulfonyl)-6-(((R)-tetrahydrofuran-3-yl)methyl)-2,6-diazaspiro[3.3]heptane;
(R)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane;
(S)-2-((2,2-dimethyltetrahydro-2H-pyran-4-yl)methyl)-6-((2-methyl-5-(trifluoromethyl)-1H-2l4-pyrazol-3-yl)sulfonyl)-2,6-diazaspiro[3.3]heptane;
(S)-2-((2,2-dimethyltetrahydro-2H-pyran-4-yl)methyl)-6-((1-methyl-3-(trifluoromethyl)-1H-pyrazol-5-yl)sulfonyl)-2,6-diazaspiro[3.3]heptane;
2-((tetrahydro-2H-pyran-4-yl)methyl)-6-((2-(trifluoromethyl)-3,4-dihydropyridin-3-yl)sulfonyl)-2,6-diazaspiro[3.3]heptane;
2-((1-methyl-3-(trifluoromethyl)-1H-pyrazol-5-yl)sulfonyl)-6-((tetrahydro-2H-pyran-4-yl)methyl)-2,6-diazaspiro[3.3]heptane;
2-(tetrahydro-2H-pyran-4-yl)-6-((6-(trifluoromethyl)-2-vinylpyridin-3-yl)sulfonyl)-2,6-diazaspiro[3.3]heptane;
2-((2-ethyl-6-(trifluoromethyl)pyridin-3-yl)sulfonyl)-6-(tetrahydro-2H-pyran-4-yl)-2,6-diazaspiro[3.3]heptane;
2-((6-(difluoromethyl)-2-ethylpyridin-3-yl)sulfonyl)-6-(tetrahydro-2H-pyran-4-yl)-2,6-diazaspiro[3.3]heptane;
2-((6-(difluoromethyl)-2-ethylpyridin-3-yl)sulfonyl)-6-(2-oxaspiro[3.3]heptan-6-yl)-2,6-diazaspiro[3.3]heptane;
(1r,4r)-4-(6-((6-(difluoromethyl)-2-ethylpyridin-3-yl)sulfonyl)-2,6-diazaspiro[3.3]heptan-2-yl)-1-methylcyclohexan-1-ol;
(1s,4s)-4-(6-((6-(difluoromethyl)-2-ethylpyridin-3-yl)sulfonyl)-2,6-diazaspiro[3.3]heptan-2-yl)-1-methylcyclohexan-1-ol;
2-((2-cyclopropyl-6-(trifluoromethyl)pyridin-3-yl)sulfonyl)-6-(oxetan-3-ylmethyl)-2,6-diazaspiro[3.3]heptane;
2-((3-(1,1-difluoroethyl)-1-methyl-1H-pyrazol-5-yl)sulfonyl)-6-((tetrahydro-2H-pyran-4-yl)methyl)-2,6-diazaspiro[3.3]heptane;
2-((2,4-bis(trifluoromethyl)phenyl)sulfonyl)-6-((tetrahydro-2H-pyran-4-yl)methyl)-2,6-diazaspiro[3.3]heptane;
(R)-2-((1-methyl-3-(trifluoromethyl)-1H-pyrazol-5-yl)sulfonyl)-6-(1-(tetrahydro-2H-pyran-4-yl)ethyl)-2,6-diazaspiro[3.3]heptane;
2-((6-chloro-2-(trifluoromethyl)-1,4-dihydropyridin-3-yl)sulfonyl)-6-(tetrahydro-2H-pyran-4-yl)-2,6-diazaspiro[3.3]heptane;
2-((2-fluoro-4-(trifluoromethoxy)phenyl)sulfonyl)-6-(tetrahydro-2H-pyran-4-yl)-2,6-diazaspiro[3.3]heptane;
(R)-2-((1,4-dioxan-2-yl)methyl)-6-((1-methyl-3-(trifluoromethyl)-1H-pyrazol-5-yl)sulfonyl)-2,6-diazaspiro[3.3]heptane;
(R)-2-((1,4-dioxan-2-yl)methyl)-6-((2-methyl-6-(trifluoromethyl)pyridin-3-yl)sulfonyl)-2,6-diazaspiro[3.3]heptane;
2-((4-methyl-6-(trifluoromethyl)pyridin-3-yl)sulfonyl)-6-((tetrahydro-2H-pyran-4-yl)methyl)-2,6-diazaspiro[3.3]heptane;
2-((1-methyl-3-(trifluoromethyl)-1H-pyrazol-5-yl)sulfonyl)-6-(oxetan-3-ylmethyl)-2,6-diazaspiro[3.3]heptane;
2-((3-cyclopropyl-1-methyl-1H-pyrazol-5-yl)sulfonyl)-6-((tetrahydro-2H-pyran-4-yl)methyl)-2,6-diazaspiro[3.3]heptane;
2-((2-fluoro-4-(trifluoromethoxy)phenyl)sulfonyl)-6-((tetrahydro-2H-pyran-4-yl)methyl)-2,6-diazaspiro[3.3]heptane;
2-((3-methyl-5-(trifluoromethyl)pyridin-2-yl)sulfonyl)-6-(tetrahydro-2H-pyran-4-yl)-2,6-diazaspiro[3.3]heptane;
(1s,4s)-1-methyl-4-(6-((2-methyl-6-(trifluoromethyl)pyridin-3-yl)sulfonyl)-2,6-diazaspiro[3.3]heptan-2-yl)cyclohexan-1-ol;
(1s,4s)-1-methyl-4-(6-((2-methyl-6-(trifluoromethyl)pyridin-3-yl)sulfonyl)-2,6-diazaspiro[3.3]heptan-2-yl)cyclohexan-1-ol;
2-((3-methyl-5-(trifluoromethyl)pyridin-2-yl)sulfonyl)-6-((tetrahydro-2H-pyran-4-yl)methyl)-2,6-diazaspiro[3.3]heptane;
2-((4-(difluoromethoxy)-2,6-difluorophenyl)sulfonyl)-6-((tetrahydro-2H-pyran-4-yl)methyl)-2,6-diazaspiro[3.3]heptane;
2-((4-(difluoromethoxy)-2-fluorophenyl)sulfonyl)-6-((tetrahydro-2H-pyran-4-yl)methyl)-2,6-diazaspiro[3.3]heptane;
2-((4-(difluoromethoxy)-2-fluorophenyl)sulfonyl)-6-(tetrahydro-2H-pyran-4-yl)-2,6-diazaspiro[3.3]heptane;
2-((6-((tetrahydro-2H-pyran-4-yl)methyl)-2,6-diazaspiro[3.3]heptan-2-yl)sulfonyl)-5-(trifluoromethyl)benzonitrile; and
2-((tetrahydro-2H-pyran-4-yl)methyl)-6-((6-(trifluoromethyl)-2,3-dihydropyridin-3-yl)sulfonyl)-2,6-diazaspiro[3.3]heptane;
or a pharmaceutically acceptable salt thereof.

68. A pharmaceutical composition comprising a compound of any one of claims 1 to 67, or a pharmaceutically acceptable salt thereof, and a pharmaceutically acceptable excipient.

69. A method of treating a disease mediated by Emopamil-Binding Protein comprising administering to a subject an effective amount of a compound of any one of claims 1 to 67, or a pharmaceutically acceptable salt thereof, or the pharmaceutical composition of claim 68.

70. A method of treating an autoimmune disease in a subject comprising administering to the subject an effective amount of a compound of any one of claims 1 to 67, or a pharmaceutically acceptable salt thereof, or the pharmaceutical composition of claim 68.

71. The method of claim 70, wherein said autoimmune disease is multiple sclerosis.

72. The method of claim 71, wherein said compound or pharmaceutical composition repairs or forms new myelin sheaths in said subject.
